SEQUENCE LISTING (133 sequences)

SEQ ID NO: 1 - protein, 413 aa, Glycine max
MGACWSSRIKAVSPSNTGFTSRSVSRDGHDIQSSSRNSSASIPMTPRSEGEILQFSNLKSYSYNELKMATKNFCPDSVLGEGGFGSVFKGWIDEHSLAVTRPGTGMVIAVKKLNQDSFQGHKEWLAEINYLGQLQNPNLVKLIGYCLEDQHRLLVYEYMPKGSVENHLFRRGSHFQQLSWTLRLKISLGAARGLAFLHSTETKVIYRDFKTSNILLDTNYNAKLSDFGLARDGPTGDKSHVSTRVMGTHGYAAPEYLATGHLTAKSDVYSFGVVLLEMLSGRRAIDKNRPSGEQCLVEWAKPYLSNKRRVFRVMDSRLEGQYSLTQAQRAATLAFQCLSVEPKYRPNMDEVVKALEQLRESNDKVKNGDHKKCRVSGSGLGHPNGLPASTSKGSIDAAKKFNYPRPSASLLYS

SEQ ID NO: 2 - protein, 371 aa, Glycine max
MSYRERNKGKGEEKEYENSTTRGLDSKSVSHNNGSIEEELLTIDENLLIDPKLLFIGSKIGEGAHGRVYEGRYRDQIVAIKVLHRGGTLEERVALENRFAREVNMMSRVHHENLVKFIGACKDPLMVIVTEMLPGLSLRKYLTTIRPKQLDPYVAIKFALDIARAMDWLHANGIIHRDLKPDNLLLTENQKSVKLADFGLAREESVTEMMTAETGTYRWMAPELYSTVTLCQGEKKHYNNKVDVYSFGIVLWELLTNRMPFEGMSNLQAAYAAAFKQERPNLPDDISPDLAFIIQSCWVEDPNMRPSFSQIIRLLNEFHFTLQQPSPSMPLEPENEPEAITSNGTITDFSTRNKVKFSFIRHLFSSKRTKS

SEQ ID NO: 3 - protein, 694 aa, Glycine max
MFPLQCSNHPMCAFSAAVTTILLLFPAATSQAQILKKETYFFGPFNQSYFTTFAVLPSAAINLGALQVTPDSTGNVSLANQSGRIFFSTPFTLWDDENLNGKLVSFNTSFLINVFRPQNNPPGEGIAFLIAPSSSTVPNNSHGQFLGLTNAATDGNATNKFIAVELDTVKQDFDPDDNHIGLDINSVRSNVSVSLTPLGFEIAPNVTRFHVLWVDYDGDRKEIDVYIAEQPDKDVPIVAKPAKPVLSSPLDLKQVLNKVSYFGFSASTGDNVELNCVLRWNITIEVFPKKNGNGKAYKIGLSVGLTLLVLIVAGVVGFRVYWIRKKKRENESQILGTLKSLPGTPREFRYQELKKATNNFDDKHKLGQGGYGVVYRGTLLPKENLQVAVKMFSRDKMKSTDDFLAELTIINRLRHKNLVRLLGWCHRNGVLLLVYDYMPNGSLDNHIFCEEGSSTTPLSWPLRYKIITGVASALNYLHNEYDQKVVHRDLKASNIMLDSNFNARLGDFGLARALENDKTSYAEMEGVHGTMGYIAPECFHTGRATRESDVYGFGAVLLEVVCGQRPWTKNEGYECLVDWVWHLHREQRILDAVNPRLGNDCVVEEAERVLKLGLACSHPIASERPKMQTIVQILSGSVHVPHLPPFKPAFVWPAMDLSSLASDLTTQTTTTEYTPMSSDTHSMHVQFSDSSSLV

SEQ ID NO: 4 - protein, 624 aa, Glycine max
MEHSSSLVFWLLGLLLLLLMEISSAALSPSGINYEVVALMAIKNDLIDPHNVLENWDINSVDPCSWRMITCSPDGSVSALGLPSQNLSGTLSPGIGNLTNLQSVLLQNNAISGRIPAAIGSLEKLQTLDLSNNTFSGEIPSSLGGLKNLNYLRLNNNSLTGSCPQSLSNIEGLTLVDLSYNNLSGSLPRISARTLKIVGNSLICGPKANNCSTILPEPLSFPPDALRGQSDSGKKSHHVALAFGASFGAAFVLVIIVGFLVWWRYRRNQQIFFDVNEHYDPEVRLGHLKRFSFKELRAATDHFNSKNILGRGGFGIVYKACLNDGSVVAVKRLKDYNAAGGEIQFQTEVETISLAVHRNLLRLSGFCSTQHERLLVYPYMSNGSVASRLKDHIHGRPALDWTRRKRIALGTARGLVYLHEQCDPKIIHRDVKAANILLDEDFEAVVGDFGLAKLLDHRDSHVTTAVRGTVGHIAPEYLSTGQSSEKTDVFGFGILLLELITGHKALDFGRAANQKGVMLDWVKKLHQDGRLSQMVDKDLKGNFDLIELEEMVQVALLCTQFNPSHRPKMSEVLKMLEGDGLAERWEASQRIETPRFRSCEPQRYSDLIEESSLVVEAMELSGPR

SEQ ID NO: 5 - protein, 581 aa, Glycine max
MATIGIGVGVTKLQQPHRPKSKSLFLGQRLRISPFWGEPQCSERRLFGVSNPPRVVQVFALGGGEWLDTVHNLFVGVGVGLPCSVMQCGDVIYRSTLPKSNGLTLTVPGVILALGTLSYLWATPGVAPGFFDMFVLAFVERLFRPTYKKDDFVLGKKLGEGSFGVVYRVSLANKPSSKEGDLVLKKATEYGAVEIWMNERVRRACASSCADFVYGFLESSSKKAAEYWLIWRFEGDATLADLMQSRDFPYNVETLILGEVQDLPKGLERENRIIQTIMRQILFALDGLHSTGIVHRDIKPQNVIFSEESRTFKIIDLGAATDLRVGINYIPKEFLLDPRYAAPEQYIMSTQTPSAPSVPVATALSPVLWQLNLPDRFDIYSAGLIFLQMAFPSLRSDNSLIQFNRQLKRCDYDLVAWRKTAEARSELRKGFELLDLDGGIGWELLKSMVRYKARQRLSAKAALAHPYFVREGLLALSFMQTLRLQLLRATQQDYSEAARWIIQLMAKSGTQKDGGFTEAQLQELREIEPKKKASAPRNALASALKLQRKIIRTIRTLNESMDELTRRRKSFWWSRWIPREE

SEQ ID NO: 6 - protein, 621 aa, Glycine max
MERSGDVALFCLALFFLWTSVAALLSPKGVNYEVQALMSIKNSLVDPHSVLNNWDTDAVDPCNWAMVTCSSDHFVIALGIPSQSISGTLSPSIGNLTNLQTVLLQDNNITGPIPFEIGRLQKLQTLDLSDNFFTGQLPDTLSYMKGLHYLRLNNNSLTGPIPSSLANMTQLAFLDISYNNLSEPVPRINAKTFNIIGNPQICATGVEKNCFRTTSIPSAPNNSQDSQSTKRPKSHKFALAFASSLSCICLLILGLGFLIWWRQRYNKQIFFDVNEQHREEVCLGNLKKFHFRELQLATNNFSSKNLIGKGGFGNVYKGYVQDGTVIAVKRLKDGNAIGGEIQFQTEVEMISLAVHRNLLRLYGFCMTATERLLVYPYMSNGSVASRLKAKPALDWATRKRIALGAGRGLLYLHEQCDPKIIHRDVKAANILLDDYCEAVVGDFGLAKLLDHRDSHVTTAVRGTVGHIAPEYLSTGQSSEKTDVFGFGILLLELISGQRALEFGKAANQKGAMLDWVKKIHQEKKIDLLVDKDLKNNYDRIELDEIVQVALLCTQYLPSHRPKMSEVVRMLEGDGLAEKWEASQSAESTRSRGNELSSSERYSDLTDDSSLLAQAMELSGPR

SEQ ID NO: 7 - protein, 598 aa, Glycine max
MGVCTSKPQKPNPYALREAEAEADPSQNPKTTLSPAGADTPRRKDDVSTGKRSPFFPFYSPSPARFLKKSPAPAGGSRSASSTPRRFFRRPFPPPSPAKHIRAVLARRQGKKASATAAIPEEGEEGAADLDKRFGFSKEFTSRLEVGEEVGRGHFGYTCSARFKKGELKGQQVAVKVIPKAKMTTAIAIEDVRREVKILRALNGHNNLIQFYDAFEDQDNVYIVMELCEGGELLDMILSRGGKYSEDDAKAVMVQILNVVAFCHLQGVVHRDLKPENFLYAKKDESSELKAIDFGLSDFVRPDERLNDIVGSAYYVAPEVLHRSYGTEADVWSIGVIAYILLCGSRPFWARTESGIFRAVLKADPSFDETPWPSLSLEAKDFVKRILNKDPRKRISAAQALSHPWIRNCNNVKVPLDILIFKLMKTYMRSSSLRKAALRALSKTLTADELYYLRGQFALLEPSKNGSISLENVNKALMKYATDAMKESRIPDFLSSLNSLQYRRMDFEEFCAAALSVHQLEALDRWEQHARCAYELFDKDGNRAIVIEELASELGLGPSIPVHVVLHDWIRHTDGKLSFLGFVKLLHGVSSRSLAKVQ

SEQ ID NO: 8 - protein, 430 aa, Glycine max
MSEKEKGNNSEGQSTLLHGKYELGRVLGHGTFAKVYHARNLNTGQHVAMKVVGKEKVIKVGMMEQVKREISVMKMVKHQNIVELHEVMASKSKIYIAMELVRGGELFNKVSKGRLKEDVARLYFQQLISAVDFCHSRGVYHRDLKPENLLLDEHGNLKVSDFGLTAFSEHLKEDGLLHTTCGTPAYVSPEVIAKKGYDGAKADIWSCGVILYVLLAGFLPFQDDNLVAMYKKIYRGDFKCPPWFSLDARKLVTKLLDPNPNTRISISKVMESSWFKKPVPRKLAAEKVDLEEEKIESQLETINAFHIISLSEGFNLSPLFEDKRREEMRFATAGTPSTVISRLEEVAKAGKFDVRSSETKVRLQGQERGRKGKLAIAADIYAVTPSFMVVEVKKDNGDTLEYNQFCSKQLRPALKDIFWNSSQNSAPASA

SEQ ID NO: 9 - protein, 338 aa, Glycine max
MERYEILKDIGSGNFAVAKLVRDNYTNELFAVKFIERGQKIDEHVQREIMNHRSLKHPNIIRFKEVLLTPTHLAIVMEYASGGELFERICNAGRFSEDEARFFFQQLISGVSYCHSMQICHRDLKLENTLLDGSTAPRVKICDFGYSKSSVLHSQPKSTVGTPAYIAPEVLTRKEYDGKIADVWSCGVTLYVMLVGAYPFEDPADPRNFKKTIGKILSVQYSVPDYVRVSMECRHLLSQIFVASPEKRITIPEIKNHPWFLRNLPMELTEGGSWQMNDVNNPSQNVEEVLSIIQEARKSLNVPKVGGLLTGGSMDLDDFDADEDLEDLETSGEFVCPI

SEQ ID NO: 10 - protein, 514 aa, Glycine max
MMSLCSSKGTCSGDTNAHVSSSTVGNYSSDKKRHNNKFITFLRKTMWEYALACVGVVPCGGNNDLNGQRKTTLEHNKAWLLADSGAELASADPRSVHSSFRFSFCSQVEVESFNMSYSASAAAAATFLMVNLDYESQVRELKWRRIQSLEKSLSPVANTLIRFSYDEILSATRNFSKERVLGRGALSCVFRGRVGIWRTAVAIKRLDKEDKECAKAFCRELMIASSLNDTNVVPLVGFCIDSEEGLFLVYKYVSGGSLEHHLHGRKKGVKGSSPLPWSVRYEVAIGIAEAVAYLHNGTERCVVHRDIKPSNILLSSKKIPKLCDFGLASWTSAPSVPFLCKTVKGTFGYLAPEYFEHGKVSDKTDVYALGVVLLELLTGRNPIEAKRPPGEENLVVWAKPLLRKGKGAIEELLDPQVKYNSSYTDQMVRMIDAASVCVTSEESRRPSIGEIVAILKGEVEHVLSRRRKSGYFGNGYMIDNYPKLQETNNEMKSHLALAMLGVPECEDDDFVYCR

SEQ ID NO: 11 - protein, 517 aa, Glycine max
MILIDLLQPIMSSNSSLNMELSKKTSFLGLKRWVLIGIGVGAFIVLILCILSIWAMFRRKCRRSLDKYSVSQIPNVSKDIDVDKVGVQSSHVQPENVVIPVHDKASDKNSDNVSVHLGKSKSGDPDNISQCSSIYHHERGFSSMSAEEGSSGNVKKQSTLSHGGLATASPLVGLPEFSHLGWGHWFTLRDLEMATNRFSSENIIGEGGYGIVYRGRLINGTEVAVKKLLNNLGQAEKEFRVEVEAIGHVRHKHLVRLLGYCVEGVHRLLVYEYVNNGNLEQWLHGNMHQYGTLTWEARMKVILGTAKALAYLHEAIEPKVIHRDIKSSNILIDDEFNAKVSDFGLAKLLDSGESHITTRVMGTFGYVAPEYANSGLLNEKSDIYSFGVLLLEAVTGRDPVDYARPANEVNLVEWLKTMVGTRRAEEVVDSSLEVKPPLRALKRTLLVALRCIDPDADKRPKMSQVVRMLEADEYPFREDRRKRKSGTASMEIETVKDISGPSDAEKMVISESHVEEG

SEQ ID NO: 12 - protein, 526 aa, Glycine max
MPSRPPPLSPPATAAASHRREIILCGAIGGALFLTALIISVMIFIYRKLSYSRTAPFEHNQRRFSYTVLRRATNSFSPSTKLGHGGFGSVHKATLPSGQTVALKVMDSPGSLQGEREFHNELTLCSNLKSPFVISLLGFSSDRRGKKLVLVYELMPNRSLQDALLDRRCPELMSWGKRFDVAVSVARGLEYLHHVCDPPVIHGDIKPSNVLLDRDFRAKIGDFGLARVKNVEDLGMVDENEKKKDEEFSVLEGESVVDVDRSPESCPVRAAEYSDASPVGGDKLSVVSDGGGCFESVDSGSVSVNKKKCGGGGGGGGSGRDWWWRQESGGGGESGREGVEQKKKRKSKGSRGSIDWWLDGLSAKIGGFSGDVPKSGGISSTPSMRGTVCYIAPEYGGGGQLSEKCDVYSFGVLLLVLVAGRRPLQVTASPISEFERANLISWARQLAHNGRLLDLVDTSIHSLDKEQALLCITIALLCLQRSPGKRPSIKEVVGMLSGEAEPPHLPFEFSPSPPSNFPFKTRKKAR

SEQ ID NO: 13 - protein, 436 aa, Glycine max
MSDSDTRQNDITKPLDLENLRVVSAVGRGAKGVVFLARTGDRSSEECVALKVIPKALILQKAKLINDVEYTRVSFEEQVLRRFDHLLLPRLRGVFETEKVVGFGIDYCHGGTLHSLRKKQTEKMFSDDTIRFYAVELVLALEYLHNLGIVYRDLKPENVMIQDNGHIMLVDFDLSKKLNPKSPHSLSQNSSPSPNSKTKQTRKQRLMRFYSFCNSGILPCDSDSEPPLSSVNSVRHTESDLVEKSNSFVGTEEYVAPEIVSGKGHGFSVDWWSYGVVLYEMLYGTTPFKGSNRKETFYRILMKEPELTGEKTALRDLIGKLLEKNPDRRIQVNEIKGHNFFKGVKWDTVLHIARPPYIPENEVENKVGFSKSDVEVFVDNVFFPTGDDGGEKTKTLLDNNNNNNNNNNNNNNNNKVWIDELSHVPIGKKNDDFLIF

SEQ ID NO: 14 - protein, 603 aa, Glycine max
MEQYEILEQIGKGAFGSALLVRHKHEKKKYVLKKIRLARQTDRTRRSAHQEMELISKVRNPFIVEYKDSWVEKGCFVCIIIGYCEGGDMAEAIKKANGINFPEEKLCKWLVQLLMALDYLHGNHILHRDVKCSNIFLTKDQDIRLGDFGLAKMLSSDDLASSVVGTPSYMCPELLADIPYGSKSDIWSLGCCIYEMAAYKPAFKAFDIQSLLIKINKCIVSPMPTMYSAAFRGLVKSMLRKNPELRPTAAELLNHPHLQPYIHKIQLKLNSPRRSTFPFQWPESNYVRRTRFVDPESVYTLSDLDKCLSFSNDMALNPSVSGTEQVSQCSTQRAHGLSTCSKEKIYELSVGCVREKYKTDKSKASKFSTVERTPRSRAVTVSATTKRHTIATSKTTHSGPKRDSLPASHAPSRKFSTPPRTRARATPNLYTNVLGSLDSLDVSINAPRIDKIVEFPMAFCEDPFSPIRGPSSTSARCSSSSAGSTADCSITKDKCTIQEDKVTLPTSITDACPAPKETKCSYEHVKDCVSSHSSTDLDQRRFDTSSYQQRAEALEGLLEFSARLLQQQRFDELGVLLKPFGLEKVSPRETAIWLTKSFKQTAV

SEQ ID NO: 15 - protein, 674 aa, Glycine max
MKKVCHCPIFLALALCLCILCVAAEAAGQNDTLALTEFRLQTDTHGNLLTNWTGADACSAAWRGVECSPNGRVVGLTLPSLNLRGPIDTLSTLTYLRFLDLHENRLNGTISPLLNCTSLELLYLSRNDFSGEIPAEISSLRLLLRLDISDNNIRGPIPTQLAKLTHLLTLRLQNNALSGHVPDLSASLLNLTVLNVTNNELRGHVPDSMLTKFGNVSFSGNHALCGSTPLPKCSETEPDTETTTITVPAKPSSFPQTSSVTVPDTPRKKGLSAGVIVAIVVAVCVAVLVATSFAVAHCCARGSTSGSVVGSETAKRKSGSSSGSEKKVYGNGGNLDRDSDGTNTETERSKLVFFDRRNQFELEDLLRASAEMLGKGSLGTVYRAVLDDGCTVAVKRLKDANPCERNEFEQYMDVVGKLKHPNIVRLRAYYYAKEEKLLVYDYLPNGSLHALLHGNRGPGRIPLDWTTRISLMLGAARGLARIHAEYNASKIPHGNVKSSNVLLDKNGVALISDFGLSLLLNPVHAIARLGGYRAPEQVEVKRLSQEADVYGFGVLLLEVLTGRAPSKEYTSPAREAEVDLPKWVKSVVKEEWTSEVFDQELLRYKNIEDELVAMLHVGLACVAAQAEKRPCMLEVVKMIEEIRVEESPLGDDYDEARSRTSLSPSLATTEDNLA

SEQ ID NO: 16 - protein, 982 aa, Glycine max
MAKGSYSISSKARSMKFIFLFMFMLNFILSDGDQHEVQLLLSFKASLHDPLHFLSNWVSFTSSATICKWHGINCDNNANSSHVNAVVLSGKNITGEVSSSIFQLPYLTNLDLSNNQLVGEITFTHSHNSLSQIRYLNLSNNNLTGSLPQPLFSVLFSNLETLDLSNNMFSGNIPDQIGLLSSLRYLDLGGNVLVGKIPNSITNMTALEYLTLASNQLVDKIPEEIGAMKSLKWIYLGYNNLSGEIPSSIGELLSLNHLDLVYNNLTGLIPHSLGHLTELQYLFLYQNKLSGPIPGSIFELKKMISLDLSDNSLSGEISERVVKLQSLEILHLFSNKFTGKIPKGVASLPRLQVLQLWSNGLTGEIPEELGKHSNLTVLDLSTNNLSGKIPDSICYSGSLFKLILFSNSFEGEIPKSLTSCRSLRRVRLQTNKFSGNLPSELSTLPRVYFLDISGNQLSGRIDDRKWDMPSLQMLSLANNNFSGEIPNSFGTQNLEDLDLSYNHFSGSIPLGFRSLPELVELMLSNNKLFGNIPEEICSCKKLVSLDLSQNQLSGEIPVKLSEMPVLGLLDLSQNQFSGQIPQNLGSVESLVQVNISHNHFHGSLPSTGAFLAINASAVIGNNLCDRDGDASSGLPPCKNNNQNPTWLFIMLCFLLALVAFAAASFLVLYVRKRKNFSEVRRVENEDGTWEVKFFYSKAARLINVDDVLKTVKEGKVVSKGTNWVWYEGKCMENDMQFVVKEISDLNSLPLSMWEETVKIRKVRHPNIINLIATCRCGKRGYLVYEHEEGEKLSEIVNSLSWQRRCKIAVGVAKALKFLHSQASSMLLVGEVSPEIVWVDAKGVPRLKVTPPLMPCLDVKGFVSSPYVAQEVIERKNVTEKSEIYGFGVMLVELLTGRSAMDIEAGNGMHKTIVEWARYCYSDCHLDTWIDPVMKGGDALRYQNDIVEMMNLALHCTATDPTARPCARDVLKALETVHRTTFC

SEQ ID NO: 17 - protein, 978 aa, Glycine max
MQALFSSMRVFLRLVWLLELLCVAVTAVNPSLNDDVLGLIVFKADIRDPKGKLASWNEDDESACGGSWVGVKCNPRSNRVVEVNLDGFSLSGRIGRGLQRLQFLRKLSLANNNLTGGINPNIARIDNLRVIDLSGNSLSGEVSEDVFRQCGSLRTVSLARNRFSGSIPSTLGACSALAAIDLSNNQFSGSVPSRVWSLSALRSLDLSDNLLEGEIPKGIEAMKNLRSVSVARNRLTGNVPYGFGSCLLLRSIDLGDNSFSGSIPGDFKELTLCGYISLRGNAFSGGVPQWIGEMRGLETLDLSNNGFTGQVPSSIGNLQSLKMLNFSGNGLTGSLPESMANCTKLLVLDVSRNSMSGWLPLWVFKSDLDKVLVSENVQSGSKKSPLFAMAELAVQSLQVLDLSHNAFSGEITSAVGGLSSLQVLNLANNSLGGPIPPAVGELKTCSSLDLSYNKLNGSIPWEIGGAVSLKELVLEKNFLNGKIPTSIENCSLLTTLILSQNKLSGPIPAAVAKLTNLQTVDVSFNNLTGALPKQLANLANLLTFNLSHNNLQGELPAGGFFNTITPSSVSGNPSLCGAAVNKSCPAVLPKPIVLNPNTSTDTGPSSLPPNLGHKRIILSISALIAIGAAAVIVIGVISITVLNLRVRSSTSRDAAALTFSAGDEFSHSPTTDANSGKLVMFSGEPDFSSGAHALLNKDCELGRGGFGAVYQTVLRDGHSVAIKKLTVSSLVKSQEDFEREVKKLGKIRHQNLVELEGYYWTPSLQLLIYEYLSGGSLYKHLHEGSGGNFLSWNERFNVILGTAKALAHLHHSNIIHYNIKSTNVLLDSYGEPKVGDFGLARLLPMLDRYVLSSKIQSALGYMAPEFACKTVKITEKCDVYGFGVLVLEIVTGKRPVEYMEDDVVVLCDMVRGALEEGRVEECIDERLQGKFPAEEAIPVMKLGLICTSQVPSNRPDMGEVVNILELIRCPSEGQEELA

SEQ ID NO: 18 - protein, 676 aa, Glycine max
MEHVLEKRFPLNAEDYTLYEEVGEGVSASVYRALCVPLNEIVAIKVLDLEKCNNDLDGIRREVQTMNLIDYPNVLRAHCSFTAGHNLWVVMPYMAGGSCLHIMKSNYPEGFEEPVIATLLHEVLKALVYLHAHGHIHRDVKAGNILLDSNGAVKLADFGVSACMFDTGDRQRSRNTFVGTPCWMAPEVMQQLHGYDFKADIWSFGITALELAHGHAPFSKYPPMKVLLMTLQNAPPGLDYERDKKFSKAFKELVATCLVKDPKKRPSSEKLLKHHFFKQARASKYLARTILEGLAPLGDRFRMLKAKEADLLVQNKALYEDKDQLSQKEYIRGISAWNFNLEDLKSQAALIQDDDIPNAEEPQRDKKQKDRLDNFKVSAERLSAGAANHSDDAPTQDKEDLHLLFQDGFNNLQDLEGSLVSFPTKPLQALKGCFDMCEDDINNSSPRDLDHDGRIDNESSRPSTSLQQNTTSQQKKFPSGSLLPDNFLFPKMVVTDGDRDYLQTKYSSERNHSGPLQYRQKRDTNNLPLVDDTSDGAFFRRRGRFTLTDLSPMGPSNSTSGPVVSPTSPPNQNFMSTAILPSLQCILQHNGLQREEIIKLIKYAEQSSGKNTESVEAGTGDMLQAPPATTRERELHFQVIQLQQSIGSLVEELQRQKMKNVQLEKQLNSMANRVEK

SEQ ID NO: 19 - protein, 518 aa, Glycine max
MGNCSSGAGAPTTYSDDHPHHNGITVLPPNSNPSPQLPKPPPTSSSSSSLGRVLGRPMEDVRSIYIFGRELGRGQFGVTYLVTHKATKEQFACKSIATRKLVNRDDIDDIRREVQIMHHLTGHRNIVELKGAYEDRHSVNLVMELCAGGELFDRIITKGHYSERAAANSCRQIVTVVHNCHSMGVMHRDLKPENFLLLNKNDDSPLKATDFGLSVFFKPGDVFRDLVGSAYYVAPEVLRRSYGPEADIWSAGVILYILLSGVPPFWAENEQGIFDAILRGHIDFASDPWPSISSSAKDLVKKMLRADPKERLSAVEVLNHPWMRVDGDAPDKPLDIAVLTRMKQFRAMNKLKKVALKVIAENLSEEEIIGLKEMFKSMDTDNSGTITFEELKAGLPKLGTKLSESEVRQLMEAADVDGNGTIDYIEFITATMHMNRMEREDHLYKAFEYFDNDKSGYITMEELESALKKYNMGDEKTIKEIIAEVDTDNDGRINYDEFVAMMRKGNPDITHITQRRRK

SEQ ID NO: 20 - protein, 379 aa, Glycine max
MGICFSIEDQNHLSISDSNAKPKPAVGHESGAPLASMNIKDLREGAGYSNVDIFTYEELRLATKHFRPDFILGEGGFGVVYKGVIDHSVRSGYKSTEVAIKELNREGFQGDREWLAEVNYLGQFSHPNLVKLIGYCCEDDHRLLVYEYMASGSLEKHLFRRVGSTLTWSKRMKIALHAARGLAFLHGAERPIIYRDFKTSNILLDADFNAKLSDFGLAKDGPMGDQTHVSTRVMGTYGYAAPEYVMTGHLTARSDVYGFGVVLLEMLIGRRALDKSRPSREHNLVEWARPLLNHNKKLLKILDPKLEGQYSSKTALKVAHLAYQCLSQNPKGRPLMSQVVEILENFQSKGENEEDQMLQTGDTSITLYEVPKGSNGTPT

SEQ ID NO: 21 - protein, 411 aa, Glycine max
MEMDPPPKSWSIHTRSEIIAKYEVMERVGSGAYADVYRGRRLSDGLTVALKEIHDYQSAFREIDALQLLEGSPNVVVLHEYFWREDEDAVLVLEFLRTDLATVIADTAKANQPLPAGELKCWMIQILSGLDACHRHMVLHRDLKPSNLLISEHGLLKIADFGQARILMEPGIDASNNHEEYSRVLDDIDNKDTITSTHDGNATCNTSDVDREEEELGCFTSCVGTRWFRAPELLYGSRNYGLEVDLWSLGCIFAELLTLQPLFPGTADIDQLSRIIGVLGNLDENAWAACSKLPDYGIISFSKVENPAGLEACLPNRSPDEVALVKKLVCYDPAKRATAMELLHDKYFSDEPLPVLVSELRVPLTRKEQDGDSPGGWGDINDMDSDSDSQFDEFGPLNITRTGTGFSIQFP

SEQ ID NO: 22 - protein, 344 aa, Glycine max
MSCFSCFVSRGKDVRRVEIDNGSRSATSSSEGKGKKSVSNKGTSTAAASFGFRELAEATRGFKEVNLLGEGGFGRVYKGRLSTGEYVAVKQLIHDGRQGFHEFVTEVLMLSLLHDSNLVKLIGYCTDGDQRLLVYEYMPMGSLEDHLFDPHPDKEPLSWSTRMKIAVGAARGLEYLHCKADPPVIYRDLKSANILLDNEFNPKLSDFGLAKLGPVGDNTHVSTRVMGTYGYCAPEYAMSGKLTLKSDIYSFGVLLLELITGRRAIDTNRRPGEQNLVSWSRQFFSDRKKFVQMIDPLLQENFPLRCLNQAMAITAMCIQEQPKFRPLIGDIVVALEYLASHSNP

SEQ ID NO: 23 - protein, 570 aa, Glycine max
MSAPAEAPSFTASPPSETTPSPNSTALSPPPPSTINATVSPPPPEAASPPTSTVNSGLSTGTVSGIVIGAVLGSVGMLIIGGIFFCFYRNWKRKKNHSQPPQPKADIAGGTLQNWQDSVPPTTDGKVGFSPKPPPGGLVNQQQSSAALLTLVVNSSNTSSSLGSEKAKSYISPSPGTSLALSQSTFTYDELSMATDGFSRSNLLGQGGFGYVHKGVLPNGKIVAVKQLKSESRQGEREFHAEVDVISRVHHRHLVSLVGYCVSDSQKMLVYEYVENDTLEFHLHGKDRLPMDWSTRMKIAIGSAKGLAYLHEDCNPKIIHRDIKASNILLDESFEAKVADFGLAKFSSDTDTHVSTRVMGTFGYMAPEYAASGKLTEKSDVFSFGVVLLELITGRKPVDKTQTFIDDSMVEWARPLLSQALENGNLNGLVDPRLQTNYNLDEMIRMTTCAATCVRYSARLRPRMSQVVRALEGNISLEDLNDGIAPGHSRVFGSFESSSYDSVQYREDLKNFKKLALESQEQGISEYSGPSSEYGRHPSVSTSSDQQNTQEMEMGNKKGSNHDSGIQVLD

SEQ ID NO: 24 - protein, 750 aa, Glycine max
MSREQQKRGKQEKGSDGAEKVIVAVKASKEIPKTALVWSLSHVVQPGDCITLLVVVPSQSSGRRLWGFPRFAGDCASGIKKYPPGTISEQKSDLTDSCSQMILQLHNVYDPNKINVRIKIVSGSPCGAVAAEAKKTQANWVVLDKQLKHEEKRCMEELQCNIVVMKRSQPKVLRLNLIGPQKKDVEEAGPSPSEQDDMPENRTKIKLDSLNSIKGPTVTPTSSPELGTPFTATEAGTSSVSSSDPGTSPFFISEMNGEFKKEETIKESQELVDTNSDTESESLSTSSASMRYQPWITELLLHQQSSQRNEERSDISHGIPQASTTRAFLEKYSRLDRGAGFEISTYRNDMDFSGNLREAIALSGNAPPGPPPLCSICQHKAPVFGKPPRWFTYSELELATGGFSQANFLAEGGFGSVHRGVLPEGQVIAVKQHKLASSQGDLEFCSEVEVLSCAQHRNVVMLIGFCIEDKRRLLVYEYICNGSLDSHLYGRQRDTLEWSARQKIAVGAARGLRYLHEECRVGCIIHRDMRPNNILITHDFEPLVGDFGLARWQPDGDTGVETRVIGTFGYLAPEYAQSGQITEKADVYSFGVVLVELVTGRKAVDLTRPKGQQCLTEWARPLLEEYAIEELIDPRLGKHYSEHEVYCMLHAASLCIQRDPQCRPRMSQVLRILEGDMVMDSNYISTPGYDAGNRSGRLWSEPLQRQQHYSGPLLEESLESFSGKLSLDKYKPSYWGDRDKARRASCEDDI

SEQ ID NO: 25 - protein, 441 aa, Glycine max
MDPQNQHPPNPRSIIFNKYEMGRVLGQGNFAKVYHARNLNTNESVAIKVIKKEKLKKERLVKQIKREVSVMRLVRHPHIVELKEVMATKGKIFLVMEYVKGGELFAKVNKGKLTEDLARKYFQQLISAVDFCHSRGVTHRDLKPENLLLDQNEDLKVSDFGLSTLPEQRRADGMLVTPCGTPAYVAPEVLKKKGYDGSKADLWSCGVILFALLCGYLPFQGENVMRIYRKAFRAEYEFPEWISPQAKNLISNLLVADPGKRYSIPDIMRDPWFQVGFMRPIAFSIKESYVEDNIDFDDVENNQEEEVTMRKPARPFYNAFEIISSLSHGFDLRSLFETRKRSPSMFICKFSASAVLAKVEAVAKKLNFRVTGKKEFVVRMQGTEEGRKGKLAMTVEVFEVAPEVAVAEFTKSAGDTLEYVKFCEEQVRPSLKDIVWSWQGD

SEQ ID NO: 26 - protein, 529 aa, Glycine max
MGCHGSKEKKSNTSYGSAATGSGSGSGSGGGYNTVQPSPPTTDQVQASAQTPENRKASPTVQKKADTSIVGKPFDDIKKYYSIGKELGRGQFGITYLCTENSSGGTYACKSILKRKLVSKADREDMKREIQIMQHLSGQPNIVEFKGAFEDRFSVHLVMELCSGGELFDRIIAQGHYSERAAASLCRSIVNVVHICHFMGVMHRDLKPENFLLSTKDDHATLKATDFGLSVFIEQGKVYHDMVGSAYYVAPEVLRRSYGKEIDIWSAGIILYILLSGVPPFWAETEKGIFNAILEGEIDFVSEPWPSISDSAKDLVRKMLTQDPKKRITSAQVLEHPWMREGGDASDKPIDSAVLSRMKQFRAMNKLKKLALKVIAENLSEEEIKGLKAMFANMDTDSSGTITYEELKTGLARIGSRLSEAEVKQLMDAADVDGNGSIDYLEFISATMHRHRLERDEHLYKAFQYFDKDNSGYITRDELETAMTQHGMGDEATIKEIISEVDTDNDGRINYEEFCAMMRSGMPHQGQLL

SEQ ID NO: 27 - protein, 461 aa, Glycine max
MGDGSGNRWSRAEWVQQYDLLGKIGEGTYGLVFLARTKGTPSKSIAIKKFKQSKDGDGVSPTAIREIMLLREITHENVVKLVNVHINHADMSLYLAFDYAEHDLYEIIRHHRDKLNHSINQYTVKSLLWQLLNGLSYLHSNWMIHRDLKPSNILVMGEGEEHGVVKIADFGLARIYQAPLKPLSDNGVVVTIWYRAPELLLGAKHYTSAVDMWAVGCIFAELLTLKPLFQGAEVKATSNPFQLDQLDKIFKVLGHPTLEKWPSLASLPHWQQDVQHIQGHKYDNAGLYNVVHLSPKSPAYDLLSKMLEYDPRKRLTAAQALEHEYFKIEPLPGRNALVPCQLGEKIVNYPTRPVDTTTDLEGTTNLPPSQTVNAVSGSMPGPHGSNRSVPRPVNVVGMQRMPPQAMAAYNLSSQAAMGDGMNPGGISKQRGVPQAHQPQQLRRKEQMGMPGYPAQQKSRRI

SEQ ID NO: 28 - protein, 381 aa, Glycine max
MGNCWCRWESSEYRVSSNVKSEQNQGTKQRHDDSKLPSNPEEVEDLRRDSAANPLIAFTYDELKIITANFRQDRVLGGGGFGRVYKGFISEELREGLPTLAVAVKVHDGDNSHQGHREWLAEVIFLGQLSHPNLVKLIGYCCEDEHRVLIYEYMSRGSVEHNLFSKILLPLPWSIRMKIAFGAAKGLAFLHEAEKPVIYRDFKTSNILLDQEYNSKLSDFGLAKDGPVGDKSHVSTRVMGTYGYAAPEYIMTGHLTPRSDVYSFGVVLLELLTGRKSLDKLRPAREQNLAEWALPLLKEKKKFLNIIDPRLDGDYPIKAVHKAAMLAYHCLNRNPKARPLMRDIVDSLEPLQAHTEVPIGKTLTIISEVPESGLKMKDDAI

SEQ ID NO: 29 - protein, 816 aa, Glycine max
MASTLLPFLFLSMVLLPFQTIAQTKSNIAIGESHTAGASTSPWLVSSPSGDFAFGFLPLEDTPDHFMLCIWYAKIQDKTIVWFANRDQPAPKGSKVVLTADDGLVLITAPNGHMLWKTGGLTLRVSSGVLNDTGNFVLQDGHSKTVWESFKDYRDTLLPYQTMEKGHKLSSKLGRNYFNKGRFVLFFQNDGSLVMHSINMPSGYANENYYQSGTIESNTNTSTSAGTQLVFDGTGDMYVLRKNNEKYNLSKGGSRASSTTQFYYLRATLDFDGVFTLYQHPKGSSGSGGWSQVWSHPDNICKDYVASAGSGVCGYNSICSLRDDKRPNCRCPKWYSLVDPNDPNGSCKPDFVQACAVDKLSNRQDLYDFEVLIDTDWPQSDYVLQRPFNEEQCRQSCMEDCMCSVAIFRLGDSCWKKKLPLSNGRVDATLNGAKAFMKVRKDNSSLIVPPIIVNKNNKNTSILVGSVLLGSSAFLNLILVGAICLSTSYVFRYKKKLRSIGRSDTIVETNLRRFTYEELKKATNDFDKVLGKGAFGIVYEGVINMCSDTRVAVKRLNTFLMEDVHKEFKNELNAIGLTHHKNLVRLLGFCETEEKRLLVYEYMSNGTLASLLFNIVEKPSWKLRLQIAIGIARGLLYLHEECSTQIIHCDIKPQNILLDDYYNARISDFGLAKLLNMNQSRTNTAIRGTKGYVALEWFKNMPITAKVDVYSYGVLLLEIVSCRKSVEFEAEDEEKAILAEWAYDCYIEGTLHALVEGDKEALDDMKTFEKLVMIALWCVQEDPSLRPTMRNVTQMLEGVVEVKMPPCPSQFSVQYS

SEQ ID NO: 30 - protein, 711 aa, Glycine max
MPVLRSGARKGRAAPKQQQQKQQQSPVVEGEAIATRTRRRRAAAAAAAAVPESNNNNNNTNNQQQQVEQVAVVNENVAVAAREEDNRVAEEEGVVRGGGAEREEVAEKKMGGCDSGGRSNGKANAAGEDDANTPQVPQKIQVGNSPSYKVEKKLGKGGFGQVYVGRRTGGNLNERTGPGAVEVALKLEHRTSKGCTYGPPYEWQVYNTLGGSHGVPQVHYKGRQGDYYVMVMDMLGPSLWDVWNNSNHHMTTEMVACIAIEAISILEKMHSRGYVHGDVKPENFLLGAPGTPDEKKLFLVDLGLATKWRDSTTGSHVEYDQRPDVFRGTVRYASVHAHLGRTASRRDDLESLAYTLIFLLRGRLPWQGFQGENKGFLVCKKKMGTSPETLCCFSPLPFKQFVEHVVNLKFDEEPNYAKYISLFDGVVGPNPDIRPINTEGAQKLIGHKRGRLVMEEEDDEQPKKKIRIGLPASQWISVYNARRPMKQRYHYNVSDTRLSQHIEKGNEDGLYISSVASCQNLWALIMDAGTGFTAQVYELSPFFLHKEWIMEHWEKNYYISAIAGAVNGSSLVVMSKGTQYLQQSYKVSDSFPFKWINKKWREGFYVTAMATSGSRWGVVMSRGAGFSDQVVELDFLYPSEGIHKRWDCGYRITATAATWDQAAFVLSVPRRKPLDETQETLRTSAFPSTHVKEKWAKNLYIASICYGRTVS

SEQ ID NO: 31 - protein, 355 aa, Glycine max
MLILVLGLASATFLVFVAVYLFYSKRRVSKYNESKDIESSEHKEDEEMAQKEDLMIFQGGEDLTICDILDAPGEVIGKSNYGTLYKALLQRSNKVSLLRFLRPVCTARGEELDEMIHFLGRIRHPNLVPLLGFYTGPRGEKLLVHPFYRHGSLTQFIRDGNGECYKWSNICRISIGIAKGLEHLHTSQEKPIIHGNLKSKNILLDRSYQPYISDSGLHLLLNPTAGQEMLESSAAQGYKAPELIKMKDASEESDIYSLGVILLELLSGKEPINEHPTPDEDFYLPNFMRNAVLGHRIADLYHPAILLRNSRDDSIPVTEECILKVFQLAMACCSPSPSVRPNIKQVLKKLEEIMF

SEQ ID NO: 32 - protein, 1034 aa, Glycine max
MARCILSHVLAFALVAGLCVFSCLVNVAQAQSANATTDPSEARALNSIFSKWDILANPTQWNISSELCSGRAIDATTTIDDTTFNPFIKCDCSYDSRTTCRITALKVYAMSIVGTIPEELWTLTYLTNLNLGQNYLTGSLPPNIGNLTRMQYLSIGINNFSGELPKELGNLTELRSLAFGSNKFRGSLPSELGKLTNLEQIYFDSSGISGPIPSTFANLKNLLHVGASDTELTGKIPDFIGNWSKLQTLRFQGNSFNGSIPSSFSNLSSLTELRISGLSNGSSSLEFLRNMKSLTILELRNNNISGSISSTIGELHNLNQLDLSFNNITGQNLGSIFNLSSLTYLFLGNNKFNGTLPMQKSSSLVNIDLSYNDLSGSLPSWVNEPNLQLNLVANNLDVSNASGLPIGLNCLQKNFPCNQGIGRYSDFAIKCGGNQIRSADGIVYEMDNQTLGPATYFVTDANRWAISNVGLFTGSSNPVYKSFVSNQFTGTVNSELFQTARLSASSLRYYGLGLENGFYNITLQFAETAILDSTRSWESLGRRVFDIYIQGTRVLKDFDIQKEAGGISYKAIQRQFRFEVTENYLEIHLFWAGKGTCCIPTQGTYGPLIQAIHAIPDFIPTVSNKPPSSNNNNIGLILGIVLGVGVVSVLSIFAIFCIIRRRRRRDDEKELLGIDTKPYTFSYSELKNATNDFNLENKLGEGGFGPVYKGTLNDGRVIAVKQLSVGSHQGKSQFITEIATISAVQHRNLVKLYGCCIEGSKRLLVYEYLENKSLDQALFGKCLTLNWSTRYDICLGVARGLTYLHEESRLRIVHRDVKASNILLDYELIPKISDFGLAKLYDDKKTHISTGVAGTIGYLAPEYAMRGHLTEKADVFSFGVVALELVSGRPNSDSSLEGEKVYLLEWAWQLHEKNCIIDLVDDRLSEFNEEEVKRVVGIALLCTQTSPTLRPSMSRVVAMLSGDIEVSTVTSKPGYLSDWKFEDVSSFMTGIEIKGSDTNYQNSSGSTSMMGGVDYYSPRDVSKPILKETLWEGR

SEQ ID NO: 33 - protein, 294 aa, Glycine max
MDQYEKVEKIGEGTYGVVYKARDRATNETIALKKIRLEQEDEGVPSTAIREISLLKEMQHRNIVRLQDVVHSEKRLYLVFEYLDLDLKKHMDSSPEFVKDPRQVKMFLYQILCGIAYCHSHRVLHRDLKPQNLLIDRRTNSLKLADFGLARAFGIPVRTFTHEVVTLWYRAPEILLGSRHYSTPVDVWSVGCIFAEMVNRRPLFPGDSEIDELFKIFRILGTPNEDTWPGVTSLPDFKSTFPKWPSKDLANVVPNLDAAGLNLLSSMLCLDPSKRITARSAVEHEYFKDIKFVP

SEQ ID NO: 34 - protein, 1281 aa, Glycine max
MGVVLALILQLVKLCIIGFVVEFQGSEGSVISPSPAFLPVIHPSGEAPAPIHHGLSWESSPPKSPSDPNEFGISPSNEIIPVDPSPSEPPGTLHPREPWRTIAPIPPEVPNGSFLPPPVTTLPPPTSAPTPQKVKGFEPSISPSPSTSTIASSPPAPYNAAHAPSTSEGSVPPSIQPSPPQSKTTPAIRPPVSTPIAPAPIAITSGNLPKTSPVSQPIEHGSLPPKIDERNKSHKPEPALPALVPIPSTILPKISPVSQPTENGSLPHREGANNGHISEPISPAPTVFSFPEHSPESQPTEHGSLPPTVPRRNINTGRTLEPVSQAPVATAPAILPKNSSVSQPTHHGNSPPDVQNRTANNGHSHTPAPEMPPSVTPPRPFPVDPPLVHPVIPAAPPPKSPAPGGEPVSAPVYKTPKPPSAIVHFHAQAPVVSPVSTPSRSFNWTKGGEPVSAPPYKTPKPLPVIVHSPAQAPSSAHKARQFHHAPEPPISSPESPFNKDHPPASSPSTTFYKHHHTRNTVTSPAPASSHSISPSTSKHQDGSNPPLPLPTSRQRHHAPPPMNTGSSVSPSGLPIQSPVSQVSPAPSPLFKISPHSTKIPLPPPKVSPSRPSSKTPKKPVRPRFQALPPPPPNEDCISLVCSDPYTSTPPGAPCKCVWPMKVGLRLSVSLYTFFPLVSEFASEIATGVFMKQSQVRIMGADAANQQPDKTIVFVDLVPLGEEFDNTTAFLTSERFWHKQVVIKTSYFGDYDVLYVTYPGLPPSPPLPPSSISIIDGGPYSGGGNNGRTIKPLGVDISKRQHKGGLSKGIIAIIALSVFLVVVLCFAAALALFKYRDHVSQPPSTPRVLPPLTKAPGAAGSVVGGGLASASTSFRSNIAAYTGSAKTFSMNDIEKATDNFHASRVLGEGGFGLVYSGTLEDGTKVAVKVLKREDHHGDREFLSEVEMLSRLHHRNLVKLIGICAEVSFRCLVYELIPNGSVESHLHGVDKENSPLDWSARLKIALGSARGLAYLHEDSSPHVIHRDFKSSNILLENDFTPKVSDFGLARTAADEGNRHISTRVMGTFGYVAPEYAMTGHLLVKSDVYSYGVVLLELLTGRKPVDMSRPPGQENLVAWARPLLSSEEGLEAMIDPSLGHDVPSDSVAKVAAIASMCVQPEVSDRPFMGEVVQALKLVCNECDEAREAGSSSSSVDLSHSRQPSDNLQGQFSATNYDSGIDIENGLLASELFSSSARYGRRVSGSFRRHSYSGPLNTGRSKRLWQIIRKLSGGSISEHGTMFKL

SEQ ID NO: 35 - protein, 462 aa, Glycine max
MTSRSGQGSSGGGSSRTRVGKYELGRTLGEGNFAKVKFARHVETRENVAIKILDKEKLLKHKMIAQIKREISTMKLIRHPNVIRMYEVMASKTKIYIVLEFVTGGELFDKIARSGRLKEDEARKYFQQLICAVDYCHSRGVFHRDLKPENLLLDANGVLKVSDFGLSALPQQVREDGLLHTTCGTPNYVAPEVINNKGYDGAKADLWSCGVILFVLMAGYLPFEETNLSALYKKIFKAEFTCPPWFSSSAKKLINKILDPNPATRITFAEVIENDWFKKGYKPPVFEQANVSLDDLDSIFSDSTDSQNLVVERREEGPMAPVAPVTMNAFELISKSQGLNLSSLFEKQMGLVKRETRFTSKCSADEIISKIEKAAGPLGFDVKKNNCKLKIQGEKTGRKGHLSVATEILEVAPSLYMVELRKSEGDTLEFHKFYKNLATGLKDIVWKAEPIDEEKDGANPSK

SEQ ID NO: 36 - protein, 440 aa, Glycine max
MMGEKSNSNSGDAINSTLLHGKYELGRLLGHGSFAKVYHARHLNTGKSVAMKVVGKEKVVKVGMMEQIKREISAMNMVKHPNIVQLHEVMASKSKIYIAMELVRGGELFNKIARGRLREETARLYFQQLISAVDFCHSRGVFHRDLKPENLLLDDDGNLKVTDFGLSTFSEHLRHDGLLHTTCGTPAYVAPEVIGKRGYDGAKADIWSCGVILYVLLAGFLPFQDENLVALYKKIYRGDFKCPPWFSSEARRLITKLLDPNPNTRITISKIMDSSWFKKPVPKNLVGKKREELNLEEKIKHQEQEVSTTMNAFHIISLSEGFDLSPLFEEKKREEKELRFATTRPASSVISRLEDLAKAVKFDVKKSETKVRLQGQENGRKGKLAIAADLYAVTPSFLVVEVKKDNGDTLEYNQFCSKELRPALKDIVWRTSPAENPTLA

SEQ ID NO: 37 - protein, 793 aa, Glycine max
MFKFLKEVVGGSGTGVKDLPYTIAEPYPSAWGSWTHSRGTSKDDGSPVSVFSLSGSNAQDGHLAAARNGVKRLRTVRHPNILSFLHSAEIETYDAGSPKVTIYIVTEPVMPLSEKIKELGLEGTQRDEYYALGLHQIAKAVSFLNNDCKLVHGNICMASTVVTPTLDWKLHALDVLSEFDGSSEASSGQMLQYAWLVGSQYKPMELAKSDWDAIKKSPPWAIDSWGMGCLIYEVFSGLRLGKTEELRNIGSIPKSLLPDYQRLLSSMPSRRLNTSKLIENSEYFQNKLVDTIHFMEILSLKDSVERDTFFRKLPNLAEQLPRQIVLKKLLPLLASALEFGSAAASALTALLKMGSWLSAEEFNVKVLPTIVKLFASNDRAIRVALLQHIDQYGESLSAQAVDEQVYPHVATGFSDTSAFLRELTLKSMLILAPKLSQRTMSGSLLKYLSKLQVDEEPAIRTNTTILLGNIGSYLNEGTRKRVLINAFTVRALRDTFPPARGAGIMALCATSSYYDITEVATRILPNVVVLTIDPDSDVRTKAFQAVDQFLQIAKQHYEKTNAADTSCGVGSSSVPGNASLLGWAMSSLTLKGKPSDHAPVASASSTAITSTSSNGTAGIETPSTAAAHVSSTADLAEHPVPTSPTSTDGWGELENGIDEEHGSDRDGWDDLEPLEETKPAPALANIQAAQRRPVSQPISHTKQASNLLSKSTPKLNKDEDDDLWGSIAAPAPKTARPLNLKSAQTDDDDPWAAIAAPAPTIKAKPLSAGRGRGAKPAAPKLGAQRINRTSSGM

SEQ ID NO: 38 - protein, 460 aa, Glycine max
MEQKGSVLMQRYELGRLLGQGTFAKVYHARNLITGMSVAIKVVDKEKILKVGMIDQIKREISVMRLIRHPHVVELYEVMASKTKIYFVMEHAKGGELFNKVVKGRLKVDVARKYFQQLISAVDYCHSRGVCHRDLKPENLLLDENENLKVSDFGLSALAESKCQDGLLHTTCGTPAYVAPEVINRKGYDGIKADIWSCGVILYVLLAGHLPFQDTNLMEMYRKIGRGEFKFPKWFAPDVRRFLSRILDPNPKARISMAKIMESSWFKKGLEKPAITVTENEELAPLDADGIFEACENDGPIAEPKQEQAKPCNLNAFDIISFSTGFDLSGLFEDTFLKKETRFMSKKPASIIVLKLEEICKRLCLKVKKKDGGLLKLEGSKEGRKGTLGVDAEIFEITPHFHMVELRKSNGDTMEYQKLFKQDIRPALKDIVWTWQGEKPQQEQEQHEVVQEEHQPSHTA

SEQ ID NO: 39 - protein, 558 aa, Glycine max
MDSARSWLQKFQPRDKTRAAGKKKEEDGNGGNQDSNEAIDEALLSSVTKQKVAAAKQYIENHYKEQMKNLQERKERRTILEKKLADADVSEEDQNNLLKFLEKKETEYMRLQRHKMGVEDFELLTMIGKGAFGEVRVCREKTSGHVYAMKKLKKSEMLRRGQVEHVKAERNLLAEVDSNCIVKLYCSFQDDEHLYLIMEYLPGGDMMTLLMRKDILTEDEARFYVGETVLAIESIHKHNYIHRDIKPDNLLLDRYGHLKLSDFGLCKPLDCSTLEENDFSVGQNVNGSTQSSTPKRSQQEQLQHWQINRRTLAYSTVGTPDYIAPEVLLKKGYGMECDWWSLGAIMYEMLVGYPPFYSDDPMLTCRKIVNWKTYLKFPEEARLSPEAKDLISKLLCNVNQRLGSKGADEIKAHPFFKGVEWNKLYQMEAAFIPEVNDELDTQNFEKFDESDSQTQSSSRSGPWRKMLSSKKDLNFVGYTYKNFEIVNDYQVPGMAELKKKQSKPKRPTIKSLFDCESETPEASDTSANDQPAQGSFLKLLPPQLEVSPHRDKNLPPRS

SEQ ID NO: 40 - protein, 378 aa, Glycine max
MKIPCFSCFSPSTTEKNNNNDYPDEEINDGSFRLFTHKQLKLATRNFHSSEKVGEGGFGSVFKGKLVDGSFVAVKVLSVEVESMRGEREFVAELATLANIKHQNLVSLKGCCVEGAYRYLVYDYMENNSLYNTFLGSEERRMRFNWEIRKDVSIGVARGLDFLHEELKPHIVHRDIKAKNILLDRNFIPKVSDFGLAKLLRDETSYISTRVAGTLGYLAPEYANSGQVSRKSDVYSFGVLLLQIVSGLAVVDAYQDIERFIVEKAWAAYQSNDLLKLVDPMLNMNFPEEEALKFLKVGLLCVQETAKLRPRMSEVVEKLTKDIDMRDVHISKPGFVADLRNIRIKQQNLNSSEESSSAGATFTSSISSSANLARYLLD

SEQ ID NO: 41 - protein, 648 aa, Glycine max
MLLLLLNYLFFLFNFSITKTEAIDNLQYLNHSCSSNKTFTPNSTYQSNLQTLLTSLSSHATTAQFFNTTTGGGDAAGENIYGSFMCRGDVSNHTCQECIKTATQQITVRCLNSKEALIWYHECMVRYSNRCFFSAVEEWPRFNFVDFNVNTNSTEGIYGYWLLSKTLSDAVGEAVKAGTKKFATKNATVFGSQRVHTLVQCTPDLSSEDCSKCLGDIMRDIPLCCLGRRGGMVLFPSCTLMFGIGQFYRDFPHGTPESKSENEKGSLRTIVIIVLLVVVVPVMLSFFSYHLIRRKARKRNYKILLRENFGQESVTIEGLQFDLDIIAAATNNFSHENKIGKGGFGEVYKGILPNGRRIAVKRLSTNSSQGSVEFKNEILSIAKLQHRNLVELIGFCLEVQEKILIYEYMSNGSLDNFLFDPQQKKLSWSQRYKIIEGTARGILYLHEHSRLKVIHRDLKPSNILLDENMNPKISDFGMARIIELNQDLGKTQRIVGTFGYMSPEYAIFGQFSEKSDVFSFGVMIIEIITGRKNINSHQLPDIVDSLMSYVWRQWKDQAPLSILDPNLEENYSQFEVIKCIHIGLLCVQENKNIRPTMTKVIFYLDGHTLDELPSPQEPPFFFRDIKDKKIPMQHFSVNKMSTSIFYPR

SEQ ID NO: 42 - protein, 624 aa, Glycine max
MERKFMALGFIWWVVVVHPLCLISANMEGDALHSLRTNLQDPNNVLQSWDPTLVNPCTWFHVTCNNDNSVIRVDLGNAALSGQLVPQLGQLKNLQYLELYSNNITGPIPSDLGNLTNLVSLDLYLNHFTGPIPDSLGKLSKLRFLRLNNNSLSGPIPMSLTNITALQVLDLSNNHLSGVVPDNGSFSLFTPISFANNMDLCGPVTGHPCPGSPPFSPPPPFVPPPPISAPGGNGATGAIAGGVAAGAALLFAAPAIAFAWWRRRKPQEFFFDVPAEEDPEVHLGQLKRFSLRELQVATDSFSNKNILGRGGFGKVYKGRLADGSLVAVKRLKEERTPGGELQFQTEVEMISMAVHRNLLRLRGFCMTPTERLLVYPYMANGSVASCLRERPPYQEPLDWPTRKRVALGSARGLSYLHDHCDPKIIHRDVKAANILLDEEFEAVVGDFGLAKLMDYKDTHVTTAVRGTIGHIAPEYLSTGKSSEKTDVFGYGIMLLELITGQRAFDLARLANDDDVMLLDWVKGLLKEKKLEMLVDPDLQTNYIETEVEQLIQVALLCTQGSPMDRPKMSEVVRMLEGDGLAERWDEWQKVEVLRQEVELAPHPNSDWIVDSTENLHAVELSGPR

SEQ ID NO: 43 - protein, 1109 aa, Glycine max
MQLLHSDEPAPERGDSPEKPDDPNADTDSLDPGTDDGAALDVTGKSVEFPAAENAGDSAESLYVYKNVYSLIPKSVSRLARLRTLKFFGNEINLFAPEFGNLTALECLQMKISSPGIGGLQLHTLKGLKELELSKGPPRPSAFPILTEISGLKCLTKLSICHFSIRYLPPEIGCLKKLEYLDLSFNKMKTLPAEISYLKGLISMKVANNKLVELPAAMSSLSRLERLDLSNNRLTSLGSLELASMHRLQELNLQYNKLLGIFQIPSWICCNMDGNDKARCKDDCSSSVEMDLYESNFQENDETLSDGPHNTSSSMLTSSSSSSRCFASRKSGKRWKRRHHLQQKARQERLNNSRKWKAVDHDDQLLSKKIHRISEPENHDSLASESCAEIVSENGSLDDNNKRISSERAVNDNAIDNDNNDEVITEKQFSGEDCCTTESKDEKEESLCSLDKRPSEQDEASCLELLECVSKSKRHLDRDLDNPKPCKSRKSISSSSLLSCKYSKISFCGIEDHLSDGFYDAGRDRLFMPLECYEQNHCLASREVILLDRKIDEELDAVMLAAQALVYNLKKLNGLSRYGNQDGVDNLQMASLLALFVSDHFGGSDRSGIVERTRKSVSGSNYNKPFVCTCSAGSSTSISSPTEPVANTIEDITLSKMSEKSLDSIKKRRNSIIIPIGSVQYGVCRHRALLFKYLCDHMEPPVPCELVRGYLDFSPHAWNIILIKRGATWVRMLIDACRPLDIREEKDPEYFCRYIPLNRTTIPISSIGSPGPDYSFPSLTTCDELETKASTTLVKCKFGSVEAAAKVRTLEEQGSSADKIKNFEYNCLGEIRILGALKHPCIVEMYGHQISCQWSVSADGNPEHRVLRSAIFMEYVEGGSLKNYLEKLSEAGEKHVPVELALHIAKDVSCALSELHSKHIIHRDIKSENILFNLDRKRDDGTPTVKLCDFDSAVPLRSTLHVCCIAHAGTPPPCICVGTPRWMAPEVMRTMYKKNSYGLEADIWSFGCLLLEMLTLQIPYSGLSDSHFLDSLQMGKRPQLTDELRVLSSMNGPTMIPSGEELEKSDAGVDMLKFLVDLFHKCVEENPSKRPTAEEIHKMVLAHTDRLQI

SEQ ID NO: 44 - protein, 696 aa, Glycine max
MAMIPLMMMILLLLFKFPSVVLATLKSDEANLELVFTYHKCNEELGNFTTETYSNNRNVLLSNMYSDKEIENGFYNSSYGEGPDKVYGIGFCRGDVKPDKCRSCLEKSSTLLTDRCPVQKEAIGWYDLCMLRYSNRSIVEQPVTDTDDIIKCSNTNATNKDRFDKELDDLVVRMRSRSAEGDSRLKFAEGEAPVQSSNETIHALLQCVPYLSHQNCTRCLEYAMSRISYWCDGKTGGWYLGRSCSLRYETYLFFELIFHDAPAPQPSQPAVTPTKDFPKKTNPSRNIIVIVVPVFAVAIVVVGLIVLIYNYFGARRPRHKPIQSEGDGEGDGEGEGELDNDIKTDELAQFEFATIKFATNNFSDANKLGQGGFGIVYKGTLSDGQEIAIKRLSINSNQGETEFKTEISLTGKLQHRNLVRLLGFCFAKRERLLIYEFVPNKSLDFFIFDPNKRGNLNWERRYNIIRGIARGLLYLHEDSRLQVVHRDLKISNILLDEELNPKISDFGMARLFEINQTEANTNTVVGTFGYMAPEYIKHGKFSVKSDVFSFGVMMLEIVCGQRNSKIRGNEENAEDLLSFAWKNWRGGTVSNIVDTTLKDYSWDEIKRCIHIGLLCVQEDINGRPTMNSVSIMLNSSSFSLAEPSEPAFLMRGKSQLPMIMLSGSEQYSEATKSSDSGSQFAQGSSNKAPITEPYPR

SEQ ID NO: 45 - protein, 691 aa, Glycine max
MFPLQCSNHPMCAFSAVTAILLLFPAATSQAQILKKETYFFGPFNQSDFTTLTVLPSAAINLGALQVTPDSTGNVSLANHSGRIFFNNPFTLWDNDDNLNGKLVSFNTSFLINVFRPQNNPPGEGITFLITASTTVPNNSHGQFLGLTNAATDGNATNKFVAVELDTVKQDFDPDDNHIGLDINSVRSNVSVSLTPLGFEIAPNVTRFHVLWVDYDGDRKEIDVYIAEQPDKDAPIVAKPAKPVLSSPLDLKQVVNKVSYFGFSASTGDNVELNCVLRWNITIEVFPKKNGIGKALKIGLSVGLTMVVLIVAGVVGWVCWLKKKKRGNESQILGTLKSLPGTPREFRYQELKKATNKFDEKHKLGQGGYGVVYRGTLPKENLEVAVKMFSRDKMKSTDDFLAELTIINRLRHKNLVRLLGWCHRNGVLLLVYDYMPNGSLDNHIFCEEGSSTTPLSWPLRYKIITGVASALNYLHNEYDQKVVHRDLKASNIMLDSDFNARLGDFGLARALENDKTSYAEMEGVHGTMGYIAPECFHTGRATRESDVYGFGAVLLEVVCGQRPWTKNEGYECLVDWVWHLHREQRILDAVDPRLGNGCVVEEAERVLKLGLACSHPIASERPKMQTIVQIISGSVNVPHVPPFKPAFVWPAMDLSSPASDLTTPTTTTEYTPMSSDTHSMHVQFSDSNSLI

SEQ ID NO: 46 - protein, 1392 aa, Glycine max
MSRQTTSSAFTKSKTLDNKYMLGDEIGKGAYGRVYKGLDLENGDFVAIKQVSLENIAQEDLNIIMQEIDLLKNLNHKNIVKYLGSSKTKSHLHIVLEYVENGSLANIIKPNKFGPFPESLVAVYIAQVLEGLVYLHEQGVIHRDIKGANILTTKEGLVKLADFGVATKLTEADVNTHSVVGTPYWMAPEVIEMAGVCAASDIWSVGCTVIELLTCVPPYYDLQPMPALFRIVQDEHPPIPDSLSPDITDFLLQCFKKDARQRPDAKTLLSHPWIQNCRRVLQSSLRHSGTLRNIEEDDSADAEVSGGYHKSAYENSSVEKEDSAKEHTTMAADGSKAHEDNAADSNFSNEQTEKADDAPSDQVLTLAIHEKSFLQAGSSKLTSNREVVNSESTGNHEISNAKDLHEVVMNGEGGSPQSRGMASKVGGKDSSVNNGNKSFAFGPRGQDNGPLKKAMKMPITVEGNELSRFSDPPGDAYLDDLFHPLDKQPGEVVAEASTSTSTSHMTKGNASAIDGVKNDLAKELRATIARKQWEKESEIGQANNGGNLLHRVMIGVLKDDVIDIDGLVFDEKLPGENLFPLQAVEFSKLVGSLKPEESEDMIVSACQKLIGIFHQRPEQKIVFVTQHGLLPLTDLLEVPKTRIICSVLQLINQIVKDNTDFQENACLVGLIPAVTSFAVPDRPREIRMEAAYFLQQLCQSSSLTLQMFIACRGIPVLVGFLEADYAKYREMVHLAIDGMWQVFKLQQSTPRNDFCRIAAKNGILLRLINTLYSLNESTRLASSSAGGGFSVDGSAQRPRSGILDPNHPYINQNETMLSSVDQQDPPKVRRAVPDHHLEPSSSNPRRSDANYPVDVDRPQSSNATADEKSLNQASRESSAGALKERENMDRWKTDPSQPRISNNRTSTDRPPKSTEPSSNGLSVTGTMHQEQVRPLLSLLDKEPPSGRFSGQLEYMRQFSGLERHESVLPLLHATEKKTNGELDFLMAEFADVSQRGRENGNLDSSARVSHKVTPKKLGTLGSSEGAASTSGIASQTASGVLSGSGVLNARPGSATSSGLLSHMVSSLNAEVAREYLEKVADLLLEFAQADTTVKSYMCSQSLLSRLFQMFNRVEPPILLKILRCINHLSTDPNCLENLQRAEAIKYLIPNLELKEGSLVSEIHHEVLNALFNLCKINKRRQEQAAENGIIPHLMLFITSNSPLKQYALPLLCDMAHASRNSREQLRAHGGLDVYLNLLEDELWSVTALDSIAVCLAHDNDNRKVEQALLKKDAVQKLVKFFQGCPEQHFVHILEPFLKIITKSARINTTLAVNGLTPLLIARLDHQDAIARLNLLRLIKAVYEHHPQPKKLIVENDLPEKLQNLIGERRDGQVLVKQMATSLLKALHINTVL

SEQ ID NO: 47 - protein, 786 aa, Glycine max
MAMDEYECHEMVHVAVGKSLKKAATLLQWCFTHFSKPQIFLLHVHQPSTMIPTLLGKLPASQASPEVVSAYRIEEKEDTKRLLEKYLSLCRAAKVKASSVIGEADQVQKGIVDLVTVHNVRKLVIGAIPENCMKIKRNSSKANYAAKNAPPFCEIWFVYNGKHIWTREASETPRSLSSRAQPETTTAESLSCRSFHDGTKELLHSECLQLNSTKTTRSMVQSEIIEAEATFSSKSSSCNSHCSPQHSAGWYLDTHSEFEEETIDSQLIETKREAKAATDKALAELLKSKRLEVKAIEAISKVNFFESAHAHEVKLRKEAEDALRATIQEQQMFLDEKEEIARELERTVRSISLLGNCAHETNHKRDEAENELSLIQASISNLWHEKQQIRQQKMEALHWLERWKSCGQVGADHCNGVIGFAEEFPELAEFSLSDLQNATCNFSESFKVMEGGYGSIYKGEMLGRTVAIRKLHPHNMQGSSEFHQEAQILGSLQHPHLVTLLGVCPEAWSFVYEYLPSGSLQDYLFRKSSFLPLTRNIRAQWIAEIATALCFLHSSKPETIIHGGLTLETVLLDSALSCKICEFGFSRLVKEESVYLPNFHFSTEPKGSFTYTDPEFQRTGVLTPKSDIYSFGIIILQLLTGRTPVGLVGEVRRAVSCGKLYPILDSSAGEWNSTMATRLAELGLQCCQLNSRVRPELTPSLVRELKQLLVLEERPVPSFFLCPIFQEIMHDPQVAADGFTYEGKAISEWLENGHETSPMTNLKLTHLNLTPNHALRLAIQGWLCKS

SEQ ID NO: 48 - protein, 661 aa, Glycine max
MTVEKRIVLVGIRIDGYSRQLLNWALAKVAEPGDCVIAVHVVKSSDYVSKNKTLIDGYLEVYEGLCGVKKVGLTGQIFTGSSIKNILVREAKKHAALALVVGGRAATAKYCAKRLQPTTNVLAIQDSRIVFRSCTNKQLPGGLILDPRPSLTIIKENLRDRAIHSSICDSIVEIEESTRKNSLESKEEAFNGSEKSKSRSISMFAGDSAEQKLGWPLLRRANSGMSQTLHARDMSVVQWVMTLPDRSPNKGSSSSSTEENPFERSISDVEYESSSNSSPSSVDIPNGLEEMLNLNSLNCKRFSLEVLKSCTSQFSSEKLVGKGGSNRVYKGVLTDGKSIAVKVMQSSKEAWKDFALEVEIISSLEHKSIAPLLGICIENNTLISVYDYFPNGSLEENLHGKNKDESILSWEVRFNVAIRIAEALDYLHREALKPVIHKDVKSSNILLSQGFEPQLSDFGLAVWGPTTSSFLTQDVVGTFGYLAPEYFMYGKVSDKIDVYAFGVVLLELISGREPINSAACKGQESLVVWAKPIIESGNVKGLLDPNLEGKFDEAQLQRMVLAASLCITRAARLRPKLSQILKILKGEEKVEYFLNSQGDNDQEDSENQENIDDEVYPNSSAELHLSLALLGVDDDSTSHSSTDHSYSEDLKEQWSRSSSFN

SEQ ID NO: 49 - protein, 419 aa, Glycine max
MDSARTAPPPWQELDLDSLKPLKVLGKGGMGTVFLVQAANNTRFALKVVDKTCVHAKLDAERRARWEIQVLSTLSHPFLPSLMGTFESPQFLAWALPYCPGGDLNVLRYRQTDRAFSPAVIRFYVAEILCALDHLHSMGIAYRDLKPENVLVQNTGHITLTDFDLSRKLNPKPKPNPQVPSIPLPNSNVPEPRRKHRRNFSRWISLFPPDGTHHNNNKNGLKKAKSARVSPVSRRKPSFSNGERSNSFVGTEEYVSPEVVRGDGHEFAVDWWALGILIYEMLYGTTPFKGKNRKETFRNVITKPPVFVGKRTALTDLIEKLLEKDPTKRLGYTRGAVEIKEHEFFRGVRWELLTEVVRPPFIPTRDDGAGDSTDRISDRNCGFDIRGYFLNLKSSPSLPGSPLPSPSCRFKKNVSLTEF

SEQ ID NO: 50 - protein, 463 aa, Glycine max
MGICLSTQIEAGLNSKHVSVDAKDLSSPSSKITKDLSNPISNKITEDLSTPISNKITEDLSTPISNKITEDLSTPISNKITEDLSTPISKVSEILVPLTPQIEGEILQSSNLKNFSLTELTAATRNFRKDSVLGGEGDFGSVFKGWIDNHSLAAAKPGTGVVVAVKRLSLDSFQGHKDRLARHGMTHEASLEAEVNYLGQLSHPHLVKLIGYCFEDKDRLLVYEFMPRGSLENHLFMRGSYFQPLSWGLRLKVALGAAKGLAFLHSAETKVIYRDFKTSNVLLDSNYNAKLADLGLAKDGPTREKSHASTRVMGTYGYAAPEYLATGNLSAKSDVFSFGVVLLEMLSGRRAVDKNRPSGQHNLVEWAKPYLSNKRKLLRVLDNRLEGQYELDEACKVATLSLRCLAIESKLRPTMDEVATDLEQLQVPHVKQNRRKSADHFTHGRIATASASPLSRDIANTHP

SEQ ID NO: 51 - protein, 829 aa, Glycine max
MRNNKPQLWLSLSLIITCFSFHTSLAALTTISANQSLSGDETLVSQHGNFELGFFNTGNNSNKFYIGMWYKKISQRTYVWVANRDQPVSDKNSAKLTILEGNLVLLDQSQNLVWSTNLSSPSSGSAVAVLLDTGNLILSNRANASVSDAMWQSFDHPTDTWLPGGKIKLDKKTKKPQYLTSWKNREDPAPGLFSLELDPAGSNAYLILWNKSEQYWTSGAWNGQIFSLVPEMRLNYIYNFTFQSNENESYFTYSMYNSSIISRFVMDGSGQIKQLSWLENAQQWNLFWSQPRQQCEVYAFCGGFGSCTENAMPYCNCLNGYEPKSQSDWNLTDYSGGCVKKTKFQCENPNSSDKEKDRFLPILNMKLPNHSQSIGAGTVGECEAKCLSNCSCTAYAHDNSGCSIWHGDLLNLQQLTQDDNSGQTLFLRLAASEFDDSNSNKGTVIGAVAGAVGGVVVLLILFVFVMLRRRKRHVGTRTSVEGSLMAFGYRDLQNATKNFSEKLGGGGFGSVFKGTLPDSSVVAVKKLESISQGEKQFRTEVSTIGTVQHVNLVRLRGFCSEGTKKLLVYDYMPNGSLESKIFHEDSSKVLLDWKVRYQIALGTARGLTYLHEKCRDCIIHCDVKPENILLDADFIPKVADFGLAKLVGRDFSRVLTTMRGTRGYLAPEWISGVAITAKADVYSYGMMLFEFVSGRRNSEASEDGQVRFFPTIAANMMHQGGNVLSLLDPRLEENADIEEVTRVIKVASWCVQDDESHRPSMGQVVQILEGFLDVTLPPIPRTLQAFVDNHENVVFFTDSSSTQTSQVKSNASAASSQAKSNISSSNSST

SEQ ID NO: 52 - protein, 420 aa, Glycine max
MASLPLGHHHHHHKPAAAAIHPSQPPQSQPQPEVPRRSSDMETDKDMSATVIEGNDAVTGHIISTTIGGKNGEPKETISYMAERVVGTGSFGVVFQAKCLETGEAVAIKKVLQDRRYKNRELQLMRLMDHPNVISLKHCFFSTTSRDELFLNLVMEYVPESMYRVIKHYTTMNQRMPLIYVKLYTYQIFRGLAYIHTALGVCHRDVKPQNLLVHPLTHQVKLCDFGSAKVLVKGESNISYICSRYYRAPELIFGATEYTASIDIWSAGCVLAELLLGQPLFPGENQVDQLVEIIKVLGTPTREEIRCMNPNYTEFRFPQIKAHPWHKVFHKRMPPEAIDLASRLLQYSPSLRCTALEACAHPFFDELREPNARLPNGRPLPPLFNFKQELAGASPELINRLIPEHIRRQMGLSFPHSAGT

SEQ ID NO: 53 - protein, 1044 aa, Glycine max
MESRMDHYEIMEQIGRGAFGAAILVNHKAEKKKYVLKKIRLARQTERCRRSAHQEMALIARIQHPYIVQFKEAWVEKGCYVCIVTGYCEGGDMAALMKKSIGVYFPEEKLCKWFTQILLAVEYLHSNFVLHRDLKCSNIFLTKDQDVRLGDFGLAKTLKADDLASSVVGTPNYMCPELLADIPYGFKSDIWSLGCCIYEMAAHRPAFKAFDMAGLISKINRSSIGPLPPCYSPSLKTLIKGMLRKNPEHRPTASEILKHPYLLPYVDQYRSSFCTPTAGSPERPISAVHHPRKNKPESQNSSSSLSPEKDSFMSSEKNTANEVKKCDRKITEIDLTSIEDDSSEQLLPEEEGNGSSRVNAKTDEKELTKQSNNVHHSNAVSKQPKPIKNVVTALKDGKLRETSSPIRGNRIKVGGVLTHKINSETVSKLPKPNFGAYDLKPNLEVPTTAPSKTTPDSAKRMQGLHTSKHQLPMIESTPKTKPRHNAIPPSGPVKQVEGREVPSKPRQKTRPSLLKPPSFPGHVRQAGFDVPNATNNTGKSSPKKMVWEPKMSHHQLTNTHLPHVSRETTREPLKTFETSSKGMQTDSSNSVSSSLSIQGFELSDFATTFIDLSEPTLPDHESLNHTENVESCPYSISCASYLHFEMSEQLSGETPVVTPCFQNITSNEKVSPSLTLDHSGQDAEVMFASDDSFSINQRTASAGSRCDNLSVDPSAEITQEIKDPQDSKEMSSAKSLQQSLLISGEKSVCEEFGPSSKGSNRLDKVSRPKLMCISTGDDKFMVRERLSSVDETAPSIISTKISSQKVLQEEKGMVLQNPAPERPAVGHLPPAFDDVIHVIRHSSYRVGSEQPGKESVEMGVQNVDVGKFINIARDDLEMRNVSTPLTLKSSNCSEAIGLKSNISDNMEIRNLSSPPNLKSSSCTDLMNIKSSFSDHLLVRKQDVKNTDSLVSKSDSSEYTKHNTPTAAEETPPKEILDVKSSRQRAEALEGLLELSADLLQQNRLEELAVVLKPFGKDKVSPRETAIWLAKSLKGLMIEESGGRS

SEQ ID NO: 54 - protein, 618 aa, Glycine max
MTTFHVVEKDLTSRYARYDELLGKGAFKTVYKAFDEVDGIEVAWNMISVEDVVQTPQQLEKLYSEIHLLKSLKHDNVIKLYNSWVDDTTGTINMITELFTSGSLRQYRNKHKNVDMKAIKNWARQILRGLCFLHCHSPPIVHRDLKCDNIFVNGNSGLVKIGDLGLAIVMQQPTARSVIGTPEFMAPELYEEEYNELVDIYSFGMCILEMVTCEYPYSECNNPAQIYKKVTSGIKPAALAKVNDPEVKQFIEKCLVPASMRLSASELLKDPFLATENTKEINHDILELPNPHTKLVNPPTCEPHPMEIDSKSRRTSPGSSMGRIEETSQVSFFDLVRMTENNKFMLRGEKNAESTISLTLRIANACGGARNIHFPFYINSDTAISIAEEMVEHLELTNEDVSVIAELINDMIAKLVPNLKPLSEKLSSGTDQLYRPSSEVQNGEQFNCHWPLQSSDYDMKPVFKDLVHSWPVDGDDLEKQESVMSDISVECGITVASDSKVVEPDIFIFDEFWEGFDAFNSTSDVRFCGQEDGHKNQSENSSGSLINSCCCPFKNFDISSICSLTLADKDPSEGLRLEIEAIDTYFEQRFRELEMMRVAAIESLKRRHGEKHTCDVMH

SEQ ID NO: 55 - protein, 515 aa, Glycine max
MDRSTGRGGGGSVDMFLRNYKLGKTLGIGSFGKVKIAEHVRTGHKVAIKILNRHKIKNMEMEEKVRREIKILRLFMHHHIIRLYEVVETPTDIYVVMEYVKSGELFDYIVEKGRLQEDEARHFFQQIISGVEYCHRNMVVHRDLKPENLLLDSKFNIKIADFGLSNIMRDGHFLKTSCGSPNYAAPEVISGKLYAGPEVDVWSCGVILYALLCGTLPFDDENIPNLFKKIKGGIYTLPSHLSPGARDLIPRMLVVDPMKRMTIPEIRQHPWFQVHLPRYLAVPPPDTLQQAKKIDEEILQEVVNMGFDRNQLVESLSNRIQNEGTVTYYLLLDNRFRVSSGYLGAEFQETMDSGFNRMHSGEVASPVVGHHSTGYMDYQGVGMRQQFPVERKWALGLQSRAQPREIMTEVLKALQELNVCWKKIGHYNMKCRWVAGTAGHHEGMINNSLHSNHYFGNDSGIIENEAVSKSNVVKFEVQLYKTREEKYLLDLQRVQGPQFLFLDLCAAFLSQLRVL

SEQ ID NO: 56 - protein, 382 aa, Glycine max
MGNACAKGKPVAHVSSSNFSGSKKPASKPKQYSNSSEQRSAPTTSELNVPKSISSNLKSFSLNDLKEATKNFRRENLIGEGGFGRVFKGWIDENTYGPTKPGTGIVVAIKNLKPESFQGHKEWLQEVNYLGMLQHENLVKLIGYCLEGKNRLLVYEFMQKGSLENHLFRKGVQPMAWVTRVNIAIGVARGLTFLHSLDQNVIFRDLKASNILLDSDFNAKLSDFGLARDGPTGDNTHVSTRVIGTQGYAAPEYVATGHLTPRSDVYSFGVVLLELLTGRRAVEDDGPGFSEETLVDWAKPFLNDNRRVLRIMDTRLGGQYSKKGAQAAAALALQCLNTDPKFRPPMVEVLAALEALNSSNSFTRTPKHESHSTKISGGPSQK

SEQ ID NO: 57 - protein, 356 aa, Glycine max
MKKGNLGLGLKLSVPVSDQSNFARFLTESGTFKDGDLLVNRDGVRIVSQNDVEAPPPIKPTDNQLTLADIDVIKVVGKGNGGVVQLVQHKWTSQFFALKVIQMNIEESMRKQIAQELKINQQAQCPYVVVCYQSFYENGVISIILEYMDGGSLADLLKKVKTIPEDYLAAICKQVLKGLVYLHHEKHIIHRDLKPSNLLINHIGEVKITDFGVSAIMESTSGQANTFIGTYNYMSPERINGSQRGYNYKSDIWSLGLILLECALGRFPYAPPDQSETWESIFELIETIVDKPPPIPPSEQFSTEFCSFISACLQKDPKDRLSAQELMAHPFVNMYDDLEVDLSAYFSNAGSPLATL

SEQ ID NO: 58 - protein, 451 aa, Glycine max
MGSSFSSCYEGESVSPSPKPTKVVATKGGSSSNRVSITDLSFPGSTLSEDLSVSLVGSNLHVFSLSELKIITQSFSSSNFLGEGGFGPVHKGFIDDKLRPGLEAQPVAVKLLDLDGSQGHKEWLTEVVFLGQLRHPHLVKLIGYCCEEEHRLLVYEYLPRGSLENQLFRRYTASLPWSTRMKIAAGAAKGLAFLHEAKKPVIYRDFKASNILLDSDYNAKLSDFGLAKDGPEGDDTHVSTRVMGTQGYAAPEYIMTGHLTAMSDVYSFGVVLLELLTGRRSVDKGRPQREQNLVEWARPALNDSRKLGRIMDPRLEGQYSEVGARKAAALAYQCLSHRPRSRPLMSTVVNVLEPLQDFDDVPIGPFVYTVPAEQHNEVAKESETPKERKRENDHHHHNRHHHHHRHNGHRHHPLKSPKTPMSSDQSQNDEHRNGRRSGSNSPDTSNASEAQ

SEQ ID NO: 59 - protein, 555 aa, Glycine max
MDSARSWLQKFQPRDKTRAAGKKKEEDGNGGNQDSNEPVDEALLSSVTKQKVAATKQYIENHYKEQMKNLQERKERRTILEKKLADADVSEEDQNNLLKFLEKKETEYMRLQRHKMGVDDFELLTMIGKGAFGEVRVCREKTSDHVYAMKKLKKSEMLRRGQVEHVKAERNLLAEVDRNCIVKLYCSFQDDEYLYLIMEYLPGGDMMTLLMRKDTLTEDEARFYVGETILAIESIHKHNYIHRDIKPDNLLLDRYGHLKLSDFGLCKPLDCSALEEKDFSVGQNVNGSTQSSTPKRSQQEQLQHWQMNRRTLAYSTVGTPDYIAPEVLLKKGYGMECDWWSLGAIMYEMLVGYPPFYSDDPMLTCRKIVNWKTYLKFPEEARLSPEAKDLISKLLCNVNQRLGSKGADEIKAHPFFKGVEWDKLYQMEAAFIPEVNDELDTQNFEKFDESDSQNQSSSRSGPWRKMLSSKDLNFVGYTYKNFEIVNDYQVPGIAELKKKQSKPKRPTIKSLFETSEGSDTDTSANDQPAQGSFLKLLPPQLEVSPHRNKNLPPRS

SEQ ID NO: 60 - protein, 688 aa, Glycine max
MMKHYHVLYMFLFFLPISTLSLHHNDTHALTLFRRQSDLHGYLLSNWTGHDACNSAWRGVLCSPNGRVTALSLPSLNLRGPLDPLTPLTHLRLLNLHDNRLNGTVSTLFSNCTNLQLLYLSSNDFSGEIPPEISSLKSLLRLDLSDNNLRGKVDVISNLTQLITLRLQNNLLSGEIPDLSSSMKNLKELNMTNNEFYGRLPSPMLKKFSSTTFSGNEGLCGASLFPGCSFTTTPPNNNDSNNNNDNNSNEKEPSQTVSSNPSSFPETSIIARPGREQQRKGLSPGAIVAIVIANCVALLVVVSFAVAHCCARGRGSSLVGSGESYGKRKSESSYNGSDEKKVYGGGESDGTSGTDRSRLVFFDRRSEFELEDLLRASAEMLGKGSLGTVYRAVLDDGCTVAVKRLKDANPCARHEFEQYMDVIGKLKHPNVVRLKAYYYAKEEKLLVYDYLSNGSLHALLHGNRGPGRIPLDWTTRISLVLGAARGLAKIHAEYSAAKVPHGNVKSSNVLLDKNGVACISDFGLSLLLNPVHAIARLGGYRAPEQEQNKRLSQQADVYSFGVLLLEVLTGRAPSSQYPSPARPRMEVEPEQAAVDLPKWVRSVVREEWTAEVFDQELLRYKNIEEELVSMLHVGLTCVVAQPEKRPTMEEVVKMIEEIRVEQSPLGEDYDVSCNSLSPSIPTTEDGLA

SEQ ID NO: 61 - protein, 1008 aa, Glycine max
MTNPKNQTTLSIIIIVYILFFSLATTLVSCLNQEGLYLYQLKLSLDDPDSKLSSWNSRDATPCNWYGVTCDAATNTTVTELDLSDTNIGGPFLSNILCRLPNLVSVNLFNNSINETLPSEISLCKNLIHLDLSQNLLTGPLPNTLPQLLNLRYLDLTGNNFSGPIPDSFGTFQNLEVLSLVSNLLEGTIPSSLGNVSTLKMLNLSYNPFFPGRIPPEIGNLTNLQVLWLTQCNLVGVIPTSLGRLGKLQDLDLALNDLYGSIPSSLTELTSLRQIELYNNSLSGELPKGMGNLTNLRLIDASMNHLTGRIPEELCSLPLESLNLYENRFEGELPASIADSPNLYELRLFGNRLTGKLPENLGRNSPLRWLDVSSNQFWGPIPATLCDKGALEELLVIYNLFSGEIPASLGTCQSLTRVRLGFNRLSGEVPAGIWGLPHVYLLELVDNSFSGSIARTIAGAANLSLLILSKNNFTGTIPDEVGWLENLVEFSASDNKFTGSLPDSIVNLGQLGILDFHKNKLSGELPKGIRSWKKLNDLNLANNEIGGRIPDEIGGLSVLNFLDLSRNRFLGKVPHGLQNLKLNQLNLSYNRLSGELPPLLAKDMYRSSFLGNPGLCGDLKGLCDGRGEEKSVGYVWLLRTIFVVATLVFLVGVVWFYFRYKNFQDSKRAIDKSKWTLMSFHKLGFSEDEILNCLDEDNVIGSGSSGKVYKVVLSSGEVVAVKKIWGGVKKEVESGDVEKGGRVQDNAFDAEVETLGKIRHKNIVKLWCCCTTRDCKLLVYEYMPNGSLGDLLHSSKGGLLDWPTRYKIAVDAAEGLSYLHHDCVPAIVHRDVKSNNILLDVDFGARVADFGVAKAVETTPKGAKSMSVIAGSCGYIAPEYAYTLRVNEKSDIYSFGVVILELVTGKRPVDPEFGEKDLVKWVCTTLDQKGVDHLIDPRLDTCFKEEICKVFNIGLMCTSPLPIHRPSMRRVVKMLQEVGTENQTKSAKKDGKLSPYYYDDASDHGSVV

SEQ ID NO: 62 - protein, 512 aa, Glycine max
MADVVSKSPTPTSNLISPNKKETSNLLLGRFEIGKLLGHGTFAKVYYARNIKTGEGVAIKVIDKEKILKGGLVAHIKREISILRRVRHPNIVQLFEVMATKSKIYFVMEYVRGGELFNKVAKGRLKEEVARKYFQQLISAVGFCHARGVYHRDLKPENLLLDENGNLKVSDFGLSAVSDQIRQDGLFHTFCGTPAYVAPEVLARKGYDGAKVDLWSCGVVLFVLMAGYLPFHDQNVMAMYKKIYRGEFRCPRWFSPDLSRLLTRLLDTKPETRIAIPEIMENKWFKKGFKQIKFYVEDDRLCNVVDDDGLMDNDDDTASIVSVASFSDYSVSESDSEIETRRRINAPLPRPPSLNAFDIISFSPGFNLSGLFEEKEDETRFVTAAPVNRIISKLEEIAQLVRFSVRKKDCRVSLEGTREGVRGPLTIAAEIFELTPSLVVVEVKKKGGDRAEYERFCNDELKPGLQNLMVEESATSSELSTPIQPSLLRGLSEPVPDISSDIETPLCIPSDD

SEQ ID NO: 63 - protein, 1000 aa, Glycine max
MLQLLPVFLLLLLEQASLISGLNQDGLYLYEWKQSLDDPDSSLSSWNNRDATPCNWAGVTCGPSNTTVTALDLSNFNLSGPFSASLLCRLPNLTSIILFNNSINQTLPLQISLCTPLLHLDLSQNLLTGFLPHTLPLLPNLLHLDLTGNNFSGPIPPSFATFPNLQTLSLVYNLLDDVVSPSLFNITTLKTLNLSFNPFLPSPIPHSLGNLTNLETLWLSGCNLVGPIPESLGNLVNLRVLDFSFNNLYGPIPSSLTRLTALTQIEFYNNSLSAEFPKGMSNLTSLRLIDVSMNHLSGTIPDELCRLPLESLNLYENRFTGELPPSIADSPNLYELRLFGNKLAGKLPENLGKNAPLKWLDVSTNRFSGGIPESLCEHGELEELLMLENEFSGEIPASLGGCRRLSRVRLGTNRLSGEVPAGMWGLPHVYLLELGNNSFSGPIARTIAGARNLSLLILSKNNFSGVIPDEIGWLENLQEFSGADNNFNGSLPGSIVNLGQLGTLDLHNNELSGELPKGIQSWKKLNDLNLANNEIGGKIPDEIGILSVLNFLDLSNNEISGNVPLGLQNLKLNLLNLSYNRLSGRLPPLLAKDMYRASFMGNPGLCGDFKGLCDGKGDDDNSKGFVWILRAIFIVASLVFVVGVVWFYFRYRNFKNAGRSVDKSKWTLMSFHKLGFSEDEILNCLDEDNVIGSGSSGKVYKVVLTSGESVAVKKIWGGVKKEIDSGDVEKGHQFRQDSSFDAEVETLGKIRHKNIVKLWCCCTTRDSKLLVYEYMPNGSLGDLLHSNKGGLLDWPTRYKIAVDAAEGLSYLHHDCVPSIVHRDVKSNNILLDGDFGARVADFGVAKVVDATGKGTKSMSVIAGSCGYIAPEYAYTLRVNEKSDIYSFGVVILELVTGRRPIDPEFGEKDLVMWACNTLDQKGVDHVIDSRLDSCFKEEICKVLNIGLMCTSPLPINRPAMRRVVKMLQEVGTENQTKPAKKDGKLSPYYYDDGSDHGSVA

SEQ ID NO: 64 - protein, 684 aa, Glycine max
MIRHKHTHHLPSILLSILFLFNSTCAIDFVFNGFNSSEVLLFGNATVDSRILTLTHQQRFSVGRALYNKKIPTKKPNSSRVYPFSTSFIFAMAPFEDTLPGHGLVFIFTPVTGIQGTSSAQHLGLFNLTNNGNSSNHVFGVEFDVFQNQEFDDIDANHVGIDINSLKSYVSHDAGYWPDGADKSFKELTLNSGENYQVWIDYEDSWINVTMAPVGMKRPSRPLLNVSLNLSQVFEDEMFVGFTSATGQLVESHKILGWSFSNEKFSLSDELITTGLPSFVLPKDSIFKSKGFVAGFTVGVFFVICLLVLLALFLIQRKREKERKRMEMEDWELEYWPHRMTYEEIEAATKGFSEENVIGVGGNGKVYKGVLRGGVEVAVKRISHENDGLREFLAEVSSLGRLKQRNLVGLRGWCKKDVGNFLLIYDYMENGSLDKRVFDCDESKMLSYEDRIRILKDVAFAVLYLHEGWEDKVVHRDIKASNVLLDKDMNGRLGDFGLARMHSHGQVASTTKLVGTVGYMAPEVFKTGRASTQTDVYMFGILILEVLCGRRPLEEGKPPLVEWIWQLMVQGQVECALDERLRAKGEFNVQEMERVMHLGLLCAYPEPKTRPTMRQVVNVLEGKNEVEDSEIENMDTYLLQQLKSRDILSEYSQYFSYTSHPTFQDIRLSSSMSLTWSESVVEGR

SEQ ID NO: 65 - protein, 821 aa, Glycine max
MELLKPLHPHHAPILRIPFHAVPSSSSSQSQSQVPLPMISKVHVAVGKSLDKVVPLLRWTLNHFRNAEIVIVHAYQPSLTIPTLLGKLPASQASPAVVSAFRKVEREQIMKLLDKYLSICRAARVKAAIIVTEADQVQKGIVDLVIKHNIEKLVIGAVPENCMKVKRNSSKANYTAKNAPPFCEVWFIYKGKHIWTREASETPCSSSSCTQPEIATTESLRCRSFQYGKNELFDSEYLWPNSARTTAVSGIRSWVQGEIIETEATFSSKASSCCSHCSPQNSSRAYFDTYLEVMEERINKQLIETKREAEAVTDEAFTELLKCEKLEVEAMEAIRKVNLFESAHVREVKLRKEADDALRDTVQEQQKLLNESEEIAGELQMTMRNIALLDSRAQEANRRRDEAADELSLIQESISTLWQERQQIRRQKTEALRWLERWRSRGQVGAAHCNGVIGFAEELPELAEFSLSDLQNATCNFSNSFKIEQGGYSCIYKGEMLGRTVAIKKFHQHNMQGPLEFRQEVQVLGSLQHPHLITLLGVCPEAWSIVYEYLPNGTLQDYLFRKSNNSPLTWNTRARMIAEIASALCFLHSFKPETIIHGDLKPETVLLDSSLGCKMCGFGLCRLVSEESLLRPSFRLSTEPKGAFTYTDPEFQRTGILTTKSDIYSFGLIILQLLTGRTPVGLAVLVRNAVSCGKLSSILDSSAGEWPSAVAMQLVELGLQCCQQYHRDRPELTPTLVRELEQLHASEERPVPSFFSCPILQEIMHDPQVAADGFTYEGDAIREWLENGHDTSPMTNLKLSHLFLTPNYALRLAIQDWLCKS

SEQ ID NO: 66 - protein, 1027 aa, Glycine max
METSLDYSKMLFLLLLGSTSLIFLSHLASAATPKLNTQEVKALKEIGSKIGKKDWNFGVDPCSGKGNWNVPDARKAFVMSSVICDCSFNHNSSCHVVSIYWKAQNLSGSLSPEFSKLHYLQKLDLSRNIITGSIPPQWGTMRLVELSLMGNKLSGPFPKVLTNITTLRNLSIEGNQFSGHIPTEIGKLTNLEKLVLSSNGFTGALPPVLSKLTKLIDLRISDNNFLGKIPDFISNWTLIEKLHMHGCSLEGPIPSSISALTRLSDLRITDLKGSKSSAFPPLNNLKSMKTLVLRKCMIKGEIPEYIGRMEKLKILDLSYNGLSGEIPESFAQLDKVDFMYLTGNKLSGIIPRWVLANNENIDISDNNFSWDSSSPTECQRGSVNLVESYSSSVNTQTKINSCLKKNFLCTASPSQYRYSLNINCGGNEANVSGNIYEADREQKGAAMLYYTSQDWALSSTGNFMDNDIDSDPYIVANTSRLNVSALNSKLYTTARVSPLALTYYGLCLINGNYTVKLHFAEIIFINDRSLNSLGRRVFDVYIQGNLVLKDFDIRREAGGTGKSIEKTFNASVTQHTLKIHFYWAGKGTTGIPTRGVYGPLVSAISVNPNFKPPSGEGKRTYLILAIIIVAGVLVVVLLVLVLLRRMGWLGGKDPVYKELRGIDLQTGLFTLRQIKAATKNFDALNKIGEGGFGCVYKGQQSDGTMIAVKQLSSKSKQGNREFVNEMGLISGLQHPNLVKLYGCCVEGNQLILIYEYMENNCLSRILFGRDPNKTKLDWPTRKKICLGIAKALAYLHEESRIKIIHRDVKASNVLLDKDFNAKVSDFGLAKLIEDEKTHISTRVAGTIGYMAPEYAMRGYLTDKADVYSFGVVALETVSGKSNTNFRPNEDFVYLLDWAYVLQERGSLLELVDPNLGSEYLTEEAMVVLNVALLCTNASPTLRPTMSQVVSMLEGWTDIQDLLSDPGYSAISSSSKHKSIRSHFWQTPSGTHSISIPSIYTDSSGSHVETEKNYHPVTVNSDGSDKSN

SEQ ID NO: 67 - protein, 710 aa, Glycine max
MAVSNGVEAVLEFLRKNGLSEAESALRQDIIENNDLGNFDYEKFFFPMVPPPPPVRVRSFSRLSELSADGNCSKSSSDEFVSIGSPTSRVSSSEFINPYGIRSSSQTQNDSASSSERLSQFGTARDYHDFEMQNEPYWYNEKDDDYFMTPSFEGPDFFACQSEDKFVMTAETENQHDNSLDLVYNSEEFLLKGNGNGGFMDKACLYNHSSVRDGNATYSKEYCHVDNNNLFEGELEGKAEKHTVACSCEVPFCKSSPGGSCSLDPTNFGYPNLKEIHLKFGDINSFDSTSELTVNQSFDYYTKNDSSKEYNGPYDLTIKVNQKDLPNGLDTYKARDGGELAEECQDPEITADGEDTTDDELLKYTQEEEYEVFDLRIIHRKNRTGFEENKELPIVLNTVLAGRYYVTEYLGSAAFSRVVQAHDLQTGIDVCLKIIKNDKDFFDQSLDEIKLLKLVNKHDPADLHHFLRLYDYFYHQEHLFIVTELLQANLYEFQKFKQESGGEEYFTLNRLQLITRQCLEALQYLHSLGIVHCDLKPENILIKSYRRCEIKVIDLGSSCFQTDNLCLYVQSRSYRAPEVMLGLQYDEKIDIWSLGCILAELCSGEVLFPNDAVVMILARMIGMFGSIDMEMLVKGQETHKYFTKEYDIYYVNEETDQLEYIIPEESSLEQHLQVTDTTFIDFVRYLLSINPKRRPTARQALRHPWLSYVY

SEQ ID NO: 68 - protein, 861 aa, Glycine max
MGMGGIAVSVVTVLLLMIGLTMGMSLSSRTEWFALRELRQSLEIRAKYWPIKAEPCGNWTGVQCRNGRVVGINVSGLRRTRWGRLNPSFEVDSLVNFTLLETFNASGFKLNGSIPEWLGERLGVLEELDLSLCSIKGSIPDSIGRLSKLKVLLLSGNFLTGRMPSTLGNLTRLSVLDLSGNSLSWPVPDSVSKLGNLSRLDLSYNFLSGSVPPELGALSSLQFLNLSGNSFTGSVPSQLGNLSKLVEVDLSMNFLSRSLSGGLFSSVVLALEVLILRGNLLDGVLPANLRSMPRLHFLDVSSNNLTGTLPNFADWNVSSAGVVFNLSNNMFYGLLNTSLDRFKMIDLSSNFLEGEVLGGGGGVSNVDLDRNCLQRIPNQRNLEDCRMFYDKRNLSSAFPESESRSRRRVIFMLVGIFGGLGFIVLLALVLMLVLKQCHNRKSLEVPRETKDGGAVEEGESPIPPKDIDFVTGVGEAYSFEQMLRLTGNFAESNVIKHGHSGDLFLGVLEGGATVVVKKVDLNLFKRESYVVELGLLSKVPHARLVPILGHCLDNENEKCIVYKYMPNRDLATSLHRVTGSDGKIQSLDWITRLKIAIGAAEGIAYLHECSPPLVHRDIQASSILLDDKFEVRLGSLSEVTAQGDLQQGVISRVFSKPPSSNQADSGKSPVTCTYDVYCFGKILLELITGNIEVSKSDDATTKEWLEQTLPYITIYDKERVTKIIDPSLIVDEDLLEEVWAMAIVANACLKPKPSKRPPMRHVLKALENPLKIVREENTSSARLRTNSSRKSWSTAFFGSWRHSSSDSVVATNKEGSNDTKKSGKVGSQSSGNDHSSSNKRSSNEIFPEPLEIQDVETGVTR

SEQ ID NO: 69 - protein, 655 aa, Glycine max
MTEVTLNSSVNILYHTAKKISMKFYSLQAHRFLFIIVILCPLVIADLSSDKQALLDFAAAVPHRRNLKWNPATPICSSWVGITCNLNDTRVVSVRLPGIGLVGTIPANDTRIDSLRNISLRANLLSGSLPADITSLPSLQYLYLQHNNLSGNIPTSLSTRLNVLDLSYNSFTGAIPKTLQNLTQLIKLNLQNNSLSGLIPNLNVTKLRRLNLSYNHLNGSIPAALQIFPNSSFEGNSLCGLPLKSCPVVPSTPPPSSTPAPPSTPARHSSKSKLSKAAIIAIAVGGGVLLLLVALIIVLCCFKKKDDGSPRATKGKGPSGGRSEKPKEEFGSGVQEPEKNKLVFFEGSSYNFDLEDLLRASAEVLGKGSYGTAYKAILEESTTVVVKRLKEAVVGKREFEQQMEIVGRVGHHPNVVPLRAYYYSKDEKLLVYDYIPSGNLSTLLHGNRASGRTPLDWNSRIKISVGIARGIAHIHSVGGPKFAHGNVKSSNVLLNQDNDGCISDFGLTPLMNVPSTPSRAAGYRAPEVIETRKHTHKSDVYSFGVLLLEMLTGKAPQQSPGRDDMVDLPRWVQSVVREEWTAEVFDVELMRYQNIEEEMVQMLQIAMACVAKVPDMRPSMEEVVRMIEEIRLSDSENRPSSEENRSKEESTAQTP

SEQ ID NO: 70 - protein, 941 aa, Glycine max
MKALALLAIIVFTLLVRSQEEEDYDDASVMLALKNSLNPPGWSDPDPCKWARVLCSDDKRVTRIQIGRLNLQGTLPTTLQKLTHLEHLELQYNNISGPLPSLNGLTSLRVFLASNNRFSAVPADFFAGMSQLQAVEIDSNPFEPWEIPQSLRNASGLQNFSANSANVGGSIPEFFGSDVFPGLTLLHLAMNNLEGTLPLSFSGSQIQSLWLNGQKSVNKLGGSVEVLQNMTFLTDVWLQSNAFTGPLPDLSGLKSLRDLSLRDNRFTGPVPVASFVGLKTLKVVNLTNNLFQGPMPVFGDGVVVDNVKDSNSFCLPSPGDCDPRVDVLLSVVGVMGYPPRFAESWKGNDPCAYWIGITCSNGYITVVNFQKMELSGVISPEFAKLKSLQRIVLADNNLTGSIPEELATLPALTQLNVANNQLYGKVPSFRKNVVVSTNGNTDIGKDKSSLSPQGLVPPMAPNAKGDSGGVSGIGGKKSSSHVGVIVFSVIGAVFVVSMIGFLVFCLFRMKQKKLSRVQSPNALVIHPRHSGSDNESVKITVAGSSVSVGAASETRTVPGSEASDIQMVEAGNMVISIQVLKNVTDNFSEKNVLGQGGFGTVYRGELHDGTRIAVKRMECGAIAGKGAAEFKSEIAVLTKVRHRHLVSLLGYCLDGNEKLLVYEYMPQGTLSRHLFDWPEEGLEPLEWNRRLTIALDVARGVEYLHGLAHQSFIHRDLKPSNILLGDDMRAKVADFGLVRLAPEGKASIETRIAGTFGYLAPEYAVTGRVTTKVDVFSFGVILMELITGRKALDETQPEDSMHLVTWFRRMSINKDSFRKAIDSTIELNEETLASIHTVAELAGHCGAREPYQRPDMGHAVNVLSSLVELWKPSDQNSEDIYGIDLDMSLPQALKKWQAYEGRSQMESSASSSLLPSLDNTQTSIPTRPYGFADSFTSADGR

SEQ ID NO: 71 - protein, 337 aa, Glycine max
MEKTGVLSAKEAFEKLEKVGEGTYGKVYRAREKATGKIVALKKTRLHEDEEGVPPTTLREVSILRMLSRDPHVVRLMDVKQGQNKEGKTVLYLVFEYMDTDLKKFIRSFRQSGETIPPHIIKVILSICPSIFNIRFFFVHLFLSIWLQSLMYQLCKGVAFCHGHGILHRDLKPHNLLMDRKTMMLKIADLGLARAFTVPIKKYTHEILTLWYRAPEVLLGATHYSMAVDMWSVGCIFAELVTKQALFPGDSELQQLLHIFRLLGTPNEDVWPGVSKLMNWHEYPQWNPQSLSTAVPSLDELGLDLLSQMLKYEPSKRISAKKAMEHVYFDDLDKRHL

SEQ ID NO: 72 - protein, 710 aa, Glycine max
MARARFPLSILLSLVFVALPLSLANTDPSDVQALEVMYNALNSPTQLTGWKIGGGDPCGESWKGVTCEGSAVVSIKLSGLGLDGTLGYLLSDLMSLRELDLSDNKIHDTIPYQLPPNLTSLNFARNNLSGNLPYSISAMVSLNYLNLSNNALSMTVGDIFASLQDLGTLDLSFNNFSGDLPPSFVALANLSSLFLQKNQLTGSLGVLVGLPLDTLNVANNNFSGWIPHELSSIRNFIYDGNSFENSPAPLPPAFTSPPPNGPHGRHHSGSGSHNKTQVSDNEKSDGHKGLTVGAVVGIVLGSVLVAAIVLLALVFCIRKQKGKKGARNFSGSLPRGVINVTPQMQEQRVKSAAVVTDLKPRPAENVTVERVAVKSGSVKQMKSPITSTLYTVASLQSATNSFSQEFIIGEGSLGRVYKADFPNGKVMAIKKIDNSALSLQEEDNFLEAVSNMSRLRHPSIVTLAGYCAEHGQRLLVYEYIANGNLHDMLHFAEDSSKALSWNARVRIALGTARALEYLHEVCLPSVVHRNFKSANILLDEELNPHLSDCGLAALTPNTERQVSTQMVGSFGYSAPEFALSGVYTVKSDVYSFGVVMLELLTGRKPLDSLRVRSEQSLVRWATPQLHDIDALAKMVDPTLNGMYPAKSLSRFADIIALCVQPEPEFRPPMSEVVQALVRLVQRASVVKRRPSEESGFGHKTPDHEAMDMPF

SEQ ID NO: 73 - protein, 960 aa, Glycine max
MEIFKCFYIKNLILVTFFMVSSAVLAIDPYSEALLSLKSELVDDDNSLHNWVVPSGGKLTGKSYACSWSGIKCNNDSTIVTSIDLSMKKLGGVVSGKQFIIFTNLTSLNLSHNFFSGQLPAEIFNLTSLTSLDISRNNFSGPFPGGIPRLQNLVVLDAFSNSFSGPLPAEFSQLENLKVLNLAGSYFRGSIPPEYGSFKSLEFLHLAGNSLTGSIPPELGHLKTVTHMEIGYNEYQGFIPPELGNMSQLQYLDIAGANLSGPIPKQLSNLTSLQSIFLFRNQLTGSIPSELSIIEPLTDLDLSDNFLIGSIPESFSELENLRLLSVMYNDMSGTVPESIAKLPSLETLLIWNNRFSGSLPPSLGRNSKLKWVDASTNDLVGSIPPDICASGELFKLILFSNKFTGGLSSISNCSSLVRLRLEDNSFSGEITLKFSHLPDILYVDLSKNNFVGGIPSDISQATQLEYFNVSYNPQLGGIIPSQTWSLPQLQNFSASSCGISSDLPLFESCKSISVIDLDSNSLSGTIPNGVSKCQALEKINLSNNNLTGHIPDELASIPVLGVVDLSNNKFNGPIPAKFGSSSNLQLLNVSFNNISGSIPTAKSFKLMGRSAFVGNSELCGAPLQPCPDSVGILGSKGTWKVTRIVLLSVGLLIVLLGLVFGILYLRRGIKSQWKMASFAGLPQFTANDILTSLSATTKPTDIQSPSVTKTVLPTGITVLVKKIELEARSIKVVSEFIMRLGNARHKNLIRLLGFCHNQHLVYLLYDYLPNGNLAEKMEMKWDWAAKFRTVVGIARGLCFLHHECYPAIPHGDLRPSNIVFDENMEPHLAEFGFKHVSRWSKGSSPTTTKWETEYNEATKEELSMDIYKFGEMILEILTRERLANSGASIHSKPWEVLLREIYNENGASSASSLQEIKLVLEVAMLCTRSRSSDRPSMEDVLKLLSGLKHLEDGRTSKEGQ

SEQ ID NO: 74 - protein, 527 aa, Glycine max
MGNETRKLSDEYEVSEVLGRGGFSVVRKGTKKSSSDTKTHVAIKTLRRVGTASNSNNPSGFPRPKGGEKKSTAAMMGFPTWRQVSVSDALLTNEILVMRRIVENVSPHPNVIDLYDVYEDSNGVHLVLELCSGGELFDRIVAQDRYSETEAAGVVRQIASGLEAIHRANIVHRDLKPENCLFLDVRRDSPLKIMDFGLSSVEEFTDPVVGLFGSIDYVSPEALSQGKITTKSDMWSLGVILYILLSGYPPFIAQNNRQKQQMIMNGNFSFYEKTWKGITRSAKQLISDLLIVDPSRRPSAQDLLSHPWVVGDKAKDDAMDPEIVSRLQSFNARRKLRAVAIASIWSTTIFLRTKKLKSLVGTHDLTEEEIENLRMSFKKICVSGDNATLSEFEEVLKAMNMPSLIPLAPRIFDLFDDNRDGTVDMREILCGFSSFKNSKGDDALRLCFQMYDTDRSGCITKEEVASMLRALPEDCLPTDITEPGKLDEIFDLMDANSDGKVTFDEFKAAMQRDSSLQDVVLSSLRPQ

SEQ ID NO: 75 - protein, 614 aa, Glycine max
MVLHSWISSSHILVNFLLLLGCGITYGTDTDIFCLKSIKESLEDPYNYLKFSWDFNNKTEGYICRFNGVECWHPDENRVLNLKLSNMGLKGQFPRGIQNCSSLTGLDLSINKLSGTIPGDISTLIPFATSIDLSTNEFSGAIPVSLANCTFLNTLKLDQNRLTGQIPPQFGVLSRIKVFSVSNNLLTGQVPIFRDGVELHYANNQGLCGGNTLAPCQATPSKSNMAVIAGAAAGGVTLAALGLGIGMFFFVRRVSFKKKEEDPEGNKWARSLKGTKRIKVSMFEKSISKMKLSDLMKATNNFSNTNIIGTGRTGTVYKAVLDDGTTLMVKRLQESQYTEKEFMSEMGTLGTVKHRNLVPLLGFCMTKRERLLVYKNMPNGNLHDQLHPADGVSTLDWTTRLKIAIGAAKGLAWLHHSCNPRIIHRNISSKCILLDADFEPKISDFGLARLMNPIDTHLSTFVNGEFGDLGYVAPEYTRTLVATPKGDIYSFGTVLLELVTGERPTNVSKAPETFKGNLVEWITELTSNAKLHDAIDESLVRKDVDSELFQFLKVACNCVSPTPKERPTMFEVYQLLRAIGGRYNFTTEDDILVPTDIGNTDNMQELIVAQEGSY

SEQ ID NO: 76 - protein, 456 aa, Glycine max
MGSSFSSCYEGESVSPSPKPTKVVATKGGSSSNRVSITDLSFPGSTLSEDLSVSLVGSNLHVFSLAELKIITQGFSSSNFLGEGGFGPVHKGFIDDKLRPGLEAQPVAVKLLDLDGSQGHKEWLTEVVFLGQLRHPHLVKLIGYCCEEEHRLLVYEYLPRGSLENQLFRRYTASLPWSTRMKIAAGAAKGLAFLHEAKKPVIYRDFKASNILLDSDYNAKLSDFGLAKDGPEGDDTHVSTRVMGTQGYAAPEYIMTGHLTAMSDVYSFGVVLLELLTGRRSVDKGRPQREQNLVEWARSALNDSRKLSRIMDPRLEGQYSEVGARKAAALAYQCLSHRPRSRPLMSTVVNVLEPLQDFDDVPIGPFVYTVPAEQQQYNEVAKESETPKERKRENGHHHNRRHHHHRHNGHRHHPLKSPKTPMPSDQSQNDEHRNGRKSGSNSPDTFNASEAQRSMG

SEQ ID NO: 77 - protein, 343 aa, Glycine max
MGCSFSGLNALYDSVNGGGDVWINENRFRIVRQLGEGGFAYVYLVKETPNDSAVAAGLSKKLKGSSHLSDDGTYAMKKVLIQNNEQLELVREEIRVSSLFNHPNLLPLLEHAIISVKPTQETSWNHEAYLLFPVHLDGTLLDNAKIMKAKKEFYSTSDVLQIFRQLCAGLKHMHSFDPPHAHNDVKPGNVLITHRKGQPPLAILMDFGSARPARKQIGSRSEALQLQEWASEHCSAPFRAPELWDCPSQADIDERTDIWSLGCTLYAIMYGVSPFEYALGESGGSLQLAIVNAQVKWPAGPKPPYPEALHQFVSWMLQPTASMRPRIDDIIIHVDKLVAKFSQ

SEQ ID NO: 78 - protein, 377 aa, Glycine max
MKCFFFKEKCKSAPELHKKKTPAVNRAANSTGSVSSPKSVKDLYREKEHSFRVFTLQELRDATNGFNRMLKLGEGGFGSVYKGSITQPDGQGGDPIPVAIKRLNTRGFQGHKEWLAEVQFLGIVNHPNLVKLLGYCSVDAERGIQRLLVYEFMPNRSLEDHLFNKNLPTLPWKTRLEIMLGAAQGLAYLHEGLEIQVIYRDFKSSNVLLDADFHPKLSDFGLAREGPQGDQTHVSTAVVGTQGYAAPEYIETGHLKVQSDMWSFGVVLYEILTGRRSLERNRPTAEQKLLDWVKQYPADTSRFVIIMDARLRNQYSLPAARKIAKLADSCLKKNPEDRPSMSQIVESLKQALQYSDTTSQDIAESSSSSRSKLVRKK

SEQ ID NO: 79 - protein, 674 aa, Glycine max
MLGTPHTLILLLFLFLITTFSHSLVFNITNFDDPAAATAISYEGDGRTTNGSIDLNKVSYLFRVGRAIYSKPLHLWDRSSDLAIDFVTRFTFSIEKLNLTEVAYGDGFAFYLAPLGYRIPPNSGGGTFGLFNATTNSNLPENHVVAVEFDTFIGSTDPPTKHVGVDDNSLTSAAFGNFDIDDNLGKKCYTLITYAASTQTLFVSWSFKAKPASTNHNDNSSSFSYQIDLKKILPEWVNIGFSASTGLSTERNTIYSWEFSSSLNGSPADFENVKLKHQSSKLALILAVLCPLVLLFVLASLVAVFLIRKKRRSHDDCMLYEVGDDELGPTSVKFDLDKGTIPRRFEYKELVDATNGFSDDRRLGQGASGQVYKGVLSYLGRVVAVKRIFADFENSERVFTNEVRIISRLIHKNLVQFIGWCHEEGEFLLVFEYMPNGSLDSHLFGNKRVLEWHLRYKIVLGVVNALHYLHEDAEQCVLHRDIKSANVLLDTEFNTKVGDFGMAKLVDPRLRTQRTGVVGTYGYLAPEYVNVGRASRESDIYSFGVVSLEMASGRRTYQDGEFHVSLMNWVWQLYVEGEIMRAADEKLNNEFEVDQMRSLLVVGLWCTNPNDKERPKAAQVIKVLNLEAPLPVLPLDMYERAPPMEIIRMPHHPSGKNHSGMSTPITSSLVSVGR

SEQ ID NO: 80 - protein, 712 aa, Glycine max
MLRDESVGSRSARHSATATATVSNKVLVAVKAEKVISNTALAWALTHVAHSTDSITLLAVYSSHKTGRRFWNFSRLAGDCTNGPAGKLPEQISDISESCAQMVLQLHNQIEVRVKIKVVTGTPSGAVAAEARWSGSHWVILDKKLKQEVKHCTDELNCSIVVMNGSQAKILRLNLRSSNELQTPFFSANSSPGIEIAKLKGRRLKHSTPVGSPEEAVYEQNPLYEGQGPEKRTDESINEPTKDFHVQPPLYFDLERDSPPPSWTRPASSVASDNKTIFWIPQNHNIVDKFQKTKNNSVIQRTKSPTSKTLLENFIRCDQEIWTNELGFDQAQSRSYVPNLGIRDNNSVPLGRTTSIPPPLCSQCKNKAPVFGKPPKRFSYKELEEATDMFSDENFLAEGRFGVVHQGILKDGQVVAVKQLKFGGSQADLDFCREVRVLSCAQHRNVVLLIGFCIESNLRILVYEYICNGSLDLYLYGDESMPLDWNSRLKIAIGTARGLRYLHEDCRVGCIAHRDLRPKNILVTHDFEPMVADFGLARWHSEWNIDTEDRVIGTSGYLAPEYLDAGNLTYKVDVYAFGIVLLELITGRRISELEQFNGHSYLSEWFHPIRMLEPGHILQNVRSLKPCFDSKESVEFNLQLQAMARAVSLCLRVDPDARPPMSKILRVLEGGNPVRPMGLDINSVGNTSGHLSGLKSHTPPKGTINHSRRLSH

SEQ ID NO: 81 - protein, 854 aa, Glycine max
MGMEGIAVSVVTMLLLMVGLTMGMSSRTEWFALRELRQSLEIRAKYWPIKAEPCGNWTGVQCRNGRVVGINVSGLRRTRWGRLNPSFEVGSLVNFTLLETFNASGFKLNGSIPEWLGESLGVLEVLDLSFCSIKGSIPDSIGWLSKLKVLLLSGNFLTGRMPSTFGNLTRLSVLNLSGNSLSGTVPDSVSKLGNLSRLDLSYNFLSGSVPPELGALSSLQFFNLSGNSFTGTFPSQLGNLSKLVDVDLSMNFLSGSLPGGSSSSGLLALKVLILRGNLFDGVLPADLWPMPRLHFLDVSSNNLTGTLPNFTSWNVSSVGFVFNLSNNLFYGLLNTSLDRFEIIDLSSNYLEGEVPGGGVNNVSLDRNCLQRIPNQRDLEDCRVFYDNRSLPFGFLKSGSRSRVIFILVGIFGGLGFIVLLALVLMLVLKQCHNRRSLGVQRGTKDGGPVQEGESPIPPKDTVFVTVGDAFSFEQMLHLTSNFAEANVIKHGHSGDLFLGVLEGGATVVVKKVDLNLFKRESYVVELGLLSKVPHARLVPILGHCLDNENEKFIVYKYMPNRDLATSLHRVTGSDGKLQSLDWITRLKIAIGAAEGIAYLHECSPPLVHRDIQASSILLDDKFEVRLGSLSEVTAQGDLQQGVISRVFSKPPSSNQADSGKSSVTCAYDIYCFGKILLELITGNIEVSKLDDASTKEWLEQTLPYITIYDKERVTKIIDPSLIVDEDLLEEVWAMAIVANACLNPKPSKRPPMRHVLKALENPLKIVREENTSSARLRSNSSRKSWSTAFFGSWRHSSSDSVVATNKEGSSDTKKSGKVGSQSSGNDHSSSNKRSSNEIFPEPLEILDVETGVAR

SEQ ID NO: 82 - protein, 510 aa, Glycine max
MSANSSLHVELSRKTSFLGLRLWVLIGIGVGVFIVVILCVLSVWVMFRRKSKRSLDKYSLSQIPHVSKDIIVDMVGVQISHDQSESVAIPVHDKPSENNSNKLFSHLHKSKSGDADNISQCSSVYHHERGFSSMSGEEGSSGTVKKQSALSFGGMVTASPLVGLPEISHLGWGHWFTLRDLELATNRFSPENVIGEGGYGVVYRGKLINGSEVAVKKILNNLGQAEKEFRVEVEAIGHVRHKNLVRLLGYCVEGVHRLLVYEYVNNGNLEQWLHGAMSQQGTLTWEARMKVITGTAKALAYLHEAIEPKVVHRDIKSSNILIDTEFNAKVSDFGLAKLLDSGESHITTRVMGTFGYVAPEYANTGLLNERSDIYSFGVLLLEAVTGKDPVDYSRPANEVNLVEWLKMMVGTRRAEEVVDSRLEVKPSIRALKRALLVALRCVDPEAEKRPKMSQVVRMLEADEYPFREDRRNRKSRTASMEIESLKDISGPSDAEKLKGSEGHEPETTQG

SEQ ID NO: 83 - protein, 649 aa, Glycine max
MEKTIHLVYFKCLVFLLMKQVIVMAEPRARTVNITCNNKLEHNTTIFVPNFVATMEKISEQMRNTGYGTAVVGTGGPDTNYGLAQCYGDLSLLDCVLCYAEARTVLPQCFPYNGGRIYLDGCFMRAENYSFYDEYIGPGDKAVCGNTTRKSTSFQAAAKKAVLSAVQAAANNKGYARKEVFVAGTTNDAAYVLANCWRSLDTRSCRACLENASSSILGCLPWSEGRALNTGCFMRYSDTDFLNKEQENGSSGGNVLVIVVAVVSSVIVLVVGIAIVVYIRKHRYIQMKRRGSNDAEKLAKSLHHNSLNFKYSTLEKATNSFDEANKLGQGGFGTVYKGVLADGREIAIKRLYFNNRHRAADFFNEVNIISSVEHKNLVRLLGCSCSGPESLLIYEYLPNRSLDRFIFDKNKGRELNWDKRYDIIIGTAEGLVYLHENSNIRIIHRDIKASNILLDAKLRAKIADFGLARSFQEDKSHISTAIAGTLGYMAPEYLAHGQLTEKADVYSFGVLLLEIITGRLNNRSKASEYSDSLVTMTWKHFQSGTAEQLIDPCLVVDDNHRSNFKNEILRVLHIGLLCTQEIPSLRPSMSKALKMLTKKEEHLDLEAPSNPPFIDESTMELHDQNDDPFYPLNAEDSLATMSHSSFYAR

SEQ ID NO: 84 - protein, 349 aa, Glycine max
MLFDRLAFCCSKHTSSSQRKYPTVIEELCHQFSLANLKKSTNNFDENGVIGYGRFGKVYKGCLQHNDGSDYSVTLKRLGVKDSRGLEQFKNEIELLCQLRHPNCVSLIGFCNHKKEKILVYEYMSNGSLHQHLRGGLLSWKKRLEICIEAAHGLHYLHTGAKRTIIHRNINPSNILLDNNMKSKLTDFRLSIQGPRYGSKPKPIKVYVIEGSLGYMPMEYIVDGTITDKFDVFSFGMVLLEVVCGRNCLIIPTETEVLEKPVEENIDQNIKGKIAPECWQVFIDIIIRCLKYEPDERPTMGEVEVQLEHALSMQEQADITNTNSDYTLFSTTTIHLGLELESNPEESDT

SEQ ID NO: 85 - protein, 424 aa, Glycine max
MVRQADLVIIGVSVGLTLGILISCLIFFGIRWYKKRAHIRRSANESSLTTLPIRTNGLGTSSDFSASLDSSIASSWSENLKRNSHFSWWNHQNKDRFASASGILKYLYKEIQKATQNFTTTLGQGSFGTVYKATMPTGEVVAVKVLAPNSKQGEKEFQTEVFLLGRLHHRNLVNLVGYCVDKGQRILVYQYMSNGSLANLLYGEEKELSWDQRLQIALDISHGIEYLHEGAVPPVIHRDLKSANILLDHSMRAKVADFGLSKEEIFDDRNSGLKGTYGYMDPAYISTSKLTTKSDIYSFGIIVFELITAIHPHQNLMEYVNLAAMDHDGVDEILDKQLVGKCNLEEVRQLAKIGHKCLHKSPRKRPSIGEVSQFISRIKQRRQRHLTEDNLSFASNNFSRAVSRLEDRQVELSRMPTINLTETV

SEQ ID NO: 86 - protein, 648 aa, Glycine max
METENGDTRSKKMEEYEVIEQIGRGAFGSAFLVLHKSEKKRYVLKKIRLAKQTEKFKRTAHQEMNLIAKLNNPYIVDYKDAWVEKEDHICIITGYCEGGDMAENIKKARGSFFPEEKVCKWLTQLLIAVDYLHSNRVIHRDLKCSNIFLTKDNNIRLGDFGLAKRLNAEDLASSVVGTPNYMCPELLADIPYGYKSDMWSLGCCMFEIAAHQPAFRAPDMAGLINKINRSSISPLPIVYSSTLKQLIKSMLRKNPEHRPTAAELLRHPLLQPYVLRCHNASSNVLPVYPLVNPKDKARRPNKSSGGKDHKDKEAGLVNCLERIHPIEGNADIQISNLPNDVVTISTSAEDNLETRMANLTSYIVESSTSISGSKDGSTTSESTICSVCKEDFKSRPAREMTNNEISSKSTQDSLHEEQRFAAKHFHKLEDDDINAVTAEVEDASCNGGLDNAEAQREDSNLEDSGKSTMSSEGSSSTDKDKSINEERSSLIVHPIRVENDTESGNRLKKSENPDVYTEVPHMNCLMSVSNDALPVKDDDIANGHILCSTHKDDNVVEVDQAPSGISLSVITEVDGDETIKTPLDSPCQQRADALESLLELCAQLLKQDKLEELAGVLRPFGKEAVSSRETAIWLAKSLISSQKFNPET

SEQ ID NO: 87 - protein, 467 aa, Glycine max
MSVMMDHVIGGKFKLGRKIGSGSFGELYIAVNIQTGEEVAVKLEPVKTKHPQLLYESKLYMLLQGGTGIPHLKWFGVEGDYNVMAIDLLGPSLEDLFNYCNRKLTLKTVLMLADQLINRVEYMHSRGFLHRDIKPDNFLMGLGRKANQVYIIDYGLAKKYRDLQTHRHIPYRENKNLTGTARYASVNTHLGIEQSRRDDLESLGYVLMYFLRGSLPWQGLKAGTKKQKYDKISEKKMSTSIEVLCKSYPSEFVSYFNYCRTLRFEDKPDYSYLKRLFRDLFIREGYQFDYVFDWTILKYPQIGGSSSRGRHESGKAAMHAGPSVQKPEKVSVGKEIREKFSGAVEAFSRRNPTSPSPRGDHSKHRSFEEVAVHKDVYRDQEKGRNSSRYGSSSRRPIISSSTRPSSSGDHTDSRTGRLTSSGNRSSASHRNIQPMYETKQPTYMRSGSTRGNRDDPLRSFELLSIRK

SEQ ID NO: 88 - protein, 721 aa, Glycine max
MVEFQRLLVCITTCILCWMPNGATAATDPNDAAAVRFLFQNMNSPPQLGWPPNGDDPCGQSWKGITCSGNRVTEIKLSNLGLTGSLPYGLQVLTSLTYVDMSSNSLGGSIPYQLPPYLQHLNLAYNNITGTVPYSISNLTALTDLNFSHNQLQQGLGVDFLNLSTLSTLDLSFNFLTGDLPQTMSSLSRITTMYLQNNQFTGTIDVLANLPLDNLNVENNNFTGWIPEQLKNINLQTGGNAWSSGPAPPPPPGTPPAPKSNQHHKSGGGSTTPSDTATGSSSIDEGKKSGTGGGAIAGIVISVIVVGAIVAFFLVKRKSKKSSSDLEKQDNQSFAPLPSNEVHEEKSMQTSSVTDLKTFDTSASINLKPPPIDRHKSFDDEEFSKRPTIVKKTVTAPANVKSYSIAELQIATGSFSVDHLVGEGSFGRVYRAQFDDGQVLAVKKIDSSILPNDLTDDFIQIISNISNLHHPNVTELVGYCSEYGQHLLVYEFHKNGSLHDFLHLSDEYSKPLIWNSRVKIALGTARALEYLHEVSSPSVVHKNIKSANILLDTELNPHLSDSGLASYIPNADQILNHNVGSGYDAPEVALSGQYTLKSDVYSFGVVMLELLSGRNPFDSSRPRSEQSLVRWATPQLHDIDALAKMVDPAMKGLYPVKSLSRFADVIALCVQPEPEFRPPMSEVVQALVRLVQRANMSKRTFSSSDHGGSQRGSDEPVLRDI

SEQ ID NO: 89 - protein, 688 aa, Glycine max
MNLFFFLKPLSSLMILSYIILFFLLVRNTSCDVDPNYVACPPKTCANNNQSISYPFYIEETQEPFCGNPGFAISCGPNGFPILNLSNTHYIIHQIFYENQTLRVSNAAFSVSRSNTTNSKGCLPVPLTHNLTLPSTPEFDIAPNQSNMRLFYGCESLLPWPEEHRVGCPNETSSVLAFYKEDKNISLVSKNCRGEVVDTIVEDGIIEGGVEEALRKGLLLTWKAGNCSECHSSGGRCGFDSIMYTFRCFCTDRVHSAKCDPDNGPVIKKGSSLKLVIGIGIPSMLAIGLLFLFLLYKRKYATSGGQLESRDSYSDSSSNPHRETSSEYFGVPLFLYEQLKEATNNFDHTKELGDGGFGTVYHGKLPDGREVAVKRLYEHNWKRVEQFMNEVKILTRLRHKYLVSLYGCTSRHSRELLLVYEYISNGTVACHLHGELAKPGSLPWSIRMKIAIETAIALTYLHASDIIHRDVKTNNILLDNNFCVKVADFGLSRDFPNNVTHVSTAPQGSPGYLDPEYYNCYQLTSKSDVYSFGVVLIELISSKPAVDMNRSRDEINLSNLAVRKIQESAISELVDPSLGFDSDNGIKGMIVSVAGLAFQCLQREKDLRPSMDEVLDELRRIESGKDEGEVQDEGDVNGAAVSHSSAHSPPPASPEWEEVRLLRNIKPTSPNTVTDKWESKCTTPNISG

SEQ ID NO: 90 - protein, 675 aa, Glycine max
MADLSCIHLFFLCCLFLKFIPQGNASMFHTAFCDNKEGNYTANSTYNTNLNTLLSSLSSHTEINYGFYNFSYGQNPDKVNAIGLCRGDVEPHECRSCLNDSRVTIKQFCPNQKKALLWLNTSKCMLRYSPRSIFGIMEIEPSQSLMNINNVTEPDKFSQALANLMRNLKGVAASGDSRRKYATDNVTASSFQTIYGMAECTPDLSEKDCNDCLDGAISKIPTCCQDKIGGRVLRPSCNIRFESASFYENTPILNPDVPPPSPAVAIPPSINSTSPKESSNTIRIVIAIVVPTVVVVPLICLCIYSRRSKARKSSLVKQHEDDDEIEIAQSLQFNFDTIRVATEDFSDSNKLGQGGFGAVYRGRLSDGQMIAVKRLSRESSQGDTEFKNEVLLVAKLQHRNLVRLLGFCLEGKERLLIYEYVPNKSLDYFIFDPTKKAQLNWEMRYKIITGVARGLLYLHEDSHLRIIHRDLKASNILLNEEMNPKIADFGMARLVLMDQTQANTNRIVGTYGYMAPEYAMHGQFSMKSDVFSFGVLVLEIISGHKNSGIRHGENVEDLLSFAWRNWREGTAVKIVDPSLNNNSRNEMLRCIHIGLLCVQENLADRPTMTTIMLMLNSYSLSLPIPSKPAFYVSSRTGSISATQSWGYSSGESRSRELTIKSAQEAENEFTDPYPR

SEQ ID NO: 91 - protein, 869 aa, Glycine max
MDTTSTQIASGTILFLLLLFFPYLSIADVIYSPVELFSINCGSSSNLSTRDGRNWTADIKFLSENKDSVAAPALTPSTLEGPYTDARLSHSQFTYSFPVSTGPKFLRLFFYSTSYQNFHRSKAYFSVKAGPYTLLQNFNASLHADAGNEPGDYLFREYCINLKDGDRLNITFIASKTSQNPDSYAFINGIEIVSMPPFLYYTNPHDVDITGLPHLVGVNTNLFPIENNFTLETKYRLRVGDQEIPASQDTGMLRSWDVDSKYVTTQSVLSLDIGPGIKLRFTKIPNYTAPDTVYRSVRNMGNNGTINMGFNLTWQLPIDSGFTYLLRLHFCQLNPEMKNPGYQSFFIFVQDQLVEKWADILSWSDKQEGVPVVKQYVVFIPGNQQETLNLSLKMHPNPQSLAKDAQINAIELFKINNSTGSLAGPNPDPDRLPETPKVPLQRPNNKSSGTTRTLAAAVAGAVSAAVLLSFIVAFFLIKRKKKMGSKEKDETPLGGGLSSLPTNLCRHFSIAEIRASTNNFDEHFVVGMGGFGNVYKGYIDDGSTRVAIKRLKPDSRQGAQEFMNEIEMLSQLRHLHLVSLVGYCYESNEMILVYDFMDRGTLREHLYDTDNPSLSWKQRLQICVGAARGLHYLHTGAKHTIIHRDVKSTNILLDEKWVAKVSDFGLSRIGPISSSMTHVSTQVKGSVGYIDPEYYKRQRLTEKSDVYSFGVVLLEVLSGRQPLLRWEEKQRISLVNWAKHCNEKGTLSEIVDAKLKGQIAPQCLQRYGEVALSCLLEDGTQRPSMNDAVRMLEFVLHLQEGAVNEVTESEDTEDVFSSSHSSLLFSDYSKSTALSMATNVGDCSYGSKDSEERSIPDHLFSEIKDPKGR

SEQ ID NO: 92 - protein, 674 aa, synthetic construct
  REGION 1..674: note = Kinase dead varient
MKKVCHCPIFLALALCLCILCVAAEAAGQNDTLALTEFRLQTDTHGNLLTNWTGADACSAAWRGVECSPNGRVVGLTLPSLNLRGPIDTLSTLTYLRFLDLHENRLNGTISPLLNCTSLELLYLSRNDFSGEIPAEISSLRLLLRLDISDNNIRGPIPTQLAKLTHLLTLRLQNNALSGHVPDLSASLLNLTVLNVTNNELRGHVPDSMLTKFGNVSFSGNHALCGSTPLPKCSETEPDTETTTITVPAKPSSFPQTSSVTVPDTPRKKGLSAGVIVAIVVAVCVAVLVATSFAVAHCCARGSTSGSVVGSETAKRKSGSSSGSEKKVYGNGGNLDRDSDGTNTETERSKLVFFDRRNQFELEDLLRASAEMLGKGSLGTVYRAVLDDGCTVAVRRLKDANPCERNEFEQYMDVVGKLKHPNIVRLRAYYYAKEEKLLVYDYLPNGSLHALLHGNRGPGRIPLDWTTRISLMLGAARGLARIHAEYNASKIPHGNVRSSNVLLDKNGVALISDFGLSLLLNPVHAIARLGGYRAPEQVEVKRLSQEADVYGFGVLLLEVLTGRAPSKEYTSPAREAEVDLPKWVKSVVKEEWTSEVFDQELLRYKNIEDELVAMLHVGLACVAAQAEKRPCMLEVVKMIEEIRVEESPLGDDYDEARSRTSLSPSLATTEDNLA

SEQ ID NO: 93 - protein, 978 aa, synthetic construct
  REGION 1..978: note = kinase dead varient
MQALFSSMRVFLRLVWLLELLCVAVTAVNPSLNDDVLGLIVFKADIRDPKGKLASWNEDDESACGGSWVGVKCNPRSNRVVEVNLDGFSLSGRIGRGLQRLQFLRKLSLANNNLTGGINPNIARIDNLRVIDLSGNSLSGEVSEDVFRQCGSLRTVSLARNRFSGSIPSTLGACSALAAIDLSNNQFSGSVPSRVWSLSALRSLDLSDNLLEGEIPKGIEAMKNLRSVSVARNRLTGNVPYGFGSCLLLRSIDLGDNSFSGSIPGDFKELTLCGYISLRGNAFSGGVPQWIGEMRGLETLDLSNNGFTGQVPSSIGNLQSLKMLNFSGNGLTGSLPESMANCTKLLVLDVSRNSMSGWLPLWVFKSDLDKVLVSENVQSGSKKSPLFAMAELAVQSLQVLDLSHNAFSGEITSAVGGLSSLQVLNLANNSLGGPIPPAVGELKTCSSLDLSYNKLNGSIPWEIGGAVSLKELVLEKNFLNGKIPTSIENCSLLTTLILSQNKLSGPIPAAVAKLTNLQTVDVSFNNLTGALPKQLANLANLLTFNLSHNNLQGELPAGGFFNTITPSSVSGNPSLCGAAVNKSCPAVLPKPIVLNPNTSTDTGPSSLPPNLGHKRIILSISALIAIGAAAVIVIGVISITVLNLRVRSSTSRDAAALTFSAGDEFSHSPTTDANSGKLVMFSGEPDFSSGAHALLNKDCELGRGGFGAVYQTVLRDGHSVAIKRLTVSSLVKSQEDFEREVKKLGKIRHQNLVELEGYYWTPSLQLLIYEYLSGGSLYKHLHEGSGGNFLSWNERFNVILGTAKALAHLHHSNIIHYNIRSTNVLLDSYGEPKVGDFGLARLLPMLDRYVLSSKIQSALGYMAPEFACKTVKITEKCDVYGFGVLVLEIVTGKRPVEYMEDDVVVLCDMVRGALEEGRVEECIDERLQGKFPAEEAIPVMKLGLICTSQVPSNRPDMGEVVNILELIRCPSEGQEELA

SEQ ID NO: 94 - protein, 419 aa, synthetic construct
  REGION 1..419: note = kinase dead varient
MDSARTAPPPWQELDLDSLKPLKVLGKGGMGTVFLVQAANNTRFALRVVDKTCVHAKLDAERRARWEIQVLSTLSHPFLPSLMGTFESPQFLAWALPYCPGGDLNVLRYRQTDRAFSPAVIRFYVAEILCALDHLHSMGIAYRDLRPENVLVQNTGHITLTDFDLSRKLNPKPKPNPQVPSIPLPNSNVPEPRRKHRRNFSRWISLFPPDGTHHNNNKNGLKKAKSARVSPVSRRKPSFSNGERSNSFVGTEEYVSPEVVRGDGHEFAVDWWALGILIYEMLYGTTPFKGKNRKETFRNVITKPPVFVGKRTALTDLIEKLLEKDPTKRLGYTRGAVEIKEHEFFRGVRWELLTEVVRPPFIPTRDDGAGDSTDRISDRNCGFDIRGYFLNLKSSPSLPGSPLPSPSCRFKKNVSLTEF

SEQ ID NO: 95 - protein, 688 aa, synthetic construct
  REGION 1..688: note = kinase dead varient
MMKHYHVLYMFLFFLPISTLSLHHNDTHALTLFRRQSDLHGYLLSNWTGHDACNSAWRGVLCSPNGRVTALSLPSLNLRGPLDPLTPLTHLRLLNLHDNRLNGTVSTLFSNCTNLQLLYLSSNDFSGEIPPEISSLKSLLRLDLSDNNLRGKVDVISNLTQLITLRLQNNLLSGEIPDLSSSMKNLKELNMTNNEFYGRLPSPMLKKFSSTTFSGNEGLCGASLFPGCSFTTTPPNNNDSNNNNDNNSNEKEPSQTVSSNPSSFPETSIIARPGREQQRKGLSPGAIVAIVIANCVALLVVVSFAVAHCCARGRGSSLVGSGESYGKRKSESSYNGSDEKKVYGGGESDGTSGTDRSRLVFFDRRSEFELEDLLRASAEMLGKGSLGTVYRAVLDDGCTVAVRRLKDANPCARHEFEQYMDVIGKLKHPNVVRLKAYYYAKEEKLLVYDYLSNGSLHALLHGNRGPGRIPLDWTTRISLVLGAARGLAKIHAEYSAAKVPHGNVRSSNVLLDKNGVACISDFGLSLLLNPVHAIARLGGYRAPEQEQNKRLSQQADVYSFGVLLLEVLTGRAPSSQYPSPARPRMEVEPEQAAVDLPKWVRSVVREEWTAEVFDQELLRYKNIEEELVSMLHVGLTCVVAQPEKRPTMEEVVKMIEEIRVEQSPLGEDYDVSCNSLSPSIPTTEDGLA

SEQ ID NO: 96 - protein, 1027 aa, synthetic construct
  REGION 1..1027: note = kinase dead varient
METSLDYSKMLFLLLLGSTSLIFLSHLASAATPKLNTQEVKALKEIGSKIGKKDWNFGVDPCSGKGNWNVPDARKAFVMSSVICDCSFNHNSSCHVVSIYWKAQNLSGSLSPEFSKLHYLQKLDLSRNIITGSIPPQWGTMRLVELSLMGNKLSGPFPKVLTNITTLRNLSIEGNQFSGHIPTEIGKLTNLEKLVLSSNGFTGALPPVLSKLTKLIDLRISDNNFLGKIPDFISNWTLIEKLHMHGCSLEGPIPSSISALTRLSDLRITDLKGSKSSAFPPLNNLKSMKTLVLRKCMIKGEIPEYIGRMEKLKILDLSYNGLSGEIPESFAQLDKVDFMYLTGNKLSGIIPRWVLANNENIDISDNNFSWDSSSPTECQRGSVNLVESYSSSVNTQTKINSCLKKNFLCTASPSQYRYSLNINCGGNEANVSGNIYEADREQKGAAMLYYTSQDWALSSTGNFMDNDIDSDPYIVANTSRLNVSALNSKLYTTARVSPLALTYYGLCLINGNYTVKLHFAEIIFINDRSLNSLGRRVFDVYIQGNLVLKDFDIRREAGGTGKSIEKTFNASVTQHTLKIHFYWAGKGTTGIPTRGVYGPLVSAISVNPNFKPPSGEGKRTYLILAIIIVAGVLVVVLLVLVLLRRMGWLGGKDPVYKELRGIDLQTGLFTLRQIKAATKNFDALNKIGEGGFGCVYKGQQSDGTMIAVRQLSSKSKQGNREFVNEMGLISGLQHPNLVKLYGCCVEGNQLILIYEYMENNCLSRILFGRDPNKTKLDWPTRKKICLGIAKALAYLHEESRIKIIHRDVRASNVLLDKDFNAKVSDFGLAKLIEDEKTHISTRVAGTIGYMAPEYAMRGYLTDKADVYSFGVVALETVSGKSNTNFRPNEDFVYLLDWAYVLQERGSLLELVDPNLGSEYLTEEAMVVLNVALLCTNASPTLRPTMSQVVSMLEGWTDIQDLLSDPGYSAISSSSKHKSIRSHFWQTPSGTHSISIPSIYTDSSGSHVETEKNYHPVTVNSDGSDKSN

SEQ ID NO: 97 - protein, 614 aa, synthetic construct
  REGION 1..614: note = kinase dead varient
MVLHSWISSSHILVNFLLLLGCGITYGTDTDIFCLKSIKESLEDPYNYLKFSWDFNNKTEGYICRFNGVECWHPDENRVLNLKLSNMGLKGQFPRGIQNCSSLTGLDLSINKLSGTIPGDISTLIPFATSIDLSTNEFSGAIPVSLANCTFLNTLKLDQNRLTGQIPPQFGVLSRIKVFSVSNNLLTGQVPIFRDGVELHYANNQGLCGGNTLAPCQATPSKSNMAVIAGAAAGGVTLAALGLGIGMFFFVRRVSFKKKEEDPEGNKWARSLKGTKRIKVSMFEKSISKMKLSDLMKATNNFSNTNIIGTGRTGTVYKAVLDDGTTLMVRRLQESQYTEKEFMSEMGTLGTVKHRNLVPLLGFCMTKRERLLVYKNMPNGNLHDQLHPADGVSTLDWTTRLKIAIGAAKGLAWLHHSCNPRIIHRNISSRCILLDADFEPKISDFGLARLMNPIDTHLSTFVNGEFGDLGYVAPEYTRTLVATPKGDIYSFGTVLLELVTGERPTNVSKAPETFKGNLVEWITELTSNAKLHDAIDESLVRKDVDSELFQFLKVACNCVSPTPKERPTMFEVYQLLRAIGGRYNFTTEDDILVPTDIGNTDNMQELIVAQEGSY

SEQ ID NO: 98 - protein, 649 aa, synthetic construct
  REGION 1..649: note = kinase dead varient
MEKTIHLVYFKCLVFLLMKQVIVMAEPRARTVNITCNNKLEHNTTIFVPNFVATMEKISEQMRNTGYGTAVVGTGGPDTNYGLAQCYGDLSLLDCVLCYAEARTVLPQCFPYNGGRIYLDGCFMRAENYSFYDEYIGPGDKAVCGNTTRKSTSFQAAAKKAVLSAVQAAANNKGYARKEVFVAGTTNDAAYVLANCWRSLDTRSCRACLENASSSILGCLPWSEGRALNTGCFMRYSDTDFLNKEQENGSSGGNVLVIVVAVVSSVIVLVVGIAIVVYIRKHRYIQMKRRGSNDAEKLAKSLHHNSLNFKYSTLEKATNSFDEANKLGQGGFGTVYKGVLADGREIAIRRLYFNNRHRAADFFNEVNIISSVEHKNLVRLLGCSCSGPESLLIYEYLPNRSLDRFIFDKNKGRELNWDKRYDIIIGTAEGLVYLHENSNIRIIHRDIRASNILLDAKLRAKIADFGLARSFQEDKSHISTAIAGTLGYMAPEYLAHGQLTEKADVYSFGVLLLEIITGRLNNRSKASEYSDSLVTMTWKHFQSGTAEQLIDPCLVVDDNHRSNFKNEILRVLHIGLLCTQEIPSLRPSMSKALKMLTKKEEHLDLEAPSNPPFIDESTMELHDQNDDPFYPLNAEDSLATMSHSSFYAR

SEQ ID NO: 99 - protein, 869 aa, synthetic construct
  REGION 1..869: note = kinase dead varient
MDTTSTQIASGTILFLLLLFFPYLSIADVIYSPVELFSINCGSSSNLSTRDGRNWTADIKFLSENKDSVAAPALTPSTLEGPYTDARLSHSQFTYSFPVSTGPKFLRLFFYSTSYQNFHRSKAYFSVKAGPYTLLQNFNASLHADAGNEPGDYLFREYCINLKDGDRLNITFIASKTSQNPDSYAFINGIEIVSMPPFLYYTNPHDVDITGLPHLVGVNTNLFPIENNFTLETKYRLRVGDQEIPASQDTGMLRSWDVDSKYVTTQSVLSLDIGPGIKLRFTKIPNYTAPDTVYRSVRNMGNNGTINMGFNLTWQLPIDSGFTYLLRLHFCQLNPEMKNPGYQSFFIFVQDQLVEKWADILSWSDKQEGVPVVKQYVVFIPGNQQETLNLSLKMHPNPQSLAKDAQINAIELFKINNSTGSLAGPNPDPDRLPETPKVPLQRPNNKSSGTTRTLAAAVAGAVSAAVLLSFIVAFFLIKRKKKMGSKEKDETPLGGGLSSLPTNLCRHFSIAEIRASTNNFDEHFVVGMGGFGNVYKGYIDDGSTRVAIRRLKPDSRQGAQEFMNEIEMLSQLRHLHLVSLVGYCYESNEMILVYDFMDRGTLREHLYDTDNPSLSWKQRLQICVGAARGLHYLHTGAKHTIIHRDVRSTNILLDEKWVAKVSDFGLSRIGPISSSMTHVSTQVKGSVGYIDPEYYKRQRLTEKSDVYSFGVVLLEVLSGRQPLLRWEEKQRISLVNWAKHCNEKGTLSEIVDAKLKGQIAPQCLQRYGEVALSCLLEDGTQRPSMNDAVRMLEFVLHLQEGAVNEVTESEDTEDVFSSSHSSLLFSDYSKSTALSMATNVGDCSYGSKDSEERSIPDHLFSEIKDPKGR

SEQ ID NO: 100 - protein, 130 aa, Glycine max
IIGTGRTGTVYKAVLDDGTTLMVKRLQESQYTEKEFMSEMGTLGTVKHRNLVPLLGFCMTKRERLLVYKNMPNGNLHDQLHPADGVSTLDWTTRLKIAIGAAKGLAWLHHSCNPRIIHRNISSKCILLDA

SEQ ID NO: 101 - protein, 126 aa, Homo sapiens
KIGEGSYGVVFKCRNRDTGQIVAIKKFLESEDDPVIKKIALREIRMLKQLKHPNLVNLLEVFRRKRRLHLVFEYCDHTVLHELDRYQRGVPEHLVKSITWQTLQAVNFCHKHNCIHRDVKPENILI

SEQ ID NO: 102 - protein, 121 aa, Gallus gallus
KLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMI

SEQ ID NO: 103 - protein, 126 aa, Homo sapiens; identical to SEQ ID NO: 101

SEQ ID NO: 104 - protein, 127 aa, Saccharomyces cerevisiae
QVGEGTYGKVYKAKNTNTEKLVALKKLRLQGEREGFPITSIREIKLLQSFDHPNVSTIKEIMVESQKTVYMIFEYADNDLSGLLLNKEVQISHSQCKHLFKQLLLGMEYLHDNKILHRDVKGSNILI

SEQ ID NO: 105 - protein, 125 aa, Rattus norvegicus
QLGDGTYGSVLMGKSNESGELVAIKRMKRKFYSWDECMNLREVKSLKKLNHANVIKLKEVIRENDHLYFIFEYMKENLYQLMKDRNKLFPESVIRNIMYQILQGLAFIHKHGFFHRDMKPENLLC

SEQ ID NO: 106 - protein, 129 aa, Rattus norvegicus
VIGNGSFGVVYQARLAETRELVAIKKVLQDKRFKNRELQIMRKLDHCNIVRLRYFFYSSGEKKDELYLNLVLEYVPETVYRVARHFTKAKLIIPIIYVKVYMYQLFRSLAYIHSQGVCHRDIKPQNLLV

SEQ ID NO: 107 - protein, 125 aa, Saccharomyces cerevisiae
VVGQGAFATVKKAIERTTGKTFAVKIISKRKVIGNMDGVTRELEVLQKLNHPRIVRLKGFYEDTESYYMVMEFVSGGDLMDFVAAHGAVGEDAGREISRQILTAIKYIHSMGISHRDLKPDNILI

SEQ ID NO: 108 - protein, 121 aa, Mus musculus
TIGVGSYSVCKRCVHKATNMEYAVKVIDKSKRDPSEEIEILLRYGQHPNIITLKDVYDDGKHVYLVTELMRGGELLDKILRQKFFSEREASFVLHTISKTVEYLHSQGVVHRDLKPSNILY

SEQ ID NO: 109 - protein, 253 aa, synthetic construct
  REGION 1..253: note = alignment of protein kinase domains
LKPLKVLGKGGMGTVFLVQAANNTRFALKVVDKTCVHAKLDAERRARWEIQVLSTLSHPFLPSLMGTFESPQFLAWALPYCPGGDLNVLRYRQTDRAFSPAVIRFYVAEILCALDHLHSMGIAYRDLKPENVLVQNTGHITLTDFDLSRKLNPKPKPNPQVPSIPLPNSNVPEPRRKHRRNFSRWISLFPPDGTHHNNNKNGLKKAKSARVSPVSRRKPSFSNGERSNSFVGTEEYVSPEVVRGDGHEFAVDW

SEQ ID NO: 110 - protein, 241 aa, synthetic construct
  REGION 1..241: note = alignment of protein kinase domains
FSNTNIIGTGRTGTVYKAVLDDGTTLMVKRLQESQYTEKEFMSEMGTLGTVKHRNLVPLLGFCMTKRERLLVYKNMPNGNLHDQLHPADGVSTLDWTTRLKIAIGAAKGLAWLHHSCNPRIIHRNISSKCILLDADFEPKISDFGLARLMNPIDTHLSTFVNGEFGDLGYVAPEYTRTLVATPKGDIYSFGTVLLELVTGERPTNVSKAPETFKGNLVEWITELTSNAKLHDAIDESLVRK

SEQ ID NO: 111 - protein, 239 aa, synthetic construct
  REGION 1..239: note = alignment of protein kinase domains
FDEHFVVGMGGFGNVYKGYIDDGSTRVAIKRLKPDSRQGAQEFMNEIEMLSQLRHLHLVSLVGYCYESNEMILVYDFMDRGTLREHLYDTDNPSLSWKQRLQICVGAARGLHYLHTGAKHTIIHRDVKSTNILLDEKWVAKVSDFGLSRIGPISSSMTHVSTQVKGSVGYIDPEYYKRQRLTEKSDVYSFGVVLLEVLSGRQPLLRWEEKQRISLVNWAKHCNEKGTLSEIVDAKLKGQ

SEQ ID NO: 112 - protein, 238 aa, synthetic construct
  REGION 1..238: note = alignment of protein kinase domains
FDALNKIGEGGFGCVYKGQQSDGTMIAVKQLSSKSKQGNREFVNEMGLISGLQHPNLVKLYGCCVEGNQLILIYEYMENNCLSRILFGRDPNKTKLDWPTRKKICLGIAKALAYLHEESRIKIIHRDVKASNVLLDKDFNAKVSDFGLAKLIEDEKTHISTRVAGTIGYMAPEYAMRGYLTDKADVYSFGVVALETVSGKSNTNFRPNEDFVYLLDWAYVLQERGSLLELVDPNLGSE

SEQ ID NO: 113 - protein, 237 aa, synthetic construct
  REGION 1..237: note = alignment of protein kinase domains
FDEANKLGQGGFGTVYKGVLADGREIAIKRLYFNNRHRAADFFNEVNIISSVEHKNLVRLLGCSCSGPESLLIYEYLPNRSLDRFIFDKNKGRELNWDKRYDIIIGTAEGLVYLHENSNIRIIHRDIKASNILLDAKLRAKIADFGLARSFQEDKSHISTAIAGTLGYMAPEYLAHGQLTEKADVYSFGVLLLEIITGRLNNRSKASEYSDSLVTMTWKHFQSGTAEQLIDPCLVVD

SEQ ID NO: 114 - protein, 236 aa, synthetic construct
  REGION 1..236: note = alignment of protein kinase domains
LNKDCELGRGGFGAVYQTVLRDGHSVAIKKLTVSSLVKSQEDFEREVKKLGKIRHQNLVELEGYYWTPSLQLLIYEYLSGGSLYKHLHEGSGGNFLSWNERFNVILGTAKALAHLHHSNIIHYNIKSTNVLLDSYGEPKVGDFGLARLLPMLDRYVLSSKIQSALGYMAPEFACKTVKITEKCDVYGFGVLVLEIVTGKRPVEYMEDDVVVLCDMVRGALEEGRVEECIDERLQGK

SEQ ID NO: 115 - protein, 239 aa, synthetic construct
  REGION 1..239: note = alignment of protein kinase domains
RASAEMLGKGSLGTVYRAVLDDGCTVAVKRLKDANPCERNEFEQYMDVVGKLKHPNIVRLRAYYYAKEEKLLVYDYLPNGSLHALLHGNRGPGRIPLDWTTRISLMLGAARGLARIHAEYNASKIPHGNVKSSNVLLDKNGVALISDFGLSLLLNPVHAIARLGGYRAPEQVEVKRLSQEADVYGFGVLLLEVLTGRAPSKEYTSPAREAEVDLPKWVKSVVKEEWTSEVFDQELLRYK

SEQ ID NO: 116 - protein, 247 aa, synthetic construct
  REGION 1..247: note = alignment of protein kinase domains
RASAEMLGKGSLGTVYRAVLDDGCTVAVKRLKDANPCARHEFEQYMDVIGKLKHPNVVRLKAYYYAKEEKLLVYDYLSNGSLHALLHGNRGPGRIPLDWTTRISLVLGAARGLAKIHAEYSAAKVPHGNVKSSNVLLDKNGVACISDFGLSLLLNPVHAIARLGGYRAPEQEQNKRLSQQADVYSFGVLLLEVLTGRAPSSQYPSPARPRMEVEPEQAAVDLPKWVRSVVREEWTAEVFDQELLRYK

SEQ ID NO: 117 - protein, 208 aa, Glycine max
RASAEMLGKGSLGTVYRAVLDDGCTVAVKRLKDANPCERNEFEQYMDVVGKLKHPNIVRLRAYYYAKEEKLLVYDYLPNGSLHALLHGNRGPGRIPLDWTTRISLMLGAARGLARIHAEYNASKIPHGNVKSSNVLLDKNGVALISDFGLSLLLNPVHAIARLGGYRAPEQVEVKRLSQEADVYGFGVLLLEVLTGRAPSKEYTSPAR

SEQ ID NO: 118 - protein, 206 aa, Glycine max
LNKDCELGRGGFGAVYQTVLRDGHSVAIKKLTVSSLVKSQEDFEREVKKLGKIRHQNLVELEGYYWTPSLQLLIYEYLSGGSLYKHLHEGSGGNFLSWNERFNVILGTAKALAHLHHSNIIHYNIKSTNVLLDSYGEPKVGDFGLARLLPMLDRYVLSSKIQSALGYMAPEFACKTVKITEKCDVYGFGVLVLEIVTGKRPVEYME

SEQ ID NO: 119 - protein, 232 aa, Glycine max
LKPLKVLGKGGMGTVFLVQAANNTRFALKVVDKTCVHAKLDAERRARWEIQVLSTLSHPFLPSLMGTFESPQFLAWALPYCPGGDLNVLRYRQTDRAFSPAVIRFYVAEILCALDHLHSMGIAYRDLKPENVLVQNTGHITLTDFDLSRKLNPKPKPNPQVPSIPLPNSNVPEPRRKHRRNFSRWISLFPPDGTHHNNNKNGLKKAKSARVSPVSRRKPSFSNGERSNSFVG

SEQ ID NO: 120 - protein, 211 aa, Glycine max
RASAEMLGKGSLGTVYRAVLDDGCTVAVKRLKDANPCARHEFEQYMDVIGKLKHPNVVRLKAYYYAKEEKLLVYDYLSNGSLHALLHGNRGPGRIPLDWTTRISLVLGAARGLAKIHAEYSAAKVPHGNVKSSNVLLDKNGVACISDFGLSLLLNPVHAIARLGGYRAPEQEQNKRLSQQADVYSFGVLLLEVLTGRAPSSQYPSPARPRM

SEQ ID NO: 121 - protein, 208 aa, Glycine max
FDALNKIGEGGFGCVYKGQQSDGTMIAVKQLSSKSKQGNREFVNEMGLISGLQHPNLVKLYGCCVEGNQLILIYEYMENNCLSRILFGRDPNKTKLDWPTRKKICLGIAKALAYLHEESRIKIIHRDVKASNVLLDKDFNAKVSDFGLAKLIEDEKTHISTRVAGTIGYMAPEYAMRGYLTDKADVYSFGVVALETVSGKSNTNFRPN

SEQ ID NO: 122 - protein, 211 aa, Glycine max
FSNTNIIGTGRTGTVYKAVLDDGTTLMVKRLQESQYTEKEFMSEMGTLGTVKHRNLVPLLGFCMTKRERLLVYKNMPNGNLHDQLHPADGVSTLDWTTRLKIAIGAAKGLAWLHHSCNPRIIHRNISSKCILLDADFEPKISDFGLARLMNPIDTHLSTFVNGEFGDLGYVAPEYTRTLVATPKGDIYSFGTVLLELVTGERPTNVSKAPE

SEQ ID NO: 123 - protein, 209 aa, Glycine max
FDEANKLGQGGFGTVYKGVLADGREIAIKRLYFNNRHRAADFFNEVNIISSVEHKNLVRLLGCSCSGPESLLIYEYLPNRSLDRFIFDKNKGRELNWDKRYDIIIGTAEGLVYLHENSNIRIIHRDIKASNILLDAKLRAKIADFGLARSFQEDKSHISTAIAGTLGYMAPEYLAHGQLTEKADVYSFGVLLLEIITGRLNNRSKASEY

SEQ ID NO: 124 - protein, 209 aa, Glycine max
FDEHFVVGMGGFGNVYKGYIDDGSTRVAIKRLKPDSRQGAQEFMNEIEMLSQLRHLHLVSLVGYCYESNEMILVYDFMDRGTLREHLYDTDNPSLSWKQRLQICVGAARGLHYLHTGAKHTIIHRDVKSTNILLDEKWVAKVSDFGLSRIGPISSSMTHVSTQVKGSVGYIDPEYYKRQRLTEKSDVYSFGVVLLEVLSGRQPLLRWEE

SEQ ID NO: 125 - protein, 206 aa, Zea mays
KVELGRGESGTVYKGVLEDDRHVAVKKLENVRQGKEVFQAELSVIGRINHMNLVRIWGFCSEGSHRLLVSEYVENGSLANILFSEGGNILLDWEGRFNIALGVAKGLAYLHHECLEWVIHCDVKPENILLDQAFEPKITDFGLVKLLNRGGSTQNVSHVRGTLGYIAPEWVSSLPITAKVDVYSYGVVLLELLTGTRVSELVGGTD

SEQ ID NO: 126 - protein, 211 aa, Homo sapiens
EEIIGIGGFGKVYRAFWIGDEVAVKAARHDPDEDISQTIENVRQEAKLFAMLKHPNIIALRGVCLKEPNLCLVMEFARGGPLNRVLSGKRIPPDILVNWAVQIARGMNYLHDEAIVPIIHRDLKSSNILILQKVENGDLSNKILKITDFGLAREWHRTTKMSAAGTYAWMAPEVIRASMFSKGSDVWSYGVLLWELLTGEVPFRGIDGLAV

SEQ ID NO: 127 - protein, 203 aa, Gallus gallus
VRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVI

SEQ ID NO: 128 - protein, 209 aa, Rattus norvegicus
IKVIGNGSFGVVYQARLAETRELVAIKKVLQDKRFKNRELQIMRKLDHCNIVRLRYFFYSSGEKKDELYLNLVLEYVPETVYRVARHFTKAKLIIPIIYVKVYMYQLFRSLAYIHSQGVCHRDIKPQNLLVDPDTAVLKLCDFGSAKQLVRGEPNVSYICSRYYRAPELIFGATDYTSSIDVWSAGCVLAELLLGQPIFPGDSGVDQLV

SEQ ID NO: 129 - protein, 199 aa, Saccharomyces cerevisiae
KQVIGRGSYGVVYKAINKHTDQVVAIKEVVYENDEELNDIMAEISLLKNLNHNNIVKYHGFIRKSYELYILLEYCANGSLRRLISRSSTGLSENESKTYVTQTLLGLKYLHGEGVIHRDIKAANILLSADNTVKLADFGVSTIVNSSALTLAGTLNWMAPEILGNRGASTLSDIWSLGATVVEMLTKNPPYHNLTDANI

SEQ ID NO: 130 - protein, 206 aa, Bos taurus
HRIIGRGGFGEVYGCRKADTGKMYAMKCLDKKRIKMKQGETLALNERIMLSLVSTGDCPFIVCMSYAFHTPDKLSFILDLMNGGDLHYHLSQHGVFSEADMRFYAAEIILGLEHMHNRFVVYRDLKPANILLDEHGHVRISDLGLACDFSKKKPHASVGTHGYMAPEVLQKGVAYDSSADWFSLGCMLFKLLRGHSPFRQHKTKDK

SEQ ID NO: 131 - protein, 205 aa, Drosophila melanogaster
VSTLGIGGFGRVELVKAHHQDRVDIFALKCLKKRHIVDTKQEEHIFSERHIMLSSRSPFICRLYRTFRDEKYVYMLLEACMGGEIWTMLRDRGSFEDNAAQFIIGCVLQAFEYLHARGIIYRDLKPENLMLDERGYVKIVDFGFAKQIGTSSKTWTFCGTPEYVAPEIILNKGHDRAVDYWALGILIHELLNGTPPFSAPDPMQT

SEQ ID NO: 132 - protein, 202 aa, Mus musculus
KETIGVGSYSVCKRCVHKATNMEYAVKVIDKSKRDPSEEIEILLRYGQHPNIITLKDVYDDGKHVYLVTELMRGGELLDKILRQKFFSEREASFVLHTISKTVEYLHSQGVVHRDLKPSNILYVDESGNPECLRICDFGFAKQLRAENGLLMTPCYTANFVAPEVLKRQGYDEGCDIWSLGILLYTMLAGYTPFANGPSDTP

SEQ ID NO: 133 - protein, 198 aa, Plasmodium falciparum
NKKIGKGSFSTAYIGTNILYGNRVVVKEVDKSKVKESNVYTEIEVLRKVMHKYIIKLISAYEQEGFVYLVLEYLKGGELFEYLNNNGPYTEQVAKKAMKRVLIALEALHSNGVVHRDLKMENLMLENPNDPSSLKIIDFGLASFLNSPSMNMRCGSPGYVAPEILKCASYGTKVDIFSLGVILFNILCGYPPFRGNNV

(19) United States
(12) Patent Application Publication
(10) Pub. No.: US 2024/0336932 A1
(43) Pub. Date: Oct. 10, 2024

(54) KINASE-DEAD MUTATION: A NEW APPROACH TO ENHANCE SOYBEAN RESISTANCE TO SOYBEAN CYST NEMATODE

(71) Applicant: UNIVERSITY OF TENNESSEE RESEARCH FOUNDATION, KNOXVILLE, TN (US)

(72) Inventors: TAREK ABDELFATTAH HEWEZI, KNOXVILLE, TN (US); SARBOTTAM PIYA, KNOXVILLE, TN (US)

(21) Appl. No.: 18/748,186

(22) Filed: Jun. 20, 2024

Related U.S. Application Data
(62) Division of application No. 17/227,472, filed on Apr. 12, 2021, now Pat. No. 12,018,270.
(60) Provisional application No. 63/009,120, filed on Apr. 13, 2020.

(51) Int. Cl. C12N 15/82; C12Q 1/6895

(52) U.S. Cl. CPC C12N 15/8285; C12Q 1/6895; C12Q 2600/13; C12Q 2600/156

(57) ABSTRACT

The invention relates to genes which may be utilized to induce resistance to soybean cyst nematode (SCN) and/or other abiotic or biotic stresses. More specifically the present disclosure provides genes that, when inactivated and/or overexpressed (for example, overexpression of genes encoding kinase-dead mutants) in a plant, particularly, a soybean plant, can confer upon the plant resistance to SCN and/or other abiotic or biotic stresses. Methods of using these genes to obtain plants, particularly soybean plants, that are resistant to SCN and/or other abiotic or biotic stresses are also provided.

### CROSS-REFERENCE TO RELATED APPLICATIONS

This application is a divisional of U.S. Ser. No. 17/227,472, filed Apr. 12, 2021, now U.S. Pat. No. 12,018,270, which claims the benefit of U.S. Provisional Application Ser. No. 63/009,120, filed Apr. 13, 2020, the disclosures of which are hereby incorporated by reference in their entirety, including all figures, tables and amino acid or nucleic acid sequences.

The Sequence Listing for this application is labeled “Seq-List.xml” which was created on Jun. 12, 2024 and is 190,134 KB. The entire content of the sequence listing is incorporated herein by reference in its entirety.

This invention was made with government support under awarded by USDA-National Institute of Food and Agriculture-Agriculture and Food Research Initiative (Grant No. 2018-67013-27822). The government has certain rights in the invention.

### BACKGROUND OF THE INVENTION

The United States ranks first in the world in soybean production, accounting for over 50% of the world's soybean production. In addition, strong global demand for soybeans is expected to continue to meet needs for vegetable oil and meal/protein for various industrial applications and animal feed. Therefore, increased soybean yield is needed to ensure adequate supplies. However, soybean yield is always threatened by pathogen infection, especially by the soybean cyst nematode (SCN, Heterodera glycines), the most damaging soybean pathogen; annual losses in the US from SCN range from 93 to 123 million bushels. Protecting soybean from SCN is a critical crop security issue. While the current best management strategy to control nematode infection in soybean fields is the use of nematode resistant cultivars coupled with effective cultural practices, SCN has the ability to adapt to these strategies and readily overcomes resistance, threatening control measures and producer profitability. Therefore, discovery of new sources of genetic resistance for engineering broad and durable resistance against SCN is of great importance for soybean producers and to the economy of the United States.

Protein kinases phosphorylate proteins for functional changes and are involved in nearly all cellular processes, thereby regulating almost all aspects of plant growth and development, and responses to biotic and abiotic stresses (Champion et al., 2004; Colcombet and Hirt, 2008). Protein kinase genes exist by the hundreds in all plant species in which they have been surveyed, and comprise more than 3% of the annotated proteins in plants (Lehti-Shiu et al., 2009; Lehti-Shiu and Shiu, 2012). The protein kinase repertoire or kinome has significantly more members in plants than other eukaryotes, including animals. Recent gene duplication events and high retention rate of duplicates in plants is likely the reason for increasing kinome growth, and has functional significance (Hanada et al., 2008; Lehti-Shiu and Shiu, 2012; Liu et al., 2015).

In this application we developed and validated a new approach for identifying bona fide target genes for SCN resistance. This novel approach relies on: (1) identifying highly interconnected kinase hub genes to prioritize SCN resistance gene candidates for further functional validation using (2) the novel kinase-dead mutation approach.

### BRIEF SUMMARY OF THE INVENTION

The instant invention pertains to the function of kinases in establishing kinase signaling cascades during SCN infection of soybeans. Kinase hub genes were identified based on gene co-expression networks and the kinase hub genes were analyzed to obtain insights into the roles of the genes during SCN infection. Mutating only one or two encoding sequences of amino acids of the kinases expressed by the kinase hub genes creates SCN-resistant soybeans. The mutations can completely abolish or significantly reduce (significantly inhibit) the activity of these enzymes but do not interfere with substrate recognition i.e., these kinases can be modified to create “kinase-dead mutants” that have no enzymatic activity or significantly reduced enzymatic activity but function antagonistically to the wild-type kinase (have dominant-negative effects). Identifying kinase hub genes and the consequence of kinase-dead mutations can identify means to inhibit SCN infection induced kinase signaling cascades. Additionally, a method of the subject invention provides a method for the analysis of co-expression networks to identify means for stress tolerance and disease resistance in plants.

### BRIEF DESCRIPTION OF THE DRAWINGS

FIGS. 1A-1B: Gene co-expression networks of soybean genes under control and stress conditions. Two independent co-expression networks were generated using control (FIG. 1A) and stress (FIG. 1B) gene expression data. Nodes indicate gene and edges indicate co-expression events between co-expressed genes. Nodes in red represent protein kinases.

FIGS. 2A-2D: Identification of kinase hub genes that are differentially expressed in SCN-induced syncytium. (FIG. 2A) Venn diagram demonstrating the number of unique and common kinase hub genes in control and stress networks. (FIG. 2B) Example of a protein kinase-hub gene (Glyma.06G145500) showing limited and high co-expression events in control and stress co-expression networks, respectively. (FIG. 2C) Bar graph showing the number of kinase-hub genes that are specific to control and stress networks. (FIG. 2D) Venn diagram demonstrating the number of kinase-hub genes that are differentially expressed in the feeding site of SCN (syncytium).

FIG. 3: Schematic illustration of kinase-dead mutation mediating a loss-of-function effects. The chemical activity of an active kinase enzyme involves transforming a phosphate group from a nucleotide triphosphate (ATP) to the target protein, a process known as phosphorylation that results in a functional changes of the target protein (substrate). In contrast, an inactive kinase (kinase-dead) binds to its substrate but without phosphorylation, thereby sequestering the activity of the substrate, causing a loss-of-function phenotype.

FIG. 4: Example of multiple sequence alignment of protein kinase domains of a syncytium-kinase hub gene (Glyma.04G222800) and other species showing the conserved lysine residues (K) in the ATP binding pocket and the substrate binding pocket (SEQ ID NOs: 100-108, respectively).

FIGS. 5A-5B: Overexpression of kinase-dead variants of syncytium kinase hub genes significantly increased soybean resistance to SCN. (FIG. 5A) Nematode susceptibility assay of transgenic hairy root plants overexpressing wild-type variants of three kinase hub genes (Glyma.04G222800, Glyma.13G150000 and Glyma.18G141500) showing increased susceptibility compared to the transgenic hairy root plants expressing the empty vector (control). (FIG. 5B) Nematode susceptibility assay of transgenic hairy root plants overexpressing kinase-dead variants of 8 kinase hub genes (Glyma.03G189800, Glyma.16G079200, Glyma.14G026300, Glyma.04G222800, Glyma.18G141500, Glyma.18G269900, Glyma.12G056000, and Glyma.13G150000) showing significantly reduced susceptibility compared to the transgenic hairy root plants expressing the empty vector (control). Asterisks indicate statistically significant differences from control plants (P <0.05) as determined by ANOVA.

FIGS. 6A-6B: Multiple sequence alignment of protein kinase domains of 8 syncytium-kinase hub genes. (FIG. 6A) Multiple sequence alignment of soybean kinase protein sequences showing 24 conserved amino acids including 2 lysine residues (K) in the ATP binding pocket and the substrate binding pocket (SEQ ID NOs: 109-116, respectively). (FIG. 6B) Multiple sequence alignment of kinase protein sequences of soybean (SEQ ID NOs: 117-124, respectively) and other species (P17801 (Zea mays (Maize)), SEQ ID NO: 125, P80192 (Homo sapiens), SEQ ID NO: 126, P21868 (Gallus gallus), SEQ ID NO: 127, P18265 (Rattus norvegicus), SEQ ID NO: 128, P27636 (Saccharomyces cerevisiae S288C), SEQ ID NO: 129, P21146 (Bos Taurus), SEQ ID NO: 130, Q03042 (Drosophila melanogaster), SEQ ID NO: 131, P18653 (Mus musculus), SEQ ID NO: 132, Q81534 (Plasmodium falciparum (isolate 3D7). SEQ ID NO: 133), showing 14 conserved amino acids including 2 lysine residues (K) in the ATP binding pocket and the substrate binding pocket. Invariant and highly conserved amino acids are shown in red lettering.

### BRIEF DESCRIPTION OF THE SEQUENCES

SEQ ID NOs: 1 to 91 provide candidate amino acid sequences that confer SCN resistance when overexpressed, inactivated, or overexpressed and inactivated.

SEQ ID NOs: 92 to 99 provide amino acid sequences of kinase-dead mutants that confer SCN resistance.

### DETAILED DISCLOSURE OF THE INVENTION

The present invention relates to novel and useful methods for introducing, in a reliable and predictable manner, SCN resistance into non-resistant soybeans. The method involves identifying highly connected kinase hub genes that change expression in the SCN feeding site. The identified kinase hub genes identified to be associated with the SCN resistance can be mutated to generate kinase-dead mutants, which have significantly reduced or no enzymatic activity but have a dominant-negative effect in sequestering substrates (for example ATP), causing a loss-of-function phenotype.

Kinases have highly conserved domains, allowing for the identification of amino acids essential for the function of the enzymes. Thus, one aspect of the invention provides kinase-dead mutants that comprise one or more amino acid mutation at conserved (invariant) or highly conserved amino acid positions. For example, single, double, or triple amino acid alterations (mutations) can inhibit enzymatic activity (for example, a lysine residue in the ATP binding pocket and a lysine residue in the substrate binding pocket of the kinase can be mutated to any other amino acid, for example an arginine or a conserved aspartic acid residue in the catalytic domain of the kinase can be mutated with any other amino acid), but the kinase-dead mutants maintain the ability to recognize/bind substrates or bind to other proteins. Altering the expression levels of kinase-dead mutants and/or wild-type kinases can enhance resistance to SCN. Because these modifications are genetic changes in the plant, offspring can inherit tolerance to SCN.

Kinase hub genes encode kinase enzymes that are involved in nearly all aspects of plant growth and development and stress tolerance. Many kinases have compensatory signaling and redundant functions. So, novel targeting approaches to select critical kinases within the kinome are imperative to efficiently disrupt signaling cascades driven by aberrant kinase activity induced by plant pathogens or other stresses. In certain embodiments of the subject invention, the method of identifying kinase genes involved in common signal transduction pathways, preferably in cells that are undergoing stress or pathogen infection, can be used to identify kinases involved in the stress event or pathogen infection (for example, SCN pathogenesis) and provide targets that can be mutated to provide resistance to the stress or pathogen infection, for example, kinase-dead mutants.

In this disclosure, the term “isolated nucleic acid” molecule means a nucleic acid molecule that is separated from other nucleic acid molecules that are usually associated with the isolated nucleic acid molecule. Thus, an “isolated nucleic acid molecule” includes, without limitation, a nucleic acid molecule that is free of nucleotide sequences that naturally flank one or both ends of the nucleic acid in the genome of the organism from which the isolated nucleic acid is derived (e.g., a cDNA or genomic DNA fragment produced by PCR or restriction endonuclease digestion). Such an isolated nucleic acid molecule is generally introduced into a vector (e.g., a cloning vector or an expression vector) for convenience of manipulation or to generate a fusion nucleic acid molecule. In addition, an isolated nucleic acid molecule can include an engineered nucleic acid molecule such as a recombinant or a synthetic nucleic acid molecule. A nucleic acid molecule existing among hundreds to millions of other nucleic acid molecules within, for example, a nucleic acid library (e.g., a cDNA or genomic library) or a gel (e.g., agarose, or polyacrylamide) containing restriction-digested genomic DNA, is not an “isolated nucleic acid”.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (cxons).

In this application, the terms “polypeptide”, “peptide”, and “protein” are used interchangeably herein to refer to a polymer of amino acids. The terms apply to amino acid polymers in which one or more amino acid residues are artificial chemical mimetic of a corresponding naturally occurring amino acids, as well as to naturally occurring amino acid polymers and non-naturally occurring amino acid polymers. As used herein, the terms encompass amino acid chains of any length, including full-length proteins, wherein the amino acid residues are linked by covalent peptide bonds.

As used in herein, the terms “identical” or percent “identity”, in the context of describing two or more polynucleotide or amino acid sequences, refer to two or more sequences or subsequences that are the same or have a specified percentage of amino acid residues or nucleotides that are the same (for example, a variant protein used in the method of this invention has at least 80% sequence identity, preferably 85%, 90%, 91%, 92%, 93, 94%, 95%, 96%, 97%, 98%, 99%, or 100% identity, to a reference sequence), when compared and aligned for maximum correspondence over a comparison window, or designated region as measured using one of the following sequence comparison algorithms or by manual alignment and visual inspection. Such sequences are then said to be “substantially identical”. With regard to polynucleotide sequences, this definition also refers to the complement of a test sequence. The comparison window, in certain embodiments, refers to the full length sequence of a given polypeptide, for example a specific kinase, or, in other embodiments, specific domain within the kinase sequence (e.g., the catalytic domain, ATP binding pocket or the substrate binding pocket of a kinase).

In this disclosure the terms “stringent hybridization conditions” and “high stringency” refer to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993) and will be readily understood by those skilled in the art. Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Current Protocols in Molecular Biology, ed. Ausubel, et al.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts the term “about” is provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

An endogenous nucleic acid is a nucleic acid that is naturally present in a cell. For example, a nucleic acid present in the genomic DNA of a cell is an endogenous nucleic acid.

An exogenous nucleic acid is any nucleic acid that is not naturally present in a cell. For example, a nucleic acid vector introduced into a cell constitutes an exogenous nucleic acid. Other examples of an exogenous nucleic acid include the vectors comprising a heterologous promoter linked to an endogenous nucleic acid, e.g., a nucleic acid encoding a kinase.

The subject invention provides for the use of “homologous nucleic acid sequences” or “homologs of nucleic acid sequences”. Homologs of nucleic acid sequences will be understood to mean any nucleotide sequence obtained by mutagenesis according to techniques well known to persons skilled in the art, and exhibiting modifications in relation to the parent sequences. For example, mutations in the regulatory and/or promoter sequences for the expression of a polypeptide that result in a modification of the level of expression of a polypeptide according to the invention provide for a “homolog of a nucleotide sequence”. Likewise, substitutions, deletions, or additions of nucleic acid to the polynucleotides of the invention provide for “homologs” of nucleotide sequences. In various embodiments, “homologs” of nucleic acid sequences have substantially the same biological activity as the corresponding reference gene, i.e., a gene homologous to a native gene would encode for a protein having the same biological activity as the corresponding protein encoded by the naturally occurring gene. Typically, a homolog of a gene shares a sequence identity with the gene of at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. These percentages are purely statistical and differences between two nucleic acid sequences can be distributed randomly and over the entire sequence length.

Likewise, the subject invention also provides for the use of kinase homologs. A kinase homolog has substantially the same biological activity as the reference kinase, i.e., the kinase homolog would have substantially the same biological activity as the reference kinase. Typically, a homolog of a reference kinase shares a sequence identity with the reference protein of at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. These percentages are purely statistical and differences between two nucleic acid sequences can be distributed randomly and over the entire sequence length.

Likewise, the subject invention also provides for “kinase-dead mutants”. Kinase-dead mutants are understood to mean any kinases obtained by mutagenesis according to techniques well known to persons skilled in the art, and exhibiting modifications in relation to the parent kinase. Such modifications in a protein sequence include substitutions, deletions, or additions of amino acids. In various embodiments, the kinase-dead mutant has one or more biological activity that is substantially different from one or more biological activity of the native protein kinase (for example, the kinase-dead mutant is unable to transfer a phosphate group to a substrate). In one embodiment, a kinase-dead mutant can comprise one or more mutation in the ATP binding pocket and/or the substrate binding pocket such that the functions of these domains differs from the native protein (for example, binding substrate in a manner that differs from the native protein). Alternatively, the catalytic domain can be mutated such that the kinase is unable to transfer a phosphate group to a substrate while the ATP binding pocket and/or the substrate binding pocket are not mutated. In one embodiment, the kinase-dead mutant can comprise mutations in all three domains.

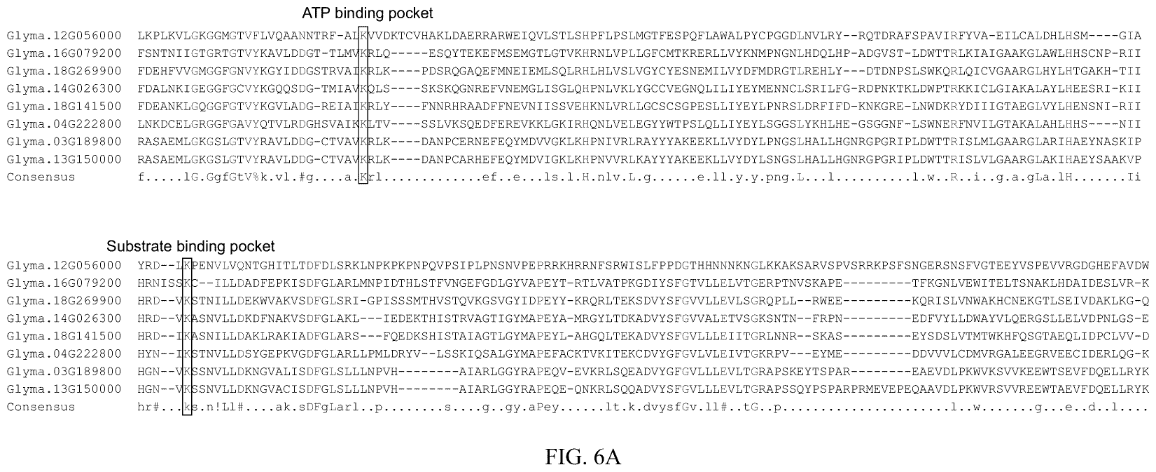

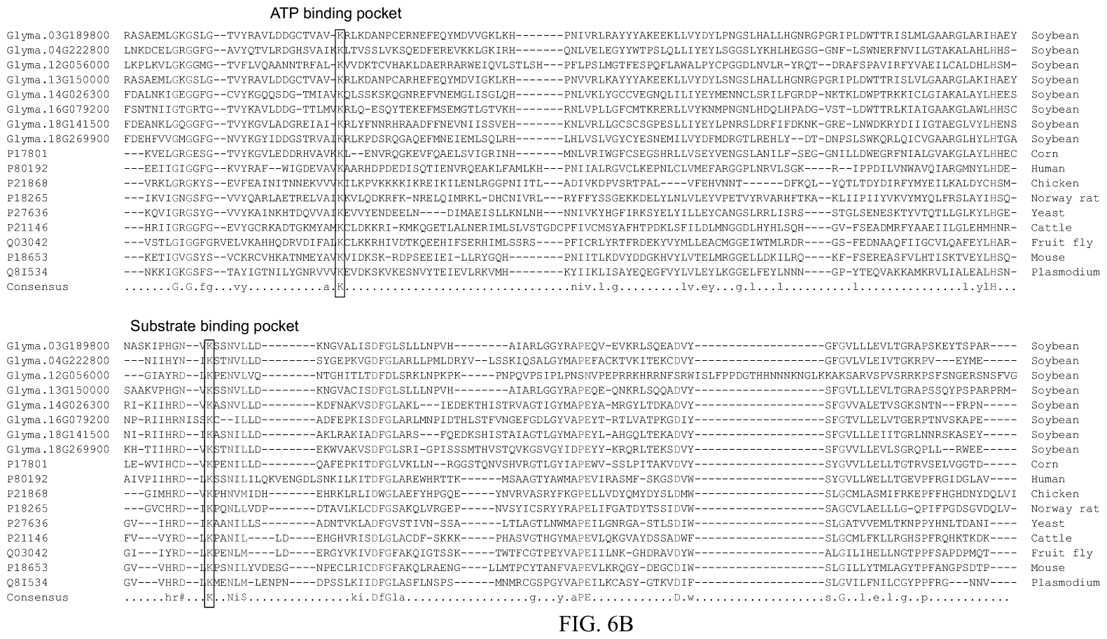

With respect to kinase-dead mutants, one or more amino acid mutations are introduced into what are identified as highly conserved and/or invariant amino acids. In the context of this invention, a highly conserved and/or invariant amino acid is one that is conserved in all kinases. Highly conserved amino acids are those in which less than 20% of the amino acids differ from a reference (consensus) sequence at a given amino acid position (see, for example, FIGS. 6A and 6B).

Kinase-dead mutants exhibit no enzymatic activity or significantly reduced enzymatic activity. In the context of this application, “significantly reduced enzymatic activity” refers to a reduction in the enzymatic activity of the kinase-dead mutant as compared to the reference kinase (the non-mutated kinase from which the kinase-dead mutant was made). “Significantly reduced enzymatic activity” refers to a reduction of substrate phosphorylation in an amount of at least (or at least about) 80%. Thus, a kinase-dead mutant has between about 80% and about 99.99% less enzymatic activity as compared to the non-mutated parent kinase of the same sequence. In some embodiments, the kinase-dead mutant exhibits no enzymatic activity.

In certain embodiments, genetic modifications are made to plants or plant cells that “significantly reduces or abolishes the expression of a gene”, for example a gene encoding a kinase. This phrase refers to a reduction of gene expression in an amount of at least (or at least about) 30% as compared to a non-genetically modified plant from which the genetically modified plant was derived (e.g., a soybean plant). Thus, plants that are genetically modified to exhibit significantly reduced or abolished expression of a gene (for example, significantly reduce or abolish the expression of an active protein from the gene) exhibit a reduction in gene expression or expression of active protein that can range from about 30% to about 99.99% or are devoid of expression (expression is abolished) of the gene or an active protein encoded by the gene.

As used herein, “kinome” is the protein kinase repertoire in an organism.

As used herein, “co-expressed” or “co-expression” is when the expression patterns of two or more genes are correlated across multiple tissues and/or stress conditions.

As used herein “hub genes” are genes that are highly connected regulatory genes.

The soybean has 2,166 putative genes that encode protein kinases. These protein kinases encompass a vast, interconnected network; however, the role of this network in nematode infection resistance remains mostly unknown.

Analysis of the gene expression landscape networks mediated by stress and control conditions can be a powerful approach to determine changes in network structures and topology. The samples used to determine the expression can be found in various plant tissues such as, for example, cotyledon, embryo, seed, whole seedling, root, lateral root, root hair, root tip, nodule, leaf, flower, and pod. The stress conditions can be abiotic stressors such as, for example, elevated or decreased CO₂ concentrations relative to normal conditions, cold, heat, drought, increased ozone concentration, elevated and decreased salt concentrations, elevated or decreased pH, iron deprivation, nitrogen deprivation, phosphorous deprivation, and potassium deprivation or biotic stressors such as, for example, Aphis glycines, Macrophomina phaseolina, Fusarium oxysporum, Heterodera glycine, Phytophthora sojae, Sclerotinia sclerotiorum, soybean mosaic virus, and other plants that are considered weeds, which compete with the soybean plant for sunlight, water, and/or nutrients.

To determine the construction of the gene expression networks, the pairwise gene expression correlation between individual soybean genes was identified using Pearson correlation coefficient (PCC) from RNA-seq datasets. The PCC value can be 0.70, with a false discovery rate of less than 0.05. The co-expression network generated using control gene expression data comprised of 61,162 edges and 8,685 nodes. Of these 8,685 nodes 806 are putative protein kinases. Similarly, the co-expression network generated using stress gene expression data comprised of 70,037 edges and 9,600 nodes. Of these 9,600 nodes 887 are kinases. This disclosure indicates that protein kinases constitute a significant part of gene co-expression nodes.

A hub gene is a gene that is co-expressed with at least twenty-five genes. To determine which genes are hub genes, control and stress gene expression data is evaluated to determine if the twenty-five gene threshold is met. In certain embodiments, the prevalence of kinase hub genes was determined. Using the twenty-five gene threshold, 392 kinase hub genes were identified in the control condition network and 464 kinase hub genes were identified in the stress condition network. 271 kinase hub genes were found to be common to both gene co-expression networks, 121 kinase hub genes were specific to the control network, and 193 kinase hub genes were specific to the stress gene co-expression network. Some of the protein kinase hub gene that were common between stressed and controlled condition networks showed altered topology as they were more densely interconnected with other genes under one condition compared to another condition.

The protein kinase hub genes can be further classified based on the relative expression difference in the stress condition network or the control condition network. The genes are assigned to the stress condition network if the number of co-expressed genes were at least two-fold higher and have at least 25 more co-expression events than the control condition network. The genes are assigned to the control condition network if the number of co-expressed genes were at least two-fold higher and have at least 25 more co-expression events than the stress condition network. In certain embodiments, 145 kinase hub genes that were highly interconnected in control condition network compared to the stress condition network were identified and 247 kinase hub genes that were highly interconnected in stress condition network compared to the control condition network were identified, for a total of 392 highly interconnected kinase hub genes.

To identify hub genes that change the topology of signaling pathways in stressed or pathogen infected tissues, the expression patterns were determined in stressed or pathogen infected tissues, preferably in the feeding site of SCN, compared to non-stressed or pathogen infected tissues. This comparison can provide one or more genes that are central to modulating signaling network structure and function in site(s) of stress or pathogen infections. In certain embodiments, the gene list of highly interconnected kinase hubs indicated above (392 genes) was compared with the reference list of syncytial differentially expressed genes (6,903 genes), and 91 protein kinases were identified as “syncytium highly connected hubs,” central to modulating signaling network structure and function in the nematode feeding site. Out of these 91 kinase hub genes, 40 kinase hub genes were highly interconnected in the control condition network and 51 kinase hub genes were highly interconnected in the stress condition network.

The disclosure provides that the amino acid sequences listed in Table 1 (SEQ ID NOS: 1 to 91) provide resistance or susceptibility to a stressor or pathogen in a plant cell or a plant, particularly, a soybean plant cell or a soybean plant. For example, overexpressing in a plant cell or a plant, particularly, a soybean plant cell or a soybean plant, one or more genes that encode an amino acid sequence selected from SEQ ID NOs: 1 to 91 or homologs thereof and/or one or more “kinase-dead” mutant renders the plant cell or the plant resistant to SCN. Also, inactivating in a plant cell or a plant, particularly, a soybean plant cell or a soybean plant, one or more genes that encode an amino acid sequence selected from SEQ ID NOs: 1 to 91 or homologs thereof renders the plant cell or the plant, particularly resistant to SCN.

In certain embodiments a kinase can be inactivated by mutation of one or more amino acids to form a kinase-dead mutant. For example, one or two amino acids can be mutated to form a kinase-dead mutant. Other embodiments provide for the mutation of at least 1, 2, 3, 4, 5, 6, 7, 8, 9, 10, 11, 12, 13, 14, 15, 20, 24, 30 or greater invariant and/or highly conserved amino acids in the ATP binding pocket and/or the substrate binding pocket of the kinase to form a kinase-dead mutant. In preferred embodiments, the mutation of an amino acid residue can result in a substitution. In certain embodiments, the conserved amino acid residue is a lysine that substituted with a different amino acid residue such as, for example, alanine, arginine, asparagine, aspartate, cysteine, glutamate, histidine, isoleucine, leucine, lysine, methionine, phenylalanine, proline, serine, threonine, tryptophan, tyrosine, or valine. The lysine can be substituted with another charged amino acid such as, for example, glutamic acid, aspartic acid, or, preferably, arginine. In preferred embodiments, the kinase-dead mutant has properties such that enzymatic activity is affected (the enzymatic activity is significantly reduced) but there is no effect on substrate recognition or binding to other proteins.

In certain embodiments, the kinase-dead mutant can interrupt the signaling networks and alter expression of downstream genes that mediate stress and/or pathogen infection, particularly SCN infection by mutating genes that encode highly interconnected kinase hub genes in the syncytium. Kinases and downstream genes can be involved in essential signaling processes needed for SCN or other pathogens to infect any soybean cultivar. In certain embodiments, resistance generated through this novel approach can be effective against all SCN HG types and other plant pathogens. Additionally, the resistance can be durable through the evolution of new SCN HG types.

TABLE 1
SEQ ID NOs: 1 to 91 providing candidate amino
acid sequences that confer SCN resistance when
overexpressed, inactivated, or overexpressed and inactivated.

| SEQ ID NO: | Glyma ID NO. |
|---|---|
| 1 | Glyma.01G098400 |
| 2 | Glyma.01G132300 |
| 3 | Glyma.01G155600 |
| 4 | Glyma.02G036500 |
| 5 | Glyma.02G100000 |
| 6 | Glyma.02G127800 |
| 7 | Glyma.02G134600 |
| 8 | Glyma.02G202900 |
| 9 | Glyma.02G208500 |
| 10 | Glyma.02G246000 |
| 11 | Glyma.02G286000 |
| 12 | Glyma.03G020700 |
| 13 | Glyma.03G088800 |
| 14 | Glyma.03G156800 |
| 15 | Glyma.03G189800 |
| 16 | Glyma.04G085200 |
| 17 | Glyma.04G222800 |
| 18 | Glyma.05G006700 |
| 19 | Glyma.05G213200 |
| 20 | Glyma.05G220600 |
| 21 | Glyma.05G229800 |
| 22 | Glyma.06G017500 |
| 23 | Glyma.07G004700 |
| 24 | Glyma.07G011300 |
| 25 | Glyma.07G023500 |
| 26 | Glyma.07G260200 |
| 27 | Glyma.08G002900 |
| 28 | Glyma.08G124800 |
| 29 | Glyma.08G176200 |
| 30 | Glyma.08G201600 |
| 31 | Glyma.08G231100 |
| 32 | Glyma.08G235900 |
| 33 | Glyma.09G030000 |
| 34 | Glyma.09G063200 |
| 35 | Glyma.09G089700 |
| 36 | Glyma.09G098000 |
| 37 | Glyma.09G252000 |
| 38 | Glyma.09G277000 |
| 39 | Glyma.10G038400 |
| 40 | Glyma.10G052500 |
| 41 | Glyma.10G109200 |
| 42 | Glyma.10G218800 |
| 43 | Glyma.10G243000 |
| 44 | Glyma.10G253200 |
| 45 | Glyma.11G089100 |
| 46 | Glyma.11G101700 |
| 47 | Glyma.11G139700 |
| 48 | Glyma.12G032900 |
| 49 | Glyma.12G056000 |
| 50 | Glyma.12G062900 |
| 51 | Glyma.12G104100 |
| 52 | Glyma.12G129600 |
| 53 | Glyma.12G187700 |
| 54 | Glyma.13G002100 |
| 55 | Glyma.13G060400 |
| 56 | Glyma.13G073900 |
| 57 | Glyma.13G106900 |
| 58 | Glyma.13G110700 |
| 59 | Glyma.13G124800 |
| 60 | Glyma.13G150000 |
| 61 | Glyma.13G174900 |
| 62 | Glyma.13G228400 |
| 63 | Glyma.13G235000 |
| 64 | Glyma.13G238700 |
| 65 | Glyma.13G335700 |
| 66 | Glyma.14G026300 |
| 67 | Glyma.14G058900 |
| 68 | Glyma.14G074700 |
| 69 | Glyma.14G206000 |
| 70 | Glyma.14G212100 |
| 71 | Glyma.14G224000 |
| 72 | Glyma.15G111600 |
| 73 | Glyma.15G203600 |
| 74 | Glyma.15G222300 |
| 75 | Glyma.16G079200 |
| 76 | Glyma.17G048900 |
| 77 | Glyma.17G083100 |
| 78 | Glyma.17G149900 |
| 79 | Glyma.17G218500 |
| 80 | Glyma.17G246300 |
| 81 | Glyma.17G250800 |
| 82 | Glyma.18G107600 |
| 83 | Glyma.18G141500 |
| 84 | Glyma.18G273100 |
| 85 | Glyma.19G036600 |
| 86 | Glyma.19G143000 |
| 87 | Glyma.19G166100 |
| 88 | Glyma.19G261700 |
| 89 | Glyma.20G120200 |
| 90 | Glyma.20G137700 |
| 91 | Glyma.18G269900 |

Accordingly, certain embodiments of the invention provide a method of producing an SCN resistant plant cell or a plant comprising expressing, underexpressing or overexpressing in the plant one or more genes encoding a sequence selected from SEQ ID NOs: 1 to 91 or homologs thereof and or one or more kinase-dead mutant, for example, one or more kinase-dead mutant of SEQ ID NOs: 1 to 91 or homologs thereof. In preferred embodiments, the sequence of a kinase-dead mutant of SEQ ID NOs: 1 to 91 is selected from SEQ ID NOs: 92-99. In preferred embodiments, the plant cell or a plant is a soybean plant cell or soybean plant. As is apparent, a kinase-dead mutant can be constructed from any of SEQ ID Nos: 1-91 by mutating highly conserved or invariant amino acids in the substrate binding pocket or the ATP binding pocket of the kinase (for example, lysine residues in each of these domains).

As used herein, the term “overexpressing a gene” or grammatical variations thereof refer to a condition in a genetically modified plant cell or a genetically modified plant wherein the gene encodes for a protein at a level higher than the parent plant cell or the plant without the genetic modification. Thus, a parent plant cell or a parent plant is genetically modified to produce a modified plant cell or modified plant that expresses a gene to produce a protein at a higher level compared to the parent plant cell or parent plant.

Typically, overexpressing a gene in a plant cell or a plant comprises introducing into the plant cell or a plant, a nucleic acid construct comprising the gene. The nucleic acid construct is designed to induce the expression of the protein encoded by the gene. Methods of producing and introducing various nucleic acid constructs comprising genes of interest into a plant cell or a plant to overexpress the genes are well known to a person of ordinary skill in the art and such embodiments are within the purview of the invention. Certain such embodiments are identified below.

A gene is referred to as “operably linked” when it is placed into a functional relationship with another DNA segment (for example, a promoter that is operably linked to a nucleic acid sequence encoding any one of SEQ ID NOs: 1 to 91 or homologs thereof or kinase-dead mutants of SEQ ID NOs: 1 to 91 or homologs thereof; in preferred embodiments, the nucleic acid encodes SEQ ID NOs: 92-99). Enhancers may be operably linked to another DNA segment but need not be contiguous with the coding sequences whose transcription they control. Linking is accomplished by ligation at convenient restriction sites or at adapters or linkers inserted in lieu thereof. The expression cassette can include one or more enhancers in addition to the promoter. By “enhancer” is intended a cis-acting sequence that increases the utilization of a promoter. Such enhancers can be native to a gene or from a heterologous gene. Further, it is recognized that some promoters can contain one or more native, enhancers or enhancer-like elements. An example of one such enhancer is the 35S enhancer, which can be a single enhancer, or duplicated. See for example, McPherson et al, U.S. Pat. No. 5,322,938, which is hereby incorporated by reference in its entirety.

The promoter for driving expression of the genes of interest may be selected based on a number of criteria including, but not limited to, what the desired use is for the operably linked polynucleotide, what location in the plant is expression of the gene of interest desired, and at what level is expression of gene of interest desired or whether it needs to be controlled in another spatial or temporal manner. In one aspect, a promoter that directs expression to particular tissue may be desirable. When referring to a promoter that directs expression to a particular tissue is meant to include promoters referred to as tissue specific or tissue preferred. Included within the scope of the invention are promoters that express highly in the plant tissue, express more in the plant tissue than in other plant tissue, or express exclusively in the plant tissue. For example, “seed-specific” promoters may be employed to drive expression. Specific-seed promoters include those promoters active during seed development, promoters active during seed germination, and/or that are expressed only in the seed. Seed-specific promoters, such as annexin, P34, beta-phascolin, alpha subunit of beta-conglycinin, oleosin, zein, napin promoters have been identified in many plant species such as maize, wheat, rice and barley. See U.S. Pat. Nos. 7,157,629, 7,129,089, and 7,109,392. Such seed-preferred promoters further include, but are not limited to, Ciml (cytokinin-induced message); cZ19BI (maize 19 kDa zein); and milps (myo-inositol-1-phosphate synthase); (see WO 00/11177, herein incorporated by reference). The 27 kDa gamma-zein promoter is a preferred endosperm-specific promoter. The maize globulin-1 and oleosin promoters are preferred embryo-specific promoters. For dicots, seed-specific promoters include, but are not limited to, bean beta phaseolin, napin, beta-conglycinin, soybean lectin, cruciferin, and the like. For monocots, seed-specific promoters include, but are not limited to, promoters of the 15 kDa beta-zein, 22 kDa alpha-zein, 27 kDa gamma-zein, waxy, shrunken 1, shrunken 2, globulin 1, an Lip1, an Ltp2, and oleosin genes. See also WO 00/12733, where seed-preferred promoters from end1 and end2 genes are disclosed; herein incorporated by reference. Each of these aforementioned references is hereby incorporated by reference in its entirety, particularly as it relates to the promoters disclosed within the reference.

In preferred embodiments, a promoter used in the present invention is a promoter for soybean ubiquitin promoters, for example, the promoters for soybean ubiquitin B (UBB)/ubiquitin C (UBC) gene. Certain examples of soybean ubiquitin promoters that could be used in the present invention are described in United States patent application publication numbers 20140053296 and 20100186119. Each of these publications is incorporated by reference in its entirety, particularly, the sequence listing.

The promoters useful in the present invention can also include constitutive, inducible or tissue-specific (preferred) promoters that are operably linked to a gene encoding a protein comprising of any one of SEQ ID NOs: 1 to 91 or homologs thereof and are heterologous to the nucleic acid sequences to which they are operably linked. In other words, the promoters are not those found operably linked to a gene encoding SEQ ID NOs: 1 to 91 or homologs thereof in their native context within a plant, such as a soybean plant. However, genes encoding kinase-dead mutants of SEQ ID NOs: 1 to 91 or homologs thereof can be operably linked to a promoter present in the native context of SEQ ID NOs: 1 to 91 or homologs thereof. In preferred embodiments, gene comprises a nucleic acid sequence that encodes SEQ ID NOs: 92-99 can be operably linked to a promoter present in the native context of SEQ ID Nos: 92-99. Constitutive promoters, generally, are active in most or all tissues of a plant; inducible promoters, which generally are inactive or exhibit a low basal level of expression, and can be induced to a relatively high activity upon contact of cells with an appropriate inducing agent; tissue-specific (or tissue-preferred) promoters, which generally are expressed in only one or a few particular cell types (e.g., root cells); and developmental- or stage-specific promoters, which are active only during a defined period during the growth or development of a plant. Often promoters can be modified, if necessary, to vary the expression level. Certain embodiments comprise promoters exogenous to the species being manipulated (e.g. a soybean plant).

Non-limiting examples of root-specific promoters (a subset of tissue-specific promoters) include root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1BIO promoter (WO06055487, published May 26, 2006), the CRWAQ81 (WO05035770, published Apr. 21, 2005) and the maize ZRP2.47 promoter (NCBI accession number: U38790; GI No. 1063664). Each of these aforementioned references is hereby incorporated by reference in its entirety, particularly as it relates to the promoters disclosed within the reference.

Exemplary constitutive promoters include the 35S cauliflower mosaic virus (CaMV) promoter (Odell et al. (1985) Nature 313:810-812), the maize ubiquitin promoter (Christensen et al. (1989) Plant Mol. Biol. 12:619-632 and Christensen et al. (1992) Plant Mol. Biol. 18:675-689); the core promoter of the Rsyn7 promoter and other constitutive promoters disclosed in WO 99/43838 and U.S. Pat. No. 6,072,050; rice actin (McElroy et al. (1990) Plant Cell 2:163-171); pEMU (Last et al. (1991) Theor. Appl. Genet. 81:581-588); MAS (Velten et al. (1984) EMBO J. 3:2723-2730); ALS promoter (U.S. Pat. No. 5,659,026); rice actin promoter (U.S. Pat. No. 5,641,876; WO 00/70067), maize histone promoter (Brignon et al., Plant Mol Bio 22 (6): 1007-1015 (1993); Rasco-Gaunt et al., Plant Cell Rep. 21 (6): 569-576 (2003)) and the like. Other constitutive promoters include, for example, those described in U.S. Pat. Nos. 5,608,144 and 6,177,611, and PCT publication WO 03/102198. Each of these aforementioned references is hereby incorporated by reference in its entirety, particularly as it relates to the promoters disclosed within the reference.

An inducible promoter/regulatory element is one that is capable of directly or indirectly activating transcription of a gene encoding one or more of SEQ ID NOs: 1 to 91 in response to an inducer. The inducer can be a chemical agent such as a protein, metabolite, growth regulator, herbicide or phenolic compound; or a physiological stress, such as that imposed directly by heat, cold, salt, or toxic elements, or indirectly through the action of a pathogen or disease agent such as a virus; or other biological or physical agent or environmental condition. A plant cell containing an inducible promoter/regulatory element may be exposed to an inducer by externally applying the inducer to the cell or plant such as by spraying, watering, heating or similar methods. An inducing agent useful for inducing expression from an inducible promoter is selected based on the particular inducible regulatory element. In response to exposure to an inducing agent, transcription from the inducible regulatory element generally is initiated de novo or is increased above a basal or constitutive level of expression.

Any inducible promoter/regulatory element can be used in the instant invention (See Ward et al., Plant Mol. Biol. 22:361-366, 1993). Non-limiting examples of such promoters/regulatory elements include: a metallothionein regulatory element, a copper-inducible regulatory element, or a tetracycline-inducible regulatory element, the transcription from which can be effected in response to divalent metal ions, copper or tetracycline, respectively (Furst et al., Cell 55:705-717, 1988; Mett et al., Proc. Natl. Acad. Sci., USA 90:4567-4571, 1993; Gatz et al., Plant J. 2:397-404, 1992; Roder et al., Mol. Gen. Genet. 243:32-38, 1994). Inducible promoters/regulatory elements also include an ecdysone regulatory element or a glucocorticoid regulatory element, the transcription from which can be effected in response to ecdysone or other steroid (Christopherson et al., Proc. Natl. Acad. Sci., USA 89:6314-6318, 1992; Schena et al., Proc. Natl. Acad. Sci., USA 88:10421-10425, 1991; U.S. Pat. No. 6,504,082); a cold responsive regulatory element or a heat shock regulatory element, the transcription of which can be effected in response to exposure to cold or heat, respectively (Takahashi et al., Plant Physiol. 99:383-390, 1992); the promoter of the alcohol dehydrogenase gene (Gerlach et al., PNAS USA 79:2981-2985 (1982); Walker et al., PNAS 84 (19): 6624-6628 (1987)), inducible by anaerobic conditions; and the light-inducible promoter derived from the pea rbcS gene or pea psaDb gene (Yamamoto et al. (1997) Plant J. 12 (2): 255-265); a light-inducible regulatory element (Feinbaum et al., Mol. Gen. Genet. 226:449, 1991; Lam and Chua, Science 248:471, 1990; Matsuoka et al. (1993) Proc. Natl. Acad. Sci. USA 90 (20): 9586-9590; Orozco et al. (1993) Plant Mol. Bio. 23 (6): 1129-1138), a plant hormone inducible regulatory element (Yamaguchi-Shinozaki et al., Plant Mol. Biol. 15:905, 1990; Kares et al., Plant Mol. Biol. 15:225, 1990), and the like. An inducible promoter/regulatory element also can be the promoter of the maize In2-1 or In2-2 gene, which responds to benzenesulfonamide herbicide safeners (Hershey et al., Mol. Gen. Gene. 227:229-237, 1991; Gatz et al., Mol. Gen. Genet. 243:32-38, 1994), and the Tet repressor of transposon Tn10 (Gatz et al., Mol. Gen. Genct. 227:229-237, 1991). Stress inducible promoters include salt/water stress-inducible promoters such as P5CS (Zang et al. (1997) Plant Sciences 129:81-89); cold-inducible promoters, such as, cor15a (Hajela et al. (1990) Plant Physiol. 93:1246-1252), cor15b (Wlihelm et al. (1993) Plant Mol Biol 23:1073-1077), wsc120 (Ouellet et al. (1998) FEBS Lett. 423-324-328), ci7 (Kirch et al. (1997) Plant Mol Biol. 33:897-909), ci21A (Schneider et al. (1997) Plant Physiol. 113:335-45); drought-inducible promoters, such as, Trg-31 (Chaudhary et al (1996) Plant Mol. Biol. 30:1247-57), rd29 (Kasuga et al. (1999) Nature Biotechnology 18:287-291); osmotic inducible promoters, such as Rab17 (Vilardell et al. (1991) Plant Mol. Biol. 17:985-93) and osmotin (Raghothama et al. (1993) Plant Mol Biol 23:1117-28); and heat inducible promoters, such as heat shock proteins (Barros et al. (1992) Plant Mol. 19:665-75; Marrs et al. (1993) Dev. Genet. 14:27-41), smHSP (Waters et al. (1996) J. Experimental Botany 47:325-338), and the heat-shock inducible element from the parsley ubiquitin promoter (WO 03/102198). Other stress-inducible promoters include rip2 (U.S. Pat. No. 5,332,808 and U.S. Publication No. 2003/0217393) and rd29a (Yamaguchi-Shinozaki et al. (1993) Mol. Gen. Genetics 236:331-340). Certain promoters are inducible by wounding, including the Agrobacterium pmas promoter (Guevara-Garcia et al. (1993) Plant J. 4 (3): 495-505) and the Agrobacterium ORF13 promoter (Hansen et al., (1997) Mol. Gen. Genet. 254 (3): 337-343). Each of these aforementioned references is hereby incorporated by reference in its entirety, particularly as it relates to the promoters disclosed within the reference.

Overexpression of a gene comprising a nucleic acid sequence encoding any one of SEQ ID NOs: 1 to 91 or homologs thereof and/or kinase-dead mutants of SEQ ID NOs: 1 to 91 or homologs thereof can also be achieved by one or one or more mutations in the endogenous promoter of the gene, wherein the one or more mutations increase the expression of the gene. In preferred embodiments, the kinase-dead mutant of SEQ ID NOs: 1 to 91 is selected from SEQ ID NOs: 92-99. For a particular gene, a skilled artisan can identify one or more mutations that would increase the expression of the gene and such embodiments are within the purview of the invention. In certain preferred embodiments, the overexpression is of a gene encoding an inactive protein.

Certain preferred embodiments of the invention provide a method of producing a plant cell or a plant that is resistant to SCN, the method comprising overexpressing in the plant cell or the plant a gene comprising a nucleic acid sequence encoding a kinase protein, particularly a kinase selected from SEQ ID NOs: 1 to 91 or homologs thereof and/or one or more kinase-dead mutant of SEQ ID NOs: 1 to 91 or homologs thereof. In preferred embodiments, the nucleic acid sequence encodes a kinase-dead mutant selected from SEQ ID NOs: 92 to 99. The overexpressed gene encoding the kinase or the kinase-dead protein can comprise a nucleic acid sequence encoding Glyma.03G189800 (SEQ ID NO: 15), Glyma.16G079200 (SEQ ID NO: 75), Glyma.14G026300 (SEQ ID NO: 66), Glyma.04G222800 (SEQ ID NO: 17), Glyma.18G141500 (SEQ ID NO: 83), Glyma.18G269900 (SEQ ID NO: 91), Glyma.12G056000 (SEQ ID NO: 49), Glyma.13G150000 (SEQ ID NO: 60), or a homolog thereof. The overexpressed gene encoding the kinase-dead protein is selected from SEQ ID NOs: 92 to 99.

TABLE 2
SEQ ID NOs: 92 to 99 provide amino acid sequences of kinase-dead
mutants of Glyma.03G189800 (SEQ ID NO: 15), Glyma.16G079200
(SEQ ID NO: 75), Glyma.14G026300 (SEQ ID NO: 66), Glyma.04G222800
(SEQ ID NO: 17), Glyma.18G141500 (SEQ ID NO: 83), Glyma.18G269900
(SEQ ID NO: 91), Glyma.12G056000 (SEQ ID NO: 49), Glyma.13G150000
(SEQ ID NO: 60) that confer SCN resistance.

| SEQ ID NO: | Kinase-dead mutant of: |
|---|---|
| 92 | Glyma.03G189800 (SEQ ID NO: 15) |
| 93 | Glyma.04G222800 (SEQ ID NO: 17) |
| 94 | Glyma.12G056000 (SEQ ID NO: 49) |
| 95 | Glyma.13G150000 (SEQ ID NO: 60) |
| 96 | Glyma.14G026300 (SEQ ID NO: 66) |
| 97 | Glyma.16G079200 (SEQ ID NO: 75) |
| 98 | Glyma.18G141500 (SEQ ID NO: 83) |
| 99 | Glyma.18G269900 (SEQ ID NO: 91) |

Additional embodiments of the invention also provide a plant cell comprising an overexpressed gene comprising a nucleic acid sequence encoding any one of SEQ ID NOs: 1 to 91 or homologs thereof. The plant cell or the plant can be a soybean plant cell or soybean plant.

Further embodiments of the invention provide a method of producing an SCN resistant plant cell or a plant comprising inactivating in the plant one or more genes comprising a nucleic acid sequence encoding any one of SEQ ID NOs: 1 to 91 or homologs thereof and/or expressing one or more kinase-dead mutant of SEQ ID NOs: 1 to 91 or homologs thereof. In preferred embodiments, the nucleic acid sequence encodes a kinase-dead mutant selected from SEQ ID NOs: 92 to 99. In preferred embodiments, the plant cell or a plant is a soybean plant cell or soybean plant.

Typically, inactivating a gene in a plant cell or a plant comprises introducing into the gene one or more mutations that inhibit, significantly reduce or abolish the expression of an active protein from the gene. Mutations in a gene that inhibit, significantly reduce or abolish the expression of a protein from the gene can be achieved either by deleting the entire coding region of the gene or a portion of the coding region of the gene, by introducing a frame shift mutation within the coding region of the gene, by introducing a missense mutation, insertion of sequences that disrupt the activity of the protein encoded by the gene, by introducing a stop codon or any combination of the aforementioned gene mutations. Inactivating a gene can also be performed by using molecular markers or other traditional breeding methods to integrate activated or inhibited genes in any soybean germplasm. Further, inactivating one or more genes can be performed by introducing and/or expressing the one or more genes under soybean endogenous promoters and/or any exogenous promoters.

In preferred embodiments, inactivating a gene in a plant cell or a plant comprises introducing into the gene one or more mutations that alters at least one, two, three, four, five, or more amino acid residues of the encoded protein. The introduced changes, preferably, either increase the expression or do not alter the expression of the gene. In preferred embodiments, the one or more mutations inactivate the enzymatic activity of the protein but do not alter substrate recognition or protein binding. An alternation of the amino acid sequence can comprise the deletion of amino acid, addition of an amino acid, or a change of an amino acid residue. Mutations of the nucleotide sequence can be achieved either by deleting the entire coding region of the gene or a portion of the coding region of the gene, by introducing a frame shift mutation within the coding region of the gene, by introducing a missense mutation, insertion of sequences that disrupt the activity of the protein encoded by the gene, by introducing a stop codon or any combination of the aforementioned gene mutations. Inactivating a gene can also be performed by using molecular markers or other traditional breeding methods to integrate activated or inhibited genes in any soybean germplasm. Further, inactivating one or more genes can be performed by introducing and/or expressing the one or more genes under soybean endogenous promoters and/or any exogenous promoters. The result of the alteration of at least one, two, three, four, five, or more amino acid resides can inactivate the enzymatic function of the protein but the protein can still bind or recognize substrates or other proteins.

Methods of inactivating a gene of interest in a plant cell or a plant to inhibit, significantly reduce, or abolish the expression of an active protein or retain expression but inactivate protein enzymatic function are well known to a person of ordinary skill in the art and such embodiments are within the purview of the invention. Certain such embodiments are identified below.

Sanagala et al. (2017), Journal of Genetic Engineering and Biotechnology; 15 (2): 317-321, describe several methods of inactivating a gene, for example, by implementing homologous recombination, zinc finger nucleases, Transcription activator-like effector nucleases (TALENs), and the clustered regularly interspaced short palindromic repeats/CRISPR-associated (CRISPR/Cas) system. The Sanagala et al. reference is incorporated herein by reference in its entirety.

In preferred embodiments, inactivating a gene of interest is performed using the CRISPR/Cas system. An example of such system to inactivate genes in a plant cell or a plant is provided by Ordon et al. (2017), The Plant Journal; 89:155-168. The Ordon et al. reference is incorporated herein by reference in its entirety.

Typically, a CRISPR/Cas system mediated inactivation of a gene involves the use of a guide RNA targeted to a gene of interest. A DNA oligomer targeted to a gene of interest can be transcribed into single guide RNA (sgRNA). sgRNA guides the Cas9 DNA endonuclease to the gene of interest by sgRNA hybridization to the target site. The endonuclease Cas9 makes a double strand break 3 bp upstream of Palindromic Adjacent Motif (PAM). The DNA breakage engages the repair mechanism, such as homologous recombination (HR) or the non-homologous end joining (NHEJ) mechanism. The NHEJ mechanism is a major double strand break repair pathway in plants and is known to be error prone. NHEJ DNA repair process introduces errors in the DNA repair, which causes irreversible mutations at the gene of interest. The chances of errors in DNA repair can be increased by providing multiple sgRNA. Based on the sequence of a gene comprising a nucleic acid sequence encoding any one of SEQ ID NOs: 1 to 91 or homologs thereof, a person of ordinary skill in the art can design and perform an inactivation of the gene using the CRISPR/Cas system and such embodiments are within the purview of the invention.

Methods of inactivating a gene of interest in a plant cell or a plant to inhibit, significantly reduce, or abolish the expression of an active protein or retain expression but inactivate protein enzymatic function also include introduction into the plant cell or the plant one or more inhibitory oligonucleotides, such as small interfering RNA (siRNA) or short hairpin RNAs (shRNA). Methods of producing and introducing inhibitory RNA are also well known to a person of ordinary skill in the art and such embodiments are within the purview of the invention.

Certain preferred embodiments of the invention provide a method of producing a plant cell or a plant that is resistant to SCN, the method comprising inactivating in the plant cell or the plant a gene encoding a kinase protein. The inactivated gene can comprise a nucleic acid sequence encoding Glyma.03G189800 (SEQ ID NO: 15), Glyma.16G079200 (SEQ ID NO: 75), Glyma.14G026300 (SEQ ID NO: 66), Glyma.04G222800 (SEQ ID NO: 17), Glyma.18G141500 (SEQ ID NO: 83), Glyma.18G269900 (SEQ ID NO: 91), Glyma.12G056000 (SEQ ID NO: 49), Glyma.13G150000 (SEQ ID NO: 60), or a homolog thereof. In certain embodiments the inactivated gene, can be overexpressed in the plant cell or the plant, which can comprise a nucleic acid sequence encoding of Glyma.03G189800 (SEQ ID NO: 15), Glyma.16G079200 (SEQ ID NO: 75), Glyma.14G026300 (SEQ ID NO: 66), Glyma.04G222800 (SEQ ID NO: 17), Glyma.18G141500 (SEQ ID NO: 83), Glyma.18G269900 (SEQ ID NO: 91), Glyma.12G056000 (SEQ ID NO: 49), Glyma.13G150000 (SEQ ID NO: 60), or a homolog thereof. Furthermore, one or more kinase-dead mutant Glyma.03G189800 (SEQ ID NO: 15), Glyma.16G079200 (SEQ ID NO: 75), Glyma.14G026300 (SEQ ID NO: 66), Glyma.04G222800 (SEQ ID NO: 17), Glyma.18G141500 (SEQ ID NO: 83), Glyma.18G269900 (SEQ ID NO: 91), Glyma.12G056000 (SEQ ID NO: 49), Glyma.13G150000 (SEQ ID NO: 60) can be expressed in a plant to confer SCN resistance. In preferred embodiments, the nucleic acid sequence encodes a kinase-dead mutant selected from SEQ ID NOs: 92 to 99.

Additional embodiments of the invention also provide a plant or a plant cell comprising an inactivated gene comprising a nucleic acid sequence encoding SEQ ID NOs: 1 to 91 or homologs thereof. The plant cell or the plant can be a soybean plant cell or soybean plant.

In the methods of producing a plant cell described herein the plant cell can be in a plant part, for example, a seed, endosperm, ovule or pollen. The plant can be a soybean plant.

Further embodiments of the invention provide methods for identifying a gene that induces SCN resistance when overexpressed, inactivated, or overexpressed and inactivated in a plant cell or a plant, the method comprising the steps of: (a) overexpressing a kinase or kinase-dead (inactivated) hub gene in a plant cell or a plant, compared to the expression in a plant cell or a plant known to be susceptible to SCN, or inactivating a kinase hub gene in a plant cell or a plant known to be resistant to SCN, (b) testing the SCN resistance in the plant cell or the plant comprising the overexpressed, inactivated, or the overexpressed and inactivated gene, and (c) identifying the gene that induces resistance in the plant cell or the plant when overexpressed, inactivated, or both overexpressed and inactivated in the plant cell or the plant when overexpressed or inactivated. In preferred embodiments, an overexpressed and/or inactivated gene comprises a nucleic acid sequence encoding any one of SEQ ID NOs: 1-91 or homologs thereof. In some embodiments, one or more genes comprising nucleic acid sequences encoding SEQ ID NOs: 1-91 or homologs thereof are overexpressed or one or more genes comprising nucleic acid sequences encoding SEQ ID NOs: 1-91 or homologs thereof are inactivated. In certain embodiments, one or more genes comprising nucleic acid sequences encoding SEQ ID NOs: 1-91 or homologs thereof are overexpressed and one or more genes comprising nucleic acid sequences encoding SEQ ID NOs: 1-91 or homologs thereof are inactivated. Yet other embodiments provide for the expression or overexpression of kinase-dead mutants of SEQ ID NOs: 1 to 91 or homologs thereof in a plant. In preferred embodiments, one or more kinase-dead mutant selected from SEQ ID NOs: 92 to 99 is expressed or overexpressed in a plant cell. The plant cell or the plant can be a soybean plant cell or a soybean plant.

The details discussed above for overexpressing and inactivating genes in a plant cell or a plant are also applicable to the methods of identifying a gene that induces SCN resistance in a plant cell or a plant when overexpressed or inactivated and such embodiments are within the purview of the invention.

As discussed above, comparing the kinomes of the parental soybean lines that are susceptible to SCN with mutant soybean lines comprising kinase-dead proteins that are resistant to SCN resulted in the identification of heritable genes related to SCN parasitism of soybean. Thus, the disclosure provides genes involved in the biochemical basis of kinase function in SCN resistance. Such comparisons can also be used to identify genes involved in conferring traits other than SCN resistance.

A method of identifying one or more kinase genes that confer a trait to a plant when expressed, inactivated, or overexpressed and inactivated, the method comprising the steps of:
a) providing a kinase gene;
b) analyzing the mRNA expression profiles of the kinase gene during controlled conditions and under stressed conditions (for example, a biotic or abiotic stress);
c) comparing the mRNA expression profiles of the kinase gene during the stress conditions to the kinase gene during controlled conditions to identify the kinase gene that are highly interconnected in control condition network compared to the stress condition network;
d) identifying the kinase hub genes based on co-expression with at least twenty-five genes specific to the stress network and specific to the controlled network;
e) comparing the interconnected kinase hub genes identified in step d) with differentially expressed genes in a plant cell;
f) identifying the protein kinase hub genes that are differentially expressed in the plant cell to identify highly connected hub kinase genes;
g) creating kinase-dead variants of the highly connected hub kinase genes;
h) expressing or overexpressing a kinase-dead variants of the identified highly connected hub kinase genes and/or inactivating highly connected hub kinase genes in the plant cell; and
i) identifying one or more genes that confer a trait to the plant when the kinase-dead variant is expressed or overexpressed and/or the highly connected hub-kinase gene is inactivated. In various embodiments, the method can further comprise the generation of a plant expressing or overexpressing the kinase-dead variant and/or inactivating the highly connected hub-kinase gene in a plant and conferring a desired trait (for example, resistance to a biotic and/or abiotic stress as disclosed herein) to the plant when the kinase-dead variant is expressed or overexpressed and/or the highly connected hub-kinase gene is inactivated.

As used herein, the phrase “a gene confers a trait” or grammatical variations thereof indicates that a plant containing the gene exhibits the trait and an otherwise genetically identical plant lacking the gene does not exhibit the trait. Thus, in two otherwise genetically identical plants, the presence of a trait in the plant containing the gene encoding the kinase-dead variant and the absence of the trait in the plant not containing the gene is attributed to the presence or the absence of the gene.

In preferred embodiments of the invention, the plant is a soybean plant and the trait is resistance to SCN. In certain embodiments, the plant cell is a syncytial cell. In certain embodiments, the kinase-dead variant is created by mutating the nucleotides encoding one or two amino acids of the kinase.

Exemplary embodiments of such methods are described in Examples 1 to 5 below. A skilled artisan can implement such methods to identify genes that confer a trait of interest in a plant and such embodiments are within the purview of the invention.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—Genome-Wide Identification and Functional Classification of Soybean Protein Kinases

With the availability of the soybean whole-genome sequences along with the annotation of the encoded proteins (Schmutz et al., 2010), we identified and functionally classified the entire soybean protein kinase repertoire or kinome, which includes 2,166 putative protein kinase genes, representing 4.67% of all soybean protein-coding genes. The soybean kinome was classified into 19 groups, 81 families and 122 subfamilies (Liu et al., 2015). Gene structure, subcellular localization prediction and gene expression patterns pointed to extensive functional divergence of protein kinase subfamilies.

### Example 2—Generation of Global Gene Co-Expression Networks and Identification of Highly Connected Kinase-Hub Genes

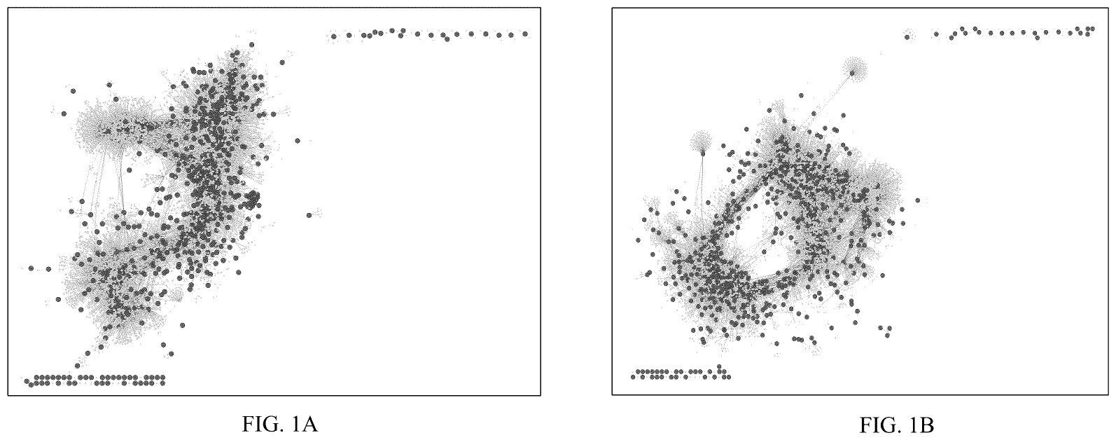

Differential networking of gene expression has emerged as a powerful approach to detect changes in network structures and topology to identify differentially connected hubs among two networks (de la Fuente, 2010; Palumbo et al., 2014). We generated two independent coexpression networks of soybean genes using control and stress response gene expression data from publicly available RNA-seq datasets (FIG. 1A and B). The coexpression networks were generated from 394 samples collected under normal conditions, and 281 samples collected under various stress conditions. These samples were from different tissues such as cotyledon, embryo, seed, whole seedling, root, lateral root, root hair, root tip, nodule, leaf, flower and pod. The RNA-seq samples collected under stressed conditions included samples that were treated with various abiotic stresses (CO₂, cold, drought, heat, ozone, salt, pH, iron deprivation, nitrogen deprivation, phosphorous deprivation and potassium deprivation), or biotic stresses (Aphis glycines, Macrophomina phaseolina, Fusarium oxysporum, Heterodera glycine, Phytophthora sojae, Sclerotinia sclerotiorum, soybean mosaic virus, and weed). Construction of the networks were performed by determining all pairwise gene expression correlations between individual soybean genes using Pearson correlation coefficient (PCC) value of 0.70 and a false discovery rate less than 0.05. The co-expression network generated using control gene expression data comprised of 61,162 edges and 8,685 nodes (FIG. 1A). Of these 8,685 nodes 806 are putative protein kinases (Red dots in FIG. 1A). Similarly, the co-expression network generated using stress gene expression data comprised of 70,037 edges and 9,600 nodes (FIG. 1B). Of these 9,600 nodes 887 are kinases (Red dots in FIG. 1B). These results indicate that protein kinases constitute a significant part of gene co-expression nodes.

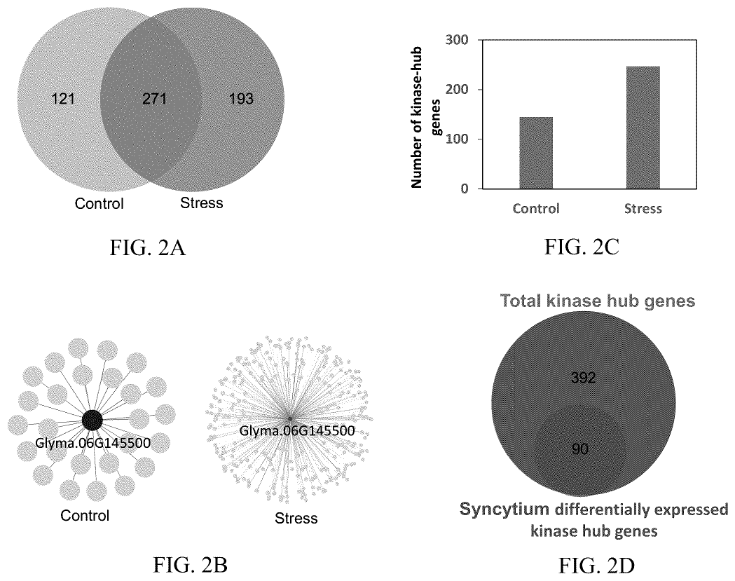

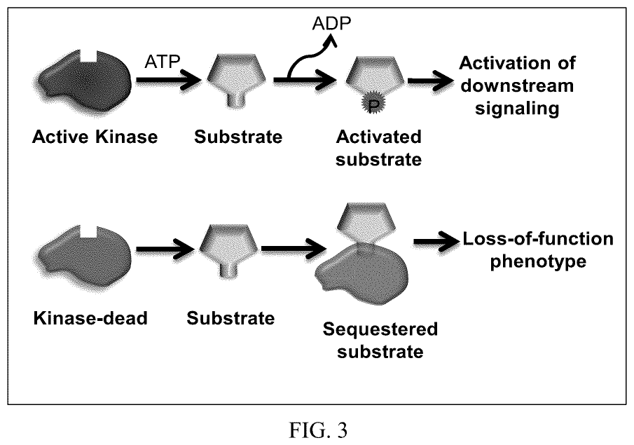

We identified all the kinase hub genes in the gene co-expression network generated using control and stress gene expression data. A protein kinase was considered a hub only if it is co-expressed with at least twenty-five genes. Using this parameter, we identified 392 kinase hub genes in control condition network and 464 kinase hub genes in stress condition network (FIG. 2A). We found that 271 kinase hub genes are common to both gene co-expression networks, 121 kinase hub genes were specific to the control network, and 193 kinase hub genes were specific to the stress gene co-expression network (FIG. 3A). Some of the protein kinase hub gene that were common between stressed and controlled condition networks showed altered topology as they were more densely interconnected with other genes under one condition compared to another condition. For example, a protein kinase Glyma.06G145500 co-expresses with 26 genes under controlled conditions, while the same protein kinase co-expresses with 309 genes under stressed condition (FIG. 2B). Therefore, we assigned these protein kinase hub genes to be specific to stressed conditions if the number of co-expressed genes were at least two-fold higher and has at least 25 more co-expression events than control condition network and vice versa. Using this criterion, we identified 145 kinase hub genes that were highly interconnected in control condition network compared to the stress condition network and 247 kinase hub genes that were highly interconnected in stress condition network compared to the control condition network (FIG. 2C). In total, we identified 392 protein kinase hub genes that are specific to control or stress condition network. Since these protein kinase hub genes show altered topology under control and stress conditions, we hypothesize that these kinase hub genes play key role in plant responses to various biotic or abiotic stresses.

### Example 3—Identification of Highly Connected Kinase Hubs that Change the Expression in the Scn Feeding Site

Highly connected hub genes have been shown to play key roles in the biology of lower organisms (yeast, fly and worm) and higher organisms (mice and humans) (Vidal et al., 2011). Identifying hub genes that change the topology of signaling pathways in the feeding site of SCN is expected to lead to identifying crucial targets for genetic manipulation. We reasoned that highly connected kinase hubs with key role in mediating plant susceptibility to SCN should exhibit differential expression in the syncytium relative to non-syncytial cells. Therefore, we compared the gene list of highly interconnected kinase hubs indicated above (392 genes) with our reference list of syncytial differentially expressed genes (6,903 genes), and identified 91 protein kinases as “syncytium highly connected hubs,” central to modulating signaling network structure and function in the nematode feeding site (FIG. 2D; Table 1). Out of these 91 kinase hub genes, 40 kinase hub genes were highly interconnected in control condition network and 51 kinase hub genes were highly interconnected in stress condition network. Subcellular localization prediction of these kinases provided suggestions for extracellular, cytoplasmic or nuclear localizations, suggesting a role of these hubs in signals perception and transmission to the nucleus to control gene expression of downstream targets.

### Example 4—Kinase-Dead Mutation: A New Approach for Functional Studies of Kinases

The robust conservation of kinase domains among kinome members has allowed the identification of critical amino acids required for the activity of these enzymes. The provided multiple sequence alignments of the protein kinase domains of 8 syncytium-kinase hub genes showing the conserved amino acids including 2 lysine residues (K) in the ATP binding pocket and the substrate binding pocket (FIGS. 6A and 6B) demonstrate sites that can be mutated to inactivate the kinase. In various embodiments, the underlined lysine residues in the sequences identified in the section entitled “Sequences found in Table 2” are substituted with arginine residues in kinase-dead mutants represented by the exemplary amino acid sequences of SEQ ID NOs: 92 to 99. Mutations in only one or two amino acids can completely abolish or significantly reduce the activity of these enzymes but do not interfere with substrate recognition or binding to other proteins. As a result, protein kinases can be modified to generate kinase-dead mutants, which have no enzymatic activity but have dominant-negative effects in sequestering the activity of the substrates/interactors, causing loss-of-function phenotype (FIG. 3). In this function, kinase-dead mutations of syncytium highly interconnected kinase hub genes will interrupt the signaling networks and alter expression of downstream genes that mediate SCN susceptibility, resulting in plant resistance to SCN. Because these kinases and downstream genes would be involved in essential signaling processes needed for SCN to infect any soybean cultivar, resistance generated through this novel approach is expected to be effective against all SCN HG types and new HG types are unlikely to evolve (the resistance will be durable). Therefore, kinase-dead mutations can be established as highly effective approach to examine the contribution of hub kinases, identified above, to soybean responses to SCN.

### Example 5—Overexpression of Kinase-Dead Variants of Syncytium Hub-Kinase Genes Significantly Enhanced Soybean Resistance to Scn

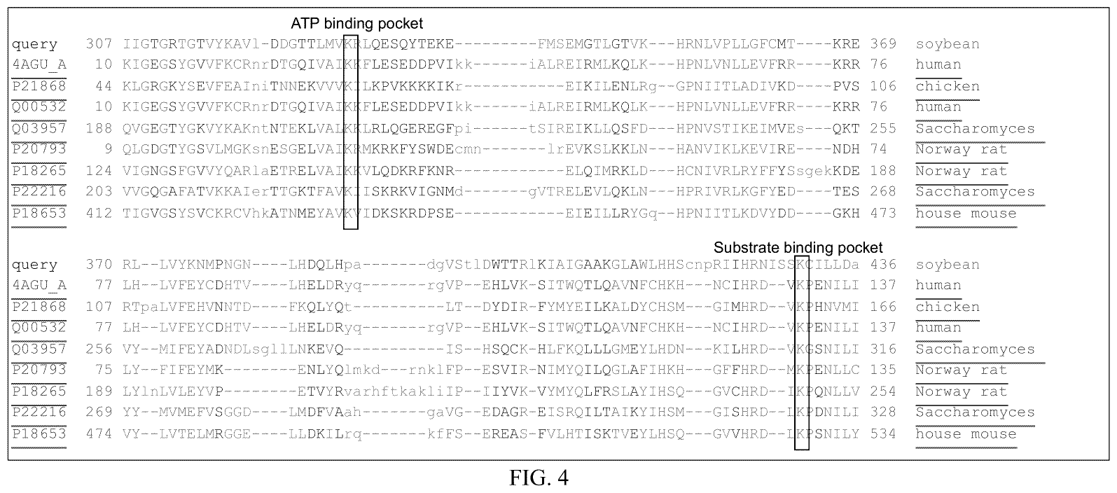

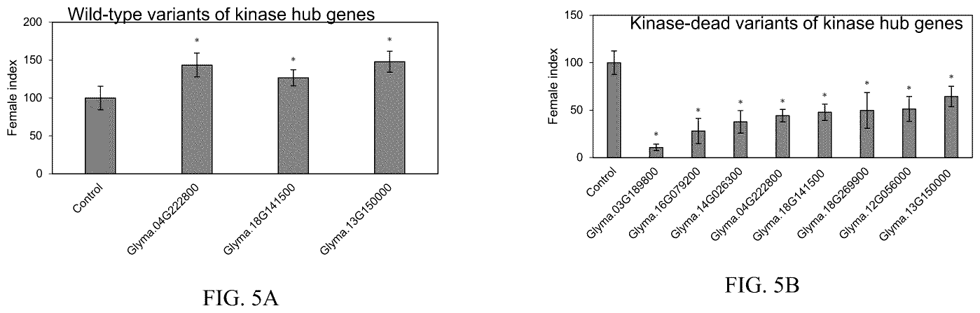

We performed a proof-of-concept study to assess the impact of manipulating kinase hub genes on SCN parasitism of soybean plants. If syncytium hub-kinase genes have functions in mediating soybean susceptibility to SCN, one can reasonably postulate that ectopic expression of inactive variants in soybean will function antagonistically to the wild-type variants, resulting in increased soybean resistance to SCN. To this end, we overexpressed the wild-type coding sequences and kinase-dead variants of 8 syncytium highly connected kinase hub genes using soybean hairy root system and challenged the transgenic roots with SCN. The kinase-inactive variants were generated by mutating the two conserved lysine residues in the ATP binding pocket and the substrate binding pocket to arginine (FIG. 4). These lysines interact directly with ATP and mutation of these residues lead to a classical “kinase-dead” mutant. Because the main function of protein kinases is the phosphorylation of specific substrates, kinase-dead mutations confer loss-of-function phenotypes. Several transgenic hairy root plants overexpressing these 8 kinase-dead variants under the control of a soybean ubiquitin promoter were generated in the SCN susceptible cv ‘Williams 82’. The transgenic roots were identified and selected using the green fluorescent protein (GFP). Transgenic plants expressing the empty vector containing only the GFP marker gene were used as control. Both transgenic and control plants were inoculated with 3000 eggs of SCN (HG type 0 or race 3) in three biologically independent experiments each with at least 5 plants. Five weeks after inoculation the number of cysts per plant were counted and used to calculate female index. The female index was calculated by dividing the average number of cysts on the transgenic hairy root lines by the average cysts on the control plants (empty vector) multiplied by 100. We found that growth, development and morphology of the transgenic hairy roots overexpressing wild-type or kinase-dead genes were indistinguishable from those overexpressing the empty vector. We found that transgenic soybean plants overexpressing the wild-type variant of various kinase hub genes (Glyma.04G222800, Glyma.13G150000 and Glyma.18G141500) showed significantly higher number of cysts per root system compared to the transgenic hairy root plants expressing the empty vector (FIG. 5A). However, when we overexpressed 8 kinase hub genes in inactive (kinase-dead) form (Glyma.03G189800, Glyma.16G079200, Glyma.14G026300, Glyma.04G222800, Glyma.18G141500, Glyma.18G269900, Glyma.12G056000, and Glyma.13G150000) the transgenic hairy roots showed significant reduction in SCN susceptibility, ranging between 35 and 90% (FIG. 5B) compared with the control plants. The amino acid sequences of the kinase-dead mutants are provided in Table 2. These results confirm: (1) kinase hubs are master regulators of soybean susceptibility to SCN; and (2) the structure-function of the kinase-dead mutation in enhancing soybean resistance to SCN.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

Sequences found in Table 1
>Glyma.01G098400
SEQ ID NO: 1
MGACWSSRIKAVSPSNTGFTSRSVSRDGHDIQSSSRNSSASIPMTPRSEGEILQFSNLKSYSYNELKMATKNFCP
DSVLGEGGFGSVFKGWIDEHSLAVTRPGTGMVIAVKKLNQDSFQGHKEWLAEINYLGQLQNPNLVKLIGYCLEDQ
HRLLVYEYMPKGSVENHLFRRGSHFQQLSWTLRLKISLGAARGLAFLHSTETKVIYRDFKTSNILLDTNYNAKLS
DFGLARDGPTGDKSHVSTRVMGTHGYAAPEYLATGHLTAKSDVYSFGVVLLEMLSGRRAIDKNRPSGEQCLVEWA
KPYLSNKRRVFRVMDSRLEGQYSLTQAQRAATLAFQCLSVEPKYRPNMDEVVKALEQLRESNDKVKNGDHKKCRV
SGSGLGHPNGLPASTSKGSIDAAKKFNYPRPSASLLYS*
>Glyma.01G132300
SEQ ID NO: 2
MSYRERNKGKGEEKEYENSTTRGLDSKSVSHNNGSIEEELLTIDENLLIDPKLLFIGSKIGEGAHGRVYEGRYRD
QIVAIKVLHRGGTLEERVALENRFAREVNMMSRVHHENLVKFIGACKDPLMVIVTEMLPGLSLRKYLTTIRPKQL
DPYVAIKFALDIARAMDWLHANGIIHRDLKPDNLLLTENQKSVKLADFGLAREESVTEMMTAETGTYRWMAPELY
STVTLCQGEKKHYNNKVDVYSFGIVLWELLTNRMPFEGMSNLQAAYAAAFKQERPNLPDDISPDLAFIIQSCWVE
DPNMRPSFSQIIRLLNEFHFTLQQPSPSMPLEPENEPEAITSNGTITDFSTRNKVKFSFIRHLFSSKRTKS*
>Glyma.01G155600
SEQ ID NO: 3
MFPLQCSNHPMCAFSAAVITILLLFPAATSQAQILKKETYFFGPFNQSYFTTFAVLPSAAINLGALQVTPDSTGN
VSLANQSGRIFFSTPFTLWDDENLNGKLVSFNTSFLINVFRPQNNPPGEGIAFLIAPSSSTVPNNSHGQFLGLTN
AATDGNATNKFIAVELDTVKQDFDPDDNHIGLDINSVRSNVSVSLTPLGFEIAPNVTRFHVLWVDYDGDRKEIDV
YIAEQPDKDVPIVAKPAKPVLSSPLDLKQVLNKVSYFGFSASTGDNVELNCVLRWNITIEVFPKKNGNGKAYKIG
LSVGLTLLVLIVAGVVGFRVYWIRKKKRENESQILGTLKSLPGTPREFRYQELKKATNNFDDKHKLGQGGYGVVY
RGTLLPKENLQVAVKMFSRDKMKSTDDFLAELTIINRLRHKNLVRLLGWCHRNGVLLLVYDYMPNGSLDNHIFCE
EGSSTTPLSWPLRYKIITGVASALNYLHNEYDQKVVHRDLKASNIMLDSNFNARLGDFGLARALENDKTSYAEME
GVHGTMGYIAPECFHTGRATRESDVYGFGAVLLEVVCGQRPWTKNEGYECLVDWVWHLHREQRILDAVNPRLGND
CVVEEAERVLKLGLACSHPIASERPKMQTIVQILSGSVHVPHLPPFKPAFVWPAMDLSSLASDLTTQTTTTEYTP
MSSDTHSMHVQFSDSSSLV*
>Glyma.02G036500
SEQ ID NO: 4
MEHSSSLVFWLLGLLLLLLMEISSAALSPSGINYEVVALMAIKNDLIDPHNVLENWDINSVDPCSWRMITCSPDG
SVSALGLPSQNLSGTLSPGIGNLTNLQSVLLQNNAISGRIPAAIGSLEKLQTLDLSNNTFSGEIPSSLGGLKNLN
YLRLNNNSLTGSCPQSLSNIEGLTLVDLSYNNLSGSLPRISARTLKIVGNSLICGPKANNCSTILPEPLSFPPDA
LRGQSDSGKKSHHVALAFGASFGAAFVLVIIVGFLVWWRYRRNQQIFFDVNEHYDPEVRLGHLKRFSFKELRAAT
DHENSKNILGRGGEGIVYKACLNDGSVVAVKRLKDYNAAGGEIQFQTEVETISLAVHRNLLRLSGFCSTQHERLL
VYPYMSNGSVASRLKDHIHGRPALDWTRRKRIALGTARGLVYLHEQCDPKIIHRDVKAANILLDEDFEAVVGDFG
LAKLLDHRDSHVTTAVRGTVGHIAPEYLSTGQSSEKTDVFGFGILLLELITGHKALDFGRAANQKGVMLDWVKKL
HQDGRLSQMVDKDLKGNFDLIELEEMVQVALLCTQFNPSHRPKMSEVLKMLEGDGLAERWEASQRIETPRFRSCE
PQRYSDLIEESSLVVEAMELSGPR*
>Glyma.02G100000
SEQ ID NO: 5
MATIGIGVGVTKLQQPHRPKSKSLFLGQRLRISPFWGEPQCSERRLFGVSNPPRVVQVFALGGGEWLDTVHNLFV
GVGVGLPCSVMQCGDVIYRSTLPKSNGLTLTVPGVILALGTLSYLWATPGVAPGFFDMFVLAFVERLFRPTYKKD
DFVLGKKLGEGSFGVVYRVSLANKPSSKEGDLVLKKATEYGAVEIWMNERVRRACASSCADFVYGFLESSSKKAA
EYWLIWRFEGDATLADLMQSRDFPYNVETLILGEVQDLPKGLERENRIIQTIMRQILFALDGLHSTGIVHRDIKP
QNVIFSEESRTFKIIDLGAATDLRVGINYIPKEFLLDPRYAAPEQYIMSTQTPSAPSVPVATALSPVLWQLNLPD
RFDIYSAGLIFLQMAFPSLRSDNSLIQFNRQLKRCDYDLVAWRKTAEARSELRKGFELLDLDGGIGWELLKSMVR
YKARQRLSAKAALAHPYFVREGLLALSFMQTLRLQLLRATQQDYSEAARWIIQLMAKSGTQKDGGFTEAQLQELR
EIEPKKKASAPRNALASALKLQRKIIRTIRTLNESMDELTRRRKSFWWSRWIPREE*
>Glyma.02G127800
SEQ ID NO: 6
MERSGDVALFCLALFFLWTSVAALLSPKGVNYEVQALMSIKNSLVDPHSVLNNWDTDAVDPCNWAMVICSSDHFV
IALGIPSQSISGTLSPSIGNLTNLQTVLLQDNNITGPIPFEIGRLQKLQTLDLSDNFFTGQLPDTLSYMKGLHYL
RLNNNSLTGPIPSSLANMTQLAFLDISYNNLSEPVPRINAKTFNIIGNPQICATGVEKNCFRTTSIPSAPNNSQD
SQSTKRPKSHKFALAFASSLSCICLLILGLGFLIWWRQRYNKQIFFDVNEQHREEVCLGNLKKFHFRELQLATNN
FSSKNLIGKGGFGNVYKGYVQDGTVIAVKRLKDGNAIGGEIQFQTEVEMISLAVHRNLLRLYGFCMTATERLLVY
PYMSNGSVASRLKAKPALDWATRKRIALGAGRGLLYLHEQCDPKIIHRDVKAANILLDDYCEAVVGDFGLAKLLD
HRDSHVTTAVRGTVGHIAPEYLSTGQSSEKTDVFGFGILLLELISGQRALEFGKAANQKGAMLDWVKKIHQEKKI
DLLVDKDLKNNYDRIELDEIVQVALLCTQYLPSHRPKMSEVVRMLEGDGLAEKWEASQSAESTRSRGNELSSSER
YSDLTDDSSLLAQAMELSGPR*
>Glyma.02G134600
SEQ ID NO: 7
MGVCTSKPQKPNPYALREAEAEADPSQNPKTTLSPAGADTPRRKDDVSTGKRSPFFPFYSPSPARFLKKSPAPAG
GSRSASSTPRRFFRRPFPPPSPAKHIRAVLARRQGKKASATAAIPEEGEEGAADLDKRFGFSKEFTSRLEVGEEV
GRGHFGYTCSARFKKGELKGQQVAVKVIPKAKMTTAIAIEDVRREVKILRALNGHNNLIQFYDAFEDQDNVYIVM
ELCEGGELLDMILSRGGKYSEDDAKAVMVQILNVVAFCHLQGVVHRDLKPENFLYAKKDESSELKAIDFGLSDFV
RPDERLNDIVGSAYYVAPEVLHRSYGTEADVWSIGVIAYILLCGSRPFWARTESGIFRAVLKADPSFDETPWPSL
SLEAKDFVKRILNKDPRKRISAAQALSHPWIRNCNNVKVPLDILIFKLMKTYMRSSSLRKAALRALSKTLTADEL
YYLRGQFALLEPSKNGSISLENVNKALMKYATDAMKESRIPDFLSSLNSLQYRRMDFEEFCAAALSVHQLEALDR
WEQHARCAYELFDKDGNRAIVIEELASELGLGPSIPVHVVLHDWIRHTDGKLSFLGFVKLLHGVSSRSLAKVQ*
>Glyma.02G202900
SEQ ID NO: 8
MSEKEKGNNSEGQSTLLHGKYELGRVLGHGTFAKVYHARNLNTGQHVAMKVVGKEKVIKVGMMEQVKREISVMKM
VKHQNIVELHEVMASKSKIYIAMELVRGGELFNKVSKGRLKEDVARLYFQQLISAVDFCHSRGVYHRDLKPENLL
LDEHGNLKVSDFGLTAFSEHLKEDGLLHTTCGTPAYVSPEVIAKKGYDGAKADIWSCGVILYVLLAGFLPFQDDN
LVAMYKKIYRGDFKCPPWFSLDARKLVTKLLDPNPNTRISISKVMESSWFKKPVPRKLAAEKVDLEEEKIESQLE
TINAFHIISLSEGFNLSPLFEDKRREEMRFATAGTPSTVISRLEEVAKAGKFDVRSSETKVRLQGQERGRKGKLA
IAADIYAVIPSFMVVEVKKDNGDTLEYNQFCSKQLRPALKDIFWNSSQNSAPASA*
>Glyma.02G208500
SEQ ID NO: 9
MERYEILKDIGSGNFAVAKLVRDNYTNELFAVKFIERGQKIDEHVQREIMNHRSLKHPNIIRFKEVLLTPTHLAI
VMEYASGGELFERICNAGRFSEDEARFFFQQLISGVSYCHSMQICHRDLKLENTLLDGSTAPRVKICDFGYSKSS
VLHSQPKSTVGTPAYIAPEVLTRKEYDGKIADVWSCGVTLYVMLVGAYPFEDPADPRNFKKTIGKILSVQYSVPD
YVRVSMECRHLLSQIFVASPEKRITIPEIKNHPWFLRNLPMELTEGGSWQMNDVNNPSQNVEEVLSIIQEARKSL
NVPKVGGLLTGGSMDLDDFDADEDLEDLETSGEFVCPI*
>Glyma.02G246000
SEQ ID NO: 10
MMSLCSSKGTCSGDTNAHVSSSTVGNYSSDKKRHNNKFITFLRKTMWEYALACVGVVPCGGNNDLNGQRKTTLEH
NKAWLLADSGAELASADPRSVHSSFRFSFCSQVEVESFNMSYSASAAAAATFLMVNLDYESQVRELKWRRIQSLE
KSLSPVANTLIRFSYDEILSATRNFSKERVLGRGALSCVFRGRVGIWRTAVAIKRLDKEDKECAKAFCRELMIAS
SLNDTNVVPLVGFCIDSEEGLFLVYKYVSGGSLEHHLHGRKKGVKGSSPLPWSVRYEVAIGIAEAVAYLHNGTER
CVVHRDIKPSNILLSSKKIPKLCDFGLASWTSAPSVPFLCKTVKGTFGYLAPEYFEHGKVSDKTDVYALGVVLLE
LLTGRNPIEAKRPPGEENLVVWAKPLLRKGKGAIEELLDPQVKYNSSYTDQMVRMIDAASVCVTSEESRRPSIGE
IVAILKGEVEHVLSRRRKSGYFGNGYMIDNYPKLQETNNEMKSHLALAMLGVPECEDDDFVYCR*
>Glyma.02G286000
SEQ ID NO: 11
MILIDLLQPIMSSNSSLNMELSKKTSFLGLKRWVLIGIGVGAFIVLILCILSIWAMFRRKCRRSLDKYSVSQIPN
VSKDIDVDKVGVQSSHVQPENVVIPVHDKASDKNSDNVSVHLGKSKSGDPDNISQCSSIYHHERGESSMSAEEGS
SGNVKKQSTLSHGGLATASPLVGLPEFSHLGWGHWFTLRDLEMATNRFSSENIIGEGGYGIVYRGRLINGTEVAV
KKLLNNLGQAEKEFRVEVEAIGHVRHKHLVRLLGYCVEGVHRLLVYEYVNNGNLEQWLHGNMHQYGTLTWEARMK
VILGTAKALAYLHEAIEPKVIHRDIKSSNILIDDEFNAKVSDEGLAKLLDSGESHITTRVMGTEGYVAPEYANSG
LLNEKSDIYSFGVLLLEAVTGRDPVDYARPANEVNLVEWLKTMVGTRRAEEVVDSSLEVKPPLRALKRTLLVALR
CIDPDADKRPKMSQVVRMLEADEYPEREDRRKRKSGTASMEIETVKDISGPSDAEKMVISESHVEEG*
>Glyma.03G020700
SEQ ID NO: 12
MPSRPPPLSPPATAAASHRREIILCGAIGGALFLTALIISVMIFIYRKLSYSRTAPFEHNQRRFSYTVLRRATNS
FSPSTKLGHGGFGSVHKATLPSGQTVALKVMDSPGSLQGEREFHNELTLCSNLKSPFVISLLGFSSDRRGKKLVL
VYELMPNRSLQDALLDRRCPELMSWGKRFDVAVSVARGLEYLHHVCDPPVIHGDIKPSNVLLDRDFRAKIGDFGL
ARVKNVEDLGMVDENEKKKDEEFSVLEGESVVDVDRSPESCPVRAAEYSDASPVGGDKLSVVSDGGGCFESVDSG
SVSVNKKKCGGGGGGGGSGRDWWWRQESGGGGESGREGVEQKKKRKSKGSRGSIDWWLDGLSAKIGGFSGDVPKS
GGISSTPSMRGTVCYIAPEYGGGGQLSEKCDVYSFGVLLLVLVAGRRPLQVTASPISEFERANLISWARQLAHNG
RLLDLVDTSIHSLDKEQALLCITIALLCLQRSPGKRPSIKEVVGMLSGEAEPPHLPFEFSPSPPSNFPFKTRKKA
R*
>Glyma.03G088800
SEQ ID NO: 13
MSDSDTRQNDITKPLDLENLRVVSAVGRGAKGVVFLARTGDRSSEECVALKVIPKALILQKAKLINDVEYTRVSF
EEQVLRRFDHLLLPRLRGVFETEKVVGFGIDYCHGGTLHSLRKKQTEKMFSDDTIRFYAVELVLALEYLHNLGIV
YRDLKPENVMIQDNGHIMLVDFDLSKKLNPKSPHSLSQNSSPSPNSKTKQTRKQRLMRFYSFCNSGILPCDSDSE
PPLSSVNSVRHTESDLVEKSNSFVGTEEYVAPEIVSGKGHGFSVDWWSYGVVLYEMLYGTTPFKGSNRKETFYRI
LMKEPELTGEKTALRDLIGKLLEKNPDRRIQVNEIKGHNFFKGVKWDTVLHIARPPYIPENEVENKVGFSKSDVE
VFVDNVFFPTGDDGGEKTKTLLDNNNNNNNNNNNNNNNNKVWIDELSHVPIGKKNDDFLIF*
>Glyma.03G156800
SEQ ID NO: 14
MEQYEILEQIGKGAFGSALLVRHKHEKKKYVLKKIRLARQTDRTRRSAHQEMELISKVRNPFIVEYKDSWVEKGC
FVCIIIGYCEGGDMAEAIKKANGINFPEEKLCKWLVQLLMALDYLHGNHILHRDVKCSNIFLIKDQDIRLGDFGL
AKMLSSDDLASSVVGTPSYMCPELLADIPYGSKSDIWSLGCCIYEMAAYKPAFKAFDIQSLLIKINKCIVSPMPT
MYSAAFRGLVKSMLRKNPELRPTAAELLNHPHLQPYIHKIQLKLNSPRRSTFPFQWPESNYVRRTRFVDPESVYT
LSDLDKCLSFSNDMALNPSVSGTEQVSQCSTQRAHGLSTCSKEKIYELSVGCVREKYKTDKSKASKFSTVERTPR
SRAVTVSATTKRHTIATSKTTHSGPKRDSLPASHAPSRKFSTPPRTRARATPNLYTNVLGSLDSLDVSINAPRID
KIVEFPMAFCEDPFSPIRGPSSTSARCSSSSAGSTADCSITKDKCTIQEDKVTLPTSITDACPAPKETKCSYEHV
KDCVSSHSSTDLDQRRFDTSSYQQRAEALEGLLEFSARLLQQQRFDELGVLLKPFGLEKVSPRETAIWLTKSFKQ
TAV*
>Glyma.03G189800
SEQ ID NO: 15
MKKVCHCPIFLALALCLCILCVAAEAAGQNDTLALTEFRLQTDTHGNLLTNWTGADACSAAWRGVECSPNGRVVG
LTLPSLNLRGPIDTLSTLTYLRFLDLHENRLNGTISPLLNCTSLELLYLSRNDFSGEIPAEISSLRLLLRLDISD
NNIRGPIPTQLAKLTHLLTLRLQNNALSGHVPDLSASLLNLTVLNVTNNELRGHVPDSMLTKFGNVSFSGNHALC
GSTPLPKCSETEPDTETTTITVPAKPSSFPQTSSVTVPDTPRKKGLSAGVIVAIVVAVCVAVLVATSFAVAHCCA
RGSTSGSVVGSETAKRKSGSSSGSEKKVYGNGGNLDRDSDGTNTETERSKLVFFDRRNQFELEDLLRASAEMLGK
GSLGTVYRAVLDDGCTVAVKRLKDANPCERNEFEQYMDVVGKLKHPNIVRLRAYYYAKEEKLLVYDYLPNGSLHA
LLHGNRGPGRIPLDWTTRISLMLGAARGLARIHAEYNASKIPHGNVKSSNVLLDKNGVALISDFGLSLLLNPVHA
IARLGGYRAPEQVEVKRLSQEADVYGFGVLLLEVLTGRAPSKEYTSPAREAEVDLPKWVKSVVKEEWTSEVFDQE
LLRYKNIEDELVAMLHVGLACVAAQAEKRPCMLEVVKMIEEIRVEESPLGDDYDEARSRTSLSPSLATTEDNLA*
>Glyma.04G085200
SEQ ID NO: 16
MAKGSYSISSKARSMKFIFLFMFMLNFILSDGDQHEVQLLLSFKASLHDPLHFLSNWVSFTSSATICKWHGINCD
NNANSSHVNAVVLSGKNITGEVSSSIFQLPYLTNLDLSNNQLVGEITFTHSHNSLSQTRYLNLSNNNLTGSLPQP
LFSVLFSNLETLDLSNNMFSGNIPDQIGLLSSLRYLDLGGNVLVGKIPNSITNMTALEYLTLASNQLVDKIPEEI
GAMKSLKWIYLGYNNLSGEIPSSIGELLSLNHLDLVYNNLTGLIPHSLGHLTELQYLFLYQNKLSGPIPGSIFEL
KKMISLDLSDNSLSGEISERVVKLQSLEILHLFSNKFTGKIPKGVASLPRLQVLQLWSNGLTGEIPEELGKHSNL
TVLDLSTNNLSGKIPDSICYSGSLFKLILFSNSFEGEIPKSLTSCRSLRRVRLQTNKFSGNLPSELSTLPRVYFL
DISGNQLSGRIDDRKWDMPSLQMLSLANNNFSGEIPNSFGTQNLEDLDLSYNHFSGSIPLGFRSLPELVELMLSN
NKLFGNIPEEICSCKKLVSLDLSQNQLSGEIPVKLSEMPVLGLLDLSQNQFSGQIPQNLGSVESLVQVNISHNHF
HGSLPSTGAFLAINASAVIGNNLCDRDGDASSGLPPCKNNNQNPTWLFIMLCFLLALVAFAAASFLVLYVRKRKN
FSEVRRVENEDGTWEVKFFYSKAARLINVDDVLKTVKEGKVVSKGTNWVWYEGKCMENDMQFVVKEISDLNSLPL
SMWEETVKIRKVRHPNIINLIATCRCGKRGYLVYEHEEGEKLSEIVNSLSWQRRCKIAVGVAKALKFLHSQASSM
LLVGEVSPEIVWVDAKGVPRLKVTPPLMPCLDVKGFVSSPYVAQEVIERKNVTEKSEIYGFGVMLVELLTGRSAM
DIEAGNGMHKTIVEWARYCYSDCHLDTWIDPVMKGGDALRYQNDIVEMMNLALHCTATDPTARPCARDVLKALET
VHRTTFC*
>Glyma.04G222800
SEQ ID NO: 17
MQALFSSMRVFLRLVWLLELLCVAVTAVNPSLNDDVLGLIVFKADIRDPKGKLASWNEDDESACGGSWVGVKCNP
RSNRVVEVNLDGFSLSGRIGRGLQRLQFLRKLSLANNNLTGGINPNIARIDNLRVIDLSGNSLSGEVSEDVFRQC
GSLRTVSLARNRFSGSIPSTLGACSALAAIDLSNNQFSGSVPSRVWSLSALRSLDLSDNLLEGEIPKGIEAMKNL
RSVSVARNRLTGNVPYGFGSCLLLRSIDLGDNSFSGSIPGDFKELTLCGYISLRGNAFSGGVPQWIGEMRGLETL
DLSNNGFTGQVPSSIGNLQSLKMLNFSGNGLTGSLPESMANCTKLLVLDVSRNSMSGWLPLWVFKSDLDKVLVSE
NVQSGSKKSPLFAMAELAVQSLQVLDLSHNAFSGEITSAVGGLSSLQVLNLANNSLGGPIPPAVGELKTCSSLDL
SYNKLNGSIPWEIGGAVSLKELVLEKNFLNGKIPTSIENCSLLTTLILSQNKLSGPIPAAVAKLTNLQTVDVSFN
NLTGALPKQLANLANLLTFNLSHNNLQGELPAGGFFNTITPSSVSGNPSLCGAAVNKSCPAVLPKPIVLNPNTST
DTGPSSLPPNLGHKRIILSISALIAIGAAAVIVIGVISITVLNLRVRSSTSRDAAALTFSAGDEFSHSPTTDANS
GKLVMFSGEPDFSSGAHALLNKDCELGRGGFGAVYQTVLRDGHSVAIKKLTVSSLVKSQEDFEREVKKLGKIRHQ
NLVELEGYYWTPSLQLLIYEYLSGGSLYKHLHEGSGGNFLSWNERFNVILGTAKALAHLHHSNIIHYNIKSTNVL
LDSYGEPKVGDFGLARLLPMLDRYVLSSKIQSALGYMAPEFACKTVKITEKCDVYGFGVLVLEIVTGKRPVEYME
DDVVVLCDMVRGALEEGRVEECIDERLQGKFPAEEAIPVMKLGLICTSQVPSNRPDMGEVVNILELIRCPSEGQE
ELA*
>Glyma.05G006700
SEQ ID NO: 18
MEHVLEKRFPLNAEDYTLYEEVGEGVSASVYRALCVPLNEIVAIKVLDLEKCNNDLDGIRREVQTMNLIDYPNVL
RAHCSFTAGHNLWVVMPYMAGGSCLHIMKSNYPEGFEEPVIATLLHEVLKALVYLHAHGHIHRDVKAGNILLDSN
GAVKLADFGVSACMFDTGDRQRSRNTFVGTPCWMAPEVMQQLHGYDFKADIWSFGITALELAHGHAPFSKYPPMK
VLLMTLQNAPPGLDYERDKKFSKAFKELVATCLVKDPKKRPSSEKLLKHHFFKQARASKYLARTILEGLAPLGDR
FRMLKAKEADLLVQNKALYEDKDQLSQKEYIRGISAWNFNLEDLKSQAALIQDDDIPNAEEPQRDKKQKDRLDNF
KVSAERLSAGAANHSDDAPTQDKEDLHLLFQDGFNNLQDLEGSLVSFPTKPLQALKGCFDMCEDDINNSSPRDLD
HDGRIDNESSRPSTSLQQNTTSQQKKFPSGSLLPDNFLFPKMVVTDGDRDYLQTKYSSERNHSGPLQYRQKRDTN
NLPLVDDTSDGAFFRRRGRFTLTDLSPMGPSNSTSGPVVSPTSPPNQNFMSTAILPSLQCILQHNGLQREEIIKL
IKYAEQSSGKNTESVEAGTGDMLQAPPATTRERELHFQVIQLQQSIGSLVEELQRQKMKNVQLEKQLNSMANRVE
K*
>Glyma.05G213200
SEQ ID NO: 19
MGNCSSGAGAPTTYSDDHPHHNGITVLPPNSNPSPQLPKPPPTSSSSSSLGRVLGRPMEDVRSIYIFGRELGRGQ
FGVTYLVTHKATKEQFACKSIATRKLVNRDDIDDIRREVQIMHHLTGHRNIVELKGAYEDRHSVNLVMELCAGGE
LFDRIITKGHYSERAAANSCRQIVTVVHNCHSMGVMHRDLKPENFLLLNKNDDSPLKATDFGLSVFFKPGDVFRD
LVGSAYYVAPEVLRRSYGPEADIWSAGVILYILLSGVPPFWAENEQGIFDAILRGHIDFASDPWPSISSSAKDLV
KKMLRADPKERLSAVEVLNHPWMRVDGDAPDKPLDIAVLTRMKQFRAMNKLKKVALKVIAENLSEEEIIGLKEMF
KSMDTDNSGTITFEELKAGLPKLGTKLSESEVRQLMEAADVDGNGTIDYIEFITATMHMNRMEREDHLYKAFEYF
DNDKSGYITMEELESALKKYNMGDEKTIKEIIAEVDTDNDGRINYDEFVAMMRKGNPDITHITQRRRK*
>Glyma.05G220600
SEQ ID NO: 20
MGICFSIEDQNHLSISDSNAKPKPAVGHESGAPLASMNIKDLREGAGYSNVDIFTYEELRLATKHFRPDFILGEG
GFGVVYKGVIDHSVRSGYKSTEVAIKELNREGFQGDREWLAEVNYLGQFSHPNLVKLIGYCCEDDHRLLVYEYMA
SGSLEKHLFRRVGSTLTWSKRMKIALHAARGLAFLHGAERPIIYRDFKTSNILLDADFNAKLSDFGLAKDGPMGD
QTHVSTRVMGTYGYAAPEYVMTGHLTARSDVYGFGVVLLEMLIGRRALDKSRPSREHNLVEWARPLLNHNKKLLK
ILDPKLEGQYSSKTALKVAHLAYQCLSQNPKGRPLMSQVVEILENFQSKGENEEDQMLQTGDISITLYEVPKGSN
GTPT*
>Glyma.05G229800
SEQ ID NO: 21
MEMDPPPKSWSIHTRSEIIAKYEVMERVGSGAYADVYRGRRLSDGLTVALKEIHDYQSAFREIDALQLLEGSPNV
VVLHEYFWREDEDAVLVLEFLRTDLATVIADTAKANQPLPAGELKCWMIQILSGLDACHRHMVLHRDLKPSNLLI
SEHGLLKIADFGQARILMEPGIDASNNHEEYSRVLDDIDNKDTITSTHDGNATCNTSDVDREEEELGCFTSCVGT
RWFRAPELLYGSRNYGLEVDLWSLGCIFAELLTLQPLFPGTADIDQLSRIIGVLGNLDENAWAACSKLPDYGIIS
FSKVENPAGLEACLPNRSPDEVALVKKLVCYDPAKRATAMELLHDKYFSDEPLPVLVSELRVPLTRKEQDGDSPG
GWGDINDMDSDSDSQFDEFGPLNITRTGTGFSIQFP*
>Glyma.06G017500
SEQ ID NO: 22
MSCFSCFVSRGKDVRRVEIDNGSRSATSSSEGKGKKSVSNKGTSTAAASFGFRELAEATRGFKEVNLLGEGGFGR
VYKGRLSTGEYVAVKQLIHDGRQGFHEFVTEVLMLSLLHDSNLVKLIGYCTDGDQRLLVYEYMPMGSLEDHLFDP
HPDKEPLSWSTRMKIAVGAARGLEYLHCKADPPVIYRDLKSANILLDNEFNPKLSDFGLAKLGPVGDNTHVSTRV
MGTYGYCAPEYAMSGKLTLKSDIYSFGVLLLELITGRRAIDTNRRPGEQNLVSWSRQFFSDRKKFVQMIDPLLQE
NFPLRCLNQAMAITAMCIQEQPKFRPLIGDIVVALEYLASHSNP*
>Glyma.07G004700
SEQ ID NO: 23
MSAPAEAPSFTASPPSETTPSPNSTALSPPPPSTINATVSPPPPEAASPPTSTVNSGLSTGTVSGTVIGAVLGSV
GMLIIGGIFFCFYRNWKRKKNHSQPPQPKADIAGGTLQNWQDSVPPTTDGKVGFSPKPPPGGLVNQQQSSAALLT
LVVNSSNTSSSLGSEKAKSYISPSPGTSLALSQSTFTYDELSMAIDGFSRSNLLGQGGFGYVHKGVLPNGKIVAV
KQLKSESRQGEREFHAEVDVISRVHHRHLVSLVGYCVSDSQKMLVYEYVENDTLEFHLHGKDRLPMDWSTRMKIA
IGSAKGLAYLHEDCNPKIIHRDIKASNILLDESFEAKVADFGLAKFSSDTDTHVSTRVMGTFGYMAPEYAASGKL
TEKSDVFSFGVVLLELITGRKPVDKTQTFIDDSMVEWARPLLSQALENGNLNGLVDPRLQTNYNLDEMIRMTTCA
ATCVRYSARLRPRMSQVVRALEGNISLEDLNDGIAPGHSRVFGSFESSSYDSVQYREDLKNFKKLALESQEQGIS
EYSGPSSEYGRHPSVSTSSDQQNTQEMEMGNKKGSNHDSGIQVLD*
>Glyma.07G011300
SEQ ID NO: 24
MSREQQKRGKQEKGSDGAEKVIVAVKASKEIPKTALVWSLSHVVQPGDCITLLVVVPSQSSGRRLWGFPRFAGDC
ASGIKKYPPGTISEQKSDLTDSCSQMILQLHNVYDPNKINVRIKIVSGSPCGAVAAEAKKTQANWVVLDKQLKHE
EKRCMEELQCNIVVMKRSQPKVLRLNLIGPQKKDVEEAGPSPSEQDDMPENRTKIKLDSLNSIKGPTVTPTSSPE
LGTPFTATEAGTSSVSSSDPGTSPFFISEMNGEFKKEETIKESQELVDTNSDTESESLSTSSASMRYQPWITELL
LHQQSSQRNEERSDISHGIPQASTTRAFLEKYSRLDRGAGFEISTYRNDMDFSGNLREAIALSGNAPPGPPPLCS
ICQHKAPVFGKPPRWFTYSELELATGGFSQANFLAEGGFGSVHRGVLPEGQVIAVKQHKLASSQGDLEFCSEVEV
LSCAQHRNVVMLIGFCIEDKRRLLVYEYICNGSLDSHLYGRQRDTLEWSARQKIAVGAARGLRYLHEECRVGCII
HRDMRPNNILITHDFEPLVGDFGLARWQPDGDTGVETRVIGTFGYLAPEYAQSGQITEKADVYSFGVVLVELVTG
RKAVDLTRPKGQQCLTEWARPLLEEYAIEELIDPRLGKHYSEHEVYCMLHAASLCIQRDPQCRPRMSQVLRILEG
DMVMDSNYISTPGYDAGNRSGRLWSEPLQRQQHYSGPLLEESLESFSGKLSLDKYKPSYWGDRDKARRASCEDDI
*
>Glyma.07G023500
SEQ ID NO: 25
MDPQNQHPPNPRSIIFNKYEMGRVLGQGNFAKVYHARNLNTNESVAIKVIKKEKLKKERLVKQIKREVSVMRLVR
HPHIVELKEVMATKGKIFLVMEYVKGGELFAKVNKGKLTEDLARKYFQQLISAVDFCHSRGVTHRDLKPENLLLD
QNEDLKVSDFGLSTLPEQRRADGMLVTPCGTPAYVAPEVLKKKGYDGSKADLWSCGVILFALLCGYLPFQGENVM
RIYRKAFRAEYEFPEWISPQAKNLISNLLVADPGKRYSIPDIMRDPWFQVGFMRPIAFSIKESYVEDNIDFDDVE
NNQEEEVTMRKPARPFYNAFEIISSLSHGFDLRSLFETRKRSPSMFICKFSASAVLAKVEAVAKKLNFRVTGKKE
FVVRMQGTEEGRKGKLAMTVEVFEVAPEVAVAEFTKSAGDTLEYVKFCEEQVRPSLKDIVWSWQGD*
>Glyma.07G260200
SEQ ID NO: 26
MGCHGSKEKKSNTSYGSAATGSGSGSGSGGGYNTVQPSPPTTDQVQASAQTPENRKASPTVQKKADTSIVGKPFD
DIKKYYSIGKELGRGQFGITYLCTENSSGGIYACKSILKRKLVSKADREDMKREIQIMQHLSGQPNIVEFKGAFE
DRFSVHLVMELCSGGELFDRIIAQGHYSERAAASLCRSIVNVVHICHFMGVMHRDLKPENFLLSTKDDHATLKAT
DFGLSVFIEQGKVYHDMVGSAYYVAPEVLRRSYGKEIDIWSAGIILYILLSGVPPFWAETEKGIFNAILEGEIDF
VSEPWPSISDSAKDLVRKMLTQDPKKRITSAQVLEHPWMREGGDASDKPIDSAVLSRMKQFRAMNKLKKLALKVI
AENLSEEEIKGLKAMFANMDTDSSGTITYEELKTGLARIGSRLSEAEVKQLMDAADVDGNGSIDYLEFISATMHR
HRLERDEHLYKAFQYFDKDNSGYITRDELETAMTQHGMGDEATIKEIISEVDTDNDGRINYEEFCAMMRSGMPHQ
GQLL*
>Glyma.08G002900
SEQ ID NO: 27
MGDGSGNRWSRAEWVQQYDLLGKIGEGTYGLVFLARTKGTPSKSIAIKKFKQSKDGDGVSPTAIREIMLLREITH
ENVVKLVNVHINHADMSLYLAFDYAEHDLYEIIRHHRDKLNHSINQYTVKSLLWQLLNGLSYLHSNWMIHRDLKP
SNILVMGEGEEHGVVKIADFGLARIYQAPLKPLSDNGVVVTIWYRAPELLLGAKHYTSAVDMWAVGCIFAELLTL
KPLFQGAEVKATSNPFQLDQLDKIFKVLGHPTLEKWPSLASLPHWQQDVQHIQGHKYDNAGLYNVVHLSPKSPAY
DLLSKMLEYDPRKRLTAAQALEHEYFKIEPLPGRNALVPCQLGEKIVNYPTRPVDTTTDLEGTTNLPPSQTVNAV
SGSMPGPHGSNRSVPRPVNVVGMQRMPPQAMAAYNLSSQAAMGDGMNPGGISKQRGVPQAHQPQQLRRKEQMGMP
GYPAQQKSRRI*
>Glyma.08G124800
SEQ ID NO: 28
MGNCWCRWESSEYRVSSNVKSEQNQGTKQRHDDSKLPSNPEEVEDLRRDSAANPLIAFTYDELKIITANFRQDRV
LGGGGFGRVYKGFISEELREGLPTLAVAVKVHDGDNSHQGHREWLAEVIFLGQLSHPNLVKLIGYCCEDEHRVLI
YEYMSRGSVEHNLFSKILLPLPWSIRMKIAFGAAKGLAFLHEAEKPVIYRDEKTSNILLDQEYNSKLSDEGLAKD
GPVGDKSHVSTRVMGTYGYAAPEYIMTGHLTPRSDVYSFGVVLLELLTGRKSLDKLRPAREQNLAEWALPLLKEK
KKFLNIIDPRLDGDYPIKAVHKAAMLAYHCLNRNPKARPLMRDIVDSLEPLQAHTEVPIGKTLTIISEVPESGLK
MKDDAI*
>Glyma.08G176200
SEQ ID NO: 29
MASTLLPFLFLSMVLLPFQTIAQTKSNIAIGESHTAGASTSPWLVSSPSGDFAFGFLPLEDTPDHFMLCIWYAKI
QDKTIVWFANRDQPAPKGSKVVLTADDGLVLITAPNGHMLWKTGGLTLRVSSGVLNDTGNFVLQDGHSKTVWESF
KDYRDTLLPYQTMEKGHKLSSKLGRNYFNKGRFVLFFQNDGSLVMHSINMPSGYANENYYQSGTIESNTNTSTSA
GTQLVEDGTGDMYVLRKNNEKYNLSKGGSRASSTTQFYYLRATLDFDGVFTLYQHPKGSSGSGGWSQVWSHPDNI
CKDYVASAGSGVCGYNSICSLRDDKRPNCRCPKWYSLVDPNDPNGSCKPDFVQACAVDKLSNRQDLYDFEVLIDT
DWPQSDYVLQRPFNEEQCRQSCMEDCMCSVAIFRLGDSCWKKKLPLSNGRVDATLNGAKAFMKVRKDNSSLIVPP
IIVNKNNKNTSILVGSVLLGSSAFLNLILVGAICLSTSYVFRYKKKLRSIGRSDTIVETNLRRFTYEELKKATND
FDKVLGKGAFGIVYEGVINMCSDTRVAVKRLNTFLMEDVHKEFKNELNAIGLTHHKNLVRLLGFCETEEKRLLVY
EYMSNGTLASLLFNIVEKPSWKLRLQIAIGIARGLLYLHEECSTQIIHCDIKPQNILLDDYYNARISDFGLAKLL
NMNQSRTNTAIRGTKGYVALEWFKNMPITAKVDVYSYGVLLLEIVSCRKSVEFEAEDEEKAILAEWAYDCYIEGT
LHALVEGDKEALDDMKTFEKLVMIALWCVQEDPSLRPTMRNVTQMLEGVVEVKMPPCPSQFSVQYS*
>Glyma.08G201600
SEQ ID NO: 30
MPVLRSGARKGRAAPKQQQQKQQQSPVVEGEAIATRIRRRRAAAAAAAAVPESNNNNNNTNNQQQQVEQVAVVNE
NVAVAAREEDNRVAEEEGVVRGGGAEREEVAEKKMGGCDSGGRSNGKANAAGEDDANTPQVPQKIQVGNSPSYKV
EKKLGKGGFGQVYVGRRTGGNLNERTGPGAVEVALKLEHRTSKGCTYGPPYEWQVYNTLGGSHGVPQVHYKGRQG
DYYVMVMDMLGPSLWDVWNNSNHHMTTEMVACIAIEAISILEKMHSRGYVHGDVKPENFLLGAPGTPDEKKLFLV
DLGLATKWRDSTTGSHVEYDQRPDVFRGTVRYASVHAHLGRTASRRDDLESLAYTLIFLLRGRLPWQGFQGENKG
FLVCKKKMGTSPETLCCFSPLPFKQFVEHVVNLKFDEEPNYAKYISLFDGVVGPNPDIRPINTEGAQKLIGHKRG
RLVMEEEDDEQPKKKIRIGLPASQWISVYNARRPMKQRYHYNVSDTRLSQHIEKGNEDGLYISSVASCQNLWALI
MDAGTGFTAQVYELSPFFLHKEWIMEHWEKNYYISAIAGAVNGSSLVVMSKGTQYLQQSYKVSDSFPFKWINKKW
REGFYVTAMATSGSRWGVVMSRGAGFSDQVVELDFLYPSEGIHKRWDCGYRITATAATWDQAAFVLSVPRRKPLD
ETQETLRTSAFPSTHVKEKWAKNLYIASICYGRTVS*
>Glyma.08G231100
SEQ ID NO: 31
MLILVLGLASATFLVFVAVYLFYSKRRVSKYNESKDIESSEHKEDEEMAQKEDLMIFQGGEDLTICDILDAPGEV
IGKSNYGTLYKALLQRSNKVSLLRFLRPVCTARGEELDEMIHFLGRIRHPNLVPLLGFYTGPRGEKLLVHPFYRH
GSLTQFIRDGNGECYKWSNICRISIGIAKGLEHLHTSQEKPIIHGNLKSKNILLDRSYQPYISDSGLHLLLNPTA
GQEMLESSAAQGYKAPELIKMKDASEESDIYSLGVILLELLSGKEPINEHPTPDEDFYLPNFMRNAVLGHRIADL
YHPAILLRNSRDDSIPVTEECILKVFQLAMACCSPSPSVRPNIKQVLKKLEEIMF*
>Glyma.08G235900
SEQ ID NO: 32
MARCILSHVLAFALVAGLCVFSCLVNVAQAQSANATTDPSEARALNSIFSKWDILANPTQWNISSELCSGRAIDA
TTTIDDTTFNPFIKCDCSYDSRTTCRITALKVYAMSIVGTIPEELWTLTYLTNLNLGQNYLTGSLPPNIGNLTRM
QYLSIGINNFSGELPKELGNLTELRSLAFGSNKFRGSLPSELGKLTNLEQIYFDSSGISGPIPSTFANLKNLLHV
GASDTELTGKIPDFIGNWSKLQTLRFQGNSFNGSIPSSFSNLSSLTELRISGLSNGSSSLEFLRNMKSLTILELR
NNNISGSISSTIGELHNLNQLDLSFNNITGQNLGSIFNLSSLTYLFLGNNKENGTLPMQKSSSLVNIDLSYNDLS
GSLPSWVNEPNLQLNLVANNLDVSNASGLPIGLNCLQKNFPCNQGIGRYSDFAIKCGGNQIRSADGIVYEMDNQT
LGPATYFVTDANRWAISNVGLFTGSSNPVYKSFVSNQFTGTVNSELFQTARLSASSLRYYGLGLENGFYNITLQF
AETAILDSTRSWESLGRRVFDIYIQGTRVLKDFDIQKEAGGISYKAIQRQFRFEVTENYLEIHLFWAGKGTCCIP
TQGTYGPLIQAIHAIPDFIPTVSNKPPSSNNNNIGLILGIVLGVGVVSVLSIFAIFCIIRRRRRRDDEKELLGID
TKPYTFSYSELKNAINDFNLENKLGEGGFGPVYKGTLNDGRVIAVKQLSVGSHQGKSQFITEIATISAVQHRNLV
KLYGCCIEGSKRLLVYEYLENKSLDQALFGKCLTLNWSTRYDICLGVARGLTYLHEESRLRIVHRDVKASNILLD
YELIPKISDFGLAKLYDDKKTHISTGVAGTIGYLAPEYAMRGHLTEKADVFSFGVVALELVSGRPNSDSSLEGEK
VYLLEWAWQLHEKNCIIDLVDDRLSEFNEEEVKRVVGIALLCTQTSPTLRPSMSRVVAMLSGDIEVSTVTSKPGY
LSDWKFEDVSSFMTGIEIKGSDTNYQNSSGSTSMMGGVDYYSPRDVSKPILKETLWEGR*
>Glyma.09G030000
SEQ ID NO: 33
MDQYEKVEKIGEGTYGVVYKARDRATNETIALKKIRLEQEDEGVPSTAIREISLLKEMQHRNIVRLQDVVHSEKR
LYLVFEYLDLDLKKHMDSSPEFVKDPRQVKMFLYQILCGIAYCHSHRVLHRDLKPQNLLIDRRTNSLKLADFGLA
RAFGIPVRTFTHEVVTLWYRAPEILLGSRHYSTPVDVWSVGCIFAEMVNRRPLFPGDSEIDELFKIFRILGTPNE
DTWPGVTSLPDFKSTFPKWPSKDLANVVPNLDAAGLNLLSSMLCLDPSKRITARSAVEHEYEKDIKFVP*
>Glyma.09G063200
SEQ ID NO: 34
MGVVLALILQLVKLCIIGFVVEFQGSEGSVISPSPAFLPVIHPSGEAPAPIHHGLSWESSPPKSPSDPNEFGISP
SNEIIPVDPSPSEPPGTLHPREPWRTIAPIPPEVPNGSFLPPPVTTLPPPTSAPTPQKVKGFEPSISPSPSTSTI
ASSPPAPYNAAHAPSTSEGSVPPSIQPSPPQSKTTPAIRPPVSTPIAPAPIAITSGNLPKTSPVSQPIEHGSLPP
KIDERNKSHKPEPALPALVPIPSTILPKISPVSQPTENGSLPHREGANNGHISEPISPAPTVFSFPEHSPESQPT
EHGSLPPTVPRRNINTGRTLEPVSQAPVATAPAILPKNSSVSQPTHHGNSPPDVQNRTANNGHSHTPAPEMPPSV
TPPRPFPVDPPLVHPVIPAAPPPKSPAPGGEPVSAPVYKTPKPPSAIVHFHAQAPVVSPVSTPSRSFNWTKGGEP
VSAPPYKTPKPLPVIVHSPAQAPSSAHKARQFHHAPEPPISSPESPFNKDHPPASSPSTTFYKHHHTRNTVTSPA
PASSHSISPSTSKHQDGSNPPLPLPTSRQRHHAPPPMNTGSSVSPSGLPIQSPVSQVSPAPSPLFKISPHSTKIP
LPPPKVSPSRPSSKTPKKPVRPRFQALPPPPPNEDCISLVCSDPYTSTPPGAPCKCVWPMKVGLRLSVSLYTFFP
LVSEFASEIATGVFMKQSQVRIMGADAANQQPDKTIVFVDLVPLGEEFDNTTAFLTSERFWHKQVVIKTSYFGDY
DVLYVTYPGLPPSPPLPPSSISIIDGGPYSGGGNNGRTIKPLGVDISKRQHKGGLSKGIIAIIALSVFLVVVLCF
AAALALFKYRDHVSQPPSTPRVLPPLTKAPGAAGSVVGGGLASASTSFRSNIAAYTGSAKTFSMNDIEKATDNFH
ASRVLGEGGFGLVYSGTLEDGTKVAVKVLKREDHHGDREFLSEVEMLSRLHHRNLVKLIGICAEVSFRCLVYELI
PNGSVESHLHGVDKENSPLDWSARLKIALGSARGLAYLHEDSSPHVIHRDFKSSNILLENDFTPKVSDFGLARTA
ADEGNRHISTRVMGTFGYVAPEYAMTGHLLVKSDVYSYGVVLLELLTGRKPVDMSRPPGQENLVAWARPLLSSEE
GLEAMIDPSLGHDVPSDSVAKVAAIASMCVQPEVSDRPFMGEVVQALKLVCNECDEAREAGSSSSSVDLSHSRQP
SDNLQGQFSATNYDSGIDIENGLLASELFSSSARYGRRVSGSFRRHSYSGPLNTGRSKRLWQIIRKLSGGSISEH
GTMFKL*
>Glyma.09G089700
SEQ ID NO: 35
MTSRSGQGSSGGGSSRTRVGKYELGRTLGEGNFAKVKFARHVETRENVAIKILDKEKLLKHKMIAQIKREISTMK
LIRHPNVIRMYEVMASKTKIYIVLEFVTGGELFDKIARSGRLKEDEARKYFQQLICAVDYCHSRGVEHRDLKPEN
LLLDANGVLKVSDFGLSALPQQVREDGLLHTTCGTPNYVAPEVINNKGYDGAKADLWSCGVILFVLMAGYLPFEE
TNLSALYKKIFKAEFTCPPWFSSSAKKLINKILDPNPATRITFAEVIENDWFKKGYKPPVFEQANVSLDDLDSIF
SDSTDSQNLVVERREEGPMAPVAPVTMNAFELISKSQGLNLSSLFEKQMGLVKRETRFTSKCSADEIISKIEKAA
GPLGFDVKKNNCKLKIQGEKTGRKGHLSVATEILEVAPSLYMVELRKSEGDTLEFHKFYKNLATGLKDIVWKAEP
IDEEKDGANPSK*
>Glyma.09G098000
SEQ ID NO: 36
MMGEKSNSNSGDAINSTLLHGKYELGRLLGHGSFAKVYHARHLNTGKSVAMKVVGKEKVVKVGMMEQIKREISAM
NMVKHPNIVQLHEVMASKSKIYIAMELVRGGELFNKIARGRLREETARLYFQQLISAVDFCHSRGVFHRDLKPEN
LLLDDDGNLKVTDFGLSTFSEHLRHDGLLHTTCGTPAYVAPEVIGKRGYDGAKADIWSCGVILYVLLAGFLPFQD
ENLVALYKKIYRGDFKCPPWFSSEARRLITKLLDPNPNTRITISKIMDSSWFKKPVPKNLVGKKREELNLEEKIK
HQEQEVSTTMNAFHIISLSEGFDLSPLFEEKKREEKELRFATTRPASSVISRLEDLAKAVKFDVKKSETKVRLQG
QENGRKGKLAIAADLYAVTPSFLVVEVKKDNGDTLEYNQFCSKELRPALKDIVWRTSPAENPTLA*
>Glyma.09G252000
SEQ ID NO: 37
MFKFLKEVVGGSGTGVKDLPYTIAEPYPSAWGSWTHSRGTSKDDGSPVSVFSLSGSNAQDGHLAAARNGVKRLRT
VRHPNILSFLHSAEIETYDAGSPKVTIYIVTEPVMPLSEKIKELGLEGTQRDEYYALGLHQIAKAVSFLNNDCKL
VHGNICMASTVVTPTLDWKLHALDVLSEFDGSSEASSGQMLQYAWLVGSQYKPMELAKSDWDAIKKSPPWAIDSW
GMGCLIYEVFSGLRLGKTEELRNIGSIPKSLLPDYQRLLSSMPSRRLNTSKLIENSEYFQNKLVDTIHFMEILSL
KDSVERDTFFRKLPNLAEQLPRQIVLKKLLPLLASALEFGSAAASALTALLKMGSWLSAEEFNVKVLPTIVKLFA
SNDRAIRVALLQHIDQYGESLSAQAVDEQVYPHVATGFSDTSAFLRELTLKSMLILAPKLSQRTMSGSLLKYLSK
LQVDEEPAIRTNTTILLGNIGSYLNEGTRKRVLINAFTVRALRDTFPPARGAGIMALCATSSYYDITEVATRILP
NVVVLTIDPDSDVRTKAFQAVDQFLQIAKQHYEKTNAADTSCGVGSSSVPGNASLLGWAMSSLTLKGKPSDHAPV
ASASSTAITSTSSNGTAGIETPSTAAAHVSSTADLAEHPVPTSPTSTDGWGELENGIDEEHGSDRDGWDDLEPLE
ETKPAPALANIQAAQRRPVSQPISHTKQASNLLSKSTPKLNKDEDDDLWGSIAAPAPKTARPLNLKSAQTDDDDP
WAAIAAPAPTIKAKPLSAGRGRGAKPAAPKLGAQRINRTSSGM*
>Glyma.09G277000
SEQ ID NO: 38
MEQKGSVLMQRYELGRLLGQGTFAKVYHARNLITGMSVAIKVVDKEKILKVGMIDQIKREISVMRLIRHPHVVEL
YEVMASKTKIYFVMEHAKGGELFNKVVKGRLKVDVARKYFQQLISAVDYCHSRGVCHRDLKPENLLLDENENLKV
SDFGLSALAESKCQDGLLHTTCGTPAYVAPEVINRKGYDGIKADIWSCGVILYVLLAGHLPFQDTNLMEMYRKIG
RGEFKFPKWFAPDVRRFLSRILDPNPKARISMAKIMESSWFKKGLEKPAITVTENEELAPLDADGIFEACENDGP
IAEPKQEQAKPCNLNAFDIISFSTGFDLSGLFEDTFLKKETRFMSKKPASIIVLKLEEICKRLCLKVKKKDGGLL
KLEGSKEGRKGTLGVDAEIFEITPHFHMVELRKSNGDTMEYQKLFKQDIRPALKDIVWTWQGEKPQQEQEQHEVV
QEEHQPSHTA*
>Glyma.10G038400
SEQ ID NO: 39
MDSARSWLQKFQPRDKTRAAGKKKEEDGNGGNQDSNEAIDEALLSSVTKQKVAAAKQYIENHYKEQMKNLQERKE
RRTILEKKLADADVSEEDQNNLLKFLEKKETEYMRLQRHKMGVEDFELLTMIGKGAFGEVRVCREKTSGHVYAMK
KLKKSEMLRRGQVEHVKAERNLLAEVDSNCIVKLYCSFQDDEHLYLIMEYLPGGDMMTLLMRKDILTEDEARFYV
GETVLAIESIHKHNYIHRDIKPDNLLLDRYGHLKLSDFGLCKPLDCSTLEENDFSVGQNVNGSTQSSTPKRSQQE
QLQHWQINRRTLAYSTVGTPDYIAPEVLLKKGYGMECDWWSLGAIMYEMLVGYPPFYSDDPMLTCRKIVNWKTYL
KFPEEARLSPEAKDLISKLLCNVNQRLGSKGADEIKAHPFFKGVEWNKLYQMEAAFIPEVNDELDTQNFEKFDES
DSQTQSSSRSGPWRKMLSSKKDLNFVGYTYKNFEIVNDYQVPGMAELKKKQSKPKRPTIKSLFDCESETPEASDT
SANDQPAQGSFLKLLPPQLEVSPHRDKNLPPRS*
>Glyma.10G052500
SEQ ID NO: 40
MKIPCFSCFSPSTTEKNNNNDYPDEEINDGSFRLFTHKQLKLATRNFHSSEKVGEGGFGSVFKGKLVDGSFVAVK
VLSVEVESMRGEREFVAELATLANIKHQNLVSLKGCCVEGAYRYLVYDYMENNSLYNTFLGSEERRMRFNWEIRK
DVSIGVARGLDFLHEELKPHIVHRDIKAKNILLDRNFIPKVSDFGLAKLLRDETSYISTRVAGTLGYLAPEYANS
GQVSRKSDVYSFGVLLLQIVSGLAVVDAYQDIERFIVEKAWAAYQSNDLLKLVDPMLNMNFPEEEALKFLKVGLL
CVQETAKLRPRMSEVVEKLTKDIDMRDVHISKPGFVADLRNIRIKQQNLNSSEESSSAGATFTSSISSSANLARY
LLD*
>Glyma.10G109200
SEQ ID NO: 41
MLLLLLNYLFFLFNFSITKTEAIDNLQYLNHSCSSNKTFTPNSTYQSNLQTLLTSLSSHATTAQFFNTTTGGGDA
AGENIYGSFMCRGDVSNHTCQECIKTATQQITVRCLNSKEALIWYHECMVRYSNRCFFSAVEEWPRFNFVDFNVN
TNSTEGIYGYWLLSKTLSDAVGEAVKAGTKKFATKNATVFGSQRVHTLVQCTPDLSSEDCSKCLGDIMRDIPLCC
LGRRGGMVLFPSCTLMEGIGQFYRDFPHGTPESKSENEKGSLRTIVIIVLLVVVVPVMLSFFSYHLIRRKARKRN
YKILLRENFGQESVTIEGLQFDLDIIAAATNNFSHENKIGKGGFGEVYKGILPNGRRIAVKRLSTNSSQGSVEFK
NEILSIAKLQHRNLVELIGFCLEVQEKILIYEYMSNGSLDNFLFDPQQKKLSWSQRYKIIEGTARGILYLHEHSR
LKVIHRDLKPSNILLDENMNPKISDFGMARIIELNQDLGKTQRIVGTFGYMSPEYAIFGQFSEKSDVFSFGVMII
EIITGRKNINSHQLPDIVDSLMSYVWRQWKDQAPLSILDPNLEENYSQFEVIKCIHIGLLCVQENKNIRPTMTKV
IFYLDGHTLDELPSPQEPPFFFRDIKDKKIPMQHFSVNKMSTSIFYPR*
>Glyma.10G218800
SEQ ID NO: 42
MERKFMALGFIWWVVVVHPLCLISANMEGDALHSLRTNLQDPNNVLQSWDPTLVNPCTWFHVTCNNDNSVIRVDL
GNAALSGQLVPQLGQLKNLQYLELYSNNITGPIPSDLGNLTNLVSLDLYLNHFTGPIPDSLGKLSKLRFLRLNNN
SLSGPIPMSLTNITALQVLDLSNNHLSGVVPDNGSFSLFTPISFANNMDLCGPVTGHPCPGSPPFSPPPPFVPPP
PISAPGGNGATGAIAGGVAAGAALLFAAPAIAFAWWRRRKPQEFFFDVPAEEDPEVHLGQLKRFSLRELQVATDS
FSNKNILGRGGFGKVYKGRLADGSLVAVKRLKEERTPGGELQFQTEVEMISMAVHRNLLRLRGFCMTPTERLLVY
PYMANGSVASCLRERPPYQEPLDWPTRKRVALGSARGLSYLHDHCDPKIIHRDVKAANILLDEEFEAVVGDFGLA
KLMDYKDTHVTTAVRGTIGHIAPEYLSTGKSSEKTDVFGYGIMLLELITGQRAFDLARLANDDDVMLLDWVKGLL
KEKKLEMLVDPDLQTNYIETEVEQLIQVALLCTQGSPMDRPKMSEVVRMLEGDGLAERWDEWQKVEVLRQEVELA
PHPNSDWIVDSTENLHAVELSGPR*
>Glyma.10G243000
SEQ ID NO: 43
MQLLHSDEPAPERGDSPEKPDDPNADTDSLDPGTDDGAALDVTGKSVEFPAAENAGDSAESLYVYKNVYSLIPKS
VSRLARLRTLKFFGNEINLFAPEFGNLTALECLQMKISSPGIGGLQLHTLKGLKELELSKGPPRPSAFPILTEIS
GLKCLTKLSICHFSIRYLPPEIGCLKKLEYLDLSFNKMKTLPAEISYLKGLISMKVANNKLVELPAAMSSLSRLE
RLDLSNNRLTSLGSLELASMHRLQELNLQYNKLLGIFQIPSWICCNMDGNDKARCKDDCSSSVEMDLYESNFQEN
DETLSDGPHNTSSSMLTSSSSSSRCFASRKSGKRWKRRHHLQQKARQERLNNSRKWKAVDHDDQLLSKKIHRISE
PENHDSLASESCAEIVSENGSLDDNNKRISSERAVNDNAIDNDNNDEVITEKQFSGEDCCTTESKDEKEESLCSL
DKRPSEQDEASCLELLECVSKSKRHLDRDLDNPKPCKSRKSISSSSLLSCKYSKISFCGIEDHLSDGFYDAGRDR
LFMPLECYEQNHCLASREVILLDRKIDEELDAVMLAAQALVYNLKKLNGLSRYGNQDGVDNLQMASLLALFVSDH
FGGSDRSGIVERTRKSVSGSNYNKPFVCTCSAGSSTSISSPTEPVANTIEDITLSKMSEKSLDSIKKRRNSIIIP
IGSVQYGVCRHRALLFKYLCDHMEPPVPCELVRGYLDFSPHAWNIILIKRGATWVRMLIDACRPLDIREEKDPEY
FCRYIPLNRTTIPISSIGSPGPDYSFPSLTTCDELETKASTTLVKCKFGSVEAAAKVRTLEEQGSSADKIKNFEY
NCLGEIRILGALKHPCIVEMYGHQISCQWSVSADGNPEHRVLRSAIFMEYVEGGSLKNYLEKLSEAGEKHVPVEL
ALHIAKDVSCALSELHSKHIIHRDIKSENILFNLDRKRDDGTPTVKLCDFDSAVPLRSTLHVCCIAHAGTPPPCI
CVGTPRWMAPEVMRTMYKKNSYGLEADIWSFGCLLLEMLTLQIPYSGLSDSHFLDSLQMGKRPQLTDELRVLSSM
NGPTMIPSGEELEKSDAGVDMLKFLVDLFHKCVEENPSKRPTAEEIHKMVLAHTDRLQI*
>Glyma.10G253200
SEQ ID NO: 44
MAMIPLMMMILLLLFKFPSVVLATLKSDEANLELVFTYHKCNEELGNFTTETYSNNRNVLLSNMYSDKEIENGFY
NSSYGEGPDKVYGIGFCRGDVKPDKCRSCLEKSSTLLTDRCPVQKEAIGWYDLCMLRYSNRSIVEQPVTDTDDII
KCSNTNATNKDRFDKELDDLVVRMRSRSAEGDSRLKFAEGEAPVQSSNETIHALLQCVPYLSHQNCTRCLEYAMS
RISYWCDGKTGGWYLGRSCSLRYETYLFFELIFHDAPAPQPSQPAVTPTKDFPKKTNPSRNIIVIVVPVFAVAIV
VVGLIVLIYNYFGARRPRHKPIQSEGDGEGDGEGEGELDNDIKTDELAQFEFATIKFATNNFSDANKLGQGGFGI
VYKGTLSDGQEIAIKRLSINSNQGETEFKTEISLTGKLQHRNLVRLLGFCFAKRERLLIYEFVPNKSLDFFIFDP
NKRGNLNWERRYNIIRGIARGLLYLHEDSRLQVVHRDLKISNILLDEELNPKISDFGMARLFEINQTEANTNTVV
GTFGYMAPEYIKHGKFSVKSDVFSFGVMMLEIVCGQRNSKIRGNEENAEDLLSFAWKNWRGGTVSNIVDTTLKDY
SWDEIKRCIHIGLLCVQEDINGRPTMNSVSIMLNSSSFSLAEPSEPAFLMRGKSQLPMIMLSGSEQYSEATKSSD
SGSQFAQGSSNKAPITEPYPR*
>Glyma.11G089100
SEQ ID NO: 45
MFPLQCSNHPMCAFSAVTAILLLFPAATSQAQILKKETYFFGPFNQSDFTTLTVLPSAAINLGALQVTPDSTGNV
SLANHSGRIFFNNPFTLWDNDDNLNGKLVSFNTSFLINVFRPQNNPPGEGITFLITASTTVPNNSHGQFLGLTNA
ATDGNATNKFVAVELDTVKQDFDPDDNHIGLDINSVRSNVSVSLTPLGFEIAPNVTRFHVLWVDYDGDRKEIDVY
IAEQPDKDAPIVAKPAKPVLSSPLDLKQVVNKVSYFGFSASTGDNVELNCVLRWNITIEVFPKKNGIGKALKIGL
SVGLTMVVLIVAGVVGWVCWLKKKKRGNESQILGTLKSLPGTPREFRYQELKKATNKFDEKHKLGQGGYGVVYRG
TLPKENLEVAVKMFSRDKMKSTDDFLAELTIINRLRHKNLVRLLGWCHRNGVLLLVYDYMPNGSLDNHIFCEEGS
STTPLSWPLRYKIITGVASALNYLHNEYDQKVVHRDLKASNIMLDSDFNARLGDFGLARALENDKTSYAEMEGVH
GTMGYIAPECFHTGRATRESDVYGFGAVLLEVVCGQRPWTKNEGYECLVDWVWHLHREQRILDAVDPRLGNGCVV
EEAERVLKLGLACSHPIASERPKMQTIVQIISGSVNVPHVPPFKPAFVWPAMDLSSPASDLTTPTTTTEYTPMSS
DTHSMHVQFSDSNSLI*
>Glyma.11G101700
SEQ ID NO: 46
MSRQTTSSAFTKSKTLDNKYMLGDEIGKGAYGRVYKGLDLENGDFVAIKQVSLENIAQEDLNIIMQEIDLLKNLN
HKNIVKYLGSSKTKSHLHIVLEYVENGSLANIIKPNKFGPFPESLVAVYIAQVLEGLVYLHEQGVIHRDIKGANI
LITKEGLVKLADFGVATKLTEADVNTHSVVGTPYWMAPEVIEMAGVCAASDIWSVGCTVIELLTCVPPYYDLQPM
PALFRIVQDEHPPIPDSLSPDITDFLLQCFKKDARQRPDAKTLLSHPWIQNCRRVLQSSLRHSGTLRNIEEDDSA
DAEVSGGYHKSAYENSSVEKEDSAKEHTTMAADGSKAHEDNAADSNFSNEQTEKADDAPSDQVLTLAIHEKSFLQ
AGSSKLTSNREVVNSESTGNHEISNAKDLHEVVMNGEGGSPQSRGMASKVGGKDSSVNNGNKSFAFGPRGQDNGP
LKKAMKMPITVEGNELSRFSDPPGDAYLDDLFHPLDKQPGEVVAEASTSTSTSHMTKGNASAIDGVKNDLAKELR
ATIARKQWEKESEIGQANNGGNLLHRVMIGVLKDDVIDIDGLVFDEKLPGENLFPLQAVEFSKLVGSLKPEESED
MIVSACQKLIGIFHQRPEQKIVFVTQHGLLPLTDLLEVPKTRIICSVLQLINQIVKDNTDFQENACLVGLIPAVT
SFAVPDRPREIRMEAAYFLQQLCQSSSLTLQMFIACRGIPVLVGFLEADYAKYREMVHLAIDGMWQVFKLQQSTP
RNDFCRIAAKNGILLRLINTLYSLNESTRLASSSAGGGFSVDGSAQRPRSGILDPNHPYINQNETMLSSVDQQDP
PKVRRAVPDHHLEPSSSNPRRSDANYPVDVDRPQSSNATADEKSLNQASRESSAGALKERENMDRWKTDPSQPRI
SNNRTSTDRPPKSTEPSSNGLSVTGTMHQEQVRPLLSLLDKEPPSGRFSGQLEYMRQFSGLERHESVLPLLHATE
KKTNGELDFLMAEFADVSQRGRENGNLDSSARVSHKVTPKKLGTLGSSEGAASTSGIASQTASGVLSGSGVLNAR
PGSATSSGLLSHMVSSLNAEVAREYLEKVADLLLEFAQADTTVKSYMCSQSLLSRLFQMFNRVEPPILLKILRCI
NHLSTDPNCLENLQRAEAIKYLIPNLELKEGSLVSEIHHEVLNALFNLCKINKRRQEQAAENGIIPHLMLFITSN
SPLKQYALPLLCDMAHASRNSREQLRAHGGLDVYLNLLEDELWSVTALDSIAVCLAHDNDNRKVEQALLKKDAVQ
KLVKFFQGCPEQHFVHILEPFLKIITKSARINTTLAVNGLTPLLIARLDHQDAIARLNLLRLIKAVYEHHPQPKK
LIVENDLPEKLQNLIGERRDGQVLVKQMATSLLKALHINTVL*
>Glyma.11G139700
SEQ ID NO: 47
MAMDEYECHEMVHVAVGKSLKKAATLLQWCFTHFSKPQIFLLHVHQPSTMIPTLLGKLPASQASPEVVSAYRIEE
KEDTKRLLEKYLSLCRAAKVKASSVIGEADQVQKGIVDLVTVHNVRKLVIGAIPENCMKIKRNSSKANYAAKNAP
PFCEIWFVYNGKHIWTREASETPRSLSSRAQPETTTAESLSCRSFHDGTKELLHSECLQLNSTKTTRSMVQSEII
EAEATFSSKSSSCNSHCSPQHSAGWYLDTHSEFEEETIDSQLIETKREAKAATDKALAELLKSKRLEVKAIEAIS
KVNFFESAHAHEVKLRKEAEDALRATIQEQQMFLDEKEEIARELERTVRSISLLGNCAHETNHKRDEAENELSLI
QASISNLWHEKQQIRQQKMEALHWLERWKSCGQVGADHCNGVIGFAEEFPELAEFSLSDLQNATCNFSESFKVME
GGYGSIYKGEMLGRTVAIRKLHPHNMQGSSEFHQEAQILGSLQHPHLVTLLGVCPEAWSFVYEYLPSGSLQDYLF
RKSSFLPLTRNIRAQWIAEIATALCFLHSSKPETIIHGGLTLETVLLDSALSCKICEFGFSRLVKEESVYLPNFH
FSTEPKGSFTYTDPEFQRTGVLTPKSDIYSFGIIILQLLTGRTPVGLVGEVRRAVSCGKLYPILDSSAGEWNSTM
ATRLAELGLQCCQLNSRVRPELTPSLVRELKQLLVLEERPVPSFFLCPIFQEIMHDPQVAADGFTYEGKAISEWL
ENGHETSPMTNLKLTHLNLTPNHALRLAIQGWLCKS*
>Glyma.12G032900
SEQ ID NO: 48
MTVEKRIVLVGIRIDGYSRQLLNWALAKVAEPGDCVIAVHVVKSSDYVSKNKTLIDGYLEVYEGLCGVKKVGLTG
QIFTGSSIKNILVREAKKHAALALVVGGRAATAKYCAKRLQPTTNVLAIQDSRIVFRSCTNKQLPGGLILDPRPS
LTIIKENLRDRAIHSSICDSIVEIEESTRKNSLESKEEAFNGSEKSKSRSISMFAGDSAEQKLGWPLLRRANSGM
SQTLHARDMSVVQWVMTLPDRSPNKGSSSSSTEENPFERSISDVEYESSSNSSPSSVDIPNGLEEMLNLNSLNCK
RFSLEVLKSCTSQFSSEKLVGKGGSNRVYKGVLTDGKSIAVKVMQSSKEAWKDFALEVEIISSLEHKSIAPLLGI
CIENNTLISVYDYFPNGSLEENLHGKNKDESILSWEVRFNVAIRIAEALDYLHREALKPVIHKDVKSSNILLSQG
FEPQLSDFGLAVWGPTTSSFLTQDVVGTFGYLAPEYFMYGKVSDKIDVYAFGVVLLELISGREPINSAACKGQES
LVVWAKPIIESGNVKGLLDPNLEGKFDEAQLQRMVLAASLCITRAARLRPKLSQILKILKGEEKVEYFLNSQGDN
DQEDSENQENIDDEVYPNSSAELHLSLALLGVDDDSTSHSSTDHSYSEDLKEQWSRSSSFN*
>Glyma.12G056000
SEQ ID NO: 49
MDSARTAPPPWQELDLDSLKPLKVLGKGGMGTVFLVQAANNTRFALKVVDKTCVHAKLDAERRARWEIQVLSTLS
HPFLPSLMGTFESPQFLAWALPYCPGGDLNVLRYRQTDRAFSPAVIRFYVAEILCALDHLHSMGIAYRDLKPENV
LVQNTGHITLTDFDLSRKLNPKPKPNPQVPSIPLPNSNVPEPRRKHRRNFSRWISLFPPDGTHHNNNKNGLKKAK
SARVSPVSRRKPSFSNGERSNSFVGTEEYVSPEVVRGDGHEFAVDWWALGILIYEMLYGTTPFKGKNRKETFRNV
ITKPPVFVGKRTALTDLIEKLLEKDPTKRLGYTRGAVEIKEHEFFRGVRWELLTEVVRPPFIPTRDDGAGDSTDR
ISDRNCGFDIRGYFLNLKSSPSLPGSPLPSPSCRFKKNVSLTEF*
>Glyma.12G062900
SEQ ID NO: 50
MGICLSTQIEAGLNSKHVSVDAKDLSSPSSKITKDLSNPISNKITEDLSTPISNKITEDLSTPISNKITEDLSTP
ISNKITEDLSTPISKVSEILVPLTPQIEGEILQSSNLKNFSLTELTAATRNFRKDSVLGGEGDFGSVFKGWIDNH
SLAAAKPGTGVVVAVKRLSLDSFQGHKDRLARHGMTHEASLEAEVNYLGQLSHPHLVKLIGYCFEDKDRLLVYEF
MPRGSLENHLFMRGSYFQPLSWGLRLKVALGAAKGLAFLHSAETKVIYRDFKTSNVLLDSNYNAKLADLGLAKDG
PTREKSHASTRVMGTYGYAAPEYLATGNLSAKSDVFSFGVVLLEMLSGRRAVDKNRPSGQHNLVEWAKPYLSNKR
KLLRVLDNRLEGQYELDEACKVATLSLRCLAIESKLRPTMDEVATDLEQLQVPHVKQNRRKSADHFTHGRIATAS
ASPLSRDIANTHP*
>Glyma.12G104100
SEQ ID NO: 51
MRNNKPQLWLSLSLIITCFSFHTSLAALTTISANQSLSGDETLVSQHGNFELGFFNTGNNSNKFYIGMWYKKISQ
RTYVWVANRDQPVSDKNSAKLTILEGNLVLLDQSQNLVWSTNLSSPSSGSAVAVLLDTGNLILSNRANASVSDAM
WQSFDHPTDTWLPGGKIKLDKKTKKPQYLTSWKNREDPAPGLFSLELDPAGSNAYLILWNKSEQYWTSGAWNGQI
FSLVPEMRLNYIYNFTFQSNENESYFTYSMYNSSIISRFVMDGSGQIKQLSWLENAQQWNLFWSQPRQQCEVYAF
CGGFGSCTENAMPYCNCLNGYEPKSQSDWNLTDYSGGCVKKTKFQCENPNSSDKEKDRFLPILNMKLPNHSQSIG
AGTVGECEAKCLSNCSCTAYAHDNSGCSIWHGDLLNLQQLTQDDNSGQTLFLRLAASEFDDSNSNKGTVIGAVAG
AVGGVVVLLILFVFVMLRRRKRHVGTRTSVEGSLMAFGYRDLQNATKNFSEKLGGGGFGSVFKGTLPDSSVVAVK
KLESISQGEKQFRTEVSTIGTVQHVNLVRLRGFCSEGTKKLLVYDYMPNGSLESKIFHEDSSKVLLDWKVRYQIA
LGTARGLTYLHEKCRDCIIHCDVKPENILLDADFIPKVADFGLAKLVGRDFSRVLTTMRGTRGYLAPEWISGVAI
TAKADVYSYGMMLFEFVSGRRNSEASEDGQVRFFPTIAANMMHQGGNVLSLLDPRLEENADIEEVTRVIKVASWC
VQDDESHRPSMGQVVQILEGFLDVTLPPIPRTLQAFVDNHENVVFFTDSSSTQTSQVKSNASAASSQAKSNISSS
NSST*
>Glyma.12G129600
SEQ ID NO: 52
MASLPLGHHHHHHKPAAAAIHPSQPPQSQPQPEVPRRSSDMETDKDMSATVIEGNDAVTGHIISTTIGGKNGEPK
ETISYMAERVVGTGSFGVVFQAKCLETGEAVAIKKVLQDRRYKNRELQLMRLMDHPNVISLKHCFFSTTSRDELF
LNLVMEYVPESMYRVIKHYTTMNQRMPLIYVKLYTYQIERGLAYIHTALGVCHRDVKPQNLLVHPLTHQVKLCDF
GSAKVLVKGESNISYICSRYYRAPELIFGATEYTASIDIWSAGCVLAELLLGQPLFPGENQVDQLVEIIKVLGTP
TREEIRCMNPNYTEFRFPQIKAHPWHKVFHKRMPPEAIDLASRLLQYSPSLRCTALEACAHPFFDELREPNARLP
NGRPLPPLFNFKQELAGASPELINRLIPEHIRRQMGLSFPHSAGT*
>Glyma.12G187700
SEQ ID NO: 53
MESRMDHYEIMEQIGRGAFGAAILVNHKAEKKKYVLKKIRLARQTERCRRSAHQEMALIARIQHPYIVQFKEAWV
EKGCYVCIVTGYCEGGDMAALMKKSIGVYFPEEKLCKWFTQILLAVEYLHSNFVLHRDLKCSNIFLTKDQDVRLG
DFGLAKTLKADDLASSVVGTPNYMCPELLADIPYGFKSDIWSLGCCIYEMAAHRPAFKAFDMAGLISKINRSSIG
PLPPCYSPSLKTLIKGMLRKNPEHRPTASEILKHPYLLPYVDQYRSSFCTPTAGSPERPISAVHHPRKNKPESQN
SSSSLSPEKDSFMSSEKNTANEVKKCDRKITEIDLTSIEDDSSEQLLPEEEGNGSSRVNAKTDEKELTKQSNNVH
HSNAVSKQPKPIKNVVTALKDGKLRETSSPIRGNRIKVGGVLTHKINSETVSKLPKPNFGAYDLKPNLEVPTTAP
SKTTPDSAKRMQGLHTSKHQLPMIESTPKTKPRHNAIPPSGPVKQVEGREVPSKPRQKTRPSLLKPPSFPGHVRQ
AGFDVPNATNNTGKSSPKKMVWEPKMSHHQLTNTHLPHVSRETTREPLKTFETSSKGMQTDSSNSVSSSLSIQGF
ELSDFATTFIDLSEPTLPDHESLNHTENVESCPYSISCASYLHFEMSEQLSGETPVVTPCFQNITSNEKVSPSLT
LDHSGQDAEVMFASDDSFSINQRTASAGSRCDNLSVDPSAEITQEIKDPQDSKEMSSAKSLQQSLLISGEKSVCE
EFGPSSKGSNRLDKVSRPKLMCISTGDDKFMVRERLSSVDETAPSIISTKISSQKVLQEEKGMVLQNPAPERPAV
GHLPPAFDDVIHVIRHSSYRVGSEQPGKESVEMGVQNVDVGKFINIARDDLEMRNVSTPLTLKSSNCSEAIGLKS
NISDNMEIRNLSSPPNLKSSSCTDLMNIKSSFSDHLLVRKQDVKNTDSLVSKSDSSEYTKHNTPTAAEETPPKEI
LDVKSSRQRAEALEGLLELSADLLQQNRLEELAVVLKPFGKDKVSPRETAIWLAKSLKGLMIEESGGRS*
>Glyma.13G002100
SEQ ID NO: 54
MTTFHVVEKDLTSRYARYDELLGKGAFKTVYKAFDEVDGIEVAWNMISVEDVVQTPQQLEKLYSEIHLLKSLKHD
NVIKLYNSWVDDTTGTINMITELFTSGSLRQYRNKHKNVDMKAIKNWARQILRGLCFLHCHSPPIVHRDLKCDNI
FVNGNSGLVKIGDLGLAIVMQQPTARSVIGTPEFMAPELYEEEYNELVDIYSEGMCILEMVTCEYPYSECNNPAQ
IYKKVTSGIKPAALAKVNDPEVKQFIEKCLVPASMRLSASELLKDPFLATENTKEINHDILELPNPHTKLVNPPT
CEPHPMEIDSKSRRTSPGSSMGRIEETSQVSFFDLVRMTENNKFMLRGEKNAESTISLTLRIANACGGARNIHFP
FYINSDTAISIAEEMVEHLELTNEDVSVIAELINDMIAKLVPNLKPLSEKLSSGTDQLYRPSSEVQNGEQFNCHW
PLQSSDYDMKPVFKDLVHSWPVDGDDLEKQESVMSDISVECGITVASDSKVVEPDIFIFDEFWEGFDAFNSTSDV
RFCGQEDGHKNQSENSSGSLINSCCCPFKNFDISSICSLTLADKDPSEGLRLEIEAIDTYFEQRFRELEMMRVAA
IESLKRRHGEKHTCDVMH*
>Glyma.13G060400
SEQ ID NO: 55
MDRSTGRGGGGSVDMFLRNYKLGKTLGIGSFGKVKIAEHVRTGHKVAIKILNRHKIKNMEMEEKVRREIKILRLF
MHHHIIRLYEVVETPTDIYVVMEYVKSGELFDYIVEKGRLQEDEARHFFQQIISGVEYCHRNMVVHRDLKPENLL
LDSKFNIKIADFGLSNIMRDGHFLKTSCGSPNYAAPEVISGKLYAGPEVDVWSCGVILYALLCGTLPFDDENIPN
LFKKIKGGIYTLPSHLSPGARDLIPRMLVVDPMKRMTIPEIRQHPWFQVHLPRYLAVPPPDTLQQAKKIDEEILQ
EVVNMGFDRNQLVESLSNRIQNEGTVTYYLLLDNRFRVSSGYLGAEFQETMDSGFNRMHSGEVASPVVGHHSTGY
MDYQGVGMRQQFPVERKWALGLQSRAQPREIMTEVLKALQELNVCWKKIGHYNMKCRWVAGTAGHHEGMINNSLH
SNHYFGNDSGIIENEAVSKSNVVKFEVQLYKTREEKYLLDLQRVQGPQFLFLDLCAAFLSQLRVL*
>Glyma.13G073900
SEQ ID NO: 56
MGNACAKGKPVAHVSSSNFSGSKKPASKPKQYSNSSEQRSAPTTSELNVPKSISSNLKSFSLNDLKEATKNFRRE
NLIGEGGFGRVFKGWIDENTYGPTKPGTGIVVAIKNLKPESFQGHKEWLQEVNYLGMLQHENLVKLIGYCLEGKN
RLLVYEFMQKGSLENHLFRKGVQPMAWVTRVNIAIGVARGLTFLHSLDQNVIFRDLKASNILLDSDFNAKLSDFG
LARDGPTGDNTHVSTRVIGTQGYAAPEYVATGHLTPRSDVYSFGVVLLELLTGRRAVEDDGPGFSEETLVDWAKP
FLNDNRRVLRIMDTRLGGQYSKKGAQAAAALALQCLNTDPKFRPPMVEVLAALEALNSSNSFTRTPKHESHSTKI
SGGPSQK*
>Glyma.13G106900
SEQ ID NO: 57
MKKGNLGLGLKLSVPVSDQSNFARFLTESGTFKDGDLLVNRDGVRIVSQNDVEAPPPIKPTDNQLTLADIDVIKV
VGKGNGGVVQLVQHKWTSQFFALKVIQMNIEESMRKQIAQELKINQQAQCPYVVVCYQSFYENGVISIILEYMDG
GSLADLLKKVKTIPEDYLAAICKQVLKGLVYLHHEKHIIHRDLKPSNLLINHIGEVKITDFGVSAIMESTSGQAN
TFIGTYNYMSPERINGSQRGYNYKSDIWSLGLILLECALGRFPYAPPDQSETWESIFELIETIVDKPPPIPPSEQ
FSTEFCSFISACLQKDPKDRLSAQELMAHPFVNMYDDLEVDLSAYESNAGSPLATL*
>Glyma.13G110700
SEQ ID NO: 58
MGSSFSSCYEGESVSPSPKPTKVVATKGGSSSNRVSITDLSFPGSTLSEDLSVSLVGSNLHVFSLSELKIITQSF
SSSNFLGEGGFGPVHKGFIDDKLRPGLEAQPVAVKLLDLDGSQGHKEWLTEVVFLGQLRHPHLVKLIGYCCEEEH
RLLVYEYLPRGSLENQLFRRYTASLPWSTRMKIAAGAAKGLAFLHEAKKPVIYRDFKASNILLDSDYNAKLSDFG
LAKDGPEGDDTHVSTRVMGTQGYAAPEYIMTGHLTAMSDVYSFGVVLLELLTGRRSVDKGRPQREQNLVEWARPA
LNDSRKLGRIMDPRLEGQYSEVGARKAAALAYQCLSHRPRSRPLMSTVVNVLEPLQDFDDVPIGPFVYTVPAEQH
NEVAKESETPKERKRENDHHHHNRHHHHHRHNGHRHHPLKSPKTPMSSDQSQNDEHRNGRRSGSNSPDTSNASEA
Q*
>Glyma.13G124800
SEQ ID NO: 59
MDSARSWLQKFQPRDKTRAAGKKKEEDGNGGNQDSNEPVDEALLSSVTKQKVAATKQYIENHYKEQMKNLQERKE
RRTILEKKLADADVSEEDQNNLLKFLEKKETEYMRLQRHKMGVDDFELLTMIGKGAFGEVRVCREKTSDHVYAMK
KLKKSEMLRRGQVEHVKAERNLLAEVDRNCIVKLYCSFQDDEYLYLIMEYLPGGDMMTLLMRKDTLTEDEARFYV
GETILAIESIHKHNYIHRDIKPDNLLLDRYGHLKLSDFGLCKPLDCSALEEKDFSVGQNVNGSTQSSTPKRSQQE
QLQHWQMNRRTLAYSTVGTPDYIAPEVLLKKGYGMECDWWSLGAIMYEMLVGYPPFYSDDPMLTCRKIVNWKTYL
KFPEEARLSPEAKDLISKLLCNVNQRLGSKGADEIKAHPFFKGVEWDKLYQMEAAFIPEVNDELDTQNFEKEDES
DSQNQSSSRSGPWRKMLSSKDLNFVGYTYKNFEIVNDYQVPGIAELKKKQSKPKRPTIKSLFETSEGSDTDTSAN
DQPAQGSFLKLLPPQLEVSPHRNKNLPPRS*
>Glyma.13G150000
SEQ ID NO: 60
MMKHYHVLYMFLFFLPISTLSLHHNDTHALTLFRRQSDLHGYLLSNWTGHDACNSAWRGVLCSPNGRVTALSLPS
LNLRGPLDPLTPLTHLRLLNLHDNRLNGTVSTLFSNCTNLQLLYLSSNDFSGEIPPEISSLKSLLRLDLSDNNLR
GKVDVISNLTQLITLRLQNNLLSGEIPDLSSSMKNLKELNMTNNEFYGRLPSPMLKKFSSTTFSGNEGLCGASLF
PGCSFTTTPPNNNDSNNNNDNNSNEKEPSQTVSSNPSSFPETSIIARPGREQQRKGLSPGAIVAIVIANCVALLV
VVSFAVAHCCARGRGSSLVGSGESYGKRKSESSYNGSDEKKVYGGGESDGTSGTDRSRLVFFDRRSEFELEDLLR
ASAEMLGKGSLGTVYRAVLDDGCTVAVKRLKDANPCARHEFEQYMDVIGKLKHPNVVRLKAYYYAKEEKLLVYDY
LSNGSLHALLHGNRGPGRIPLDWTTRISLVLGAARGLAKIHAEYSAAKVPHGNVKSSNVLLDKNGVACISDFGLS
LLLNPVHAIARLGGYRAPEQEQNKRLSQQADVYSFGVLLLEVLTGRAPSSQYPSPARPRMEVEPEQAAVDLPKWV
RSVVREEWTAEVFDQELLRYKNIEEELVSMLHVGLTCVVAQPEKRPTMEEVVKMIEEIRVEQSPLGEDYDVSCNS
LSPSIPTTEDGLA*
>Glyma.13G174900
SEQ ID NO: 61
MTNPKNQTTLSIIIIVYILFFSLATTLVSCLNQEGLYLYQLKLSLDDPDSKLSSWNSRDATPCNWYGVTCDAATN
TTVTELDLSDTNIGGPFLSNILCRLPNLVSVNLFNNSINETLPSEISLCKNLIHLDLSQNLLTGPLPNTLPQLLN
LRYLDLTGNNFSGPIPDSFGTFQNLEVLSLVSNLLEGTIPSSLGNVSTLKMLNLSYNPFFPGRIPPEIGNLTNLQ
VLWLTQCNLVGVIPTSLGRLGKLQDLDLALNDLYGSIPSSLTELTSLRQIELYNNSLSGELPKGMGNLTNLRLID
ASMNHLTGRIPEELCSLPLESLNLYENRFEGELPASIADSPNLYELRLFGNRLTGKLPENLGRNSPLRWLDVSSN
QFWGPIPATLCDKGALEELLVIYNLFSGEIPASLGTCQSLTRVRLGFNRLSGEVPAGIWGLPHVYLLELVDNSFS
GSIARTIAGAANLSLLILSKNNFTGTIPDEVGWLENLVEFSASDNKFTGSLPDSIVNLGQLGILDFHKNKLSGEL
PKGIRSWKKLNDLNLANNEIGGRIPDEIGGLSVLNFLDLSRNRFLGKVPHGLQNLKLNQLNLSYNRLSGELPPLL
AKDMYRSSFLGNPGLCGDLKGLCDGRGEEKSVGYVWLLRTIFVVATLVFLVGVVWFYFRYKNFQDSKRAIDKSKW
TLMSFHKLGFSEDEILNCLDEDNVIGSGSSGKVYKVVLSSGEVVAVKKIWGGVKKEVESGDVEKGGRVQDNAFDA
EVETLGKIRHKNIVKLWCCCTTRDCKLLVYEYMPNGSLGDLLHSSKGGLLDWPTRYKIAVDAAEGLSYLHHDCVP
AIVHRDVKSNNILLDVDFGARVADFGVAKAVETTPKGAKSMSVIAGSCGYIAPEYAYTLRVNEKSDIYSFGVVIL
ELVIGKRPVDPEFGEKDLVKWVCTTLDQKGVDHLIDPRLDTCFKEEICKVFNIGLMCTSPLPIHRPSMRRVVKML
QEVGTENQTKSAKKDGKLSPYYYDDASDHGSVV*
>Glyma.13G228400
SEQ ID NO: 62
MADVVSKSPTPTSNLISPNKKETSNLLLGRFEIGKLLGHGTFAKVYYARNIKTGEGVAIKVIDKEKILKGGLVAH
IKREISILRRVRHPNIVQLFEVMATKSKIYFVMEYVRGGELFNKVAKGRLKEEVARKYFQQLISAVGFCHARGVY
HRDLKPENLLLDENGNLKVSDFGLSAVSDQIRQDGLFHTFCGTPAYVAPEVLARKGYDGAKVDLWSCGVVLFVLM
AGYLPFHDQNVMAMYKKIYRGEFRCPRWFSPDLSRLLTRLLDTKPETRIAIPEIMENKWFKKGFKQIKFYVEDDR
LCNVVDDDGLMDNDDDTASIVSVASFSDYSVSESDSEIETRRRINAPLPRPPSLNAFDIISFSPGFNLSGLFEEK
EDETRFVTAAPVNRIISKLEEIAQLVRFSVRKKDCRVSLEGTREGVRGPLTIAAEIFELTPSLVVVEVKKKGGDR
AEYERFCNDELKPGLQNLMVEESATSSELSTPIQPSLLRGLSEPVPDISSDIETPLCIPSDD*
>Glyma.13G235000
SEQ ID NO: 63
MLQLLPVFLLLLLEQASLISGLNQDGLYLYEWKQSLDDPDSSLSSWNNRDATPCNWAGVTCGPSNTTVTALDLSN
FNLSGPFSASLLCRLPNLTSIILFNNSINQTLPLQISLCTPLLHLDLSQNLLTGFLPHTLPLLPNLLHLDLTGNN
FSGPIPPSFATFPNLQTLSLVYNLLDDVVSPSLFNITTLKTLNLSFNPFLPSPIPHSLGNLTNLETLWLSGCNLV
GPIPESLGNLVNLRVLDFSFNNLYGPIPSSLTRLTALTQIEFYNNSLSAEFPKGMSNLTSLRLIDVSMNHLSGTI
PDELCRLPLESLNLYENRFTGELPPSIADSPNLYELRLFGNKLAGKLPENLGKNAPLKWLDVSTNRFSGGIPESL
CEHGELEELLMLENEFSGEIPASLGGCRRLSRVRLGTNRLSGEVPAGMWGLPHVYLLELGNNSFSGPIARTIAGA
RNLSLLILSKNNFSGVIPDEIGWLENLQEFSGADNNFNGSLPGSIVNLGQLGTLDLHNNELSGELPKGIQSWKKL
NDLNLANNEIGGKIPDEIGILSVLNFLDLSNNEISGNVPLGLQNLKLNLLNLSYNRLSGRLPPLLAKDMYRASFM
GNPGLCGDFKGLCDGKGDDDNSKGFVWILRAIFIVASLVFVVGVVWFYFRYRNFKNAGRSVDKSKWTLMSFHKLG
FSEDEILNCLDEDNVIGSGSSGKVYKVVLTSGESVAVKKIWGGVKKEIDSGDVEKGHQFRQDSSFDAEVETLGKI
RHKNIVKLWCCCTTRDSKLLVYEYMPNGSLGDLLHSNKGGLLDWPTRYKIAVDAAEGLSYLHHDCVPSIVHRDVK
SNNILLDGDFGARVADFGVAKVVDATGKGTKSMSVIAGSCGYIAPEYAYTLRVNEKSDIYSFGVVILELVTGRRP
IDPEFGEKDLVMWACNTLDQKGVDHVIDSRLDSCFKEEICKVLNIGLMCTSPLPINRPAMRRVVKMLQEVGTENQ
TKPAKKDGKLSPYYYDDGSDHGSVA*
>Glyma.13G238700
SEQ ID NO: 64
MIRHKHTHHLPSILLSILFLFNSTCAIDFVFNGFNSSEVLLFGNATVDSRILTLTHQQRFSVGRALYNKKIPTKK
PNSSRVYPFSTSFIFAMAPFEDTLPGHGLVFIFTPVTGIQGTSSAQHLGLENLTNNGNSSNHVFGVEFDVFQNQE
FDDIDANHVGIDINSLKSYVSHDAGYWPDGADKSFKELTLNSGENYQVWIDYEDSWINVTMAPVGMKRPSRPLLN
VSLNLSQVFEDEMFVGFTSATGQLVESHKILGWSFSNEKFSLSDELITTGLPSFVLPKDSIFKSKGFVAGFTVGV
FFVICLLVLLALFLIQRKREKERKRMEMEDWELEYWPHRMTYEEIEAATKGFSEENVIGVGGNGKVYKGVLRGGV
EVAVKRISHENDGLREFLAEVSSLGRLKQRNLVGLRGWCKKDVGNFLLIYDYMENGSLDKRVFDCDESKMLSYED
RIRILKDVAFAVLYLHEGWEDKVVHRDIKASNVLLDKDMNGRLGDFGLARMHSHGQVASTTKLVGTVGYMAPEVF
KTGRASTQTDVYMFGILILEVLCGRRPLEEGKPPLVEWIWQLMVQGQVECALDERLRAKGEFNVQEMERVMHLGL
LCAYPEPKTRPTMRQVVNVLEGKNEVEDSEIENMDTYLLQQLKSRDILSEYSQYFSYTSHPTFQDIRLSSSMSLT
WSESVVEGR*
>Glyma.13G335700
SEQ ID NO: 65
MELLKPLHPHHAPILRIPFHAVPSSSSSQSQSQVPLPMISKVHVAVGKSLDKVVPLLRWTLNHFRNAEIVIVHAY
QPSLTIPTLLGKLPASQASPAVVSAFRKVEREQIMKLLDKYLSICRAARVKAAIIVTEADQVQKGIVDLVIKHNI
EKLVIGAVPENCMKVKRNSSKANYTAKNAPPFCEVWFIYKGKHIWTREASETPCSSSSCTQPEIATTESLRCRSF
QYGKNELFDSEYLWPNSARTTAVSGIRSWVQGEIIETEATFSSKASSCCSHCSPQNSSRAYFDTYLEVMEERINK
QLIETKREAEAVTDEAFTELLKCEKLEVEAMEAIRKVNLFESAHVREVKLRKEADDALRDTVQEQQKLLNESEEI
AGELQMTMRNIALLDSRAQEANRRRDEAADELSLIQESISTLWQERQQIRRQKTEALRWLERWRSRGQVGAAHCN
GVIGFAEELPELAEFSLSDLQNATCNFSNSFKIEQGGYSCIYKGEMLGRTVAIKKFHQHNMQGPLEFRQEVQVLG
SLQHPHLITLLGVCPEAWSIVYEYLPNGTLQDYLFRKSNNSPLTWNTRARMIAEIASALCFLHSFKPETIIHGDL
KPETVLLDSSLGCKMCGFGLCRLVSEESLLRPSFRLSTEPKGAFTYTDPEFQRTGILTTKSDIYSFGLIILQLLT
GRTPVGLAVLVRNAVSCGKLSSILDSSAGEWPSAVAMQLVELGLQCCQQYHRDRPELTPTLVRELEQLHASEERP
VPSFFSCPILQEIMHDPQVAADGFTYEGDAIREWLENGHDTSPMTNLKLSHLFLTPNYALRLAIQDWLCKS*
>Glyma.14G026300
SEQ ID NO: 66
METSLDYSKMLFLLLLGSTSLIFLSHLASAATPKLNTQEVKALKEIGSKIGKKDWNFGVDPCSGKGNWNVPDARK
AFVMSSVICDCSFNHNSSCHVVSIYWKAQNLSGSLSPEFSKLHYLQKLDLSRNIITGSIPPQWGTMRLVELSLMG
NKLSGPFPKVLTNITTLRNLSIEGNQFSGHIPTEIGKLTNLEKLVLSSNGFTGALPPVLSKLTKLIDLRISDNNF
LGKIPDFISNWTLIEKLHMHGCSLEGPIPSSISALTRLSDLRITDLKGSKSSAFPPLNNLKSMKTLVLRKCMIKG
EIPEYIGRMEKLKILDLSYNGLSGEIPESFAQLDKVDEMYLTGNKLSGIIPRWVLANNENIDISDNNFSWDSSSP
TECQRGSVNLVESYSSSVNTQTKINSCLKKNFLCTASPSQYRYSLNINCGGNEANVSGNIYEADREQKGAAMLYY
TSQDWALSSTGNFMDNDIDSDPYIVANTSRLNVSALNSKLYTTARVSPLALTYYGLCLINGNYTVKLHFAEIIFI
NDRSLNSLGRRVFDVYIQGNLVLKDFDIRREAGGTGKSIEKTFNASVTQHTLKIHFYWAGKGTTGIPTRGVYGPL
VSAISVNPNFKPPSGEGKRTYLILAIIIVAGVLVVVLLVLVLLRRMGWLGGKDPVYKELRGIDLQTGLFTLRQIK
AATKNFDALNKIGEGGFGCVYKGQQSDGTMIAVKQLSSKSKQGNREFVNEMGLISGLQHPNLVKLYGCCVEGNQL
ILIYEYMENNCLSRILFGRDPNKTKLDWPTRKKICLGIAKALAYLHEESRIKIIHRDVKASNVLLDKDFNAKVSD
FGLAKLIEDEKTHISTRVAGTIGYMAPEYAMRGYLTDKADVYSFGVVALETVSGKSNTNFRPNEDFVYLLDWAYV
LQERGSLLELVDPNLGSEYLTEEAMVVLNVALLCTNASPTLRPTMSQVVSMLEGWTDIQDLLSDPGYSAISSSSK
HKSIRSHFWQTPSGTHSISIPSIYTDSSGSHVETEKNYHPVTVNSDGSDKSN*
>Glyma.14G058900
SEQ ID NO: 67
MAVSNGVEAVLEFLRKNGLSEAESALRQDIIENNDLGNFDYEKFFFPMVPPPPPVRVRSFSRLSELSADGNCSKS
SSDEFVSIGSPISRVSSSEFINPYGIRSSSQTQNDSASSSERLSQFGTARDYHDFEMQNEPYWYNEKDDDYFMTP
SFEGPDFFACQSEDKFVMTAETENQHDNSLDLVYNSEEFLLKGNGNGGEMDKACLYNHSSVRDGNATYSKEYCHV
DNNNLFEGELEGKAEKHTVACSCEVPFCKSSPGGSCSLDPTNFGYPNLKEIHLKFGDINSFDSTSELTVNQSFDY
YTKNDSSKEYNGPYDLTIKVNQKDLPNGLDTYKARDGGELAEECQDPEITADGEDTTDDELLKYTQEEEYEVEDL
RIIHRKNRTGFEENKELPIVLNTVLAGRYYVTEYLGSAAFSRVVQAHDLQTGIDVCLKIIKNDKDFFDQSLDEIK
LLKLVNKHDPADLHHFLRLYDYFYHQEHLFIVTELLQANLYEFQKFKQESGGEEYFTLNRLQLITRQCLEALQYL
HSLGIVHCDLKPENILIKSYRRCEIKVIDLGSSCFQTDNLCLYVQSRSYRAPEVMLGLQYDEKIDIWSLGCILAE
LCSGEVLFPNDAVVMILARMIGMFGSIDMEMLVKGQETHKYFTKEYDIYYVNEETDQLEYIIPEESSLEQHLQVT
DTTFIDFVRYLLSINPKRRPTARQALRHPWLSYVY*
>Glyma.14G074700
SEQ ID NO: 68
MGMGGIAVSVVTVLLLMIGLTMGMSLSSRTEWFALRELRQSLEIRAKYWPIKAEPCGNWTGVQCRNGRVVGINVS
GLRRTRWGRLNPSFEVDSLVNFTLLETFNASGFKLNGSIPEWLGERLGVLEELDLSLCSIKGSIPDSIGRLSKLK
VLLLSGNFLTGRMPSTLGNLTRLSVLDLSGNSLSWPVPDSVSKLGNLSRLDLSYNFLSGSVPPELGALSSLQFLN
LSGNSFTGSVPSQLGNLSKLVEVDLSMNFLSRSLSGGLFSSVVLALEVLILRGNLLDGVLPANLRSMPRLHFLDV
SSNNLTGTLPNFADWNVSSAGVVFNLSNNMFYGLLNTSLDRFKMIDLSSNFLEGEVLGGGGGVSNVDLDRNCLQR
IPNQRNLEDCRMFYDKRNLSSAFPESESRSRRRVIFMLVGIFGGLGFIVLLALVLMLVLKQCHNRKSLEVPRETK
DGGAVEEGESPIPPKDIDFVTGVGEAYSFEQMLRLTGNFAESNVIKHGHSGDLFLGVLEGGATVVVKKVDLNLFK
RESYVVELGLLSKVPHARLVPILGHCLDNENEKCIVYKYMPNRDLATSLHRVTGSDGKIQSLDWITRLKIAIGAA
EGIAYLHECSPPLVHRDIQASSILLDDKFEVRLGSLSEVTAQGDLQQGVISRVFSKPPSSNQADSGKSPVTCTYD
VYCFGKILLELITGNIEVSKSDDATTKEWLEQTLPYITIYDKERVTKIIDPSLIVDEDLLEEVWAMAIVANACLK
PKPSKRPPMRHVLKALENPLKIVREENTSSARLRTNSSRKSWSTAFFGSWRHSSSDSVVATNKEGSNDTKKSGKV
GSQSSGNDHSSSNKRSSNEIFPEPLEIQDVETGVTR*
>Glyma.14G206000
SEQ ID NO: 69
MTEVTLNSSVNILYHTAKKISMKFYSLQAHRFLFIIVILCPLVIADLSSDKQALLDFAAAVPHRRNLKWNPATPI
CSSWVGITCNLNDIRVVSVRLPGIGLVGTIPANDTRIDSLRNISLRANLLSGSLPADITSLPSLQYLYLQHNNLS
GNIPTSLSTRLNVLDLSYNSFTGAIPKTLQNLTQLIKLNLQNNSLSGLIPNLNVTKLRRLNLSYNHLNGSIPAAL
QIFPNSSFEGNSLCGLPLKSCPVVPSTPPPSSTPAPPSTPARHSSKSKLSKAAIIAIAVGGGVLLLLVALIIVLC
CFKKKDDGSPRATKGKGPSGGRSEKPKEEFGSGVQEPEKNKLVFFEGSSYNFDLEDLLRASAEVLGKGSYGTAYK
AILEESTTVVVKRLKEAVVGKREFEQQMEIVGRVGHHPNVVPLRAYYYSKDEKLLVYDYIPSGNLSTLLHGNRAS
GRTPLDWNSRIKISVGIARGIAHIHSVGGPKFAHGNVKSSNVLLNQDNDGCISDFGLTPLMNVPSTPSRAAGYRA
PEVIETRKHTHKSDVYSFGVLLLEMLTGKAPQQSPGRDDMVDLPRWVQSVVREEWTAEVFDVELMRYQNIEEEMV
QMLQIAMACVAKVPDMRPSMEEVVRMIEEIRLSDSENRPSSEENRSKEESTAQTP*
>Glyma.14G212100
SEQ ID NO: 70
MKALALLAIIVFTLLVRSQEEEDYDDASVMLALKNSLNPPGWSDPDPCKWARVLCSDDKRVTRIQIGRLNLQGTL
PTTLQKLTHLEHLELQYNNISGPLPSLNGLTSLRVFLASNNRFSAVPADFFAGMSQLQAVEIDSNPFEPWEIPQS
LRNASGLQNFSANSANVGGSIPEFFGSDVFPGLTLLHLAMNNLEGTLPLSFSGSQIQSLWLNGQKSVNKLGGSVE
VLQNMTFLTDVWLQSNAFTGPLPDLSGLKSLRDLSLRDNRFTGPVPVASFVGLKTLKVVNLTNNLFQGPMPVFGD
GVVVDNVKDSNSFCLPSPGDCDPRVDVLLSVVGVMGYPPRFAESWKGNDPCAYWIGITCSNGYITVVNFQKMELS
GVISPEFAKLKSLQRIVLADNNLTGSIPEELATLPALTQLNVANNQLYGKVPSFRKNVVVSTNGNTDIGKDKSSL
SPQGLVPPMAPNAKGDSGGVSGIGGKKSSSHVGVIVFSVIGAVFVVSMIGFLVFCLFRMKQKKLSRVQSPNALVI
HPRHSGSDNESVKITVAGSSVSVGAASETRTVPGSEASDIQMVEAGNMVISIQVLKNVTDNFSEKNVLGQGGFGT
VYRGELHDGTRIAVKRMECGAIAGKGAAEFKSEIAVLTKVRHRHLVSLLGYCLDGNEKLLVYEYMPQGTLSRHLF
DWPEEGLEPLEWNRRLTIALDVARGVEYLHGLAHQSFIHRDLKPSNILLGDDMRAKVADFGLVRLAPEGKASIET
RIAGTFGYLAPEYAVTGRVTTKVDVFSFGVILMELITGRKALDETQPEDSMHLVTWFRRMSINKDSFRKAIDSTI
ELNEETLASIHTVAELAGHCGAREPYQRPDMGHAVNVLSSLVELWKPSDQNSEDIYGIDLDMSLPQALKKWQAYE
GRSQMESSASSSLLPSLDNTQTSIPTRPYGFADSFTSADGR*
>Glyma.14G224000
SEQ ID NO: 71
MEKTGVLSAKEAFEKLEKVGEGTYGKVYRAREKAIGKIVALKKTRLHEDEEGVPPTTLREVSILRMLSRDPHVVR
LMDVKQGQNKEGKTVLYLVFEYMDTDLKKFIRSFRQSGETIPPHIIKVILSICPSIFNIRFFFVHLFLSIWLQSL
MYQLCKGVAFCHGHGILHRDLKPHNLLMDRKTMMLKIADLGLARAFTVPIKKYTHEILTLWYRAPEVLLGATHYS
MAVDMWSVGCIFAELVTKQALFPGDSELQQLLHIFRLLGTPNEDVWPGVSKLMNWHEYPQWNPQSLSTAVPSLDE
LGLDLLSQMLKYEPSKRISAKKAMEHVYFDDLDKRHL*
>Glyma.15G111600
SEQ ID NO: 72
MARARFPLSILLSLVFVALPLSLANTDPSDVQALEVMYNALNSPTQLTGWKIGGGDPCGESWKGVTCEGSAVVSI
KLSGLGLDGTLGYLLSDLMSLRELDLSDNKIHDTIPYQLPPNLTSLNFARNNLSGNLPYSISAMVSLNYLNLSNN
ALSMTVGDIFASLQDLGTLDLSFNNFSGDLPPSFVALANLSSLFLQKNQLTGSLGVLVGLPLDTLNVANNNFSGW
IPHELSSIRNFIYDGNSFENSPAPLPPAFTSPPPNGPHGRHHSGSGSHNKTQVSDNEKSDGHKGLTVGAVVGIVL
GSVLVAAIVLLALVFCIRKQKGKKGARNFSGSLPRGVINVTPQMQEQRVKSAAVVTDLKPRPAENVTVERVAVKS
GSVKQMKSPITSTLYTVASLQSATNSFSQEFIIGEGSLGRVYKADFPNGKVMAIKKIDNSALSLQEEDNFLEAVS
NMSRLRHPSIVTLAGYCAEHGQRLLVYEYIANGNLHDMLHFAEDSSKALSWNARVRIALGTARALEYLHEVCLPS
VVHRNFKSANILLDEELNPHLSDCGLAALTPNTERQVSTQMVGSFGYSAPEFALSGVYTVKSDVYSFGVVMLELL
TGRKPLDSLRVRSEQSLVRWATPQLHDIDALAKMVDPTLNGMYPAKSLSRFADIIALCVQPEPEFRPPMSEVVQA
LVRLVQRASVVKRRPSEESGFGHKTPDHEAMDMPF*
>Glyma.15G203600
SEQ ID NO: 73
MEIFKCFYIKNLILVTFFMVSSAVLAIDPYSEALLSLKSELVDDDNSLHNWVVPSGGKLTGKSYACSWSGIKCNN
DSTIVTSIDLSMKKLGGVVSGKQFIIFTNLTSLNLSHNFFSGQLPAEIFNLTSLTSLDISRNNFSGPFPGGIPRL
QNLVVLDAFSNSFSGPLPAEFSQLENLKVLNLAGSYFRGSIPPEYGSFKSLEFLHLAGNSLTGSIPPELGHLKTV
THMEIGYNEYQGFIPPELGNMSQLQYLDIAGANLSGPIPKQLSNLTSLQSIFLFRNQLTGSIPSELSIIEPLTDL
DLSDNFLIGSIPESFSELENLRLLSVMYNDMSGTVPESIAKLPSLETLLIWNNRFSGSLPPSLGRNSKLKWVDAS
TNDLVGSIPPDICASGELFKLILFSNKFTGGLSSISNCSSLVRLRLEDNSFSGEITLKFSHLPDILYVDLSKNNF
VGGIPSDISQATQLEYFNVSYNPQLGGIIPSQTWSLPQLQNFSASSCGISSDLPLFESCKSISVIDLDSNSLSGT
IPNGVSKCQALEKINLSNNNLTGHIPDELASIPVLGVVDLSNNKFNGPIPAKFGSSSNLQLLNVSFNNISGSIPT
AKSFKLMGRSAFVGNSELCGAPLQPCPDSVGILGSKGTWKVTRIVLLSVGLLIVLLGLVFGILYLRRGIKSQWKM
ASFAGLPQFTANDILTSLSATTKPTDIQSPSVTKTVLPTGITVLVKKIELEARSIKVVSEFIMRLGNARHKNLIR
LLGFCHNQHLVYLLYDYLPNGNLAEKMEMKWDWAAKFRTVVGIARGLCFLHHECYPAIPHGDLRPSNIVFDENME
PHLAEFGFKHVSRWSKGSSPTTTKWETEYNEATKEELSMDIYKFGEMILEILTRERLANSGASIHSKPWEVLLRE
IYNENGASSASSLQEIKLVLEVAMLCTRSRSSDRPSMEDVLKLLSGLKHLEDGRTSKEGQ*
>Glyma.15G222300
SEQ ID NO: 74
MGNETRKLSDEYEVSEVLGRGGFSVVRKGTKKSSSDTKTHVAIKTLRRVGTASNSNNPSGFPRPKGGEKKSTAAM
MGFPTWRQVSVSDALLTNEILVMRRIVENVSPHPNVIDLYDVYEDSNGVHLVLELCSGGELFDRIVAQDRYSETE
AAGVVRQIASGLEAIHRANIVHRDLKPENCLFLDVRRDSPLKIMDFGLSSVEEFTDPVVGLFGSIDYVSPEALSQ
GKITTKSDMWSLGVILYILLSGYPPFIAQNNRQKQQMIMNGNFSFYEKTWKGITRSAKQLISDLLIVDPSRRPSA
QDLLSHPWVVGDKAKDDAMDPEIVSRLQSFNARRKLRAVAIASIWSTTIFLRTKKLKSLVGTHDLTEEEIENLRM
SFKKICVSGDNATLSEFEEVLKAMNMPSLIPLAPRIFDLFDDNRDGTVDMREILCGFSSFKNSKGDDALRLCFQM
YDTDRSGCITKEEVASMLRALPEDCLPTDITEPGKLDEIFDLMDANSDGKVTFDEFKAAMQRDSSLQDVVLSSLR
PQ*
>Glyma.16G079200
SEQ ID NO: 75
MVLHSWISSSHILVNFLLLLGCGITYGTDTDIFCLKSIKESLEDPYNYLKFSWDFNNKTEGYICRFNGVECWHPD
ENRVLNLKLSNMGLKGQFPRGIQNCSSLTGLDLSINKLSGTIPGDISTLIPFATSIDLSTNEFSGAIPVSLANCT
FLNTLKLDQNRLTGQIPPQFGVLSRIKVFSVSNNLLTGQVPIFRDGVELHYANNQGLCGGNTLAPCQATPSKSNM
AVIAGAAAGGVTLAALGLGIGMFFFVRRVSFKKKEEDPEGNKWARSLKGTKRIKVSMFEKSISKMKLSDLMKATN
NFSNTNIIGTGRTGTVYKAVLDDGTTLMVKRLQESQYTEKEFMSEMGTLGTVKHRNLVPLLGFCMTKRERLLVYK
NMPNGNLHDQLHPADGVSTLDWTTRLKIAIGAAKGLAWLHHSCNPRIIHRNISSKCILLDADFEPKISDFGLARL
MNPIDTHLSTFVNGEFGDLGYVAPEYTRTLVATPKGDIYSEGTVLLELVTGERPTNVSKAPETFKGNLVEWITEL
TSNAKLHDAIDESLVRKDVDSELFQFLKVACNCVSPTPKERPTMFEVYQLLRAIGGRYNFTTEDDILVPTDIGNT
DNMQELIVAQEGSY*
>Glyma.17G048900
SEQ ID NO: 76
MGSSFSSCYEGESVSPSPKPTKVVATKGGSSSNRVSITDLSFPGSTLSEDLSVSLVGSNLHVFSLAELKIITQGF
SSSNFLGEGGFGPVHKGFIDDKLRPGLEAQPVAVKLLDLDGSQGHKEWLTEVVFLGQLRHPHLVKLIGYCCEEEH
RLLVYEYLPRGSLENQLFRRYTASLPWSTRMKIAAGAAKGLAFLHEAKKPVIYRDFKASNILLDSDYNAKLSDFG
LAKDGPEGDDTHVSTRVMGTQGYAAPEYIMTGHLTAMSDVYSFGVVLLELLTGRRSVDKGRPQREQNLVEWARSA
LNDSRKLSRIMDPRLEGQYSEVGARKAAALAYQCLSHRPRSRPLMSTVVNVLEPLQDFDDVPIGPFVYTVPAEQQ
QYNEVAKESETPKERKRENGHHHNRRHHHHRHNGHRHHPLKSPKTPMPSDQSQNDEHRNGRKSGSNSPDTFNASE
AQRSMG*
>Glyma.17G083100
SEQ ID NO: 77
MGCSFSGLNALYDSVNGGGDVWINENRFRIVRQLGEGGFAYVYLVKETPNDSAVAAGLSKKLKGSSHLSDDGTYA
MKKVLIQNNEQLELVREEIRVSSLFNHPNLLPLLEHAIISVKPTQETSWNHEAYLLFPVHLDGTLLDNAKIMKAK
KEFYSTSDVLQIFRQLCAGLKHMHSFDPPHAHNDVKPGNVLITHRKGQPPLAILMDFGSARPARKQIGSRSEALQ
LQEWASEHCSAPFRAPELWDCPSQADIDERTDIWSLGCTLYAIMYGVSPFEYALGESGGSLQLAIVNAQVKWPAG
PKPPYPEALHQFVSWMLQPTASMRPRIDDIIIHVDKLVAKESQ*
>Glyma.17G149900
SEQ ID NO: 78
MKCEFFKEKCKSAPELHKKKTPAVNRAANSTGSVSSPKSVKDLYREKEHSFRVFTLQELRDATNGFNRMLKLGEG
GFGSVYKGSITQPDGQGGDPIPVAIKRLNTRGFQGHKEWLAEVQFLGIVNHPNLVKLLGYCSVDAERGIQRLLVY
EFMPNRSLEDHLFNKNLPTLPWKTRLEIMLGAAQGLAYLHEGLEIQVIYRDFKSSNVLLDADFHPKLSDFGLARE
GPQGDQTHVSTAVVGIQGYAAPEYIETGHLKVQSDMWSFGVVLYEILTGRRSLERNRPTAEQKLLDWVKQYPADT
SRFVIIMDARLRNQYSLPAARKIAKLADSCLKKNPEDRPSMSQIVESLKQALQYSDTTSQDIAESSSSSRSKLVR
KK*
>Glyma.17G218500
SEQ ID NO: 79
MLGTPHTLILLLFLFLITTFSHSLVFNITNFDDPAAATAISYEGDGRTTNGSIDLNKVSYLFRVGRAIYSKPLHL
WDRSSDLAIDFVTRFTFSIEKLNLTEVAYGDGFAFYLAPLGYRIPPNSGGGTFGLFNATTNSNLPENHVVAVEFD
TFIGSTDPPTKHVGVDDNSLTSAAFGNFDIDDNLGKKCYTLITYAASTQTLFVSWSFKAKPASTNHNDNSSSFSY
QIDLKKILPEWVNIGFSASTGLSTERNTIYSWEFSSSLNGSPADFENVKLKHQSSKLALILAVLCPLVLLFVLAS
LVAVFLIRKKRRSHDDCMLYEVGDDELGPTSVKFDLDKGTIPRRFEYKELVDATNGFSDDRRLGQGASGQVYKGV
LSYLGRVVAVKRIFADFENSERVFTNEVRIISRLIHKNLVQFIGWCHEEGEFLLVFEYMPNGSLDSHLFGNKRVL
EWHLRYKIVLGVVNALHYLHEDAEQCVLHRDIKSANVLLDTEFNTKVGDFGMAKLVDPRLRTQRTGVVGTYGYLA
PEYVNVGRASRESDIYSFGVVSLEMASGRRTYQDGEFHVSLMNWVWQLYVEGEIMRAADEKLNNEFEVDQMRSLL
VVGLWCTNPNDKERPKAAQVIKVLNLEAPLPVLPLDMYERAPPMEIIRMPHHPSGKNHSGMSTPITSSLVSVGR*
>Glyma.17G246300
SEQ ID NO: 80
MLRDESVGSRSARHSATATATVSNKVLVAVKAEKVISNTALAWALTHVAHSTDSITLLAVYSSHKTGRRFWNFSR
LAGDCTNGPAGKLPEQISDISESCAQMVLQLHNQIEVRVKIKVVTGTPSGAVAAEARWSGSHWVILDKKLKQEVK
HCTDELNCSIVVMNGSQAKILRLNLRSSNELQTPFFSANSSPGIEIAKLKGRRLKHSTPVGSPEEAVYEQNPLYE
GQGPEKRTDESINEPTKDFHVQPPLYFDLERDSPPPSWTRPASSVASDNKTIFWIPQNHNIVDKFQKTKNNSVIQ
RTKSPTSKTLLENFIRCDQEIWTNELGFDQAQSRSYVPNLGIRDNNSVPLGRTTSIPPPLCSQCKNKAPVFGKPP
KRFSYKELEEATDMFSDENFLAEGRFGVVHQGILKDGQVVAVKQLKFGGSQADLDFCREVRVLSCAQHRNVVLLI
GFCIESNLRILVYEYICNGSLDLYLYGDESMPLDWNSRLKIAIGTARGLRYLHEDCRVGCIAHRDLRPKNILVTH
DFEPMVADFGLARWHSEWNIDTEDRVIGTSGYLAPEYLDAGNLTYKVDVYAFGIVLLELITGRRISELEQFNGHS
YLSEWFHPIRMLEPGHILQNVRSLKPCFDSKESVEFNLQLQAMARAVSLCLRVDPDARPPMSKILRVLEGGNPVR
PMGLDINSVGNTSGHLSGLKSHTPPKGTINHSRRLSH*
>Glyma.17G250800
SEQ ID NO: 81
MGMEGIAVSVVTMLLLMVGLTMGMSSRTEWFALRELRQSLEIRAKYWPIKAEPCGNWTGVQCRNGRVVGINVSGL
RRTRWGRLNPSFEVGSLVNFTLLETFNASGFKLNGSIPEWLGESLGVLEVLDLSFCSIKGSIPDSIGWLSKLKVL
LLSGNFLTGRMPSTFGNLTRLSVLNLSGNSLSGTVPDSVSKLGNLSRLDLSYNFLSGSVPPELGALSSLQFFNLS
GNSFTGTFPSQLGNLSKLVDVDLSMNFLSGSLPGGSSSSGLLALKVLILRGNLFDGVLPADLWPMPRLHFLDVSS
NNLTGTLPNFTSWNVSSVGFVFNLSNNLFYGLLNTSLDRFEIIDLSSNYLEGEVPGGGVNNVSLDRNCLQRIPNQ
RDLEDCRVFYDNRSLPFGFLKSGSRSRVIFILVGIFGGLGFIVLLALVLMLVLKQCHNRRSLGVQRGTKDGGPVQ
EGESPIPPKDTVFVTVGDAFSFEQMLHLTSNFAEANVIKHGHSGDLFLGVLEGGATVVVKKVDLNLFKRESYVVE
LGLLSKVPHARLVPILGHCLDNENEKFIVYKYMPNRDLATSLHRVTGSDGKLQSLDWITRLKIAIGAAEGIAYLH
ECSPPLVHRDIQASSILLDDKFEVRLGSLSEVTAQGDLQQGVISRVFSKPPSSNQADSGKSSVTCAYDIYCFGKI
LLELITGNIEVSKLDDASTKEWLEQTLPYITIYDKERVTKIIDPSLIVDEDLLEEVWAMAIVANACLNPKPSKRP
PMRHVLKALENPLKIVREENTSSARLRSNSSRKSWSTAFFGSWRHSSSDSVVATNKEGSSDTKKSGKVGSQSSGN
DHSSSNKRSSNEIFPEPLEILDVETGVAR*
>Glyma.18G107600
SEQ ID NO: 82
MSANSSLHVELSRKTSFLGLRLWVLIGIGVGVFIVVILCVLSVWVMFRRKSKRSLDKYSLSQIPHVSKDIIVDMV
GVQISHDQSESVAIPVHDKPSENNSNKLFSHLHKSKSGDADNISQCSSVYHHERGFSSMSGEEGSSGTVKKQSAL
SFGGMVTASPLVGLPEISHLGWGHWFTLRDLELATNRFSPENVIGEGGYGVVYRGKLINGSEVAVKKILNNLGQA
EKEFRVEVEAIGHVRHKNLVRLLGYCVEGVHRLLVYEYVNNGNLEQWLHGAMSQQGTLTWEARMKVITGTAKALA
YLHEAIEPKVVHRDIKSSNILIDTEFNAKVSDFGLAKLLDSGESHITTRVMGTFGYVAPEYANTGLLNERSDIYS
FGVLLLEAVTGKDPVDYSRPANEVNLVEWLKMMVGTRRAEEVVDSRLEVKPSIRALKRALLVALRCVDPEAEKRP
KMSQVVRMLEADEYPEREDRRNRKSRTASMEIESLKDISGPSDAEKLKGSEGHEPETTQG*
>Glyma.18G141500
SEQ ID NO: 83
MEKTIHLVYFKCLVFLLMKQVIVMAEPRARTVNITCNNKLEHNTTIFVPNFVATMEKISEQMRNTGYGTAVVGTG
GPDTNYGLAQCYGDLSLLDCVLCYAEARTVLPQCFPYNGGRIYLDGCFMRAENYSFYDEYIGPGDKAVCGNTTRK
STSFQAAAKKAVLSAVQAAANNKGYARKEVEVAGTTNDAAYVLANCWRSLDTRSCRACLENASSSILGCLPWSEG
RALNTGCFMRYSDTDFLNKEQENGSSGGNVLVIVVAVVSSVIVLVVGIAIVVYIRKHRYIQMKRRGSNDAEKLAK
SLHHNSLNFKYSTLEKATNSFDEANKLGQGGFGTVYKGVLADGREIAIKRLYFNNRHRAADFFNEVNIISSVEHK
NLVRLLGCSCSGPESLLIYEYLPNRSLDRFIFDKNKGRELNWDKRYDIIIGTAEGLVYLHENSNIRIIHRDIKAS
NILLDAKLRAKIADFGLARSFQEDKSHISTAIAGTLGYMAPEYLAHGQLTEKADVYSFGVLLLEIITGRLNNRSK
ASEYSDSLVTMTWKHFQSGTAEQLIDPCLVVDDNHRSNFKNEILRVLHIGLLCTQEIPSLRPSMSKALKMLTKKE
EHLDLEAPSNPPFIDESTMELHDQNDDPFYPLNAEDSLATMSHSSFYAR*
>Glyma.18G273100
SEQ ID NO: 84
MLFDRLAFCCSKHTSSSQRKYPTVIEELCHQFSLANLKKSTNNFDENGVIGYGRFGKVYKGCLQHNDGSDYSVTL
KRLGVKDSRGLEQFKNEIELLCQLRHPNCVSLIGFCNHKKEKILVYEYMSNGSLHQHLRGGLLSWKKRLEICIEA
AHGLHYLHTGAKRTIIHRNINPSNILLDNNMKSKLTDFRLSIQGPRYGSKPKPIKVYVIEGSLGYMPMEYIVDGT
ITDKFDVFSFGMVLLEVVCGRNCLIIPTETEVLEKPVEENIDQNIKGKIAPECWQVFIDIIIRCLKYEPDERPTM
GEVEVQLEHALSMQEQADITNTNSDYTLFSTTTIHLGLELESNPEESDT*
>Glyma.19G036600
SEQ ID NO: 85
MVRQADLVIIGVSVGLTLGILISCLIFFGIRWYKKRAHIRRSANESSLTTLPIRTNGLGTSSDFSASLDSSIASS
WSENLKRNSHFSWWNHQNKDRFASASGILKYLYKEIQKATQNFTTTLGQGSFGTVYKATMPTGEVVAVKVLAPNS
KQGEKEFQTEVFLLGRLHHRNLVNLVGYCVDKGQRILVYQYMSNGSLANLLYGEEKELSWDQRLQIALDISHGIE
YLHEGAVPPVIHRDLKSANILLDHSMRAKVADFGLSKEEIFDDRNSGLKGTYGYMDPAYISTSKLTTKSDIYSFG
IIVFELITAIHPHQNLMEYVNLAAMDHDGVDEILDKQLVGKCNLEEVRQLAKIGHKCLHKSPRKRPSIGEVSQFI
SRIKQRRQRHLTEDNLSFASNNFSRAVSRLEDRQVELSRMPTINLTETV*
>Glyma.19G143000
SEQ ID NO: 86
METENGDTRSKKMEEYEVIEQIGRGAFGSAFLVLHKSEKKRYVLKKIRLAKQTEKFKRTAHQEMNLIAKLNNPYI
VDYKDAWVEKEDHICIITGYCEGGDMAENIKKARGSFFPEEKVCKWLTQLLIAVDYLHSNRVIHRDLKCSNIFLT
KDNNIRLGDFGLAKRLNAEDLASSVVGTPNYMCPELLADIPYGYKSDMWSLGCCMFEIAAHQPAFRAPDMAGLIN
KINRSSISPLPIVYSSTLKQLIKSMLRKNPEHRPTAAELLRHPLLQPYVLRCHNASSNVLPVYPLVNPKDKARRP
NKSSGGKDHKDKEAGLVNCLERIHPIEGNADIQISNLPNDVVTISTSAEDNLETRMANLTSYIVESSTSISGSKD
GSTTSESTICSVCKEDFKSRPAREMTNNEISSKSTQDSLHEEQRFAAKHFHKLEDDDINAVTAEVEDASCNGGLD
NAEAQREDSNLEDSGKSTMSSEGSSSTDKDKSINEERSSLIVHPIRVENDTESGNRLKKSENPDVYTEVPHMNCL
MSVSNDALPVKDDDIANGHILCSTHKDDNVVEVDQAPSGISLSVITEVDGDETIKTPLDSPCQQRADALESLLEL
CAQLLKQDKLEELAGVLRPFGKEAVSSRETAIWLAKSLISSQKFNPET*
>Glyma.19G166100
SEQ ID NO: 87
MSVMMDHVIGGKFKLGRKIGSGSFGELYIAVNIQTGEEVAVKLEPVKTKHPQLLYESKLYMLLQGGTGIPHLKWF
GVEGDYNVMAIDLLGPSLEDLFNYCNRKLTLKTVLMLADQLINRVEYMHSRGFLHRDIKPDNFLMGLGRKANQVY
IIDYGLAKKYRDLQTHRHIPYRENKNLTGTARYASVNTHLGIEQSRRDDLESLGYVLMYFLRGSLPWQGLKAGTK
KQKYDKISEKKMSTSIEVLCKSYPSEFVSYFNYCRTLRFEDKPDYSYLKRLFRDLFIREGYQFDYVFDWTILKYP
QIGGSSSRGRHESGKAAMHAGPSVQKPEKVSVGKEIREKFSGAVEAFSRRNPTSPSPRGDHSKHRSFEEVAVHKD
VYRDQEKGRNSSRYGSSSRRPIISSSTRPSSSGDHTDSRTGRLTSSGNRSSASHRNIQPMYETKQPTYMRSGSTR
GNRDDPLRSFELLSIRK*
>Glyma.19G261700
SEQ ID NO: 88
MVEFQRLLVCITTCILCWMPNGATAATDPNDAAAVRFLFQNMNSPPQLGWPPNGDDPCGQSWKGITCSGNRVTEI
KLSNLGLTGSLPYGLQVLTSLTYVDMSSNSLGGSIPYQLPPYLQHLNLAYNNITGTVPYSISNLTALTDLNFSHN
QLQQGLGVDFLNLSTLSTLDLSFNFLTGDLPQTMSSLSRITTMYLQNNQFTGTIDVLANLPLDNLNVENNNFTGW
IPEQLKNINLQTGGNAWSSGPAPPPPPGTPPAPKSNQHHKSGGGSTTPSDTATGSSSIDEGKKSGTGGGAIAGIV
ISVIVVGAIVAFFLVKRKSKKSSSDLEKQDNQSFAPLPSNEVHEEKSMQTSSVTDLKTFDTSASINLKPPPIDRH
KSFDDEEFSKRPTIVKKTVTAPANVKSYSIAELQIATGSFSVDHLVGEGSFGRVYRAQFDDGQVLAVKKIDSSIL
PNDLTDDFIQIISNISNLHHPNVTELVGYCSEYGQHLLVYEFHKNGSLHDFLHLSDEYSKPLIWNSRVKIALGTA
RALEYLHEVSSPSVVHKNIKSANILLDTELNPHLSDSGLASYIPNADQILNHNVGSGYDAPEVALSGQYTLKSDV
YSFGVVMLELLSGRNPFDSSRPRSEQSLVRWATPQLHDIDALAKMVDPAMKGLYPVKSLSRFADVIALCVQPEPE
FRPPMSEVVQALVRLVQRANMSKRTFSSSDHGGSQRGSDEPVLRDI*
>Glyma.20G120200
SEQ ID NO: 89
MNLFFFLKPLSSLMILSYIILFFLLVRNTSCDVDPNYVACPPKTCANNNQSISYPFYIEETQEPFCGNPGFAISC
GPNGFPILNLSNTHYIIHQIFYENQTLRVSNAAFSVSRSNTTNSKGCLPVPLTHNLTLPSTPEFDIAPNQSNMRL
FYGCESLLPWPEEHRVGCPNETSSVLAFYKEDKNISLVSKNCRGEVVDTIVEDGIIEGGVEEALRKGLLLTWKAG
NCSECHSSGGRCGFDSIMYTFRCFCTDRVHSAKCDPDNGPVIKKGSSLKLVIGIGIPSMLAIGLLFLFLLYKRKY
ATSGGQLESRDSYSDSSSNPHRETSSEYFGVPLFLYEQLKEATNNFDHTKELGDGGFGTVYHGKLPDGREVAVKR
LYEHNWKRVEQFMNEVKILTRLRHKYLVSLYGCTSRHSRELLLVYEYISNGTVACHLHGELAKPGSLPWSIRMKI
AIETAIALTYLHASDIIHRDVKTNNILLDNNFCVKVADFGLSRDFPNNVTHVSTAPQGSPGYLDPEYYNCYQLTS
KSDVYSFGVVLIELISSKPAVDMNRSRDEINLSNLAVRKIQESAISELVDPSLGFDSDNGIKGMIVSVAGLAFQC
LQREKDLRPSMDEVLDELRRIESGKDEGEVQDEGDVNGAAVSHSSAHSPPPASPEWEEVRLLRNIKPTSPNTVTD
KWESKCTTPNISG*
>Glyma.20G137700
SEQ ID NO: 90
MADLSCIHLFFLCCLFLKFIPQGNASMFHTAFCDNKEGNYTANSTYNTNLNTLLSSLSSHTEINYGFYNFSYGQN
PDKVNAIGLCRGDVEPHECRSCLNDSRVTIKQFCPNQKKALLWLNTSKCMLRYSPRSIFGIMEIEPSQSLMNINN
VTEPDKFSQALANLMRNLKGVAASGDSRRKYATDNVTASSFQTIYGMAECTPDLSEKDCNDCLDGAISKIPTCCQ
DKIGGRVLRPSCNIRFESASFYENTPILNPDVPPPSPAVAIPPSINSTSPKESSNTIRIVIAIVVPTVVVVPLIC
LCIYSRRSKARKSSLVKQHEDDDEIEIAQSLQFNFDTIRVATEDFSDSNKLGQGGFGAVYRGRLSDGQMIAVKRL
SRESSQGDTEFKNEVLLVAKLQHRNLVRLLGFCLEGKERLLIYEYVPNKSLDYFIFDPTKKAQLNWEMRYKIITG
VARGLLYLHEDSHLRIIHRDLKASNILLNEEMNPKIADFGMARLVLMDQTQANTNRIVGTYGYMAPEYAMHGQFS
MKSDVFSFGVLVLEIISGHKNSGIRHGENVEDLLSFAWRNWREGTAVKIVDPSLNNNSRNEMLRCIHIGLLCVQE
NLADRPTMTTIMLMLNSYSLSLPIPSKPAFYVSSRTGSISATQSWGYSSGESRSRELTIKSAQEAENEFTDPYPR*
>Glyma.18G269900
SEQ ID NO: 91
MDTTSTQIASGTILFLLLLFFPYLSIADVIYSPVELFSINCGSSSNLSTRDGRNWTADIKFLSENKDSVAAPALT
PSTLEGPYTDARLSHSQFTYSFPVSTGPKFLRLFFYSTSYQNFHRSKAYFSVKAGPYTLLQNFNASLHADAGNEP
GDYLFREYCINLKDGDRLNITFIASKTSQNPDSYAFINGIEIVSMPPFLYYTNPHDVDITGLPHLVGVNTNLFPI
ENNFTLETKYRLRVGDQEIPASQDTGMLRSWDVDSKYVTTQSVLSLDIGPGIKLRFTKIPNYTAPDTVYRSVRNM
GNNGTINMGFNLTWQLPIDSGFTYLLRLHFCQLNPEMKNPGYQSFFIFVQDQLVEKWADILSWSDKQEGVPVVKQ
YVVFIPGNQQETLNLSLKMHPNPQSLAKDAQINAIELFKINNSTGSLAGPNPDPDRLPETPKVPLQRPNNKSSGT
TRTLAAAVAGAVSAAVLLSFIVAFFLIKRKKKMGSKEKDETPLGGGLSSLPTNLCRHFSIAEIRASTNNFDEHFV
VGMGGFGNVYKGYIDDGSTRVAIKRLKPDSRQGAQEFMNEIEMLSQLRHLHLVSLVGYCYESNEMILVYDFMDRG
TLREHLYDTDNPSLSWKQRLQICVGAARGLHYLHTGAKHTIIHRDVKSTNILLDEKWVAKVSDFGLSRIGPISSS
MTHVSTQVKGSVGYIDPEYYKRQRLTEKSDVYSFGVVLLEVLSGRQPLLRWEEKQRISLVNWAKHCNEKGTLSEI
VDAKLKGQIAPQCLQRYGEVALSCLLEDGTQRPSMNDAVRMLEFVLHLQEGAVNEVTESEDTEDVFSSSHSSLLF
SDYSKSTALSMATNVGDCSYGSKDSEERSIPDHLFSEIKDPKGR*
Sequences found in Table 2
>Glyma.03G189800 (wild type)
SEQ ID NO: 15
MKKVCHCPIFLALALCLCILCVAAEAAGQNDTLALTEFRLQTDTHGNLLTNWTGADACSAAWRGVECS
PNGRVVGLTLPSLNLRGPIDTLSTLTYLRFLDLHENRLNGTISPLLNCTSLELLYLSRNDFSGEIPAE
ISSLRLLLRLDISDNNIRGPIPTQLAKLTHLLTLRLQNNALSGHVPDLSASLLNLTVLNVTNNELRGH
VPDSMLTKFGNVSFSGNHALCGSTPLPKCSETEPDTETTTITVPAKPSSFPQTSSVTVPDTPRKKGLS
AGVIVAIVVAVCVAVLVATSFAVAHCCARGSTSGSVVGSETAKRKSGSSSGSEKKVYGNGGNLDRDSD
GTNTETERSKLVFFDRRNQFELEDLLRASAEMLGKGSLGTVYRAVLDDGCTVAVKRLKDANPCERNEF
EQYMDVVGKLKHPNIVRLRAYYYAKEEKLLVYDYLPNGSLHALLHGNRGPGRIPLDWTTRISLMLGAA
RGLARIHAEYNASKIPHGNVKSSNVLLDKNGVALISDFGLSLLLNPVHAIARLGGYRAPEQVEVKRLS
QEADVYGEGVLLLEVLIGRAPSKEYTSPAREAEVDLPKWVKSVVKEEWTSEVEDQELLRYKNIEDELV
AMLHVGLACVAAQAEKRPCMLEVVKMIEEIRVEESPLGDDYDEARSRTSLSPSLATTEDNLA
>Glyma.04G222800 (wild type)
SEQ ID NO: 17
MQALFSSMRVFLRLVWLLELLCVAVTAVNPSLNDDVLGLIVFKADIRDPKGKLASWNEDDESACGGSW
VGVKCNPRSNRVVEVNLDGFSLSGRIGRGLQRLQFLRKLSLANNNLTGGINPNIARIDNLRVIDLSGN
SLSGEVSEDVFRQCGSLRTVSLARNRFSGSIPSTLGACSALAAIDLSNNQFSGSVPSRVWSLSALRSL
DLSDNLLEGEIPKGIEAMKNLRSVSVARNRLTGNVPYGFGSCLLLRSIDLGDNSFSGSIPGDFKELTL
CGYISLRGNAFSGGVPQWIGEMRGLETLDLSNNGFTGQVPSSIGNLQSLKMLNFSGNGLTGSLPESMA
NCTKLLVLDVSRNSMSGWLPLWVFKSDLDKVLVSENVQSGSKKSPLFAMAELAVQSLQVLDLSHNAFS
GEITSAVGGLSSLQVLNLANNSLGGPIPPAVGELKTCSSLDLSYNKLNGSIPWEIGGAVSLKELVLEK
NFLNGKIPTSIENCSLLTTLILSQNKLSGPIPAAVAKLTNLQTVDVSFNNLIGALPKQLANLANLLTF
NLSHNNLQGELPAGGFFNTITPSSVSGNPSLCGAAVNKSCPAVLPKPIVLNPNTSTDTGPSSLPPNLG
HKRIILSISALIAIGAAAVIVIGVISITVLNLRVRSSTSRDAAALTFSAGDEFSHSPTTDANSGKLVM
FSGEPDFSSGAHALLNKDCELGRGGFGAVYQTVLRDGHSVAIKKLTVSSLVKSQEDFEREVKKLGKIR
HQNLVELEGYYWTPSLQLLIYEYLSGGSLYKHLHEGSGGNFLSWNERFNVILGTAKALAHLHHSNIIH
YNIKSTNVLLDSYGEPKVGDFGLARLLPMLDRYVLSSKIQSALGYMAPEFACKTVKITEKCDVYGFGV
LVLEIVTGKRPVEYMEDDVVVLCDMVRGALEEGRVEECIDERLQGKFPAEEAIPVMKLGLICTSQVPS
NRPDMGEVVNILELIRCPSEGQEELA
>Glyma.12G056000 (wild type)
SEQ ID NO: 49
MDSARTAPPPWQELDLDSLKPLKVLGKGGMGTVFLVQAANNTRFALKVVDKTCVHAKLDAERRARWEI
QVLSTLSHPFLPSLMGTFESPQFLAWALPYCPGGDLNVLRYRQTDRAFSPAVIRFYVAEILCALDHLH
SMGIAYRDLKPENVLVQNTGHITLTDFDLSRKLNPKPKPNPQVPSIPLPNSNVPEPRRKHRRNFSRWI
SLFPPDGTHHNNNKNGLKKAKSARVSPVSRRKPSFSNGERSNSFVGTEEYVSPEVVRGDGHEFAVDWW
ALGILIYEMLYGTTPFKGKNRKETFRNVITKPPVFVGKRTALTDLIEKLLEKDPTKRLGYTRGAVEIK
EHEFFRGVRWELLTEVVRPPFIPTRDDGAGDSTDRISDRNCGFDIRGYFLNLKSSPSLPGSPLPSPSC
RFKKNVSLTEF
>Glyma.13G150000 (wild type)
SEQ ID NO: 60
MMKHYHVLYMFLFFLPISTLSLHHNDTHALTLFRRQSDLHGYLLSNWTGHDACNSAWRGVLCSPNGRV
TALSLPSLNLRGPLDPLTPLTHLRLLNLHDNRLNGTVSTLFSNCTNLQLLYLSSNDFSGEIPPEISSL
KSLLRLDLSDNNLRGKVDVISNLTQLITLRLQNNLLSGEIPDLSSSMKNLKELNMTNNEFYGRLPSPM
LKKFSSTTFSGNEGLCGASLFPGCSFTTTPPNNNDSNNNNDNNSNEKEPSQTVSSNPSSFPETSIIAR
PGREQQRKGLSPGAIVAIVIANCVALLVVVSFAVAHCCARGRGSSLVGSGESYGKRKSESSYNGSDEK
KVYGGGESDGTSGTDRSRLVFFDRRSEFELEDLLRASAEMLGKGSLGTVYRAVLDDGCTVAVKRLKDA
NPCARHEFEQYMDVIGKLKHPNVVRLKAYYYAKEEKLLVYDYLSNGSLHALLHGNRGPGRIPLDWTTR
ISLVLGAARGLAKIHAEYSAAKVPHGNVKSSNVLLDKNGVACISDFGLSLLLNPVHAIARLGGYRAPE
QEQNKRLSQQADVYSFGVLLLEVLIGRAPSSQYPSPARPRMEVEPEQAAVDLPKWVRSVVREEWTAEV
FDQELLRYKNIEEELVSMLHVGLTCVVAQPEKRPTMEEVVKMIEEIRVEQSPLGEDYDVSCNSLSPSI
PTTEDGLA
>Glyma.14G026300 (wild type)
SEQ ID NO: 66
METSLDYSKMLFLLLLGSTSLIFLSHLASAATPKLNTQEVKALKEIGSKIGKKDWNFGVDPCSGKGNW
NVPDARKAFVMSSVICDCSFNHNSSCHVVSIYWKAQNLSGSLSPEFSKLHYLQKLDLSRNIITGSIPP
QWGTMRLVELSLMGNKLSGPFPKVLTNITTLRNLSIEGNQFSGHIPTEIGKLTNLEKLVLSSNGFTGA
LPPVLSKLTKLIDLRISDNNFLGKIPDFISNWTLIEKLHMHGCSLEGPIPSSISALTRLSDLRITDLK
GSKSSAFPPLNNLKSMKTLVLRKCMIKGEIPEYIGRMEKLKILDLSYNGLSGEIPESFAQLDKVDFMY
LTGNKLSGIIPRWVLANNENIDISDNNFSWDSSSPTECQRGSVNLVESYSSSVNTQTKINSCLKKNFL
CTASPSQYRYSLNINCGGNEANVSGNIYEADREQKGAAMLYYTSQDWALSSTGNFMDNDIDSDPYIVA
NTSRLNVSALNSKLYTTARVSPLALTYYGLCLINGNYTVKLHFAEIIFINDRSLNSLGRRVFDVYIQG
NLVLKDFDIRREAGGTGKSIEKTFNASVTQHTLKIHFYWAGKGTTGIPTRGVYGPLVSAISVNPNFKP
PSGEGKRTYLILAIIIVAGVLVVVLLVLVLLRRMGWLGGKDPVYKELRGIDLQTGLFTLRQIKAATKN
FDALNKIGEGGFGCVYKGQQSDGTMIAVKQLSSKSKQGNREFVNEMGLISGLQHPNLVKLYGCCVEGN
QLILIYEYMENNCLSRILFGRDPNKTKLDWPTRKKICLGIAKALAYLHEESRIKIIHRDVKASNVLLD
KDFNAKVSDFGLAKLIEDEKTHISTRVAGTIGYMAPEYAMRGYLTDKADVYSFGVVALETVSGKSNTN
FRPNEDFVYLLDWAYVLQERGSLLELVDPNLGSEYLTEEAMVVLNVALLCTNASPTLRPTMSQVVSML
EGWTDIQDLLSDPGYSAISSSSKHKSIRSHFWQTPSGTHSISIPSIYTDSSGSHVETEKNYHPVTVNS
DGSDKSN
>Glyma.16G079200 (wild type)
SEQ ID NO: 75
MVLHSWISSSHILVNFLLLLGCGITYGTDTDIFCLKSIKESLEDPYNYLKFSWDFNNKTEGYICRFNG
VECWHPDENRVLNLKLSNMGLKGQFPRGIQNCSSLTGLDLSINKLSGTIPGDISTLIPFATSIDLSTN
EFSGAIPVSLANCTFLNTLKLDQNRLTGQIPPQFGVLSRIKVFSVSNNLLTGQVPIFRDGVELHYANN
QGLCGGNTLAPCQATPSKSNMAVIAGAAAGGVTLAALGLGIGMFFFVRRVSFKKKEEDPEGNKWARSL
KGTKRIKVSMFEKSISKMKLSDLMKATNNFSNTNIIGTGRTGTVYKAVLDDGTTLMVKRLQESQYTEK
EFMSEMGTLGTVKHRNLVPLLGFCMTKRERLLVYKNMPNGNLHDQLHPADGVSTLDWTTRLKIAIGAA
KGLAWLHHSCNPRIIHRNISSKCILLDADFEPKISDFGLARLMNPIDTHLSTFVNGEFGDLGYVAPEY
TRTLVATPKGDIYSFGTVLLELVTGERPTNVSKAPETFKGNLVEWITELTSNAKLHDAIDESLVRKDV
DSELFQFLKVACNCVSPTPKERPTMFEVYQLLRAIGGRYNFTTEDDILVPTDIGNTDNMQELIVAQEG
SY
>Glyma.18G141500 (wild type)
SEQ ID NO: 83
MEKTIHLVYFKCLVFLLMKQVIVMAEPRARTVNITCNNKLEHNTTIFVPNEVATMEKISEQMRNTGYG
TAVVGTGGPDTNYGLAQCYGDLSLLDCVLCYAEARTVLPQCFPYNGGRIYLDGCFMRAENYSFYDEYI
GPGDKAVCGNTTRKSTSFQAAAKKAVLSAVQAAANNKGYARKEVEVAGTTNDAAYVLANCWRSLDTRS
CRACLENASSSILGCLPWSEGRALNTGCFMRYSDTDFLNKEQENGSSGGNVLVIVVAVVSSVIVLVVG
IAIVVYIRKHRYIQMKRRGSNDAEKLAKSLHHNSLNFKYSTLEKATNSFDEANKLGQGGFGTVYKGVL
ADGREIAIKRLYFNNRHRAADFFNEVNIISSVEHKNLVRLLGCSCSGPESLLIYEYLPNRSLDRFIFD
KNKGRELNWDKRYDIIIGTAEGLVYLHENSNIRIIHRDIKASNILLDAKLRAKIADEGLARSFQEDKS
HISTAIAGTLGYMAPEYLAHGQLTEKADVYSFGVLLLEIITGRLNNRSKASEYSDSLVTMTWKHFQSG
TAEQLIDPCLVVDDNHRSNFKNEILRVLHIGLLCTQEIPSLRPSMSKALKMLTKKEEHLDLEAPSNPP
FIDESTMELHDQNDDPFYPLNAEDSLATMSHSSFYAR
>Glyma.18G269900 (wild type)
SEQ ID NO: 91
MDTTSTQIASGTILFLLLLFFPYLSIADVIYSPVELFSINCGSSSNLSTRDGRNWTADIKFLSENKDS
VAAPALTPSTLEGPYTDARLSHSQFTYSFPVSTGPKFLRLFFYSTSYQNFHRSKAYFSVKAGPYTLLQ
NFNASLHADAGNEPGDYLFREYCINLKDGDRLNITFIASKTSQNPDSYAFINGIEIVSMPPFLYYTNP
HDVDITGLPHLVGVNTNLFPIENNFTLETKYRLRVGDQEIPASQDTGMLRSWDVDSKYVTTQSVLSLD
IGPGIKLRFTKIPNYTAPDTVYRSVRNMGNNGTINMGFNLTWQLPIDSGFTYLLRLHFCQLNPEMKNP
GYQSFFIFVQDQLVEKWADILSWSDKQEGVPVVKQYVVFIPGNQQETLNLSLKMHPNPQSLAKDAQIN
AIELFKINNSTGSLAGPNPDPDRLPETPKVPLQRPNNKSSGTTRTLAAAVAGAVSAAVLLSFIVAFFL
IKRKKKMGSKEKDETPLGGGLSSLPTNLCRHFSIAEIRASTNNFDEHFVVGMGGFGNVYKGYIDDGST
RVAIKRLKPDSRQGAQEFMNEIEMLSQLRHLHLVSLVGYCYESNEMILVYDFMDRGTLREHLYDTDNP
SLSWKQRLQICVGAARGLHYLHTGAKHTIIHRDVKSTNILLDEKWVAKVSDFGLSRIGPISSSMTHVS
TQVKGSVGYIDPEYYKRQRLTEKSDVYSFGVVLLEVLSGRQPLLRWEEKQRISLVNWAKHCNEKGTLS
EIVDAKLKGQIAPQCLQRYGEVALSCLLEDGTQRPSMNDAVRMLEFVLHLQEGAVNEVTESEDTEDVF
SSSHSSLLFSDYSKSTALSMATNVGDCSYGSKDSEERSIPDHLFSEIKDPKGR
>Glyma.03G189800 (kinase-dead)
SEQ ID NO: 92
MKKVCHCPIFLALALCLCILCVAAEAAGQNDTLALTEFRLQTDTHGNLLTNWTGADACSAAWRGVECS
PNGRVVGLTLPSLNLRGPIDTLSTLTYLRFLDLHENRLNGTISPLLNCTSLELLYLSRNDFSGEIPAE
ISSLRLLLRLDISDNNIRGPIPTQLAKLTHLLTLRLQNNALSGHVPDLSASLLNLTVLNVTNNELRGH
VPDSMLTKFGNVSFSGNHALCGSTPLPKCSETEPDTETTTITVPAKPSSFPQTSSVTVPDTPRKKGLS
AGVIVAIVVAVCVAVLVATSFAVAHCCARGSTSGSVVGSETAKRKSGSSSGSEKKVYGNGGNLDRDSD
GTNTETERSKLVFFDRRNQFELEDLLRASAEMLGKGSLGTVYRAVLDDGCTVAVRRLKDANPCERNEF
EQYMDVVGKLKHPNIVRLRAYYYAKEEKLLVYDYLPNGSLHALLHGNRGPGRIPLDWTTRISLMLGAA
RGLARIHAEYNASKIPHGNVRSSNVLLDKNGVALISDFGLSLLLNPVHAIARLGGYRAPEQVEVKRLS
QEADVYGFGVLLLEVLTGRAPSKEYTSPAREAEVDLPKWVKSVVKEEWTSEVFDQELLRYKNIEDELV
AMLHVGLACVAAQAEKRPCMLEVVKMIEEIRVEESPLGDDYDEARSRTSLSPSLATTEDNLA
>Glyma.04G222800 (kinase-dead)
SEQ ID NO: 93
MQALFSSMRVFLRLVWLLELLCVAVTAVNPSLNDDVLGLIVFKADIRDPKGKLASWNEDDESACGGSW
VGVKCNPRSNRVVEVNLDGFSLSGRIGRGLQRLQFLRKLSLANNNLTGGINPNIARIDNLRVIDLSGN
SLSGEVSEDVFRQCGSLRTVSLARNRFSGSIPSTLGACSALAAIDLSNNQFSGSVPSRVWSLSALRSL
DLSDNLLEGEIPKGIEAMKNLRSVSVARNRLTGNVPYGFGSCLLLRSIDLGDNSFSGSIPGDFKELTL
CGYISLRGNAFSGGVPQWIGEMRGLETLDLSNNGFTGQVPSSIGNLQSLKMLNFSGNGLTGSLPESMA
NCTKLLVLDVSRNSMSGWLPLWVFKSDLDKVLVSENVQSGSKKSPLFAMAELAVQSLQVLDLSHNAFS
GEITSAVGGLSSLQVLNLANNSLGGPIPPAVGELKTCSSLDLSYNKLNGSIPWEIGGAVSLKELVLEK
NFLNGKIPTSIENCSLLTTLILSQNKLSGPIPAAVAKLTNLQTVDVSFNNLTGALPKQLANLANLLTF
NLSHNNLQGELPAGGFFNTITPSSVSGNPSLCGAAVNKSCPAVLPKPIVLNPNTSTDTGPSSLPPNLG
HKRIILSISALIAIGAAAVIVIGVISITVLNLRVRSSTSRDAAALTFSAGDEFSHSPTTDANSGKLVM
FSGEPDFSSGAHALLNKDCELGRGGFGAVYQTVLRDGHSVAIKRLTVSSLVKSQEDFEREVKKLGKIR
HQNLVELEGYYWTPSLQLLIYEYLSGGSLYKHLHEGSGGNFLSWNERFNVILGTAKALAHLHHSNIIH
YNIRSTNVLLDSYGEPKVGDFGLARLLPMLDRYVLSSKIQSALGYMAPEFACKTVKITEKCDVYGFGV
LVLEIVTGKRPVEYMEDDVVVLCDMVRGALEEGRVEECIDERLQGKFPAEEAIPVMKLGLICTSQVPS
NRPDMGEVVNILELIRCPSEGQEELA
>Glyma.12G056000 (kinase-dead)
SEQ ID NO: 94
MDSARTAPPPWQELDLDSLKPLKVLGKGGMGTVFLVQAANNTRFALRVVDKTCVHAKLDAERRARWEI
QVLSTLSHPFLPSLMGTFESPQFLAWALPYCPGGDLNVLRYRQTDRAFSPAVIRFYVAEILCALDHLH
SMGIAYRDLRPENVLVQNTGHITLTDFDLSRKLNPKPKPNPQVPSIPLPNSNVPEPRRKHRRNFSRWI
SLFPPDGTHHNNNKNGLKKAKSARVSPVSRRKPSFSNGERSNSFVGTEEYVSPEVVRGDGHEFAVDWW
ALGILIYEMLYGTTPFKGKNRKETFRNVITKPPVFVGKRTALTDLIEKLLEKDPTKRLGYTRGAVEIK
EHEFFRGVRWELLTEVVRPPFIPTRDDGAGDSTDRISDRNCGFDIRGYFLNLKSSPSLPGSPLPSPSC
RFKKNVSLTEF
>Glyma.13G150000 (kinase-dead)
SEQ ID NO: 95
MMKHYHVLYMFLFFLPISTLSLHHNDTHALTLFRRQSDLHGYLLSNWTGHDACNSAWRGVLCSPNGRV
TALSLPSLNLRGPLDPLTPLTHLRLLNLHDNRLNGTVSTLFSNCTNLQLLYLSSNDFSGEIPPEISSL
KSLLRLDLSDNNLRGKVDVISNLTQLITLRLQNNLLSGEIPDLSSSMKNLKELNMTNNEFYGRLPSPM
LKKFSSTTFSGNEGLCGASLFPGCSFTTTPPNNNDSNNNNDNNSNEKEPSQTVSSNPSSFPETSIIAR
PGREQQRKGLSPGAIVAIVIANCVALLVVVSFAVAHCCARGRGSSLVGSGESYGKRKSESSYNGSDEK
KVYGGGESDGTSGTDRSRLVFFDRRSEFELEDLLRASAEMLGKGSLGTVYRAVLDDGCTVAVRRLKDA
NPCARHEFEQYMDVIGKLKHPNVVRLKAYYYAKEEKLLVYDYLSNGSLHALLHGNRGPGRIPLDWTTR
ISLVLGAARGLAKIHAEYSAAKVPHGNVRSSNVLLDKNGVACISDFGLSLLLNPVHAIARLGGYRAPE
QEQNKRLSQQADVYSFGVLLLEVLTGRAPSSQYPSPARPRMEVEPEQAAVDLPKWVRSVVREEWTAEV
FDQELLRYKNIEEELVSMLHVGLTCVVAQPEKRPTMEEVVKMIEEIRVEQSPLGEDYDVSCNSLSPSI
PTTEDGLA
>Glyma.14G026300 (kinase-dead)
SEQ ID NO: 96
METSLDYSKMLFLLLLGSTSLIFLSHLASAATPKLNTQEVKALKEIGSKIGKKDWNFGVDPCSGKGNW
NVPDARKAFVMSSVICDCSFNHNSSCHVVSIYWKAQNLSGSLSPEFSKLHYLQKLDLSRNIITGSIPP
QWGTMRLVELSLMGNKLSGPFPKVLTNITTLRNLSIEGNQFSGHIPTEIGKLTNLEKLVLSSNGFTGA
LPPVLSKLTKLIDLRISDNNFLGKIPDFISNWTLIEKLHMHGCSLEGPIPSSISALTRLSDLRITDLK
GSKSSAFPPLNNLKSMKTLVLRKCMIKGEIPEYIGRMEKLKILDLSYNGLSGEIPESFAQLDKVDFMY
LTGNKLSGIIPRWVLANNENIDISDNNFSWDSSSPTECQRGSVNLVESYSSSVNTQTKINSCLKKNFL
CTASPSQYRYSLNINCGGNEANVSGNIYEADREQKGAAMLYYTSQDWALSSTGNFMDNDIDSDPYIVA
NTSRLNVSALNSKLYTTARVSPLALTYYGLCLINGNYTVKLHFAETIFINDRSLNSLGRRVFDVYIQG
NLVLKDFDIRREAGGTGKSIEKTFNASVTQHTLKIHFYWAGKGTTGIPTRGVYGPLVSAISVNPNFKP
PSGEGKRTYLILAIIIVAGVLVVVLLVLVLLRRMGWLGGKDPVYKELRGIDLQTGLFTLRQIKAATKN
FDALNKIGEGGFGCVYKGQQSDGTMIAVRQLSSKSKQGNREFVNEMGLISGLQHPNLVKLYGCCVEGN
QLILIYEYMENNCLSRILFGRDPNKTKLDWPTRKKICLGIAKALAYLHEESRIKIIHRDVRASNVLLD
KDFNAKVSDFGLAKLIEDEKTHISTRVAGTIGYMAPEYAMRGYLTDKADVYSFGVVALETVSGKSNTN
FRPNEDFVYLLDWAYVLQERGSLLELVDPNLGSEYLTEEAMVVLNVALLCTNASPTLRPTMSQVVSML
EGWTDIQDLLSDPGYSAISSSSKHKSIRSHFWQTPSGTHSISIPSIYTDSSGSHVETEKNYHPVTVNS
DGSDKSN
>Glyma.16G079200 (kinase-dead)
SEQ ID NO: 97
MVLHSWISSSHILVNFLLLLGCGITYGTDTDIFCLKSIKESLEDPYNYLKFSWDFNNKTEGYICRFNG
VECWHPDENRVLNLKLSNMGLKGQFPRGIQNCSSLTGLDLSINKLSGTIPGDISTLIPFATSIDLSTN
EFSGAIPVSLANCTFLNTLKLDQNRLTGQIPPQFGVLSRIKVFSVSNNLLTGQVPIFRDGVELHYANN
QGLCGGNTLAPCQATPSKSNMAVIAGAAAGGVTLAALGLGIGMFFFVRRVSFKKKEEDPEGNKWARSL
KGTKRIKVSMFEKSISKMKLSDLMKATNNFSNTNIIGTGRTGTVYKAVLDDGTTLMVRRLQESQYTEK
EFMSEMGTLGTVKHRNLVPLLGFCMTKRERLLVYKNMPNGNLHDQLHPADGVSTLDWTTRLKIAIGAA
KGLAWLHHSCNPRIIHRNISSRCILLDADFEPKISDFGLARLMNPIDTHLSTFVNGEFGDLGYVAPEY
TRTLVATPKGDIYSFGTVLLELVTGERPTNVSKAPETFKGNLVEWITELTSNAKLHDAIDESLVRKDV
DSELFQFLKVACNCVSPTPKERPTMFEVYQLLRAIGGRYNFTTEDDILVPTDIGNTDNMQELIVAQEG
SY
>Glyma.18G141500 (kinase-dead)
SEQ ID NO: 98
MEKTIHLVYFKCLVFLLMKQVIVMAEPRARTVNITCNNKLEHNTTIFVFNFVATMEKISEQMRNTGYG
TAVVGTGGPDTNYGLAQCYGDLSLLDCVLCYAEARTVLPQCFPYNGGRIYLDGCFMRAENYSFYDEYI
GPGDKAVCGNTTRKSTSFQAAAKKAVLSAVQAAANNKGYARKEVFVAGTTNDAAYVLANCWRSLDTRS
CRACLENASSSILGCLPWSEGRALNTGCFMRYSDTDFLNKEQENGSSGGNVLVIVVAVVSSVIVLVVG
IAIVVYIRKHRYIQMKRRGSNDAEKLAKSLHHNSLNFKYSTLEKATNSFDEANKLGQGGFGTVYKGVL
ADGREIAIRRLYFNNRHRAADFFNEVNIISSVEHKNLVRLLGCSCSGPESLLIYEYLPNRSLDRFIFD
KNKGRELNWDKRYDIIIGTAEGLVYLHENSNIRIIHRDIRASNILLDAKLRAKIADFGLARSFQEDKS
HISTAIAGTLGYMAPEYLAHGQLTEKADVYSFGVLLLEIITGRLNNRSKASEYSDSLVTMTWKHFQSG
TAEQLIDPCLVVDDNHRSNFKNEILRVLHIGLLCTQEIPSLRPSMSKALKMLTKKEEHLDLEAPSNPP
FIDESTMELHDQNDDPFYPLNAEDSLATMSHSSFYAR
>Glyma.18G269900 (kinase-dead)
SEQ ID NO: 99
MDTTSTQIASGTILFLLLLFFPYLSIADVIYSPVELFSINCGSSSNLSTRDGRNWTADIKFLSENKDS
VAAPALTPSTLEGPYTDARLSHSQFTYSFPVSTGPKFLRLFFYSTSYQNFHRSKAYFSVKAGPYTLLQ
NFNASLHADAGNEPGDYLFREYCINLKDGDRLNITFIASKTSQNPDSYAFINGIEIVSMPPFLYYTNP
HDVDITGLPHLVGVNTNLFPIENNFTLETKYRLRVGDQEIPASQDTGMLRSWDVDSKYVTTQSVLSLD
IGPGIKLRFTKIPNYTAPDTVYRSVRNMGNNGTINMGFNLTWQLPIDSGFTYLLRLHFCQLNPEMKNP
GYQSFFIFVQDQLVEKWADILSWSDKQEGVPVVKQYVVFIPGNQQETLNLSLKMHPNPQSLAKDAQIN
AIELFKINNSTGSLAGPNPDPDRLPETPKVPLQRPNNKSSGTTRTLAAAVAGAVSAAVLLSFIVAFFL
IKRKKKMGSKEKDETPLGGGLSSLPTNLCRHFSIAEIRASTNNFDEHFVVGMGGFGNVYKGYIDDGST
RVAIRRLKPDSRQGAQEFMNEIEMLSQLRHLHLVSLVGYCYESNEMILVYDFMDRGTLREHLYDTDNP
SLSWKQRLQICVGAARGLHYLHTGAKHTIIHRDVRSTNILLDEKWVAKVSDFGLSRIGPISSSMTHVS
TQVKGSVGYIDPEYYKRQRLTEKSDVYSFGVVLLEVLSGRQPLLRWEEKQRISLVNWAKHCNEKGTLS
EIVDAKLKGQIAPQCLQRYGEVALSCLLEDGTQRPSMNDAVRMLEFVLHLQEGAVNEVTESEDTEDVF
SSSHSSLLFSDYSKSTALSMATNVGDCSYGSKDSEERSIPDHLFSEIKDPKGR

### REFERENCES

1. ADDIN EN.REFLIST Block A, Li G, Fu Z Q, Alfano J R (2008) Phytopathogen type III effector weaponry and their plant targets. Current Opin Plant Biology 11:396-403.
2. Champion A, Kreis M, Mockaitis K, Picaud A, Henry Y (2004) Arabidopsis kinome: after the casting. Funct Integr Genomic 4:163-187.
3. Colcombet J, Hirt H (2008) Arabidopsis MAPKs: a complex signalling network involved in multiple biological processes. Biochem J 413:217-226.
4. de la Fuente A (2010) From ‘differential expression’ to ‘differential networking’-identification of dysfunctional regulatory networks in diseases. Trends Genet 26:326-333.
5. Dou D, Zhou J M (2012) Phytopathogen effectors subverting host immunity: different foes, similar battleground. Cell Host Microbe 12:484-495.
6. Gao L L, Xue H W (2012) Global analysis of expression profiles of rice receptor-like kinase genes. Mol Plant 5:143-153.
7. Gish L A, Clark S E (2011) The RLK/Pelle family of kinases. Plant J 66:117-127.
8. Hanada K, Zou C, Lehti-Shiu M D, Shinozaki K, Shiu S-H (2008) Importance of lineage-specific expansion of plant tandem duplicates in the adaptive response to environmental stimuli. Plant Physiol 148:993-1003.
9. Hanks S K, Hunter T (1995) Protein kinases 6. The eukaryotic protein kinase superfamily: kinase (catalytic) domain structure and classification. FASEB J 9:576-596.
10. Hewezi T (2015) Cellular signaling pathways and posttranslational modifications mediated by nematode effector proteins. Plant Physiol 169:1018-1026.
11. Hewezi T, Baum T J (2013) Manipulation of plant cells by cyst and root-knot nematode effectors. Mol Plant Microbe In 26:9-16.
12. Hewezi T, Juvale P S, Piya S, Maier T R, Rambani A, Rice J H, Mitchum M G, Davis E L, Hussey R S, Baum T J (2015) The cyst nematode effector protein 10A07 targets and recruits host posttranslational machinery to mediate its nuclear trafficking and to promote parasitism in Arabidopsis. Plant Cell 27:891-907.
13. Hogenhout S A, Van der Hoorn R A, Terauchi R, Kamoun S (2009) Emerging concepts in effector biology of plant-associated organisms. Mol Plant Microbe In 22:115-122.
14. Kandoth P K, Ithal N, Recknor J, Maier T, Nettleton D, Baum T J, Mitchum M G (2011) The soybean Rhg1 locus for resistance to the soybean cyst nematode Heterodera glycines regulates the expression of a large number of stress- and defense-related genes in degenerating feeding cells. Plant Physiol 155:1960-1975.
15. Klink V P, Hosseini P, Matsye P, Alkharouf N W, Matthews BF (2009) A gene expression analysis of syncytia laser microdissected from the roots of the Glycine max (soybean) genotype PI 548402 (Peking) undergoing a resistant reaction after infection by Heterodera glycines (soybean cyst nematode). Plant Mol Biol 71:525-567.
16. Klink V P, Hosseini P, Matsye P D, Alkharouf N W, Matthews B F (2010) Syncytium gene expression in Glycine max ([PI 88788]) roots undergoing a resistant reaction to the parasitic nematode Heterodera glycines. Plant Physiol Biochem 48:176-193.
17. Klink V P, Overall C C, Alkharouf N W, MacDonald M H, Matthews B F (2007) Laser capture microdissection (LCM) and comparative microarray expression analysis of syncytial cells isolated from incompatible and compatible soybean (Glycine max) roots infected by the soybean cyst nematode (Heterodera glycines). Planta 226:1389-1409.
18. Lehti-Shiu M D, Shiu S H (2012) Diversity, classification and function of the plant protein kinase superfamily. Philos Trans R Soc Lond B Biol Sci 367:2619-2639.
19. Lehti-Shiu M D, Zou C, Hanada K, Shiu S H (2009) Evolutionary history and stress regulation of plant receptor-like kinase/pelle genes. Plant Physiol 150:12-26.
20. Liu J Y, Chen N N, Grant J N, Cheng Z M, Stewart C N, Hewezi T (2015) Soybean kinome: functional classification and gene expression patterns. J Exp Bot 66:1919-1934.
21. Mendy B, Wang′ombe M W, Radakovic Z S, Holbein J, Ilyas M, Chopra D, Holton N, Zipfel C, Grundler F M, Siddique S (2017) Arabidopsis leucine-rich repeat receptor-like kinase NILR1 is required for induction of innate immunity to parasitic nematodes. PLOS Patho 13.
22. Morillo S A, Tax F E (2006) Functional analysis of receptor-like kinases in monocots and dicots. Curr Opin Plant Biol 9:460-469.
23. Palumbo M C, Zenoni S, Fasoli M, Massonnet M, Farina L, Castiglione F, Pezzotti M, Paci P (2014) Integrated network analysis identifies fight-club nodes as a class of hubs encompassing key putative switch genes that induce major transcriptome reprogramming during grapevine development. Plant Cell 26:4617-4635.
24. Rodriguez M C, Petersen M, Mundy J (2010) Mitogen-activated protein kinase signaling in plants. Annu Rev Plant Biol 61:621-649.
25. Schmutz J, Cannon S B, Schlueter J, Ma J X, Mitros T, Nelson W, Hyten D L, Song Q J, Thelen J J, Cheng J L, Xu D, Hellsten U, May G D, Yu Y, Sakurai T, Umezawa T, Bhattacharyya M K, Sandhu D, Valliyodan B, Lindquist E, Peto M, Grant D, Shu S Q, Goodstein D, Barry K, Futrell-Griggs M, Abernathy B, Du J C, Tian Z X, Zhu L C, Gill N, Joshi T, Libault M, Sethuraman A, Zhang X C, Shinozaki K, Nguyen H T, Wing R A, Cregan P, Specht J, Grimwood J, Rokhsar D, Stacey G, Shoemaker R C, Jackson S A (2010) Genome sequence of the palacopolyploid soybean. Nature 463:178-183.
26. Shiu S H, Bleecker A B (2003) Expansion of the receptor-like kinase/Pelle gene family and receptor-like proteins in Arabidopsis. Plant Physiol 132:530-543.
27. Shiu S H, Karlowski W M, Pan R S, Tzeng Y H, Mayer K F X, Li W H (2004) Comparative analysis of the receptor-like kinase family in Arabidopsis and rice. Plant Cell 16:1220-1234.
28. Stuart J M, Segal E, Koller D, Kim S K (2003) A gene-coexpression network for global discovery of conserved genetic modules. Science 302:249-255.
29. Tena G, Boudsocq M, Sheen J (2011) Protein kinase signaling networks in plant innate immunity. Curr Opin Plant Biol 14:519-529.
30. Vidal M, Cusick M E, Barabasi A L (2011) Interactome networks and human disease. Cell 144:986-998.
31. Vij S, Giri J, Dansana P K, Kapoor S, Tyagi A K (2008) The receptor-like cytoplasmic kinase (OsRLCK) gene family in rice: Organization, phylogenetic relationship, and expression during development and stress. Mol Plant 1:732-750.
32. Vuong T D, Jiao Y, Shannon J G, Nguyen H T. 2013. Nematode resistance in soybean. In: Varshney R, Tuberosa R, eds. Translational genomics for crop breeding: Biotic stress. Wiley-Blackwell, 95-124.
33. Wei K F, Wang Y M, Xie D X (2014) Identification and expression profile analysis of the protein kinase gene superfamily in maize development. Mol Breeding 33:155-172.
34. Wolfe C J, Kohane I S, Butte A J (2005) Systematic survey reveals general applicability of “guilt-by-association” within gene coexpression networks. BMC Bioinformatics 6:227.

We claim:

1. A method for identifying a kinase hub gene that induces soybean cyst nematode (SCN) resistance when overexpressed, inactivated, or overexpressed and inactivated in a plant cell or a plant, the method comprising the steps of: (a) overexpressing a kinase or kinase-dead (inactivated) hub gene in a plant cell or a plant, compared to the expression in a plant cell or a plant known to be susceptible to SCN, or inactivating a kinase hub gene in a plant cell or a plant known to be resistant to SCN, (b) testing the SCN resistance in the plant cell or the plant comprising the overexpressed, inactivated, or the overexpressed and inactivated gene, and (c) identifying the gene that induces resistance in the plant cell or the plant when overexpressed, inactivated, or both overexpressed and inactivated.

2. A method of identifying one or more kinase genes that confer a trait to a plant when expressed, inactivated, or overexpressed an inactivated gene, the method comprising the steps of:
a) providing a kinase gene;
b) analyzing the mRNA expression profiles of the kinase gene during controlled conditions and under stressed conditions;
c) comparing the mRNA expression profiles of the kinase gene during the stress conditions to the kinase gene during controlled conditions to identify the kinase gene that are highly interconnected in control condition network compared to the stress condition network;
d) identifying the kinase hub genes based on co-expression with at least twenty-five genes specific to the stress network and specific to the controlled network;
e) comparing the interconnected kinase hub genes identified in step d) with differentially expressed genes in a plant cell;
f) identifying protein kinase hub genes that are differentially expressed in the plant cell to identify highly connected hub kinase genes;
g) creating kinase-dead variants of the highly connected hub kinase genes;
h) expressing or overexpressing a kinase-dead variants of the identified highly connected hub kinase genes or inactivating highly connected hub kinase genes in the plant cell; and
i) identifying one or more genes that confer a trait to the plant when the kinase-dead variant is expressed or overexpressed or the highly connected hub-kinase gene is inactivated.

3. A kinase-dead mutant comprising:
a) SEQ ID NOs: 92 or 94 to 99; or
b) any one of SEQ ID NOs: 1-91, said mutant comprising one or more amino acid mutations at a highly conserved or invariant amino acid in a domain selected from the ATP binding pocket of the kinase, the substrate binding pocket of the kinase of the kinase, the catalytic domain of the kinase, and combinations thereof.

4. An isolated nucleic acid sequence encoding a kinase dead mutant according to claim 3.

5. A vector comprising the nucleic acid of claim 3.

6. A plant cell or plant comprising a nucleic acid of claim 4 or a vector comprising said nucleic acid.

7. The plant cell or plant of claim 6, said plant cell or plant being a soybean plant cell or soybean plant.

8. The plant cell of claim 6, wherein the plant cell is in a plant part.

9. The plant cell of claim 8, wherein the plant part is a seed, endosperm, ovule or pollen.

10. The plant cell of claim 6, wherein the plant is a soybean plant or other cyst nematode-host plant.
